SEQUENCE LISTING (72 sequences)

SEQ ID NO: 1 - protein, 11 aa, synthetic construct
RASQSVGSNLA

SEQ ID NO: 2 - protein, 7 aa, synthetic construct
GASTRAT

SEQ ID NO: 3 - protein, 10 aa, synthetic construct
QQYNFYPPIT

SEQ ID NO: 4 - protein, 23 aa, synthetic construct
EIVLTQSPATLSVSPGERATLSC

SEQ ID NO: 5 - protein, 15 aa, synthetic construct
WYQQKPGQAPRLLIY

SEQ ID NO: 6 - protein, 32 aa, synthetic construct
GIPARFSGSGSGTEFTLTISSLQSEDFAVYYC

SEQ ID NO: 7 - protein, 10 aa, synthetic construct
FGGGTKVEIK

SEQ ID NO: 8 - protein, 108 aa, synthetic construct
EIVLTQSPATLSVSPGERATLSCRASQSVGSNLAWYQQKPGQAPRLLIYGASTRATGIPARFSGSGSGTEFTLTISSLQSEDFAVYYCQQYNFYPPITFGGGTKVEIK

SEQ ID NO: 9 - protein, 215 aa, synthetic construct
EIVLTQSPATLSVSPGERATLSCRASQSVGSNLAWYQQKPGQAPRLLIYGASTRATGIPARFSGSGSGTEFTLTISSLQSEDFAVYYCQQYNFYPPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 324 bp, synthetic construct
  source 1..324: mol_type = other DNA
gaaatagtgttgacgcagtctccagccaccctgtctgtgtctccaggggaaagagccaccctctcctgcagggccagtcagagtgttggcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtacaacttctaccctcctatcacttttggcggagggaccaaggttgagatcaaa

SEQ ID NO: 11 - protein, 12 aa, synthetic construct
RASQSVSSSFLA

SEQ ID NO: 12 - protein, 7 aa, synthetic construct
GASSRAT

SEQ ID NO: 13 - protein, 9 aa, synthetic construct
QQYHSSPFT

SEQ ID NO: 14 - protein, 23 aa, synthetic construct
EIVLTQSPGTLSLSPGERATLSC

SEQ ID NO: 15 - protein, 32 aa, synthetic construct
GIPDRFSGSGSGTDFTLTISRLEPEDFAVYYC

SEQ ID NO: 16 - protein, 108 aa, synthetic construct
EIVLTQSPGTLSLSPGERATLSCRASQSVSSSFLAWYQQKPGQAPRLLIYGASSRATGIPDRFSGSGSGTDFTLTISRLEPEDFAVYYCQQYHSSPFTFGGGTKVEIK

SEQ ID NO: 17 - protein, 215 aa, synthetic construct
EIVLTQSPGTLSLSPGERATLSCRASQSVSSSFLAWYQQKPGQAPRLLIYGASSRATGIPDRFSGSGSGTDFTLTISRLEPEDFAVYYCQQYHSSPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagcttcttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtaccacagctcccctttcacttttggcggagggaccaaggttgagatcaaa

SEQ ID NO: 19 - protein, 107 aa, synthetic construct [sequence omitted]

SEQ ID NO: 20 - protein, 11 aa, synthetic construct
GSISSSSYYWG

SEQ ID NO: 21 - protein, 16 aa, synthetic construct
SISYSGSTYYNPSLKS

SEQ ID NO: 22 - protein, 12 aa, synthetic construct
AREGYDSYSYDV

SEQ ID NO: 23 - protein, 26 aa, synthetic construct
QLQLQESGPGLVKPSETLSLTCTVSG

SEQ ID NO: 24 - protein, 14 aa, synthetic construct
WIRQPPGKGLEWIG

SEQ ID NO: 25 - protein, 30 aa, synthetic construct
RVTISVDTSKNQFSLKLSSVTAADTAVYYC

SEQ ID NO: 26 - protein, 11 aa, synthetic construct
WGQGTMVTVSS

SEQ ID NO: 27 - protein, 120 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCAREGYDSYSYDVWGQGTMVTVSS

SEQ ID NO: 28 - protein, 450 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCAREGYDSYSYDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 360 bp, synthetic construct
  source 1..360: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagagggatacgacagctacagctatgacgtatggggtcagggtacaatggtcaccgtctcctca

SEQ ID NO: 30 - protein, 11 aa, synthetic construct
GSISSQQYYWG

SEQ ID NO: 31 - protein, 16 aa, synthetic construct
SISYAASTYYNPSLKS

SEQ ID NO: 32 - protein, 120 aa, synthetic construct
QLQLQESGPGLVKPSETLSLTCTVSGGSISSQQYYWGWIRQPPGKGLEWIGSISYAASTYYNPSLKSRVTISVDTSKNQFSLKLSSVTAADTAVYYCAREGYDSYSYDVWGQGTMVTVSS

SEQ ID NO: 33 - protein, 450 aa, synthetic construct
QLQLQESGPGLVKPSETLSLTCTVSGGSISSQQYYWGWIRQPPGKGLEWIGSISYAASTYYNPSLKSRVTISVDTSKNQFSLKLSSVTAADTAVYYCAREGYDSYSYDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 360 bp, synthetic construct
  source 1..360: mol_type = other DNA
cagctgcagctgcaggagtcgggcccaggactggtgaagccttcggagaccctgtccctcacctgcactgtctctggtggctccatcagcagtcaacaatactactggggctggatccgccagcccccagggaaggggctggagtggattgggagtatctcctatgccgcaagcacctactacaacccgtccctcaagagtcgagtcaccatatccgtagacacgtccaagaaccagttctccctgaagctgagttctgtgaccgccgcagacacggcggtgtactactgcgccagagagggatacgacagctacagctatgacgtatggggtcagggtacaatggtcaccgtctcctca

SEQ ID NO: 35 - protein, 11 aa, synthetic construct
GSISSYDPYWG

SEQ ID NO: 36 - protein, 16 aa, synthetic construct
SISYSGSTLYNPSLKS

SEQ ID NO: 37 - protein, 16 aa, synthetic construct
AREVSYLGSRRYGMDV

SEQ ID NO: 38 - protein, 11 aa, synthetic construct
WGQGTTVTVSS

SEQ ID NO: 39 - protein, 124 aa, synthetic construct
QLQLQESGPGLVKPSETLSLTCTVSGGSISSYDPYWGWIRQPPGKGLEWIGSISYSGSTLYNPSLKSRVTISVDTSKNQFSLKLSSVTAADTAVYYCAREVSYLGSRRYGMDVWGQGTTVTVSS

SEQ ID NO: 40 - protein, 454 aa, synthetic construct
QLQLQESGPGLVKPSETLSLTCTVSGGSISSYDPYWGWIRQPPGKGLEWIGSISYSGSTLYNPSLKSRVTISVDTSKNQFSLKLSSVTAADTAVYYCAREVSYLGSRRYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 372 bp, synthetic construct
  source 1..372: mol_type = other DNA
cagctgcagctgcaggagtcgggcccaggactggtgaagccttcggagaccctgtccctcacctgcactgtctctggtggctccatcagcagttacgacccctactggggctggatccgccagcccccagggaaggggctggagtggattgggagtatctcctatagtgggagcaccctctacaacccgtccctcaagagtcgagtcaccatatccgtagacacgtccaagaaccagttctccctgaagctgagttctgtgaccgccgcagacacggcggtgtactactgcgccagagaggtgagctatctaggttcacgtagatacggaatggacgtatggggccagggaacaactgtcaccgtctcctca

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 21 aa, Homo sapiens
CPPCPAPELLGGPSVFLFPPK

SEQ ID NO: 44 - protein, 21 aa, synthetic construct
CPPCPAPELAGAPSVFLFPPK

SEQ ID NO: 45 - protein, 21 aa, synthetic construct
CPPCPAPEFEGGPSVFLFPPK

SEQ ID NO: 46 - protein, 21 aa, synthetic construct
CPPCPAPEAAGGPSVFLFPPK

SEQ ID NO: 47 - protein, 21 aa, synthetic construct
CPPCPAPEAAGAPSVFLFPPK

SEQ ID NO: 48 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 49 - protein, 20 aa, Homo sapiens
MPLLWLRGFLLASCWIIVRS

SEQ ID NO: 50 - protein, 116 aa, Homo sapiens [sequence omitted]

SEQ ID NO: 51 - DNA, 6105 bp, Homo sapiens
  source 1..6105: mol_type = genomic DNA
agtacagtataaaacttcacagtgccaataccatgaagaggagctcagacagctcttaccacatgatacaagagccggctggtggaagagtggggaccagaaagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaaaaaaaaacaaaaaaaaacaaaagtaaattaaaaacaaaacctgatgaaacagatgaaggaagatgtggaaaaaatccttagccagggctcagagatgaagcagtgaaagagacaggaattgggagggaaagggagaatggtgtaccctttatttcttctgaaatcacactgatgacatcagttgtttaaacggggtattgtcctttccccccttgaggttcccttgtgagccttgaatcaaccaatctagtctgcagtagtgtggactagaacaacccaaatagcatctagaaagccatgagtttgaaagggcccatcacaggcactttcctacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcctttttctgtaccacttttgcaacaaaacaaaacaaacaacattaaaaaattgagaacaagtatggaaagaatgaaagatcaaggaaaaaagaataccaagttacatttcgttaaggtgcttatgatcttagaactatgcaacctaataggtttgaaactgtttacctgagagagaacaaaaagagagacttttttgtattggaagtaatctgattaatttttattttcttcaaggagagatacttgaaaggaatatgtttgtccatctgttggatccaaacatttctatattttgtaaatgttgttgttgttttttttttaatcgtttactatttgcactacaatggtgtttgacctgtctaatccttatttaacaagtattttctttggttgggggtgggggtggggtttaagagctgcacttaatgtgagctataaaagaactgctacagcacacaaaatagctatttttattattataattataattattattattattttgtaccttaaaaaatagacacatacaccaaagacatttgtgtgagcctttaaacagtctgtctgtggttggtatcattcaccatcaatgagtcaggggttgggattcaaggttgagtagtgtggattgtgttcaggcttaaaagacctgagaagtttggtttttgactccttttacatccatgaaacaggacatttcatactggatgtacagtagttgtacactgttggatatcaagttcaatcaaattcatggaactacatgcttgtatgtgtatatatacattgcttgtgcatatgcatatctgtatgtatatatacatgtattgtaccatgtccatacacattttaagcacttcaggctgtcattttttaatgttcttaaagcaatgaatgtttgtgtgcaaaacacagtatttttaagaaggataggctatagtttttgcttttactctgaactaggtgggcgcatttcaaaaattcggatgggaaaaagcctggaaattccagtgaatattcagcaaggccctctttcattgtacagggatcaaatttcctcctcttttttgtgccccctcccacttctacaagttatcccctgtggggaaaacaggatgataatcaaaactctgggctgatgtttttccaacttagtgtctattggaatcaatcttaaatcagaagctttttcagaaaaataatatttaggccagaattagagttgagtgtattttttaaaaatgattaaggcttggttgtgagaaatattacctgtaccagctgggaaaaataatgtcatcactaactaaaagataattaatttgagagaaagtgttaagagagggagagtaaggaagagaacagttaagaggaggcagaggtgagggcagtagtaaaaatctctaaaattttaatttacagccaaaattcttcatgtgtaaatttgtattgattcagatgcagaaatgaaaaaaaaacacctttgttttataaatatcaaagtacatgcttaaagccaagtttttatctagtttattctagtacttagcttgcctggaatagctaataaattattcatgtatgtgcttttgaaaatccagagccctatttttacacacttgtgtgaagttggcaaacattttgaaaaatggaaaaaagtttctaataattgggaacaattacattaattaatattttgtaaaatattgaagcttttagccctatgtcaatttgtagattaaaataaattaattataggaaaggaagataacagtgagaaaccaaacattacaaaaggtggtttagctctccttgaaaaatatactaagttggtatactataacacttggctatatgtaggcaatgtcactactgggcaaatacacttactgtgttctagaggcagccctttcttatgcagaaaatacaatacgcactgcatgagaagcttgagagtggattctaatccaggtctgtcgaccttggatatcatgcatgtgggaaggtgggtgtggtgagaaaagttttaaggcaagagtagatggccatgttcaactttacaaaatttcttggaaaactggcagtattttgaactgcatcttctttggtaccggaacctgcagaaacagtgtgagaaattaagtcctggttcactgcgcagtagcaaagatggtcaaggccatggaaaaagcagaaatttaccaagaaagctgatacccatgtatagttcccactcatctcaaatacatctgctatctttttaagctaagtcctagacatatcggggataacatgggggttgattagtgaccacagttatcagaagcagagaaatgtaattccatattttatttgaaacttattccatattttaattggatattgagtgattgggttatcaaacacccacaaactttaattttgttaaatttatatggctttgaaatagaagtataagttgctaccattttttgataacattgaaagatagtattttaccatctttaatcatcttggaaaatacaagtcctgtgaacaaccactctttcacctagcagcatgaggccaaaagtaaaggctttaaattataacatatgggattcttagtagtatgtttttttcttgaaactcagtggctctatctaaccttactatctcctcactctttctctaagactaaactctaggctcttaaaaatctgcccacaccaatcttagaagctctgaaaagaatttgtctttaaatatcttttaatagtaacatgtattttatggaccaaattgacattttcgactattttttccaaaaaagtcaggtgaatttcagcacactgagttgggaatttcttatcccagaagaccaaccaatttcatatttatttaagattgattccatactccgttttcaaggagaatccctgcagtctccttaaaggtagaacaaatactttctatttttttttcaccattgtgggattggactttaagaggtgactctaaaaaaacagagaacaaatatgtctcagttgtattaagcacggacccatattatcatattcacttaaaaaaatgatttcctgtgcaccttttggcaacttctcttttcaatgtagggaaaaacttagtcaccctgaaaacccacaaaataaataaaacttgtagatgtgggcagaaggtttgggggtggacattgtatgtgtttaaattaaaccctgtatcactgagaagctgttgtatgggtcagagaaaatgaatgcttagaagctgttcacatcttcaagagcagaagcaaaccacatgtctcagctatattattatttattttttatgcataaagtgaatcatttcttctgtattaatttccaaagggttttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttgttttgcatactacatgcagttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggccttatgttaaataaaaagacctgtttgggatgtaaaaaaaaaaaaaaaaaaaaaaaaaaaaaaaaaaaaaaaaaa

SEQ ID NO: 52 - protein, 407 aa, Homo sapiens
MDGLPGRALGAACLLLLAAGWLGPEAWGSPTPPPTPAAPPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 53 - protein, 292 aa, Homo sapiens
MDGLPGRALGAACLLLLAAGWLGPEAWGSPTPPPTPAAPPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKR

SEQ ID NO: 54 - protein, 115 aa, Homo sapiens
GLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 55 - DNA, 3206 bp, Homo sapiens
  source 1..3206: mol_type = genomic DNA
cggctcgactcggctcgcctcgcggcgggcgccctcgtcgccagcggcgcaccatggacgggctgcccggtcgggcgctgggggccgcctgccttctgctgctggcggccggctggctggggcctgaggcctggggctcacccacgcccccgccgacgcctgccgcgccgccgccacccccgccacccggatccccgggtggctcgcaggacacctgtacgtcgtgcggcggcttccggcggccagaggagctcggccgagtggacggcgacttcctggaggcggtgaagcggcacatcttgagccgcctgcagatgcggggccggcccaacatcacgcacgccgtgcctaaggccgccatggtcacggccctgcgcaagctgcacgcgggcaaggtgcgcgaggacggccgcgtggagatcccgcacctcgacggccacgccagcccgggcgccgacggccaggagcgcgtttccgaaatcatcagcttcgccgagacagatggcctcgcctcctcccgggtccgcctatacttcttcatctccaacgaaggcaaccagaacctgtttgtggtccaggccagcctgtggctttacctgaaactcctgccctacgtcctggagaagggcagccggcggaaggtgcgggtcaaagtgtacttccaggagcagggccacggtgacaggtggaacatggtggagaagagggtggacctcaagcgcagcggctggcataccttcccactcacggaggccatccaggccttgtttgagcggggcgagcggcgactcaacctagacgtgcagtgtgacagctgccaggagctggccgtggtgccggtgttcgtggacccaggcgaagagtcgcaccggccctttgtggtggtgcaggctcggctgggcgacagcaggcaccgcattcgcaagcgaggcctggagtgcgatggccggaccaacctctgttgcaggcaacagttcttcattgacttccgcctcatcggctggaacgactggatcatagcacccaccggctactacgggaactactgtgagggcagctgcccagcctacctggcaggggtccccggctctgcctcctccttccacacggctgtggtgaaccagtaccgcatgcggggtctgaaccccggcacggtgaactcctgctgcattcccaccaagctgagcaccatgtccatgctgtacttcgatgatgagtacaacatcgtcaagcgggacgtgcccaacatgattgtggaggagtgcggctgcgcctgacagtgcaaggcaggggcacggtggtggggcacggagggcagtcccgggtgggcttcttccagcccccgcgggaacgggggtacacggtgggctgagtacagtcattctgttgggctgtggagatagtgccagggtgcggcctgagatatttttctacagcttcatagagcaaccagtcaaaaccagagcgagaaccctcaactgacatgaaatactttaaaatgcacacgtagccacgcacagccagacgcatcctgccacccacacagcagcctccaggataccagcaaatggatgcggtgacaaatggcagcttagctacaaatgcctgtcagtcggagagaatggggtgagcagccaccattcccaccagctggcccggccactctgaattgcgccttccgagcacacataaaagcacaaagacagagacgcagagagagagagagagccacggagaggaaaagcagatgcaggggtggggagcgcagctcggcggaggctgcgtgtgccccgtggcttttaccaggcctgctctgcctggctcgatgtctgcttcttccccagcctgggatccttcgtgcttcaaggcctggggagcctgtccttccatgcccttgtcgagggaaagagacccagaaaggacacaacccgtcagagacctgggagcaggggcaatgaccgtttgactgtttgtggcttgggcctctgacatgacttatgtgtgtgtgtgtttttggggtggggagggagggagagaagagggggctaaatttgatgctttaactgatctccaacagttgacaggtcatccttgccagttgtataactgaaaaaggacttttctaccaggtatgaccttttaagtgaaaatctgaattgttctaaatggaaagaaaaaaagttgcaatctgtgcccttcattggggacattcctctaggactggtttggggacgggtgggaatgacccctaggcaaggggatgagaccgcaggaggaaatggcggggaggaggcattcttgaactgctgaggatggggggtgtcccctcagcggaggccaagggaggggagcagcctagttggtcttggagagatggggaaggctttcagctgatttgcagaagttgcccatgtgggccccagccatcagggctggccgtggacgtggcccctgcccactcacctgcccgcctgcccgcccgcccgcatagcacttgcagacctgcctgaacgcacatgacatagcacttgccgatctgcgtgtgtccagaagtggcccttggccgagcgccgaactcgctcgccctctagatgtccaagtgccacgtgaactatgcaatttaaagggttgacccacactagacgaaactggactcgtacgactctttttatattttttatacttgaaatgaaatcctttgcttcttttttaagcgaatgattgcttttaatgtttgcactgatttagttgcatgattagtcagaaactgccatttgaaaaaaagttatttttatagcagcaaaaaaaaaaaaaaaagaatacagttaaatgtattatacataattttggaaccaaagaggccaacagatcagttttaattttattagacggtgaggccatctgagatgaggtggacgttctgagcagtcccttgagtggcctgccaacgtttcagggtatgaatggattttgtttattcggtttgatgtgtcttttccatccttacacacccagaaggtagagtaaaaatgactatgatagaatgcaggtgtgtatccttaaatcctcatctttatgtttatttaataaagctccccttagattctgtttcataataatttaaaaccaaacaattttcccatagacttgctgttaaagtattgtacgtttgtgtacagtttaagaaaataaaagattgagtgcca

SEQ ID NO: 56 - protein, 426 aa, Macaca mulatta
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTTRKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGDGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 57 - protein, 424 aa, Canis lupus
MPLLWLRGFLVASCWIIVRSSPTPGPEGPGAAPACPACALTALPRDAPNSQPEMVEAVKKHILNMLHLKKRPEVTQPVPKAALLNAIRKLHVGKVGENGFVEIEDDIGRRAEMNELMEQTSEIITFAESGTARKTLHFEISKEGSDLSVVERAEVWLFLKVPKANRTRTKVTIRLLQKHPQGSLDAGEEAEDMGFPEERNEVLISEKVVDARKSTWHIFPVSSSIQRLLDQGRSSLDVRIACEQCHETGASLVLLGKKKKKEEEGEGKKKDGGDAGAGGDEDKEQSHRPFLMLQARQSEDHPHRRRRRGLECDGKVNICCKKQFFVSFKDIGWNDWIIAPSGYHANYCEGGCPSHIAGTSGSSLSFHSTVINHYRLRGHSPFTNLKSCCVPTKLRPMSMLYYDDGQNIIKKDIQNMIVEECGCS

SEQ ID NO: 58 - protein, 424 aa, Felis catus
MPLLWLRGFLLASCWIIVRSSPTPGSEGPGAAPDCPSCALATLPKDVPNSQPEMVEAVKKHILNMLHLKKRPEVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIQLLQKQPQGGVDAGEEAEEMGLMEERNEVLISEKVVDARKSTWHIFPVSSSIQRLLDQGKSSLDVRIACEQCHETGASLVLLGKKKKKEEEGEGKKKDGGDGGAGADEDKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 59 - protein, 106 aa, Homo sapiens [sequence omitted]

SEQ ID NO: 60 - protein, 407 aa, Macaca mulatta
MDGLPGRALGAACLLLLAAGWLGPEAWGSPTPPPSPAAPPPPPPPGAPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGAVNSCCIPTKLSTMSMLYFDDEYNIVKRDVPNMIVEECGCA

SEQ ID NO: 61 - protein, 292 aa, Macaca mulatta
MDGLPGRALGAACLLLLAAGWLGPEAWGSPTPPPSPAAPPPPPPPGAPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKR

SEQ ID NO: 62 - protein, 115 aa, Macaca mulatta
GLECDGRTNLCCRQQFFIDFRLIGWNDWIIAPTGYYGNYCEGSCPAYLAGVPGSASSFHTAVVNQYRMRGLNPGAVNSCCIPTKLSTMSMLYFDDEYNIVKRDVPNMIVEECGCA

SEQ ID NO: 63 - protein, 407 aa, Canis lupus
MDGLPGRALGAACLLLLAAGWLGPEAWGSPTPPPSPAAPPPPPPPGAPGGSQDTCTSCGGFRRPEELGRVDGDFLEAVKRHILSRLQMRGRPNITHAVPKAAMVTALRKLHAGKVREDGRVEIPHLDGHASPGSDGQERVSEIISFAETDGLASSRVRLYFFVSNEGNQNLFVVQASLWLYLKLLPYVLEKGSRRKVRVKVYFQEQGQGDRWNAVEKKVDLKRSGWHTFPLTEAIQALFERGERRLSLDVQCDGCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 64 - protein, 292 aa, Canis lupus
MDGLPGRALGAACLLLLAAGWLGPEAWGSPTPPPSPAAPPPPPPPGAPGGSQDTCTSCGGFRRPEELGRVDGDFLEAVKRHILSRLQMRGRPNITHAVPKAAMVTALRKLHAGKVREDGRVEIPHLDGHASPGSDGQERVSEIISFAETDGLASSRVRLYFFVSNEGNQNLFVVQASLWLYLKLLPYVLEKGSRRKVRVKVYFQEQGQGDRWNAVEKKVDLKRSGWHTFPLTEAIQALFERGERRLSLDVQCDGCQELAVVPVFVDPGEESHRPFVVVQARLGDSRHRIRKR

SEQ ID NO: 65 - protein, 115 aa, Canis lupus; identical to SEQ ID NO: 54

SEQ ID NO: 66 - protein, 407 aa, Felis catus
MDGLPGRALGAACLLLLAAGWLGPEAWGSPTPPPSPAAPPPPPPPGAPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNVVEKKVDLKRSGWHTFPLTEAIQSLFERGERRLNLDVQCDGCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 67 - protein, 292 aa, Felis catus
MDGLPGRALGAACLLLLAAGWLGPEAWGSPTPPPSPAAPPPPPPPGAPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNVVEKKVDLKRSGWHTFPLTEAIQSLFERGERRLNLDVQCDGCQELAVVPVFVDPGEESHRPFVVVQARLGDSRHRIRKR

SEQ ID NO: 68 - protein, 115 aa, Felis catus; identical to SEQ ID NO: 54

SEQ ID NO: 69 - protein, 4 aa, synthetic construct
LAGA

SEQ ID NO: 70 - protein, 4 aa, synthetic construct
FEGG

SEQ ID NO: 71 - protein, 4 aa, synthetic construct
AAGG

SEQ ID NO: 72 - protein, 4 aa, synthetic construct
AAGA

(19) United States
(12) Patent Application Publication
(10) Pub. No.: US 2026/0285962 A1
(43) Pub. Date: Sep. 24, 2026

(54) ANTI-ACTIVIN A/B ANTIBODIES AND USES THEREOF

(71) Applicants: BYOMass Inc., Sudbury, MA (US); Adimab, LLC, Lebanon, NH (US)

(72) Inventors: Vivienne Margaret Jackson, Sudbury, MA (US); Nels P. Nielson, Lebanon, NH (US)

(21) Appl. No.: 19/130,496

(22) PCT Filed: Nov. 17, 2023
(86) PCT No.: PCT/US23/80341

§ 371 Date: May 15, 2025

Related U.S. Application Data
(60) Provisional application No. 63/426,477, filed on Nov. 18, 2022.

(51) Int. Cl. C07K 16/22; A61K 39/00; A61P 1/16; A61P 35/04

(52) U.S. Cl. CPC C07K 16/22; A61P 1/16; A61P 35/04; A61K 2039/505; C07K 2317/14; C07K 2317/52; C07K 2317/565; C07K 2317/569; C07K 2317/622; C07K 2317/76; C07K 2317/92

(57) ABSTRACT

Provided herein are antibody agents that bind specifically to Activin-A and Activin-B, as well as compositions comprising Activin A/B antibody agents, and methods of making and using the same.

### CROSS-REFERENCE TO RELATED APPLICATIONS

This application claims priority to U.S. Provisional Patent Application 63/426,477 filed on Nov. 18, 2022, the entire contents of which is hereby incorporated by reference.

### BACKGROUND

Activin-A and Activin-B are members of the transforming growth factor beta (TGFb) superfamily. Activin-A and Activin-B can be expressed in a variety of cells and the expression of Activin-A and Activin-B can be upregulated in response to stimuli such as inflammation.

### SUMMARY

The present disclosure provides new, high-affinity Activin A/B antibody agents. Among other things, provided Activin A/B antibody agents can be used, e.g., to bind to Activin-A, Activin-B, and/or to one or more complexes that include one or both Activin-A and Activin-B, and/or to reduce an activity and/or level of Activin-A and/or Activin-B in a relevant system (e.g., in vitro, in a cell, in a tissue, in a sample from a subject and/or in a subject).

For example, in some embodiments, the present disclosure provides novel Activin A/B antibody agents which have desirable binding kinetics, binding affinity, pharmacokinetics and/or function. In some embodiments, Activin A/B antibody agents provided herein have improved characteristics compared to a reference antibody agent, e.g., as described herein. In some embodiments, a reference antibody agent comprises an Activin-A antibody and/or an Activin-B antibody.

Without wishing to be bound by any particular theory, in some embodiments, novel Activin A/B antibody agents provided herein can bind to Activin-A, Activin-B, and/or to one or more complexes that include one or both Activin-A and Activin-B, and inhibit binding of Activin-A, Activin-B, and/or one or more complexes comprising the same to an Activin receptor (e.g., as described herein). In some embodiments, inhibiting binding of Activin-A, Activin-B, and/or one or more complexes comprising the same to an Activin receptor inhibits canonical signaling from and Activin receptor, e.g., SMAD2/3 signaling or non-canocial signaling. In some embodiments, inhibition of SMAD2/3 signaling can prevent fibrosis and/or organ damage.

In some embodiments, an Activin A/B antibody agent disclosed herein binds to Activin-A with high specificity. In some embodiments, an Activin A/B antibody agent disclosed herein binds to Activin-B with high specificity.

In some embodiments, an Activin A/B antibody agent disclosed herein binds to its target with high specificity. In some embodiments, a target of an Activin A/B antibody agent is Activin A and/or Activin B.

In some embodiments, an Activin A/B antibody agent binds to Activin-A with a K_{D} of about 83 pM to about 1700 pM (e.g., about 83.2 pM to about 1640 pM), e.g., as assessed in an assay that measures antibody binding affinity, e.g., a Surface Plasmon Resonance assay (Biacore). In some embodiments, an Activin A/B antibody agent binds to Activin-B with a K_{D} of about 760 pM to about 900 pM (e.g., about 760 pM to about 861 pM), e.g., as assessed in an assay that measures antibody binding affinity, e.g., an Octet assay.

In some embodiments, a provided Activin A/B antibody agent may show preferential binding to Activin-A and/or Activin-B relative to one or more TGFbeta family members other than Activin-A or Activin-B. In some such embodiments, preferential binding may be assessed, for example, by simultaneously contacting an Activin A/B antibody agent with Activin A, Activin-B, and one or more other TGFbeta family members. Alternatively or additionally, in some embodiments, preferential binding may be assessed relative to an appropriate reference Activin A/B antibody agent and, e.g., may reflect a higher level of binding to Activin-A and/or Activin-B relative to the one or more other TGFbeta family member than is observed with the reference antibody.

In some embodiments, an Activin A/B antibody agent disclosed herein inhibits an activity of Activin-A and/or Activin-B and/or reduces a level of Activin-A and/or Activin-B (e.g., a level of free and/or active Activin-A and/or Activin-B, e.g., in a blood, plasma, serum, tumor, and/or urine sample) when administered to a cell, tissue or subject. In some embodiments, an Activin A/B antibody agent can be used (e.g., alone or in combination with one or more additional agents [e.g., as described herein]) to prevent, reduce, treat and/or reverse any one, all, or a combination of: mass loss (e.g., muscle mass loss, lean mass loss, fat mass loss, organ mass loss, and/or bone mass loss), weight loss, senescence, liver damage, kidney damage, heart damage, pancreatic damage, or cancer, cancer metastases, chemotherapy-induced toxicity (e.g. nephrotoxicity), chemoresistance and/or hypertension.

In some embodiments, an Activin A/B antibody agent disclosed herein can be used to prevent, reduce, treat and/or reverse a condition or disease associated with increased Activin-A and/or Activin-B, e.g., any one or all or a combination of: (i) disorders of the hypothalamic pituitary gonadal axis (e.g., with increased FSH levels); (ii) fibrodysplasia ossificans progressive (FOP); (iii) pulmonary arterial hypertension (PAH) and/or hypertension (e.g., preeclampsia); (iv) Anorexia-Cachexia Disorders associated with chronic diseases such as cancer, chronic kidney disease, chronic heart failure, COPD, elderly and muscle immobility, HIV patients, ICU patients (e.g., loss of body weight, loss of food intake, fat mass loss, organ mass loss, muscle wasting (e.g., muscle atrophy), loss of functional muscle mass and strength, bone loss, anemia, fatigue, and/or organ damage); (v) anemia; (vi) metabolic disorders (e.g., obesity, type 2 diabetes, metabolic syndrome and/or type 1 diabetes, lipodystrophy); (vii) inflammatory disorders (e.g. inflammatory bowel disease, pancreatitis, allergic asthma, COVID, acute respiratory distress syndrome, atopic dermatitis, preeclampsia, acute liver injury, acute kidney injury, Tubulointerstitial nephritis, osteoarthritis and inflammatory-induced anemia); (viii) auto-immune disorders (e.g., SLE or rheumatoid arthritis); (ix) fibrotic diseases (e.g. idiopathic pulmonary fibrosis (IPF), NASH, liver cirrhosis, chronic kidney disease, viral hepatitis, biliary disorders, Alport Syndrome, Uterine Fibroids, Endometriosis); (x) cancer; (xi) cancer-treatment related toxicities (e.g., chemotherapy-induced nephrotoxicity, hepatotoxicity); (xii) cancer related metastases (e.g., changes in cell adhesion, migration and/or invasion); (xiii) aging (e.g., senescence); (xiv) Myalgic Encephalomyelitis or Chronic Fatigue Syndrome; (xv) reproductive disorders (e.g., with elevated FSH, hypogonadotropic hypothalamic amenorrhea, polycystic ovary syndrome (PCOS), menopause, ovarian aging and/or ovarian cancers); (xvi) chemoresistance; or (xvii) heart failure. Among other things, this disclosure provides compositions comprising new and improved Activin A/B antibody agents, as well as methods of making and using the same.

In some embodiments, a condition or disease associated with increased Activin-A and/or Activin-B which can be prevented and/or treated with an Activin A/B antibody agent disclosed herein is or comprises preeclampsia.

In some embodiments, a condition or disease associated with increased Activin-A and/or Activin-B which can be prevented and/or treated with an Activin A/B antibody agent disclosed herein is or comprises weight loss (e.g., cachexia, lipodystrophy).

In some embodiments, a condition or disease associated with increased Activin-A and/or Activin-B which can be prevented and/or treated with an Activin A/B antibody agent disclosed herein is or comprises organ damage (e.g., multi-organ damage). In some embodiments, organ damage comprises: a change in function of an organ, a reduction in size of an organ (e.g., atrophy), a change in cellularity of an organ (e.g., increased infiltration of immune cells), a reduction in weight of an organ, or any combination thereof. In some embodiments, organ damage comprises damage to one or more, or all of: a liver, kidney, heart, adipose or pancreas.

In some embodiments, a condition or disease associated with increased Activin-A and/or Activin-B which can be prevented, reversed, and/or treated with an Activin A/B antibody agent disclosed herein comprises liver damage. In some embodiments, liver damage comprises a change in function of a liver, a reduction in size of a liver (e.g., atrophy), a change in cellularity of a liver (e.g., increased infiltration of immune cells), or a reduction in weight of a liver, or any combination thereof. In some embodiments, liver damage comprises fibrosis. In some embodiments, liver damage comprises necrosis. In some embodiments, liver damage comprises degeneration. In some embodiments, liver damage comprises hepatitis.

In some embodiments, a condition or disease associated with increased Activin-A and/or Activin-B which can be prevented, reversed, and/or treated with an Activin A/B antibody agent disclosed herein comprises kidney damage. In some embodiments, kidney damage comprises a change in function of a kidney, a reduction in size of a kidney (e.g., atrophy), a change in cellularity of a kidney (e.g., increased infiltration of immune cells), or a reduction in weight of a kidney, or any combination thereof. In some embodiments, kidney damage comprises fibrosis. In some embodiments, kidney damage comprises tubular degeneration.

In some embodiments, a condition or disease associated with increased Activin-A and/or Activin-B which can be prevented, reversed, and/or treated with an Activin A/B antibody agent disclosed herein comprises damage to a heart. In some embodiments, heart damage comprises a change in function of a heart, a reduction in size of a heart (e.g., atrophy), a change in cellularity of a heart (e.g., increased infiltration of immune cells), or a reduction in weight of a heart, or any combination thereof. In some embodiments, heart damage comprises fibrosis.

In some embodiments, a condition or disease associated with increased Activin-A and/or Activin-B which can be prevented, reversed, and/or treated with an Activin A/B antibody agent disclosed herein comprises pancreatic damage. In some embodiments, pancreatic damage comprises a change in function of a pancreas, a reduction in size of a pancreas (e.g., atrophy), a change in cellularity of a pancreas (e.g., increased infiltration of immune cells), or a reduction in weight of a pancreas, or any combination thereof. In some embodiments, pancreatic damage comprises pancreatitis.

In some embodiments, an Activin A/B antibody agent disclosed herein can be used to prevent, reduce, treat and/or reverse an effect (e.g., an unwanted side effect) associated with (e.g., caused by) an additional agent (e.g., an additional therapeutic agent disclosed herein). In some embodiments, an Activin A/B antibody agent disclosed herein can be used to prevent, reduce, treat and/or reverse a side effect associated with an additional agent, e.g., that results in weight loss; or which is used to treat a metabolic disorder (e.g., diabetes). In some embodiments, a side effect is a loss of muscle mass. In some embodiments, a side effect is heart damage. In some embodiments, a side effect is liver damage. In some embodiments, a side effect is kidney damage. In some embodiments, an additional agent is chosen from: a Glucagon-like peptide-1 (GLP-1) receptor modulator (e.g., an agonist or an antagonist); a glucose-dependent insulinotropic polypeptide (GIP) receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of peptide YY (PYY), an agent that increases a level and/or activity of GDF15, an agent that increases a level and/or activity of GFRAL, or any combination thereof.

Accordingly, disclosed herein are methods of preventing, reducing, treating and/or reversing an effect (e.g., an unwanted side effect) associated with (e.g., caused by) an additional agent (e.g., an additional therapeutic agent disclosed herein) by administering an Activin A/B antibody agent disclosed herein to a subject who has received, is receiving or will receive an additional agent. In some embodiments, an additional agent comprises: a Glucagon-like peptide-1 (GLP-1) receptor modulator (e.g., an agonist or an antagonist); a glucose-dependent insulinotropic polypeptide (GIP) receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of PYY, an agent that increases a level and/or activity of GDF15, an agent that increases a level and/or activity of GFRAL, or any combination thereof.

In some embodiments, provided herein is a method for preventing, reducing, treating and/or reversing muscle mass loss, e.g., resulting from an additional agent, by administering to a subject an Activin A/B antibody agent disclosed herein. In some embodiments, a subject has received or is receiving an additional agent. In some embodiments, an Activin A/B antibody agent disclosed herein is administered concurrently with an additional agent. In some embodiments, an Activin A/B antibody agent disclosed herein is administered prior to administration of an additional agent. In some embodiments, an Activin A/B antibody agent disclosed herein is administered after administration of an additional agent. In some embodiments, an additional agent comprises: a Glucagon-like peptide-1 (GLP-1) receptor modulator (e.g., an agonist or an antagonist); a glucose-dependent insulinotropic polypeptide (GIP) receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of PYY, an agent that increases a level and/or activity of GDF15, an agent that increases a level and/or activity of GFRAL, or any combination thereof.

In some embodiments, provided herein is a method for preventing, reducing, treating and/or reversing heart damage, e.g., resulting from an additional agent, by administering to a subject an Activin A/B antibody agent disclosed herein. In some embodiments, a subject has received or is receiving an additional agent. In some embodiments, an Activin A/B antibody agent disclosed herein is administered concurrently with an additional agent. In some embodiments, an Activin A/B antibody agent disclosed herein is administered prior to administration of an additional agent. In some embodiments, an Activin A/B antibody agent disclosed herein is administered after administration of an additional agent. In some embodiments, an additional agent comprises: a Glucagon-like peptide-1 (GLP-1) receptor modulator (e.g., an agonist or an antagonist); a glucose-dependent insulinotropic polypeptide (GIP) receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of PYY, an agent that increases a level and/or activity of GDF15, an agent that increases a level and/or activity of GFRAL, or any combination thereof. In some embodiments, heart damage comprises a change in function of a heart, a reduction in size of a heart (e.g., atrophy), a change in cellularity of a heart (e.g., increased infiltration of immune cells), or a reduction in weight of a heart, or any combination thereof. In some embodiments, heart damage comprises fibrosis.

In some embodiments, provided herein is a method for preventing, reducing, treating and/or reversing kidney disease, e.g., resulting from an additional agent, by administering to a subject an Activin A/B antibody agent disclosed herein. In some embodiments, a subject has received or is receiving an additional agent. In some embodiments, an Activin A/B antibody agent disclosed herein is administered concurrently with an additional agent. In some embodiments, an Activin A/B antibody agent disclosed herein is administered prior to administration of an additional agent. In some embodiments, an Activin A/B antibody agent disclosed herein is administered after administration of an additional agent. In some embodiments, an additional agent comprises: a Glucagon-like peptide-1 (GLP-1) receptor modulator (e.g., an agonist or an antagonist); a glucose-dependent insulinotropic polypeptide (GIP) receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of PYY, an agent that increases a level and/or activity of GDF15, an agent that increases a level and/or activity of GFRAL, or any combination thereof. In some embodiments, kidney disease is chronic kidney disease.

In some embodiments, provided herein is a method for preventing, reducing, treating and/or reversing liver disease, e.g., resulting from an additional agent, by administering to a subject an Activin A/B antibody agent disclosed herein. In some embodiments, a subject has received or is receiving an additional agent. In some embodiments, an Activin A/B antibody agent disclosed herein is administered concurrently with an additional agent. In some embodiments, an Activin A/B antibody agent disclosed herein is administered prior to administration of an additional agent. In some embodiments, an Activin A/B antibody agent disclosed herein is administered after administration of an additional agent. In some embodiments, an additional agent comprises a Glucagon-like peptide-1 (GLP-1) receptor modulator (e.g., an agonist or an antagonist); a glucose-dependent insulinotropic polypeptide (GIP) receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of PYY, an agent that increases a level and/or activity of GDF15, an agent that increases a level and/or activity of GFRAL, or any combination thereof. In some embodiments, liver disease is liver cirrhosis or NASH.

In some embodiments, provided herein is an antibody agent comprising a polypeptide that binds to Activin-A, Activin-B, and/or to one or more complexes that include one or both Activin-A and Activin-B, comprising at least one light chain complementarity determining region (LC CDR) and/or at least one heavy chain complementary determining region (HC CDR).

In some embodiments, an antibody agent is or comprises: (i) an intact IgA, IgG, IgD, IgE or IgM antibody; (ii) an antibody fragment; (iii) a single domain antibody; (iv) a single chain Fv; or (v) a polypeptide comprising an antigen binding domain fused to a Fc domain.

In some embodiments, an antibody agent binds to human Activin-A, Activin-B, and/or to one or more complexes that include one or both Activin-A and Activin-B with a binding affinity (K_{D}) of about 0.83 X 10 (−10) M to about 16.4 X 10 (−10) M, e.g., with a Fab format. In some embodiments, a binding affinity is determined with a binding affinity determining assay such as a surface plasmon resonance assay, an Octet assay or a comparable assay.

In some embodiments, an antibody agent comprises one, two, or three LC CDRs, e.g., as provided in Table 1 e.g., an LC CDR1, LC CDR2 and/or LC CDR3 of any one of clones A-C. In some embodiments, an antibody agent comprising a LC CDR1, LC CDR2 and/or LC CDR3 is capable of binding specifically to Activin-A and/or Activin-B.

In some embodiments, an antibody agent comprises (i) an LC CDR1, LC CDR2, and LC CDR3 sequence provided in Table 1; (ii) a sequence with at least 85%, 86%, 87%, 88%, 89%, 90%, 91%, 92%, 93%, 94%, 95%, 96%, 97%, 98%, 99% identity to an LC CDR1, LC CDR2, and LC CDR3 sequence provided in Table 1; or (iii) a sequence having at least 5, 10, or 20 substitutions relative to an LC CDR1, LC CDR2, and LC CDR3 sequence provided in Table 1.

In some embodiments, an antibody agent comprises a light chain comprising: (i) an LC CDR1 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR1 provided in Table 1; (ii) an LC CDR2 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR2 provided in Table 1; and/or (iii) an LC CDR3 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR3 provided in Table 1.

In some embodiments, an antibody agent comprises one, two or three HC CDRs, e.g., as provided in Table 2, e.g., an HC CDR1, an HC CDR2, and/or an HC CDR3 of any one of clones A-C. In some embodiments, an antibody agent comprising a HC CDR1, HC CDR2 and/or HC CDR3 is capable of binding specifically to Activin-A and/or Activin-B.

In some embodiments, an antibody agent comprises (i) an HC CDR1, HC CDR2, and HC CDR3 sequence provided in Table 2; (ii) a sequence with at least 85%, 86%, 87%, 88%, 89%, 90%, 91%, 92%, 93%, 94%, 95%, 96%, 97%, 98%, or 99% identity to an HC CDR1, HC CDR2, and HC CDR3 sequence provided in Table 2; (iii) a sequence having at least 5, 10, or 20 substitutions relative to an HC CDR1, HC CDR2, and HC CDR3 sequence provided in Table 2.

In some embodiments, an antibody agent comprises a heavy chain comprising: (i) a HC CDR1 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR1 provided in Table 2; (ii) a HC CDR2 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR2 provided in Table 2; and/or (iii) a HC CDR3 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR3 provided in Table 2.

In some embodiments, an antibody agent comprises one, two, or three LC CDRs (e.g., as provided in Table 1, e.g., an LC CDR1, LC CDR2 and/or LC CDR3 of any one of clones A-C) and one, two, or three HC CDRs (e.g., as provided in Table 2, e.g., an HC CDR1, an HC CDR2, and/or an HC CDR3 of any one of clones A-C). In some embodiments, an antibody agent comprising a LC CDR1, LC CDR2 and/or LC CDR3; and a HC CDR1, HC CDR2 and/or HC CDR3 is capable of binding specifically to Activin-A and/or Activin-B.

In some embodiments, an antibody agent comprises one, two, or three LC CDRs provided in Table 1 or a sequence with at least 85%, 86%, 87%, 88%, 89%, 90%, 91%, 92%, 93%, 94%, 95%, 96%, 97%, 98%, or 99% identity thereto; and one, two, or three HC CDRs provided in Table 2, or a sequence with at least 85%, 86%, 87%, 88%, 89%, 90%, 91%, 92%, 93%, 94%, 95%, 96%, 97%, 98%, or 99% identity thereto.

In some embodiments, an antibody agent comprises: (a) a light chain comprising: (i) an LC CDR 1 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR1 provided in Table 1; (ii) an LC CDR2 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR2 provided in Table 1; and/or (iii) an LC CDR3 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR3 provided in Table 1; and (b) a heavy chain comprising: (i) a HC CDR1 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR1 provided in Table 2; (ii) a HC CDR2 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR2 provided in Table 2; and/or (iii) a HC CDR3 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR3 provided in Table 2.

In some embodiments, an antibody agent comprising a light chain comprising a variable region (VL) comprising at least one LC CDR provided in Table 1 comprises at least one framework region (FR) provided in Table 1 or a sequence with at least 92% identity thereto. In some embodiments, an antibody agent comprising a VL comprises one, two, three or four FR regions, e.g., as provided in Table 1, or a sequence with at least 92% identity thereto.

In some embodiments, an antibody agent comprising a light chain comprising a variable region (VL) comprises: (i) the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; or (ii) the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16.

In some embodiments, an antibody agent comprising a heavy chain comprising a variable region (VH) comprising at least one HC CDR provided in Table 2 comprises at least one framework region (FR) provided in Table 2 or a sequence with at least 92% identity thereto. In some embodiments, an antibody agent comprising a VH comprises one, two, three or four FR regions, e.g., as provided in Table 2, or a sequence with at least 92% identity thereto.

In some embodiments, an antibody agent comprising a VH comprises: (i) the sequence of SEQ ID NO: 27, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 27; (ii) the sequence of SEQ ID NO: 32, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 32; or (iii) the sequence of SEQ ID NO: 39, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 39.

In some embodiments, an antibody agent comprises a VL polypeptide and a VH polypeptide. In some embodiments, an antibody agent comprises a VL polypeptide provided in Table 1, a sequence with at least 85%, 86%, 87%, 88%, 89%, 90%, 91%, 92%, 93%, 94%, 95%, 96%, 97%, 98%, or 99% to a VL polypeptide provided in Table 1, or a sequence having at least 5, 10, or 20 substitutions relative to a VL polypeptide provided in Table 1; and a VH polypeptide provided in Table 2, a sequence with at least 85%, 86%, 87%, 88%, 89%, 90%, 91%, 92%, 93%, 94%, 95%, 96%, 97%, 98%, or 99% to a VH polypeptide provided in Table 2, or a sequence having at least 5, 10, or 20 substitutions relative to a VH polypeptide provided in Table 2. In some embodiments, an antibody agent comprises: (i) the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; and the sequence of SEQ ID NO: 27, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 27; (ii) the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; and the sequence of SEQ ID NO: 32, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 32; (iii) the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16; and the sequence of SEQ ID NO: 39, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 39; (iv) the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; and the sequence of SEQ ID NO: 39, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 39; (v) the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16; and the sequence of SEQ ID NO: 27, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 27; or (vi) the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16; and the sequence of SEQ ID NO: 32, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 32.

In some embodiments, an antibody agent comprising a VH comprises a sequence for at least one constant region (CH). In some embodiments, at least one constant region comprises an Fc domain. In some embodiments, an Fc domain comprises a mammalian Fc domain, e.g., a mouse, a rat, a rabbit, a primate, a human, or a domestic animal Fc domain (e.g., a dog, a cat, a cow, or a horse Fc domain). In some embodiments, an Fc domain is chosen from an Fc domain of an immunoglobulin isotype. In some embodiments, an immunoglobulin isotype comprises IgA, IgG, IgM, or IgE.

In some embodiments, an antibody agent comprises (i) a light chain (LC) comprising: (a) one, two or three LC CDRs provided in Table 1 or a sequence with at least 85%, 86%, 87%, 88%, 89%, 90%, 91%, 92%, 93%, 94%, 95%, 96%, 97%, 98%, or 99% identity thereto; (b) at least one FR provided in Table 1 or sequence with at least 92% identity thereto; (c) a constant region (CL); and (ii) a heavy chain (HC) comprising: (a) one, two or three HC CDRs provided in Table 2 or a sequence with at least 85%, 86%, 87%, 88%, 89%, 90%, 91%, 92%, 93%, 94%, 95%, 96%, 97%, 98%, or 99% identity thereto; (b) at least one FR provided in Table 1 or a sequence with at least 92% identity thereto; (c) at least one constant region.

In some embodiments, an antibody agent comprises a LC polypeptide provided in Table 1, a sequence with at least 85%, 86%, 87%, 88%, 89%, 90%, 91%, 92%, 93%, 94%, 95%, 96%, 97%, 98%, or 99% to a LC polypeptide provided in Table 1, or a sequence having at least 5, 10, or 20 substitutions relative to a LC polypeptide provided in Table 1; and a HC polypeptide provided in Table 2, a sequence with at least 85%, 86%, 87%, 88%, 89%, 90%, 91%, 92%, 93%, 94%, 95%, 96%, 97%, 98%, or 99% to a HC polypeptide provided in Table 2, or a sequence having at least 5, 10, or 20 substitutions relative to a HC polypeptide provided in Table 2.

In some embodiments, an antibody agent comprises (i) the sequence of SEQ ID NO: 9, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 9; and the sequence of SEQ ID NO: 28, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 28; (ii) the sequence of SEQ ID NO: 9, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 9; and the sequence of SEQ ID NO: 33, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 33; (iii) the sequence of SEQ ID NO: 17, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 17; and the sequence of SEQ ID NO: 40, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 40; (iv) the sequence of SEQ ID NO: 9, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 9; and the sequence of SEQ ID NO: 40, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 40; (v) the sequence of SEQ ID NO: 17, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 17; and the sequence of SEQ ID NO: 28, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 28; or (vi) the sequence of SEQ ID NO: 17, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 17; and the sequence of SEQ ID NO: 33, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO:33.

In some embodiments, an Activin A/B antibody agent is characterized in that when tested in an assay that evaluates Activin-A and/or Activin-B activity and/or level, the antibody agent reduces Activin-A and/or Activin-B activity and/or level, or prevents an increase in Activin-A and/or Activin-B activity and/or level relative to a comparator.

In some embodiments, Activin-A and/or Activin-B is or comprises free and/or active Activin-A and/or Activin-B.

In some embodiments, an Activin A/B antibody agent reduces a plasma, blood, serum and/or urine level of Activin-A and/or Activin-B. In some embodiments, an Activin A/B antibody agent reduces a level of Activin-A and/or Activin-B to less than about 500 pg/mL.

In some embodiments, an Activin A/B antibody agent prevents an increase in Activin-A and/or Activin-B levels in plasma, blood, serum and/or urine. In some embodiments, an Activin A/B antibody agent prevents an increase in Activin-A and/or Activin-B level above about 500 pg/mL.

In some embodiments, an Activin A/B antibody agent reduces, e.g., inhibits, an Activin-A and/or Activin-B activity. In some embodiments, an Activin A/B antibody agent reduces, e.g., inhibits, the activity and/or level of Activin-A and/or Activin-B (e.g., plasma and/or urine Activin-A and/or Activin-B, e.g., free and/or active Activin-A and/or Activin-B) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, disclosed herein is a nucleic acid encoding an Activin A/B antibody agent. In some embodiments, a nucleic acid encoding an Activin A/B antibody agent is a DNA. In some embodiments, a nucleic acid encoding an Activin A/B antibody agent is an RNA, e.g., mRNA.

In some embodiments, also disclosed herein is a vector comprising a nucleic acid encoding an Activin A/B antibody agent.

In some embodiments, this disclosure provides a host cell comprising a vector disclosed herein.

In some embodiments, disclosed herein is a composition comprising an Activin A/B antibody agent. In some embodiments, a composition is or comprises a pharmaceutical composition.

In some embodiments, disclosed herein is a method comprising contacting an Activin A/B antibody agent (e.g., a composition comprising an Activin A/B antibody agent, e.g., a pharmaceutical composition), to a cell, tissue or subject. In some embodiments, a method comprises administering an Activin A/B antibody agent (e.g., a composition comprising an Activin A/B antibody agent, e.g., a pharmaceutical composition), to a cell, tissue or subject.

In some embodiments a method is a treatment method.

In some embodiments, a method is a prevention method.

In some embodiments, a subject disclosed herein has a condition or disorder associated with increased Activin A and/or Activin B. In some embodiments, condition or disorder is chosen from (i) disorders of the hypothalamic pituitary gonadal axis (e.g., with increased FSH levels); (ii) fibrodysplasia ossificans progressive (FOP); (iii) pulmonary arterial hypertension (PAH) and/or hypertension (e.g., preeclampsia); (iv) Anorexia-Cachexia Disorders associated with chronic diseases such as cancer, chronic kidney disease, chronic heart failure, COPD, elderly and muscle immobility, HIV patients, ICU patients (e.g., loss of body weight, loss of food intake, fat mass loss, organ mass loss, muscle wasting (e.g., muscle atrophy), loss of functional muscle mass and strength, bone loss, anemia, fatigue, and/or organ damage); (v) anemia; (vi) metabolic disorders (e.g., obesity, type 2 diabetes, metabolic syndrome and/or type 1 diabetes, lipodystrophy); (vii) inflammatory disorders (e.g. inflammatory bowel disease, pancreatitis, allergic asthma, COVID, acute respiratory distress syndrome, atopic dermatitis, preeclampsia, acute liver injury, acute kidney injury, Tubulointerstitial nephritis, osteoarthritis and inflammatory-induced anemia); (viii) auto-immune disorders (e.g., SLE or rheumatoid arthritis); (ix) fibrotic diseases (e.g. idiopathic pulmonary fibrosis (IPF), NASH, liver cirrhosis, chronic kidney disease, viral hepatitis, biliary disorders, Alport Syndrome, Uterine Fibroids, Endometriosis); (x) cancer; (xi) cancer-treatment related toxicities (e.g., chemotherapy-induced nephrotoxicity, hepatotoxicity); (xii) cancer related metastases (e.g., changes in cell adhesion, migration and/or invasion); (xiii) aging (e.g., senescence); (xiv) Myalgic Encephalomyelitis or Chronic Fatigue Syndrome; (xv) reproductive disorders (e.g., with elevated FSH, hypogonadotropic hypothalamic amenorrhea, polycystic ovary syndrome (PCOS), menopause, ovarian aging and/or ovarian cancers); (xvi) chemoresistance; or (xvii) heart failure.

In some embodiments, disclosed herein is a method of treating and/or reversing mass loss in a subject, comprising administering to a subject an Activin A/B antibody agent (e.g., a composition comprising an Activin A/B antibody agent, e.g., a pharmaceutical composition).

In some embodiments, mass comprises fat mass, lean mass, muscle mass, bone mass, or a combination thereof.

In some embodiments, disclosed herein is a method of treating and/or reversing weight loss (e.g., cachexia) comprising administering to a subject an Activin A/B antibody agent (e.g., a composition comprising an Activin A/B antibody agent, e.g., a pharmaceutical composition).

In some embodiments, disclosed herein is a method of treating and/or reversing organ damage (e.g., damage of one or more organs, or multi-organ damage) comprising administering to a subject an Activin A/B antibody agent (e.g., a composition comprising an Activin A/B antibody agent, e.g., a pharmaceutical composition). In some embodiments, organ damage comprises: a change in function of an organ, a reduction in size of an organ (e.g., atrophy), a change in cellularity of an organ (e.g., increased infiltration of immune cells), a reduction in weight of an organ, or any combination thereof. In some embodiments, organ damage comprises damage to one or more, or all of: a liver, kidney, heart, or pancreas.

In some embodiments, disclosed herein is a method of treating and/or reversing liver damage comprising administering to a subject an Activin A/B antibody agent (e.g., a composition comprising an Activin A/B antibody agent, e.g., a pharmaceutical composition). In some embodiments, administration of an Activin A/B antibody agent promotes liver regeneration. In some embodiments, liver damage comprises a change in function of a liver, a reduction in size of a liver (e.g., atrophy), a change in cellularity of a liver (e.g., increased infiltration of immune cells), or a reduction in weight of a liver, or any combination thereof. In some embodiments, liver damage comprises fibrosis. In some embodiments, liver damage comprises necrosis. In some embodiments, liver damage comprises degeneration. In some embodiments, liver damage comprises hepatitis.

In some embodiments, disclosed herein is a method of treating and/or reversing kidney damage comprising administering to a subject an Activin A/B antibody agent (e.g., a composition comprising an Activin A/B antibody agent, e.g., a pharmaceutical composition). In some embodiments, kidney damage comprises a change in function of a kidney, a reduction in size of a kidney (e.g., atrophy), a change in cellularity of a kidney (e.g., increased infiltration of immune cells), or a reduction in weight of a kidney, or any combination thereof. In some embodiments, kidney damage comprises fibrosis. In some embodiments, kidney damage comprises tubular degeneration.

In some embodiments, disclosed herein is a method of treating and/or reversing heart damage comprising administering to a subject an Activin A/B antibody agent (e.g., a composition comprising an Activin A/B antibody agent, e.g., a pharmaceutical composition). In some embodiments, heart damage comprises a change in function of a heart, a reduction in size of a heart (e.g., atrophy), a change in cellularity of a heart (e.g., increased infiltration of immune cells), or a reduction in weight of a heart, or any combination thereof. In some embodiments, heart damage comprises fibrosis.

In some embodiments, disclosed herein is a method of treating and/or reversing pancreatic damage comprising administering to a subject an Activin A/B antibody agent (e.g., a composition comprising an Activin A/B antibody agent, e.g., a pharmaceutical composition). In some embodiments, pancreatic damage comprises a change in function of a pancreas, a reduction in size of a pancreas (e.g., atrophy), a change in cellularity of a pancreas (e.g., increased infiltration of immune cells), or a reduction in weight of a pancreas, or any combination thereof. In some embodiments, pancreatic damage comprises pancreatitis.

In some embodiments, disclosed herein is a method of treating and/or reversing weight loss in a subject, comprising administering to a subject an Activin A/B antibody agent (e.g., a composition comprising an Activin A/B antibody agent, e.g., a pharmaceutical composition).

In some embodiments, disclosed herein is a method of treating and/or reversing senescence in a subject, comprising administering to a subject an Activin A/B antibody agent (e.g., a composition comprising an Activin A/B antibody agent, e.g., a pharmaceutical composition).

In some embodiments, disclosed herein is a method of treating and/or preventing liver damage in a subject, comprising administering to a subject an Activin A/B antibody agent (e.g., a composition comprising an Activin A/B antibody agent, e.g., a pharmaceutical composition). In some embodiments, liver damage comprises: acute liver injury, liver necrosis, liver fibrosis, liver inflammation, alteration in liver function, elevation one or more liver enzymes, or a combination thereof.

In some embodiments, disclosed herein is a method of treating and/or preventing kidney damage in a subject, comprising administering to a subject an Activin A/B antibody agent (e.g., a composition comprising an Activin A/B antibody agent, e.g., a pharmaceutical composition). In some embodiments, kidney damage comprises acute kidney injury, kidney fibrosis, kidney inflammation, alteration in kidney function, or a combination thereof.

In some embodiments, disclosed herein is a method of treating and/or preventing SAR-COV-2 infection in a subject, comprising administering to a subject an Activin A/B antibody agent (e.g., a composition comprising an Activin A/B antibody agent, e.g., a pharmaceutical composition).

In some embodiments, disclosed herein is a method of preventing and/or reducing migration of cells in vivo in a subject, comprising administering to a subject an Activin A/B antibody agent (e.g., a composition comprising an Activin A/B antibody agent, e.g., a pharmaceutical composition).

In some embodiments, disclosed herein is a method of reducing and/or preventing metastasis in a subject, comprising administering to a subject an Activin A/B antibody agent (e.g., a composition comprising an Activin A/B antibody agent, e.g., a pharmaceutical composition). In some embodiments, administration of an Activin A/B antibody agent reduces and/or prevent liver metastasis.

In some embodiments of any of the methods disclosed herein, an Activin A/B antibody agent reduces, e.g., inhibits, an activity of Activin-A and/or Activin-B. In some embodiments, an Activin A/B antibody agent reduces, e.g., inhibits, the activity and/or level of Activin-A and/or Activin-B (e.g., plasma and/or urine Activin A, e.g., free and/or active Activin-A and/or Activin-B) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments of any of the methods disclosed herein, Activin-A and/or Activin-B is or comprises free and/or active Activin-A and/or Activin-B.

In some embodiments of any of the methods disclosed herein, an Activin A/B antibody agent reduces a plasma, blood, serum and/or urine level of Activin-A and/or Activin-B. In some embodiments, an Activin A/B antibody agent reduces a level of Activin-A and/or Activin-B to less than about 500 pg/mL.

In some embodiments of any of the methods disclosed herein, an Activin A/B antibody agent prevents an increase in Activin-A and/or Activin-B levels in plasma, blood, serum and/or urine. In some embodiments, an Activin A/B antibody agent prevents an increase in Activin-A and/or Activin-B level above about 500 pg/mL.

### BRIEF DESCRIPTION OF THE DRAWING

FIGS. 1A-1C are graphs showing binding affinity for Activin A/B antibody agents (Clone A-C) to biontinylated Activin-A as measured with surface plasmon resonance assay. FIG. 1A is a graph showing data for biotinylated human Activin-A Fc bound to the chip for Activin A/B antibody Clone A Fab in solution (Top concentration 27 nM and 3-fold dilutions, 85 minutes dissociation). FIG. 1B is a graph showing data for biotinylated human Activin-A Fc bound to the chip for Activin A/B antibody Clone B Fab in solution (Top concentration 27 nM and 3-fold dilutions, 85 minutes dissociation). FIG. 1C is a graph showing data for biotinylated human Activin-A Fc bound to the chip for Activin A/B antibody Clone C Fab in solution (Top concentration 9 nM and 3-fold dilutions, 40 minutes dissociation).

FIG. 2 depicts binding affinity for Activin A/B antibody agents (Clone A-C avid) to Activin-A (left panel, 100 nM) or Activin-B (right panel, 100 nM) as measured with an Octet assay.

FIGS. 3A-3C depict inhibition of Activin-A or Activin-B activity with Activin A/B antibody agents (Clone A-C). The assay used to measure Activin A activity and/or Activin-B activity is the Activin 2B Receptor/SMAD reporter assay as described in Example 2. FIG. 3A is a graph showing the inhibition of Activin-A activity (top panel) and Activin-B activity (bottom panel) with Activin A/B antibody Clone A. FIG. 3B is a graph showing the inhibition of Activin-A activity (top panel) and Activin-B activity (bottom panel) with Activin A/B antibody Clone B. FIG. 3C is a graph showing the inhibition of Activin-A activity (top panel) and Activin-B activity (bottom panel) with Activin A/B antibody Clone C.

FIGS. 4A-4B depict pharmacokinetic profiles of exemplary with Activin A/B antibody agents administered intravenously or subcutaneously. FIG. 4A depicts mouse pharmacokinetic data and FIG. 4B depicts rat pharmacokinetic data.

FIGS. 5A-5B depict increase in Activin B and liver enzymes in a mouse in vivo disease model of liver toxicity. FIG. 5A shows alanine aminotransferase (ALT) enzyme activity (left panel) and aspartate transferase (AST) enzyme activity (right panel) serum levels with Carbon Tetrachloride (CCl₄) compared to control. FIG. 5B shows Activin A (left panel) and Activin B serum levels (right panel) with CCl₄ compared to control. Comparisons versus control are by one-way ANOVA; ***p<0.001.

FIGS. 6A-6C depict that AAV-overexpression of Activin-A and Activin-B in mice causes rapid weight loss. FIG. 6A shows that overexpression of Activin-A and/or Activin-B causes rapid weight loss, as compared to a reference (mice administered a null AAV vector). FIG. 6B shows that weight loss in mice overexpressing Activin-A was associated with fat and/or muscle loss. In each of the Activin A bar graphs, the first bar (closest to the Y-axis) represents mice administered a null AAV vector and the second bar represents mice administered AAV-Activin A. FIG. 6C shows that weight loss in mice overexpressing Activin-B was associated with fat and/or muscle loss. In each of the Activin B bar graphs, the first bar (closest to the Y-axis) represents mice administered a null AAV vector and the second bar represents mice administered AAV-Activin B. Body weight data analysises by ANCOVA with bodyweight on day 1 as a covariate. Comparisons versus the null control are by the multiple t test; **p<0.01; ***p<0.001.

FIGS. 7A-7E depict AAV-overexpression of Activin-A and Activin-B in mice causes liver damage. FIG. 7A shows alanine aminotransferase (ALT) levels are increased in mice administered Activin-A and/or Activin-B viral vectors, as compared to a reference (mice administered a null viral vector). FIG. 7B shows aspartate transferase (AST) levels increased in mice administered Activin-A and/or Activin-B viral vectors, as compared to a reference (mice administered a null viral vector). FIG. 7C depicts increased inflammatory cell infiltration score in mice administered Activin-A and/or Activin-B viral vectors, as compared to a reference (mice administered a null viral vector). FIG. 7D shows increased fibrosis score (as measured by collagen disposition) in mice administered Activin-A and/or Activin-B viral vectors, as compared to a reference (mice administered a null viral vector). FIG. 7E shows increased hepatocellular necrosis score in mice administered Activin-A and/or Activin-B viral vectors, as compared to a reference (mice administered a null viral vector). For liver enzymes statistical analysis, comparisons versus null vector group are by Kruskal-Wallis (non-parametric ANOVA) with separate pairwise comparisons (Dunn's test); **p<0.01; ***p<0.001. *p<0.05, **p<0.01, ***p<0.001 compared to null control via exact Wilcoxon rank sum test for histology.

FIG. 8 shows liver fibrosis in mice with chronic overexpression of Activin B for 4 weeks. Comparison to null control is by exact Wilcoxon rank sum test, *p<0.05.

FIGS. 9A-9B show tubular degeneration (FIG. 9A) and kidney fibrosis (FIG. 9B) in mice with acute overexpression (6 days) of Activin A. Comparison to null control is by exact Wilcoxon rank sum test; **p<0.01, ***p<0.001.

FIGS. 10A-10B show inflammation (FIG. 10A) and tubular degeneration (FIG. 10B) in mice with chronic overexpression of Activin B for 32 days. Comparison to null control is by exact Wilcoxon rank sum test; *p<0.05.

FIGS. 11A-11C show anemia in mice with chronic overexpression of Activin B. FIG. 11A shows red blood cell counts, FIG. 11B shows hematocrit levels, and FIG. 11C shows hemoglobin levels in Activin B overexpressing and control animals. Comparison to null control are by ANOVA; ***p<0.001.

FIGS. 12A-12B depict chronic AAV-overexpression of Activin-A and/or Activin-B in mice increases water intake. FIG. 12A shows increased water intake in mice administered Activin-A viral vectors, as compared to a reference (mice adminstered a null viral vector) over 38 days. FIG. 12B shows increased water intake in mice administered Activin-B viral vectors over 31 days, as compared to a reference (mice administered a null viral vector). Data analysis bu ANCOVA with baseline water intake as the covariate and compared to the null control by the multiple t test; *p<0.05, **p<0.01, ***p<0.001.

FIGS. 13A-13D show reversal of Activin A or Activin B overexpression-induced weight loss and cachexia with administration of an exemplary Activin A/B antibody agent. FIGS. 13A-13B depict reversal of weight loss with overexpression of Activin A with administration of 10 mg/kg (FIG. 13A) or 50 mg/kg (FIG. 13B) of an exemplary Activin A/B antibody agent. FIGS. 13C-13D depict reversal of weight loss with overexpression of Activin B with administration of 10 mg/kg (FIG. 13C) or 50 mg/kg (FIG. 13D) of an exemplary Activin A/B antibody agent. Arrows denote administration of exemplary Activin A/B antibody. Analyses performed by Mixed Model Repeated Measures analyses and compared to isoptype control; *p<0.05, ***p<0.001.

FIGS. 14A-14D show reversal of Activin A overexpression-induced multi-organ damage and decreased organ weight with administration of an exemplary Activin A/B antibody agent. Organ weights for heart (FIG. 14A), liver (FIG. 14B), pancreas (FIG. 14C) and kidneys (FIG. 14D) in control animals and animals with overexpression of Activin A with or without administration of an exemplary Activin A/B antibody agent are provided. Analysis compared to isotype control and corrected for body weight loss; *p<0.05, **p<0.01; ***p<0.001.

FIG. 15 shows reversal of Activin B overexpression-induced decreased pancreas weight and cachexia with administration of an exemplary Activin A/B antibody agent in mice. Analysis compared to isotype control and corrected for body weight loss; *p<0.05, **p<0.01.

FIGS. 16A-16B show chronic overexpression of Activin A induced liver damage in mice and reversibility with an exemplary anti-Activin A/B antibody. FIG. 16A is a graph showing circulating AST in null control mice or mice overexpressing Activin A (AAV-Activin A) after 26 days. FIG. 16B is a graph showing circulating ALT in null control mice or mice overexpressing Activin A (AAV-Activin A) after 26 days. The AAV-Activin A animals were either therapeutically treated with a control antibody or were treated with an exemplary anti-Activin A/B antibody. Antibody was administered at day 12 and 18 in Activin A overexpressing mice and day 18 and 26 in Activin B overpressing mice. Exemplary Anti-Activin A/B antibody fully reversed increased circulating ALT and AST induced by AAV-Activin A.***p<0.005 comparison to AAV-Activin A with isotype control via ANOVA.

FIGS. 17A-17F show prevention of weight loss, fat mass loss and muscle mass loss in an orthotopic MC38 colorectal cancer model. Mice were either left untreated (sham), intrasplenically injected with MC38 cancer cells and administered an exemplary anti-Activin A antibody, or intrasplenically injected with MC38 cancer cells and administered a control antibody. FIG. 17A shows body weight changes, FIG. 17B shows carcass weight, FIG. 17C shows adipose tissue weight, FIG. 17D shows gastrocnemius muscle weight, FIG. 17E shows tibialis muscle weight, and FIG. 17F shows food intake. Data analysis using ANOVA*p<0.05, **p<0.01 and ***p<0.005 compared to MC38 mice with IgG1 isotype control.

FIGS. 18A-18C show primary tumor and liver metastasis in the MC38 colorectal cancer model. FIG. 18A shows tumor weight in the spleen of mice not inoculated with MC38 cells (left bar), mice inoculated with MC38 tumor cells into the spleen and treated with a control antibody (middle bar) and mice inoculated with MC38 tumor cells and treated with an exemplary anti-Activin A antibody (right bar). Exemplary anti-Activin A reduced primary tumor weight. Data analysis using ANOVA, *p<0.05 compared to MC38 mice with IgG1 isotype control. FIG. 18B and FIG. 18C each contain three image panels showing representative livers from: two sham mice (left panels), two MC38 inoculated mice treated with anti-Activin A antibody (middle panels), and two MC38 inoculated mice treated with a control antibody (right panels). Exemplary anti-Activin A antibody prevented liver metastases.

FIGS. 19A-19L show over-expression of Activin A causes fibrosis and necrosis in mouse liver. FIG. 19A is a graph from acute over-expression of Activin A (6 days) showing inflammatory cell infiltration in control mice and Activin A overexpressing mice. FIG. 19B is a graph from acute over-expression of Activin A (6 days) showing fibrosis (collagen deposition) in control mice and Activin A overexpressing mice. FIG. 19C is a graph from acute over-expression of Activin A (6 days) showing hepatocellular necrosis in control mice and Activin A overexpressing mice. *** P<0.005 compared to null vector via exact Wilcoxon rank sum test for histology. FIGS. 19D-19L show gene expression data from chronic over-expression of Activin A. COL1A (FIG. 19D), COL3A (FIG. 19E), COL4A (FIG. 19F), smooth muscle actin (ATAC2, FIG. 19G), Fibronectin (FN1, FIG. 19H), and TGF-beta (FIG. 19I) levels were increased in the liver of mice chronically overexpressing Activin A for 39-51 days. FIGS. 19J-19L show levels of senescence markers p16 and p21 in the liver of mice chronically overexpressing Activin A for 39-51 days. The acute senescence marker p15 was unchanged. Comparisons to the null vector group are by the multiple t test at Day 39, *P<0.05, **P<0.01, *** P<0.001

FIG. 20 shows chronic overexpression of Activin causes bone loss. FIG. 20 shows femur bone density images using X-ray computed tomography from individual control mice with a null vector (n=8) or mice overexpressing Activin A for 39 (n=7) or 51 (n=3) days.

FIGS. 21A-21E summarizes the chronic overexpression of Activin A causes bone loss as measured by X-ray computed tomography and Dual-Energy X-ray Absorptiometry (DEXA). Using X-ray computed tomography, FIG. 21A shows reduction in femur trabecular bone volume/total volume, FIG. 21B shows reduction of femur trabecular number, FIG. 21C shows reduction in femur trabecular thickness and FIG. 21D shows reduction in femur trabecular spacing induced by Activin A overexpression compared to mice expressing the null vector. Using DEXA, FIG. 21E shows reduction in whole body bone density in mice caused by Activin A overexpression compared to null vector control mice

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Activin-A: The term “Activin-A or Activin A” is used herein in reference to Activin-A polypeptides as understood in the art. Activin-A is a member of the TGFbeta superfamily. Activin-A is also known as Activin beta-A chain or Inhibin beta-A chain. Under physiological conditions, Activin-A can form a homodimer composed of two β subunits of inhibin linked by a disulfide bridge referred to as a BA/BA homodimer. Activin-A protein is encoded by the INHBA gene. Amino acid sequences for full-length Activin-A, and/or for nucleic acids that encode it can be found in a public database such as GenBank, UniProt and Swiss-Prot. For example, an amino acid sequence of human Activin-A (SEQ ID NO: 48, for which residues 1-20 represent the signal peptide, and residues 311-426 represent a TGF beta family signature sequence, can be found as UniProt/Swiss-Prot Accession No. P08476; a nucleic acid sequence (SEQ ID NO: 51) encoding human Activin-A can be found at Accession No. NM_002192.3.Those skilled in the art will appreciate that sequences presented in SEQ ID NOs: 48 and 51 are exemplary, and certain variations (including, for example, conservative substitutions in SEQ ID NO:48, codon-optimized variants of SEQ ID NO:51, etc) are understood to also be or encode human Activin-A; additionally, those skilled in the art will appreciate that homologs and orthologs of human Activin-A are known and/or knowable through the exercise or ordinary skill, for example, based on degree of sequence identity, presence of one or more characteristic sequence elements, and/or one or more shared activities. As will be clear from context, the term “Activin-A or Activin A” can be used in reference to monomeric Activin-A and/or to homodimeric Activin-A.

Activin-A polypeptide: The phrase “Activin-A polypeptide or Activin A polypeptide”, is used herein to refer to polypeptides that share significant sequence identity and/or at least one characteristic sequence element with an appropriate reference polypeptide such as, for example: (a) human Activin-A, for example, as set forth in SEQ ID NO:48; (b) Rhesus macaque Activin-A, for example as set forth in SEQ ID NO: 56 (see XP_028701686.1); (c) dog Activin-A, for example as set forth in SEQ ID NO: 57 (see XP_038279632.1); and/or (d) cat Activin-A for example as set forth in SEQ ID NO: 58 (see NP_001009856.1). In some embodiments, a Activin-A polypeptide is or comprises a fragment of a parental Activin-A polypeptide (e.g., of SEQ ID NO:48 or a homolog, ortholog, or variant [e.g., a functional variant] thereof). In some embodiments, a Activin-A polypeptide shares at least one characteristic sequence element with a reference Activin-A polypeptide (e.g., of SEQ ID NO:48 or a homolog, ortholog, or variant [e.g., a functional variant] thereof). Alternatively or additionally, in some embodiments, a Activin-A polypeptide shares significant amino acid sequence identity with a relevant reference polypeptide (e.g., of SEQ ID NO:48 or a homolog, ortholog, or variant [e.g., a functional variant] thereof). For example, in some embodiments, a Activin-A polypeptide shares at least 50% with a reference Activin-A. In some embodiments, a Activin-A polypeptide is characterized by an ability to activate a receptor that binds Activin-A, e.g., a Type II receptor or a Type I receptor; in some such embodiments, such ability is comparable to that of an appropriate reference Activin-A (e.g., of SEQ ID NO:48 or a homolog, ortholog, or variant [e.g., a functional variant] thereof). For example, in some embodiments, a Activin-A polypeptide activates a Type II receptor or a Type I receptor with a binding affinity that is reasonably comparable to that of an appropriate reference Activin-A (e.g., of SEQ ID NO:48 or a homolog, ortholog, or variant [e.g., a functional variant] thereof); in some embodiments, a Activin-A polypeptide is characterized in that it competes with an appropriate reference Activin-A (e.g., of SEQ ID NO:48 or a homolog, ortholog, or variant [e.g., a functional variant] thereof) for binding and/or activation of a Type II receptor or a Type I receptor; in some such embodiments, such competition is observed over a range of concentrations (e.g., which range may, for example, extend over 2 fold, 3 fold, 4 fold, 5 fold, 10 fold, or more). In some embodiments, an Activin-A polypeptide is or comprises a polypeptide with at least 50% identity to SEQ ID NO: 48.

Activin-B: The term “Activin-B or Activin B” is used herein in reference to Activin-B polypeptides as understood in the art. Activin-B is a member of the TGFbeta superfamily. Activin-B is also known as Activin beta-B chain or Inhibin beta-B chain. Under physiological conditions, Activin-B can form a homodimer composed of two β subunits of inhibin linked by a disulfide bridge referred to as a BB/BB homodimer. Activin-B protein is encoded by the INHBB gene. Amino acid sequences for full-length Activin-B, and/or for nucleic acids that encode it can be found in a public database such as GenBank, UniProt and Swiss-Prot. For example, an amino acid sequence of human Activin-B (SEQ ID NO: 52, for which residues 1-20 represent the signal peptide, and can be found as UniProt/Swiss-Prot Accession No. P09529; a nucleic acid sequence (SEQ ID NO: 55) encoding human Activin-B can be found at Accession No NM 002193.4. Those skilled in the art will appreciate that sequences presented in SEQ ID NOs: 52 and 55 are exemplary, and certain variations (including, for example, conservative substitutions in SEQ ID NO: 52, codon-optimized variants of SEQ ID NO:55, etc) are understood to also be or encode human Activin-B; additionally, those skilled in the art will appreciate that homologs and orthologs of human Activin-B are known and/or knowable through the exercise or ordinary skill, for example, based on degree of sequence identity, presence of one or more characteristic sequence elements, and/or one or more shared activities. As will be clear from context, the term “Activin-B or Activin B” can be used in reference to monomeric Activin-B and/or to homodimeric Activin-B.

Activin-B polypeptide: The phrase “Activin-B polypeptide or Activin B polypeptide”, is used herein to refer to polypeptides that share significant sequence identity and/or at least one characteristic sequence element with an appropriate reference polypeptide such as, for example: (a) human Activin-B, for example, as set forth in SEQ ID NO:52; (b) Rhesus macaque Activin-B, for example as set forth in SEQ ID NO: 60; (c) dog Activin-B, for example as set forth in SEQ ID NO: 63; and/or (d) cat Activin-B for example as set forth in SEQ ID NO: 66. In some embodiments, an Activin-B polypeptide is or comprises a fragment of a parental Activin-B polypeptide (e.g., of SEQ ID NO: 52 or a homolog, ortholog, or variant [e.g., a functional variant] thereof). In some embodiments, a Activin-B polypeptide shares at least one characteristic sequence element with a reference Activin-B polypeptide (e.g., of SEQ ID NO: 52 or a homolog, ortholog, or variant [e.g., a functional variant] thereof). Alternatively or additionally, in some embodiments, a Activin-B polypeptide shares significant amino acid sequence identity with a relevant reference polypeptide (e.g., of SEQ ID NO: 52 or a homolog, ortholog, or variant [e.g., a functional variant] thereof). For example, in some embodiments, a Activin-B polypeptide shares at least 50% with a reference Activin-B. In some embodiments, a Activin-B polypeptide is characterized by an ability to activate a receptor that binds Activin-B, e.g., a Type II receptor or a Type I receptor; in some such embodiments, such ability is comparable to that of an appropriate reference Activin-B (e.g., of SEQ ID NO: 52 or a homolog, ortholog, or variant [e.g., a functional variant] thereof). For example, in some embodiments, a Activin-B polypeptide activates a Type II receptor or a Type I receptor with a binding affinity that is reasonably comparable to that of an appropriate reference Activin-B (e.g., of SEQ ID NO: 52 or a homolog, ortholog, or variant [e.g., a functional variant] thereof); in some embodiments, a Activin-B polypeptide is characterized in that it competes with an appropriate reference Activin-B (e.g., of SEQ ID NO:52 or a homolog, ortholog, or variant [e.g., a functional variant] thereof) for binding and/or activation of a Type II receptor or a Type I receptor; in some such embodiments, such competition is observed over a range of concentrations (e.g., which range may, for example, extend over 2 fold, 3 fold, 4 fold, 5 fold, 10 fold, or more). In some embodiments, a Activin-B polypeptide is or comprises a polypeptide with at least 50% identity to SEQ ID NO: 52.

Activin-A/B antibody agent: The terms “Activin A/B antibody agent” is used herein in reference to an antibody agent that binds specifically to Activin A (e.g., as described herein) and/or Activin B (e.g., as described herein), e.g., to a particular form thereof (e.g., free, in a complex, etc). In some embodiments, an Acitvin A/B antibody agent binds to Activin A. In some embodiments, an Acitvin A/B antibody agent binds to Activin B. In some embodiments, an Acitvin A/B antibody agent binds to both Activin A and Activin B. In some embodiments, an Activin A/B antibody agent binds to one or more complexes that include one or both Activin A and Activin B. Without wishing to be bound by any theory, an Activin A/B antibody agent that may bind Activin-A and/or Activin-B monomers and homodimers may also bind AB heterodimers.

Administration: As used herein, the term “administration” typically refers to the administration of a composition to a subject or system, for example to achieve delivery of an agent that is, or is included in or otherwise delivered by, the composition. Those of ordinary skill in the art will be aware of a variety of routes that may, in appropriate circumstances, be utilized for administration to a subject, for example an animal or a human. In some embodiments, an animal is a domestic animal, such as a companion animal, e.g., a dog or a cat; in some embodiments, an animal is an animal used in agriculture (e.g., farming [e.g., a cow, a sheep or a horse]) or for recreation. For example, in some embodiments, administration may be systemic or local. Those skilled in the art will be aware of appropriate administration routes for use with particular therapies described herein, for example which include bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be by injection (e.g., intramuscular, intravenous, or subcutaneous injection). In some embodiments, injection may involve bolus injection, drip, perfusion, or infusion. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, an antibody agent can be formulated for oral delivery. For example, one with skill in the art will understand that an antibody agent disclosed herein can be formulated for oral delivery using technologies developed by Oramed (https://www.oramed.com/) or Premas (https://www.premasbiotech.com/).

Adult: As used herein, the term “adult” refers to a human eighteen years of age or older. In some embodiments, a human adult has a weight within the range of about 90 pounds to about 250 pounds.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference “]. In some embodiments, affinity may be assessed relative to a contemporaneous reference; in some embodiments, affinity may be assessed relative to a historical reference. Typically, when affinity is assessed relative to a reference, it is assessed under comparable conditions.

Affinity matured” (or “affinity matured antibody”): as used herein, refers to an antibody with one or more alterations in one or more CDRs or FR thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al., BioTechnology 10:779-783 (1992) describes affinity maturation by VH and VL domain shuffling. Random mutagenesis of CDR and/or framework residues is described by: Barbas et al. Proc. Nat. Acad. Sci. U.S.A 91:3809-3813 (1994); Schier et al., Gene 169:147-155 (1995); Yelton et al., J. Immunol. 155:1994-2004 (1995); Jackson et al., J. Immunol. 154 (7): 3310-9 (1995); and Hawkins et al., J. Mol. Biol. 226:889-896 (1992).

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Agonist: Those skilled in the art will appreciate that the term “agonist” may be used to refer to an agent, condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered).

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: as used herein refers to a member of the animal kingdom. In some embodiments, “animal” refers to humans; unless otherwise specified, in many embodiments, a human may be of either gender and/or at any stage of development. In some embodiments, “animal” refers to non-human animals; unless otherwise specified, in many embodiments, a non-human animal may be of any gender and/or at any stage of development. In certain embodiments, a non-human animal is a mammal (e.g., a rodent, a mouse, a rat, a rabbit, a monkey, a dog, a cat, a sheep, cattle, a primate, and/or a pig). In some embodiments, an animal may be, for example, a mammals, a bird, a reptile, an amphibian, a fish, an insect, a worm, etc., In some embodiments, an animal may be a transgenic animal, genetically engineered animal, and/or a clone.

Antagonist: Those skilled in the art will appreciate that the term “antagonist”, as used herein, may be used to refer to an agent, condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. In some embodiments, antibodies produced and/or utilized in accordance with the present disclosure include one or more modifications on an Fc domain, e.g., an effector null mutation, e.g., a LALA, LAGA, FEGG, AAGG, or AAGA mutation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of dog, cat, mouse, rabbit, primate, or human antibodies. In some embodiments, antibody sequence elements are human, humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies, etc. In some embodiments, relevant formats may be or include: Adnectins®; Affibodies®; Affilins®; Anticalins®; Avimers®; BiTE®s; cameloid antibodies; Centyrins®; ankyrin repeat proteins or DARPINS®; dual-affinity re-targeting (DART) agents; Fynomers®; shark single domain antibodies such as IgNAR; immune mobilixing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of dog, cat, mouse, rabbit, primate, or human antibodies. In some embodiments, an antibody agent may include one or more sequence elements that are human, humanized, primatized, chimeric, etc, as is known in the art. In some embodiments, an antibody agent may include one or more complementarity determining regions that are human and/or one or more constant region sequences that are characteristic of human antibodies. In many embodiments, the term “antibody agent” is used to refer to one or more of the art-known or developed constructs or formats for utilizing antibody structural and functional features in alternative presentation. For example, in some embodiments, an antibody agent utilized in accordance with the present disclosure is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide comprising an antigen binding specificity fused to an Fc; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 96%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, we observe that ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (i.e., by expression of an engineered nucleic acid sequence. In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Antibody polypeptide: As used herein, the term “antibody polypeptide” refers to a polypeptide(s) that includes characteristic sequence element(s) (e.g., one or more CDRs, or a set of CDRs such as each of a CDR1, 2, and 3 as found in reference antibody chain and/or one or more FR regions and/or a set of FR regions, such as, for example, a complete variable region of a heavy or light chain of a reference antibody) of an antibody; in many embodiments, an antibody polypeptide includes sufficient such sequence element(s) that it binds to an epitope (e.g., an epitope bound by a reference antibody including the characteristic sequence element). In some embodiments, an antibody polypeptide is a full-length antibody or heavy or light chain thereof. In some embodiments, an antibody polypeptide is or comprises a complete heavy and/or light chain variable region of a reference antibody; in some such embodiments, an antibody polypeptide includes characteristic antibody sequence element(s) sufficient to confer specific binding to a relevant epitope—i.e., so that the antibody polypeptide includes at least one binding site. In some embodiments, an “antibody polypeptide” may include a binding domain which is homologous or largely homologous (e.g., shows significant sequence homology and/or in some embodiments significant sequence identity) to an immunoglobulin-binding domain. In some embodiments, an antibody polypeptide shows at least 99% identity with an immunoglobulin binding domain. In some embodiments, an “antibody polypeptide” has a binding domain that shows at least 70%, 80%, 85%, 90%, or 95% identity with an immuoglobulin binding domain, for example a reference immunoglobulin binding domain. In some embodiments, an “antibody polypeptide” may have an amino acid sequence identical to that of an antibody, or chain, or variable region thereof (or combination of variable region(s)) that is found in a natural source. In some embodiments, an antibody polypeptide may be prepared by, for example, isolation from a natural source or antibody library, recombinant production in or with a host system, chemical synthesis, etc., or combinations thereof. In some embodiments, an antibody polypeptide is an antibody agent as described herein.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, ovarian and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed subject matter.

CDR-grafted antibody: as used herein, refers to an antibody whose amino acid sequence comprises heavy and light chain variable region sequences from one species but in which the sequences of one or more of the CDR regions of VH and/or VL are replaced with CDR sequences of another species, such as antibodies having murine VH and VL regions in which one or more of the murine CDRs (e.g., CDR3) has been replaced with human CDR sequences. Likewise, a “CDR-grafted antibody” may also refer to antibodies having human VH and VL regions in which one or more of the human CDRs (e.g., CDR3) has been replaced with mouse CDR sequences.

Child: As used herein, the term “child” refers to a human between 1 day and 18 years of age. In some embodiments, a child may be an infant (e.g., may be less than or equal to about 12 months, 11 months, 10 months, 9 months, 8 months, 7 months, 6 months, 5 months, 4 months, 3 months, 2 months or 1 month old); in some embodiments, a child may be older than an infant. In some embodiments, a child may be a toddler (e.g., about 1 to about 3 years old); in some embodiments, a child may be younger than or older than a toddler. In some embodiments, a child may be a teen (e.g., between about 12 and about 18 years old); in some embodiments, a child may be younger than a teen (and/or older or younger than a toddler or older than an infant). Body weight can vary widely across ages and specific children, with a typical range being 4 pounds to 150 pounds.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, alpha-helix character, alpha-sheet character, coiled-coil character, random coil character, etc.), and/or by a particular functional feature (e.g., binding activity, enzymatic activity, folding activity, signaling activity, etc.).

Effector function: as used herein refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

Effector cell: as used herein refers to a cell of the immune system that expresses one or more Fc receptors and mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

“Framework” or “Framework Region”: as used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

High affinity binding: The term “high affinity binding”, as used herein refers to a high degree of tightness with which a particular ligand binds to its partner. Affinities can be measured by any available method, including those known in the art. In some embodiments, binding is considered to be high affinity if the K_{d} is about 1700 pM or less (e.g., below about 1600 pM, about 1500 pM, about 1400 pM, about 1300 pM, about 1200 pM, about 1100 pM, about 1000 pM, about 900 pM, about 800 pM, about 700 pM, about 600 pM, about 500 pM, about 400 pM, about 300 pM, about 200 pM, about 100 pM, about 90 pM, about 89 pM, about 88 pM, about 87 pM, about 86 pM, about 85 pM, about 84 pM, about 83 pM, etc.) in binding assays. In some embodiments, binding is considered to be high affinity if the affinity is stronger (e.g., the K_{d} is lower) for a polypeptide of interest than for a selected reference polypeptide. In some embodiments, binding is considered to be high affinity if the ratio of the K_{d} for a polypeptide of interest to the K_{d} for a selected reference polypeptide is 1:1 or less (e.g., 0.9:1, 0.8:1, 0.7:1, 0.6:1, 0.5:1. 0.4:1, 0.3:1, 0.2:1, 0.1:1, 0.05:1, 0.01:1, or less). In some embodiments, binding is considered to be high affinity if the K_{d} for a polypeptide of interest is about 100% or less (e.g., about 99%, about 98%, about 97%, about 96%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1% or less) of the K_{d} for a selected reference polypeptide.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Human: In some embodiments, a human is an embryo, a fetus, an infant, a child, a teenager, an adult, or a senior citizen.

Humanized: as is known in the art, the term “humanized” is commonly used to refer to antibodies (or antibody components) whose amino acid sequence includes VH and VL region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna. CMP matrix.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

K_{D}: as used herein, refers to the dissociation constant of a binding agent (e.g., an antibody or binding component thereof) from a complex with its partner (e.g., the epitope to which the antibody or binding component thereof binds).

Low affinity binding: The term “low affinity binding”, as used herein refers to a low degree of tightness with which a particular ligand binds to its partner. As described herein, affinities can be measured by any available method, including methods known in the art. In some embodiments, binding is considered to be low affinity if the K_{d} is about 1701 pM or more (e.g., above about 1701 pM, 1800 pM, 1900 pM, 2000 pM, 2100 pM, 2.2 nM, 2.3.nM, 2.4 nM, 2.5 nM, 2.6 nM, 2.7 nM, etc.) In some embodiments, binding is considered to be low affinity if the affinity is the same or lower (e.g., the K_{d} is about the same or higher) for a polypeptide of interest than for a selected reference polypeptide. In some embodiments, binding is considered to be low affinity if the ratio of the K_{d} for a polypeptide of interest to the K_{d} for a selected reference polypeptide is 1:1 or more (e.g., 1.1:1, 1.2:1, 1.3:1, 1.4:1, 1.5:1. 1.6:1, 1.7:1, 1.8:1, 1.9:1, 2:1, 3:1, 4:1, 5:1, 10:1 or more). In some embodiments, binding is considered to be low affinity if the K_{d} for a polypeptide of interest is 100% or more (e.g., 100%, 105%, 110%, 115%, 120%, 125%, 130%, 135%, 140%, 145%, 150%, 155%, 160%, 165%, 170%, 175%, 180%, 185%, 190%, 195%, 200%, 300%, 400%, 500%, 1000%, or more) of the K_{d} for a selected reference polypeptide.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc, which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215 (3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having not more than a small number (e.g., 3, 2, or 1) an amino acid sequence changes relative to that of a reference CDR. In some embodiments, a CDR that is substantially identical to a reference CDR differs from that reference CDR by one or more amino acid changes at the end of the reference CDR; in some such embodiments, the relevant CDR is identical to the reference CDR other than at one or both ends. As is known in the art, CDR elements typically have a length within a range of a few amino acids (e.g., 3, 4, 5, 6, or 7) to about 20 or 30 amino acids (see, for example, Collis et al. J. Mol. Biol. 325:337, 2003, incorporated herein by reference); thus, in some embodiments, a CDR may be considered to be substantially identical to a reference CDR when it shares at least about 80% (or less for a shorter CDR), at least about 85%, at least about 90%, at least about 95%, at least about 98%, at least about 99%, or at least about 100% identity with the reference CDR.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids., and/or as having “polar” or “non-polar” side chains Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215 (3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Treat: As used herein, the term “treat,” “treatment,” or “treating” is used to refer to one or more of partial or complete alleviation, amelioration, relief, inhibition, prevention, delay of onset of, reduction in severity of and/or reduction in frequency (e.g., incidence) of one or more symptoms or features of a disease, disorder, and/or condition. In some embodiments, treatment may be prophylactic; for example may be administered to a subject who does not exhibit signs of a disease, disorder, and/or condition. In some embodiments, treatment may be administered to a subject who exhibits only early signs of the disease, disorder, and/or condition, and may, for example, decrease risk of developing pathology associated with the disease, disorder, and/or condition and/or delay onset and/or decrease rate of development or worsening of one or more features of a disease, disorder and/or condition.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors, e.g., that are statistically correlated with increased risk of development of the relevant disease, disorder, and/or condition. Thus, in some embodiments, treatment may be prophylactic; in some embodiments, treatment may be therapeutic.

Variant: The term “variant”, as used herein, refers to a molecule or entity (e.g., that are or comprise a nucleic acid, protein, or small molecule) that shows significant structural identity with a reference molecule or entity but differs structurally from the reference molecule or entity, e.g., in the presence or absence or in the level of one or more chemical moieties as compared to the reference molecule or entity. In some embodiments, a variant also differs functionally from its reference molecule or entity. In many embodiments, whether a particular molecule or entity is properly considered to be a “variant” of a reference is based on its degree of structural identity with the reference molecule. As will be appreciated by those skilled in the art, a biological or chemical reference molecule in typically characterized by certain characteristic structural elements. A variant, by definition, is a distinct molecule or entity that shares one or more such characteristic structural elements but differs in at least one aspect from the reference molecule or entity. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION

The present disclosure provides, among other things, novel Activin A/B antibody agents which have improved binding kinetics, binding affinity, pharmacokinetics, and/or function, e.g., compared to an appropriate reference anti-Activin A/B antibody. In some embodiments, an Activin A/B antibody agent disclosed herein binds to Activin-A, Activin-B, and/or to one or more complexes that include one or both Activin-A and Activin-B with high affinity and/or high specificity. For example, in some embodiments, an Activin A/B antibody agent binds to Activin-A, Activin-B, and/or to one or more complexes that include one or both Activin-A and Activin-B with a K_{D} of about 83 pM to about 1700 pM (e.g., about 83.2 pM to about 1640 pM), e.g., as assessed in an assay that measures antibody binding affinity, e.g., a Surface Plasmon Resonance assay (Biacore) and/or an Octet assay.

In some embodiments, a provided Activin A/B antibody agent may show preferential binding to Activin-A and Activin-B relative to one or more TGFbeta family members other than Activin-A or Activin-B. In some such embodiments, preferential binding may be assessed, for example, by simultaneously contacting an Activin A/B antibody agent with Activin-A, Activin-B, and one or more other TGFbeta family members. Alternatively or additionally, in some embodiments, preferential binding may be assessed relative to an appropriate reference Activin-A and/or Activin-B antibody agent and, e.g., may reflect a higher level of binding to Activin-A and/or Activin-B relative to the one or more other TGFbeta family member than is observed with the reference antibody. In some embodiments, an Activin A/B antibody agent does not bind to one or more TGFbeta family members other than Activin-A and Activin-B. In some embodiments, an Activin A/B antibody agent disclosed herein binds to Activin-A, Activin-B, and/or to one or more complexes that include one or both Activin-A andActivin-B, and also binds to one or more other TGFbeta family members. For example, in some embodiments, a provided Activin A/B antibody agent binds to Activin-A, Activin-B, and/or to one or more complexes that include one or both Activin-A and Activin-B, and also GDF10 and/or GDF11.

In some embodiments, an Activin A/B antibody agent disclosed herein inhibits an activity of Activin-A and Activin-B and/or reduces a level of Activin-A and/or Activin-B (e.g., reduces a level of Activin-A and/or Activin-B in blood, plasma, serum, and/or urine) when administered to a cell, tissue or subject. In some embodiments, an Activin A/B antibody agent disclosed herein can be used to treat a condition or disease associated with increased Activin-A and/or Activin-B. In some embodiments, an Activin A/B antibody agent disclosed herein can be used to treat a symptom of a condition or disease associated with increased Activin-A and/or Activin-B. In some embodiments, a symptom comprises any one, all, or a combination of: weight loss, loss of appetite, fatigue, muscle mass loss, fat mass loss, organ mass loss, lean mass loss, lean mass atrophy, functional muscle mass loss, loss of muscle strength, bone loss, anemia, or fibrosis. Also provided herein are compositions comprising Activin A/B antibody agents disclosed herein, as well as methods of making and using the same.

### Activin-A

Activin-A is a protein of approximately 25 kDa that forms a homodimer via a disulfide bridge with another Activin-A protein and is a member of the transforming growth factor beta (TGFbeta) superfamily (Morianos I. et al (2019) Journal of Autoimmunity, Volume 104). Activin-A protein is synthesized as a precursor polypeptide and contains an amino terminal prodomain with 250-350 residues and a carboxy terminal mature domain (Bloise E. et al., (2018) Physiological Reviews, 99 (1)). The precursor polypeptide is cleaved by a furin-like protease releasing the mature protein which is biologically active (see Bloise E et al (2018)).

A human Activin-A polypeptide sequence is provided
herein as SEQ ID NO: 48:
MPLLWLRGFLLASCWIIVRSSPTPGSEGHSAAPDCPSCALAALPKDVPN
SQPEMVEAVKKHILNMLHLKKRPDVTQPVPKAALLNAIRKLHVGKVGEN
GYVEIEDDIGRRAEMNELMEQTSEIITFAESGTARKTLHFEISKEGSDL
SVVERAEVWLFLKVPKANRTRTKVTIRLFQQQKHPQGSLDTGEEAEEVG
LKGERSELLLSEKVVDARKSTWHVFPVSSSIQRLLDQGKSSLDVRIACE
QCQESGASLVLLGKKKKKEEEGEGKKKGGGEGGAGADEEKEQSHRPFLM
LQARQSEDHPHRRRRRGLECDGKVNICCKKQFFVSFKDIGWNDWIIAPS
GYHANYCEGECPSHIAGTSGSSLSFHSTVINHYRMRGHSPFANLKSCCV
PTKLRPMSMLYYDDGQNIIKKDIQNMIVEECGCS

The amino acid sequence of human Activin-A (SEQ ID NO: 48) comprises a signal peptide (residues 1-20) and a mature Activin-A sequence (residues 311-426).

Human Activin-A signal peptide: MPLLWLRGFLLASCWIIVRS (SEQ ID NO: 49) Mature Human Activin-A sequence:

(SEQ ID NO: 50)
GLECDGKVNICCKKQFFVSFKDIGWNDWIIAPSGYHANYCEGECPSHIA
GTSGSSLSFHSTVINHYRMRGHSPFANLKSCCVPTKLRPMSMLYYDDGQ
NIIKKDIQNMIVEECGCS

Human Activin-A can be encoded by the following nucleic acid sequence from the INHBA gene (SEQ ID NO: 51):

| | |
|---|---|
| 1 | agtacagtat aaaacttcac agtgccaata ccatgaagag gagctcagac agctcttacc |
| 61 | acatgataca agagccggct ggtggaagag tggggaccag aaagagaatt tgctgaagag |
| 121 | gagaaggaaa aaaaaaacac caaaaaaaaa aataaaaaaa tccacacaca caaaaaaacc |
| 181 | tgcgcgtgag gggggaggaa aagcagggcc ttttaaaaag gcaatcacaa caacttttgc |
| 241 | tgccaggatg cccttgcttt ggctgagagg atttctgttg gcaagttgct ggattatagt |
| 301 | gaggagttcc cccaccccag gatccgaggg gcacagcgcg gcccccgact gtccgtcctg |
| 361 | tgcgctggcc gccctcccaa aggatgtacc caactctcag ccagagatgg tggaggccgt |
| 421 | caagaagcac attttaaaca tgctgcactt gaagaagaga cccgatgtca cccagccggt |
| 481 | acccaaggcg gcgcttctga acgcgatcag aaagcttcat gtgggcaaag tcggggagaa |
| 541 | cgggtatgtg gagatagagg atgacattgg aaggagggca gaaatgaatg aacttatgga |
| 601 | gcagacctcg gagatcatca cgtttgccga gtcaggaaca gccaggaaga cgctgcactt |
| 661 | cgagatttcc aaggaaggca gtgacctgtc agtggtggag cgtgcagaag tctggctctt |
| 721 | cctaaaagtc cccaaggcca acaggaccag gaccaaagtc accatccgcc tcttccagca |
| 781 | gcagaagcac ccgcagggca gcttggacac aggggaagag gccgaggaag tgggcttaaa |
| 841 | gggggagagg agtgaactgt tgctctctga aaaagtagta gacgctcgga agagcacctg |
| 901 | gcatgtcttc cctgtctcca gcagcatcca gcggttgctg gaccagggca agagctccct |
| 961 | ggacgttcgg attgcctgtg agcagtgcca ggagagtggc gccagcttgg ttctcctggg |
| 1021 | caagaagaag aagaaagaag aggaggggga agggaaaaag aagggcggag gtgaaggtgg |
| 1081 | ggcaggagca gatgaggaaa aggagcagtc gcacagacct ttcctcatgc tgcaggcccg |
| 1141 | gcagtctgaa gaccaccctc atcgccggcg tcggcggggc ttggagtgtg atggcaaggt |
| 1201 | caacatctgc tgtaagaaac agttctttgt cagtttcaag gacatcggct ggaatgactg |
| 1261 | gatcattgct ccctctggct atcatgccaa ctactgcgag ggtgagtgcc cgagccatat |
| 1321 | agcaggcacg tccgggtcct cactgtcctt ccactcaaca gtcatcaacc actaccgcat |
| 1381 | gcggggccat agcccctttg ccaacctcaa atcgtgctgt gtgcccacca agctgagacc |
| 1441 | catgtccatg ttgtactatg atgatggtca aaacatcatc aaaaaggaca ttcagaacat |
| 1501 | gatcgtggag gagtgtgggt gctcatagag ttgcccagcc cagggggaaa gggagcaaga |
| 1561 | gttgtccaga gaagacagtg gcaaaatgaa gaaattttta aggtttctga gttaaccaga |
| 1621 | aaaatagaaa ttaaaaacaa aacaaaaaaa aaaacaaaaa aaaacaaaag taaattaaaa |
| 1681 | acaaaacctg atgaaacaga tgaaggaaga tgtggaaaaa atccttagcc agggctcaga |
| 1741 | gatgaagcag tgaaagagac aggaattggg agggaaaggg agaatggtgt accctttatt |
| 1801 | tcttctgaaa tcacactgat gacatcagtt gtttaaacgg ggtattgtcc tttcccccct |
| 1861 | tgaggttccc ttgtgagcct tgaatcaacc aatctagtct gcagtagtgt ggactagaac |
| 1921 | aacccaaata gcatctagaa agccatgagt ttgaaagggc ccatcacagg cactttccta |
| 1981 | cccaattacc caggtcataa ggtatgtctg tgtgacactt atctctgtgt atatcagcat |
| 2041 | acacacacac acacacacac acacacacac acacaggcat ttccacacat tacatatata |
| 2101 | cacatactgg taaaagaaca atcgtgtgca ggtggtcaca cttccttttt ctgtaccact |
| 2161 | tttgcaacaa aacaaaacaa acaacattaa aaaattgaga acaagtatgg aaagaatgaa |
| 2221 | agatcaagga aaaaagaata ccaagttaca tttcgttaag gtgcttatga tcttagaact |
| 2281 | atgcaaccta ataggtttga aactgtttac ctgagagaga acaaaaagag agactttttt |
| 2341 | gtattggaag taatctgatt aatttttatt ttcttcaagg agagatactt gaaaggaata |
| 2401 | tgtttgtcca tctgttggat ccaaacattt ctatattttg taaatgttgt tgttgttttt |
| 2461 | tttttaatcg tttactattt gcactacaat ggtgtttgac ctgtctaatc cttatttaac |
| 2521 | aagtattttc tttggttggg ggtgggggtg gggtttaaga gctgcactta atgtgagcta |
| 2581 | taaaagaact gctacagcac acaaaatagc tatttttatt attataatta taattattat |
| 2641 | tattattttg taccttaaaa aatagacaca tacaccaaag acatttgtgt gagcctttaa |
| 2701 | acagtctgtc tgtggttggt atcattcacc atcaatgagt caggggttgg gattcaaggt |
| 2761 | tgagtagtgt ggattgtgtt caggcttaaa agacctgaga agtttggttt ttgactcctt |
| 2821 | ttacatccat gaaacaggac atttcatact ggatgtacag tagttgtaca ctgttggata |
| 2881 | tcaagttcaa tcaaattcat ggaactacat gcttgtatgt gtatatatac attgcttgtg |
| 2941 | catatgcata tctgtatgta tatatacatg tattgtacca tgtccataca cattttaagc |
| 3001 | acttcaggct gtcatttttt aatgttctta aagcaatgaa tgtttgtgtg caaaacacag |
| 3061 | tatttttaag aaggataggc tatagttttt gcttttactc tgaactaggt gggcgcattt |
| 3121 | caaaaattcg gatgggaaaa agcctggaaa ttccagtgaa tattcagcaa ggccctcttt |
| 3181 | cattgtacag ggatcaaatt tcctcctctt ttttgtgccc cctcccactt ctacaagtta |
| 3241 | tcccctgtgg ggaaaacagg atgataatca aaactctggg ctgatgtttt tccaacttag |
| 3301 | tgtctattgg aatcaatctt aaatcagaag ctttttcaga aaaataatat ttaggccaga |
| 3361 | attagagttg agtgtatttt ttaaaaatga ttaaggcttg gttgtgagaa atattacctg |
| 3421 | taccagctgg gaaaaataat gtcatcacta actaaaagat aattaatttg agagaaagtg |
| 3481 | ttaagagagg gagagtaagg aagagaacag ttaagaggag gcagaggtga gggcagtagt |
| 3541 | aaaaatctct aaaattttaa tttacagcca aaattcttca tgtgtaaatt tgtattgatt |
| 3601 | cagatgcaga aatgaaaaaa aaacaccttt gttttataaa tatcaaagta catgcttaaa |
| 3661 | gccaagtttt tatctagttt attctagtac ttagcttgcc tggaatagct aataaattat |
| 3721 | tcatgtatgt gcttttgaaa atccagagcc ctatttttac acacttgtgt gaagttggca |
| 3781 | aacattttga aaaatggaaa aaagtttcta ataattggga acaattacat taattaatat |
| 3841 | tttgtaaaat attgaagctt ttagccctat gtcaatttgt agattaaaat aaattaatta |
| 3901 | taggaaagga agataacagt gagaaaccaa acattacaaa aggtggttta gctctccttg |
| 3961 | aaaaatatac taagttggta tactataaca cttggctata tgtaggcaat gtcactactg |
| 4021 | ggcaaataca cttactgtgt tctagaggca gccctttctt atgcagaaaa tacaatacgc |
| 4081 | actgcatgag aagcttgaga gtggattcta atccaggtct gtcgaccttg gatatcatgc |
| 4141 | atgtgggaag gtgggtgtgg tgagaaaagt tttaaggcaa gagtagatgg ccatgttcaa |
| 4201 | ctttacaaaa tttcttggaa aactggcagt attttgaact gcatcttctt tggtaccgga |
| 4261 | acctgcagaa acagtgtgag aaattaagtc ctggttcact gcgcagtagc aaagatggtc |
| 4321 | aaggccatgg aaaaagcaga aatttaccaa gaaagctgat acccatgtat agttcccact |
| 4381 | catctcaaat acatctgcta tctttttaag ctaagtccta gacatatcgg ggataacatg |
| 4441 | ggggttgatt agtgaccaca gttatcagaa gcagagaaat gtaattccat attttatttg |
| 4501 | aaacttattc catattttaa ttggatattg agtgattggg ttatcaaaca cccacaaact |
| 4561 | ttaattttgt taaatttata tggctttgaa atagaagtat aagttgctac cattttttga |
| 4621 | taacattgaa agatagtatt ttaccatctt taatcatctt ggaaaataca agtcctgtga |
| 4681 | acaaccactc tttcacctag cagcatgagg ccaaaagtaa aggctttaaa ttataacata |
| 4741 | tgggattctt agtagtatgt ttttttcttg aaactcagtg gctctatcta accttactat |
| 4801 | ctcctcactc tttctctaag actaaactct aggctcttaa aaatctgccc acaccaatct |
| 4861 | tagaagctct gaaaagaatt tgtctttaaa tatcttttaa tagtaacatg tattttatgg |
| 4921 | accaaattga cattttcgac tattttttcc aaaaaagtca ggtgaatttc agcacactga |
| 4981 | gttgggaatt tcttatccca gaagaccaac caatttcata tttatttaag attgattcca |
| 5041 | tactccgttt tcaaggagaa tccctgcagt ctccttaaag gtagaacaaa tactttctat |
| 5101 | ttttttttca ccattgtggg attggacttt aagaggtgac tctaaaaaaa cagagaacaa |
| 5161 | atatgtctca gttgtattaa gcacggaccc atattatcat attcacttaa aaaaatgatt |
| 5221 | tcctgtgcac cttttggcaa cttctctttt caatgtaggg aaaaacttag tcaccctgaa |
| 5281 | aacccacaaa ataaataaaa cttgtagatg tgggcagaag gtttgggggt ggacattgta |
| 5341 | tgtgtttaaa ttaaaccctg tatcactgag aagctgttgt atgggtcaga gaaaatgaat |
| 5401 | gcttagaagc tgttcacatc ttcaagagca gaagcaaacc acatgtctca gctatattat |
| 5461 | tatttatttt ttatgcataa agtgaatcat ttcttctgta ttaatttcca aagggtttta |
| 5521 | ccctctattt aaatgctttg aaaaacagtg cattgacaat gggttgatat ttttctttaa |
| 5581 | aagaaaaata taattatgaa agccaagata atctgaagcc tgttttattt taaaactttt |
| 5641 | tatgttctgt ggttgatgtt gtttgtttgt ttgtttctat tttgttggtt ttttactttg |
| 5701 | ttttttgttt tgttttgttt tgttttgcat actacatgca gttctttaac caatgtctgt |
| 5761 | ttggctaatg taattaaagt tgttaattta tatgagtgca tttcaactat gtcaatggtt |
| 5821 | tcttaatatt tattgtgtag aagtactggt aattttttta tttacaatat gtttaaagag |
| 5881 | ataacagttt gatatgtttt catgtgttta tagcagaagt tatttatttc tatggcattc |
| 5941 | cagcggatat tttggtgttt gcgaggcatg cagtcaatat tttgtacagt tagtggacag |
| 6001 | tattcagcaa cgcctgatag cttctttggc cttatgttaa ataaaaagac ctgtttggga |
| 6061 | tgtaaaaaaa aaaaaaaaaa aaaaaaaaaa aaaaaaaaaa aaaaa |

Activin-A can bind to a Type I receptor or a Type II receptor. Binding of Activin-A to a Type II receptor, e.g., ActRIIA or AcRIIB, recruits and phosphorylates a Type I receptor (e.g., ActRI or ALK4). Upon activation of a Type I receptor, Smad2 and Smad 3 proteins are phosphorylated and appropriate downstream signal cascades are activated. In addition, activation of a Type I receptor can also result in activation of pathways such as the p38 MAPK pathway, ERK1/2 pathway, and JNK pathway.

Without wishing to be bound by any particular theory, the present disclosure provides an insight that an Activin A/B antibody agent disclosed herein binds to Activin-A and inhibits binding of endogenous Activin-A (e.g., active Activin-A) to an Activin-A receptor (e.g., a Type II receptor [e.g., ACTRIIA or ACTRIIB] and/or a Type I receptor [e.g., ActRI or ALK4]) thus preventing activation of a Type II and/or Type I receptor and/or one or more downstream signaling pathways. In some embodiments, modulation of an Activin-A receptor with an Activin A/B antibody agent disclosed herein allows for treatment of conditions, diseases or disorder associate with (e.g., mediated by) Activin-A.

Without wishing to be bound by any particular theory, the present disclosure provides a further insight that an Activin A/B antibody agent disclosed herein binds to Activin-A and inhibits binding of endogenous Activin-A to a receptor, e.g., a receptor that binds to Activin-A, e.g., an Activin-A receptor (e.g., a Type II receptor [e.g., ACTRIIA or ACTRIIB] and/or a Type I receptor [e.g., ActRI or ALK4]). In some embodiments, an Activin-A receptor is accessible to an antibody agent, e.g., as disclosed herein.

While certain anti-Activin A antibodies have been previously developed (see, e.g., WO2015017576), none of the art known antibodies have so far been demonstrated as feasible therapeutic options for preventing and treating conditions, diseases or disorders, or symptoms thereof associated with (e.g., mediated by) elevated levels of Activin-A; moreover, none could teach or suggest the particular antibodies or antibody agents described herein, which show remarkable and unexpected properties, including as documented herein. In some embodiments, an elevated level of Activin-A is about 500 pg/ml or more, e.g., as evaluated in a sample from a subject, e.g., a blood, plasma, serum or urine sample. In some embodiments, a provided antibody agent blocks and/or reverses weight loss even in dramatic weight loss contexts (e.g., models) including, for example, potently and/or completely blocks functional signaling via the Activin-A receptor; alternatively or additionally, in some embodiments, a provided antibody agent prevents or reverses mass loss (e.g., muscle mass loss, lean mass loss, fat mass loss, organ mass loss, and/or bone mass loss), weight loss, senescence, liver damage, kidney damage, or cancer. Thus, the present disclosure provides novel Activin-A antibody agents which can be used to meet the unmet need of treating and/or preventing conditions, diseases or disorders, or symptoms thereof, that are associated with elevated Activin-A.

### Activin-B

Activin-B shares a high (64%) sequence identity with Activin-A, however, they are differentially regulated during development (Thompson et. al., Molecular and Cellular Endocrinology, Volume 225). Biologically active Activin-B is a protein of approximately 25 kDa that forms a homodimer via a disulfide bridge with another Activin-B protein and is a member of the transforming growth factor beta (TGFbeta) superfamily (Harrison C. et al (2006) Endocrinology, Volume 147). Activin-B protein is synthesized as a large precursor polypeptide contain an amino terminal prodomain with 250-350 residues and a carboxy terminal mature domain (Bloise E. et al., (2018) Physiological Reviews, 99 (1)). The precursor polypeptide is cleaved by a furin-like protease releasing the mature protein which is biologically active (see Bloise E et al (2018)).

A human Activin-B polypeptide sequence is provided
herein as SEQ ID NO: 52:
MDGLPGRALGAACLLLLAAGWLGPEAWGSPTPPPTPAAPPPPPPPGSPG
GSQDTCTSCGGFRRPEELGRVDGDFLEAVKRHILSRLQMRGRPNITHAV
PKAAMVTALRKLHAGKVREDGRVEIPHLDGHASPGADGQERVSEIISFA
ETDGLASSRVRLYFFISNEGNQNLFVVQASLWLYLKLLPYVLEKGSRRK
VRVKVYFQEQGHGDRWNMVEKRVDLKRSGWHTFPLTEAIQALFERGERR
LNLDVQCDSCQELAVVPVFVDPGEESHRPFVVVQARLGDSRHRIRKRGL
ECDGRTNLCCRQQFFIDFRLIGWNDWIIAPTGYYGNYCEGSCPAYLAGV
PGSASSFHTAVVNQYRMRGLNPGTVNSCCIPTKLSTMSMLYFDDEYNIV
KRDVPNMIVEECGCA
Human Activin-B signal peptide:
(SEQ ID NO: 53)
MDGLPGRALGAACLLLLAAGWLGPEAWGSPTPPPTPAAPPPPPPPGSPG
GSQDTCTSCGGFRRPEELGRVDGDFLEAVKRHILSRLQMRGRPNITHAV
PKAAMVTALRKLHAGKVREDGRVEIPHLDGHASPGADGQERVSEIISFA
ETDGLASSRVRLYFFISNEGNQNLFVVQASLWLYLKLLPYVLEKGSRRK
VRVKVYFQEQGHGDRWNMVEKRVDLKRSGWHTFPLTEAIQALFERGERR
LNLDVQCDSCQELAVVPVFVDPGEESHRPFVVVQARLGDSRHRIRKR

Mature Human Activin-B sequence:

(SEQ ID NO: 54)
GLECDGRTNLCCRQQFFIDFRLIGWNDWIIAPTGYYGNYCEGSCPAYLA
GVPGSASSFHTAVVNQYRMRGLNPGTVNSCCIPTKLSTMSMLYFDDEYN
IVKRDVPNMIVEECGCA

Human Activin-B can be encoded by the following nucleic acid sequence from the INHBB gene (SEQ ID NO: 55):

| | |
|---|---|
| 1 | CGGCTCGACT CGGCTCGCCT CGCGGCGGGC GCCCTCGTCG CCAGCGGCGC ACCATGGACG |
| 61 | ggctgcccgg tcgggcgctg ggggccgcct gccttctgct gctggcggcc ggctggctgg |
| 121 | ggcctgaggc ctggggctca cccacgcccc cgccgacgcc tgccgcgccg ccgccacccc |
| 181 | cgccacccgg atccccgggt ggctcgcagg acacctgtac gtcgtgcggc ggcttccggc |
| 241 | ggccagagga gctcggccga gtggacggcg acttcctgga ggcggtgaag cggcacatct |
| 301 | tgagccgcct gcagatgcgg ggccggccca acatcacgca cgccgtgcct aaggccgcca |
| 361 | tggtcacggc cctgcgcaag ctgcacgcgg gcaaggtgcg cgaggacggc cgcgtggaga |
| 421 | tcccgcacct cgacggccac gccagcccgg gcgccgacgg ccaggagcgc gtttccgaaa |
| 481 | tcatcagctt cgccgagaca gatggcctcg cctcctcccg ggtccgccta tacttcttca |
| 541 | tctccaacga aggcaaccag aacctgtttg tggtccaggc cagcctgtgg ctttacctga |
| 601 | aactcctgcc ctacgtcctg gagaagggca gccggcggaa ggtgcgggtc aaagtgtact |
| 661 | tccaggagca gggccacggt gacaggtgga acatggtgga gaagagggtg gacctcaagc |
| 721 | gcagcggctg gcataccttc ccactcacgg aggccatcca ggccttgttt gagcggggcg |
| 781 | agcggcgact caacctagac gtgcagtgtg acagctgcca ggagctggcc gtggtgccgg |
| 841 | tgttcgtgga cccaggcgaa gagtcgcacc ggccctttgt ggtggtgcag gctcggctgg |
| 901 | gcgacagcag gcaccgcatt cgcaagcgag gcctggagtg cgatggccgg accaacctct |
| 961 | gttgcaggca acagttcttc attgacttcc gcctcatcgg ctggaacgac tggatcatag |
| 1021 | cacccaccgg ctactacggg aactactgtg agggcagctg cccagcctac ctggcagggg |
| 1081 | tccccggctc tgcctcctcc ttccacacgg ctgtggtgaa ccagtaccgc atgcggggtc |
| 1141 | tgaaccccgg cacggtgaac tcctgctgca ttcccaccaa gctgagcacc atgtccatgc |
| 1201 | tgtacttcga tgatgagtac aacatcgtca agcgggacgt gcccaacatg attgtggagg |
| 1261 | agtgcggctg cgcctgacag tgcaaggcag gggcacggtg gtggggcacg gagggcagtc |
| 1321 | ccgggtgggc ttcttccagc ccccgcggga acgggggtac acggtgggct gagtacagtc |
| 1381 | attctgttgg gctgtggaga tagtgccagg gtgcggcctg agatattttt ctacagcttc |
| 1441 | atagagcaac cagtcaaaac cagagcgaga accctcaact gacatgaaat actttaaaat |
| 1501 | gcacacgtag ccacgcacag ccagacgcat cctgccaccc acacagcagc ctccaggata |
| 1561 | ccagcaaatg gatgcggtga caaatggcag cttagctaca aatgcctgtc agtcggagag |
| 1621 | aatggggtga gcagccacca ttcccaccag ctggcccggc cactctgaat tgcgccttcc |
| 1681 | gagcacacat aaaagcacaa agacagagac gcagagagag agagagagcc acggagagga |
| 1741 | aaagcagatg caggggtggg gagcgcagct cggcggaggc tgcgtgtgcc ccgtggcttt |
| 1801 | taccaggcct gctctgcctg gctcgatgtc tgcttcttcc ccagcctggg atccttcgtg |
| 1861 | cttcaaggcc tggggagcct gtccttccat gcccttgtcg agggaaagag acccagaaag |
| 1921 | gacacaaccc gtcagagacc tgggagcagg ggcaatgacc gtttgactgt ttgtggcttg |
| 1981 | ggcctctgac atgacttatg tgtgtgtgtg tttttggggt ggggagggag ggagagaaga |
| 2041 | gggggctaaa tttgatgctt taactgatct ccaacagttg acaggtcatc cttgccagtt |
| 2101 | gtataactga aaaaggactt ttctaccagg tatgaccttt taagtgaaaa tctgaattgt |
| 2161 | tctaaatgga aagaaaaaaa gttgcaatct gtgcccttca ttggggacat tcctctagga |
| 2221 | ctggtttggg gacgggtggg aatgacccct aggcaagggg atgagaccgc aggaggaaat |
| 2281 | ggcggggagg aggcattctt gaactgctga ggatgggggg tgtcccctca gcggaggcca |
| 2341 | agggagggga gcagcctagt tggtcttgga gagatgggga aggctttcag ctgatttgca |
| 2401 | gaagttgccc atgtgggccc cagccatcag ggctggccgt ggacgtggcc cctgcccact |
| 2461 | cacctgcccg cctgcccgcc cgcccgcata gcacttgcag acctgcctga acgcacatga |
| 2521 | catagcactt gccgatctgc gtgtgtccag aagtggccct tggccgagcg ccgaactcgc |
| 2581 | tcgccctcta gatgtccaag tgccacgtga actatgcaat ttaaagggtt gacccacact |
| 2641 | agacgaaact ggactcgtac gactcttttt atatttttta tacttgaaat gaaatccttt |
| 2701 | gcttcttttt taagcgaatg attgctttta atgtttgcac tgatttagtt gcatgattag |
| 2761 | tcagaaactg ccatttgaaa aaaagttatt tttatagcag caaaaaaaaa aaaaaaagaa |
| 2821 | tacagttaaa tgtattatac ataattttgg aaccaaagag gccaacagat cagttttaat |
| 2881 | tttattagac ggtgaggcca tctgagatga ggtggacgtt ctgagcagtc ccttgagtgg |
| 2941 | cctgccaacg tttcagggta tgaatggatt ttgtttattc ggtttgatgt gtcttttcca |
| 3001 | tccttacaca cccagaaggt agagtaaaaa tgactatgat agaatgcagg tgtgtatcct |
| 3061 | taaatcctca tctttatgtt tatttaataa agctcccctt agattctgtt tcataataat |
| 3121 | ttaaaaccaa acaattttcc catagacttg ctgttaaagt attgtacgtt tgtgtacagt |
| 3181 | ttaagaaaat aaaagattga gtgcca |

A Rhesus macaque Activin-B polypeptide sequence is provided herein as SEQ ID NO: 60:

MDGLPGRALGAACLLLLAAGWLGPEAWGSPTPPPSPAAPPPPPPPGAPGGSQ
DTCTSCGGFRRPEELGRVDGDFLEAVKRHILSRLQMRGRPNITHAVPKAAMVTALRKL
HAGKVREDGRVEIPHLDGHASPGADGQERVSEIISFAETDGLASSRVRLYFFISNEGNQN
LFVVQASLWLYLKLLPYVLEKGSRRKVRVKVYFQEQGHGDRWNMVEKRVDLKRSGW
HTFPLTEAIQALFERGERRLNLDVQCDSCQELAVVPVFVDPGEESHRPFVVVQARLGDS
RHRIRKRGLECDGRTNLCCRQQFFIDFRLIGWNDWIIAPTGYYGNYCEGSCPAYLAGVP
GSASSFHTAVVNQYRMRGLNPGAVNSCCIPTKLSTMSMLYFDDEYNIVKRDVPNMIVE
ECGCA
Rhesus macaque Activin-B signal peptide:
SEQ ID NO: 61
MDGLPGRALGAACLLLLAAGWLGPEAWGSPTPPPSPAAPPPPPPPGAPGGSQ
DTCTSCGGFRRPEELGRVDGDFLEAVKRHILSRLQMRGRPNITHAVPKAAMVTALRKL
HAGKVREDGRVEIPHLDGHASPGADGQERVSEIISFAETDGLASSRVRLYFFISNEGNQN
LFVVQASLWLYLKLLPYVLEKGSRRKVRVKVYFQEQGHGDRWNMVEKRVDLKRSGW
HTFPLTEAIQALFERGERRLNLDVQCDSCQELAVVPVFVDPGEESHRPFVVVQARLGDS
RHRIRKR
Rhesus macaque Activin-B mature protein:
SEQ ID NO: 62
GLECDGRTNLCCRQQFFIDFRLIGWNDWIIAPTGYYGNYCEGSCPAYLAGVP
GSASSFHTAVVNQYRMRGLNPGAVNSCCIPTKLSTMSMLYFDDEYNIVKRDVPNMIVE
ECGCA
A dog Activin-B polypeptide sequence is provided herein SEQ ID NO: 63:
MDGLPGRALGAACLLLLAAGWLGPEAWGSPTPPPSPAAPPPPPPPGAPGGSQ
DTCTSCGGFRRPEELGRVDGDFLEAVKRHILSRLQMRGRPNITHAVPKAAMVTALRKL
HAGKVREDGRVEIPHLDGHASPGSDGQERVSEIISFAETDGLASSRVRLYFFVSNEGNQN
LFVVQASLWLYLKLLPYVLEKGSRRKVRVKVYFQEQGQGDRWNAVEKKVDLKRSGW
HTFPLTEAIQALFERGERRLSLDVQCDGCQELAVVPVFVDPGEESHRPFVVVQARLGDS
RHRIRKRGLECDGRTNLCCRQQFFIDFRLIGWNDWIIAPTGYYGNYCEGSCPAYLAGVP
GSASSFHTAVVNQYRMRGLNPGTVNSCCIPTKLSTMSMLYFDDEYNIVKRDVPNMIVEE
CGCA
Dog Activin-B signal peptide:
SEQ ID NO: 64
MDGLPGRALGAACLLLLAAGWLGPEAWGSPTPPPSPAAPPPPPPPGAPGGSQ
DTCTSCGGFRRPEELGRVDGDFLEAVKRHILSRLQMRGRPNITHAVPKAAMVTALRKL
HAGKVREDGRVEIPHLDGHASPGSDGQERVSEIISFAETDGLASSRVRLYFFVSNEGNQN
LFVVQASLWLYLKLLPYVLEKGSRRKVRVKVYFQEQGQGDRWNAVEKKVDLKRSGW
HTFPLTEAIQALFERGERRLSLDVQCDGCQELAVVPVFVDPGEESHRPFVVVQARLGDS
RHRIRKR
Dog Activin-B mature protein:
SEQ ID NO: 65
GLECDGRTNLCCRQQFFIDFRLIGWNDWIIAPTGYYGNYCEGSCPAYLAGVP
GSASSFHTAVVNQYRMRGLNPGTVNSCCIPTKLSTMSMLYFDDEYNIVKRDVPNMIVEE
CGCA
A cat Activin-B polypeptide sequence is provided herein
SEQ ID NO: 66
MDGLPGRALGAACLLLLAAGWLGPEAWGSPTPPPSPAAPPPPPPPGAPGGSQ
DTCTSCGGFRRPEELGRVDGDFLEAVKRHILSRLQMRGRPNITHAVPKAAMVTALRKL
HAGKVREDGRVEIPHLDGHASPGADGQERVSEIISFAETDGLASSRVRLYFFISNEGNQN
LFVVQASLWLYLKLLPYVLEKGSRRKVRVKVYFQEQGHGDRWNVVEKKVDLKRSGW
HTFPLTEAIQSLFERGERRLNLDVQCDGCQELAVVPVFVDPGEESHRPFVVVQARLGDS
RHRIRKRGLECDGRTNLCCRQQFFIDFRLIGWNDWIIAPTGYYGNYCEGSCPAYLAGVP
GSASSFHTAVVNQYRMRGLNPGTVNSCCIPTKLSTMSMLYFDDEYNIVKRDVPNMIVEE
CGCA
Cat Activin-B signal peptide:
SEQ ID NO: 67
MDGLPGRALGAACLLLLAAGWLGPEAWGSPTPPPSPAAPPPPPPPGAPGGSQ
DTCTSCGGFRRPEELGRVDGDFLEAVKRHILSRLQMRGRPNITHAVPKAAMVTALRKL
HAGKVREDGRVEIPHLDGHASPGADGQERVSEIISFAETDGLASSRVRLYFFISNEGNQN
LFVVQASLWLYLKLLPYVLEKGSRRKVRVKVYFQEQGHGDRWNVVEKKVDLKRSGW
HTFPLTEAIQSLFERGERRLNLDVQCDGCQELAVVPVFVDPGEESHRPFVVVQARLGDS
RHRIRKR
Cat Activin-B mature protein:
SEQ ID NO: 68
GLECDGRTNLCCRQQFFIDFRLIGWNDWIIAPTGYYGNYCEGSCPAYLAGVP
GSASSFHTAVVNQYRMRGLNPGTVNSCCIPTKLSTMSMLYFDDEYNIVKRDVPNMIVEE
CGCA

### Activin A/B Antibody Agent

Disclosed herein are Activin A/B antibody agents which bind, e.g., specifically bind, to Activin-A and/or Activin-B, e.g., with high affinity. An anti-Activin A/B antibody disclosed herein, may be effective in the plasma and/or multiple tissue compartments, where Activin-A and/or Activin-B can act on its target cells, e.g., a cell expressing a receptor for Activin-A and/or Activin-B.

In some embodiments, an Activin-A and/or Activin-B target cell is or comprises a cell expressing an Activin receptor (e.g., a Type II receptor [e.g., ACTRIIA or ACTRIIB] and/or a Type I receptor [e.g., ActRI, ALK4, or ALK7]). In some embodiments, an Activin A/B antibody agent can modulate an Activin receptor pathway to inhibit one or more activities of Activin-A and/or Activin-B or to reduce a level of Activin-A and/or Activin-B, e.g., reduce a level of free and/or active Activin-A and/or Activin-B, e.g., in blood, plasma, serum and/or urine. In some embodiments, Activin A/B antibody agents disclosed herein binds to Activin-A, Activin-B, and/or to one or more complexes that include one or both Activin-A and Activin-B and inhibits an activity and/or reduces a level of Activin-A and/or Activin-B, e.g., reduce a level of free and/or active Activin-A and/or Activin-B, e.g., in blood, plasma, serum and/or urine. In some embodiments, Activin A/B antibody agents disclosed herein bind to Activin-A or Activin-B homodimers and inhibits an activity and/or reduces a level of Activin-A or Activin-B, e.g., reduce a level of free and/or active Activin-A or Activin-B, e.g., in blood, plasma, serum and/or urine. Without wishing to be bound by any particular theory, the present disclosure proposes that an Activin A/B antibody agents disclosed herein binds to Activin-AB heterodimers and inhibits an activity and/or reduces a level of Activin-A and/or Activin-B, e.g., reduce a level of free and/or active Activin-A and/or Activin-B, e.g., in blood, plasma, serum and/or urine

In some embodiments, an Activin A/B antibody agent disclosed herein binds to Activin-A, Activin-B, and/or to one or more complexes that include one or both Activin-A and Activin-B and prevents binding of Activin-A and/or Activin-B to an Activin receptor (e.g., a Type II receptor [e.g., ACTRIIA or ACTRIIB] and/or a Type I receptor [e.g., ActRI, ALK4, or ALK7]). In some embodiments, binding of an Activin-A and/or Activin-B antibody agent to Activin-A and/or Activin-B prevents activation of an Activin receptor (e.g., a Type II receptor [e.g., ACTRIIA or ACTRIIB] and/or a Type I receptor [e.g., ActRI, ALK4, ALK7]).

In some embodiments, an Activin A/B antibody agent disclosed herein binds to Activin-A, Activin-B, and/or to one or more complexes that include one or both Activin-A and Activin-B and prevents binding of Activin-A and/or Activin-B to a BMP receptor.

In some embodiments, an Activin A/B antibody agent disclosed herein binds to Activin-A, Activin-B, and/or to one or more complexes that include one or both Activin-A and Activin-B and prevents binding of Activin-A and/or Activin-B to a TGF-beta receptor (e.g., a Type I receptor, a Type II receptor or both).

In some embodiments, an Activin-A and/or Activin-B target cell is or comprises a cell expressing an Activin receptor. In some embodiments, an Activin-A and/or Activin-B antibody agent disclosed herein binds to Activin-A, Activin-B, and/or to one or more complexes that include one or both Activin-A and Activin-B and inhibits an activity and/or reduces a level of Activin-A and/or Activin-B, e.g., reduce a level of free and/or active Activin A. In some embodiments, an Activin A/B antibody agent disclosed herein binds to Activin-A, Activin-B, and/or to one or more complexes that include one or both Activin-A andr Activin-B and prevents binding of Activin-A and/or Activin-B to an Activin receptor (e.g., a Type II receptor [e.g., ACTRIIA or ACTRIIB] and/or a Type I receptor [e.g., ActRIALK4; Act1ALK7]).

In some embodiments, an Activin A/B antibody agent disclosed herein binds to any or all forms of Activin-A and/or Activin-B, e.g., heterodimeric Activin-AB, homodimeric Activin-A or Activin-B, monomeric Activin-A or Activin-B, intracellular Activin-A and/or Activin-B, soluble Activin-A and/or Activin-B, ECM-bound Activin-A and/or Activin-B, mature Activin-A and/or Activin-B, pro-protein Activin-A and/or Activin-B (e.g., preprocessed) and/or active Activin-A and/or Activin-B.

In some embodiments, a level of Activin-A and/or Activin-B (e.g., free and/or active Activin-A and/or Activin-B) is reduced relative to a comparator. In some embodiments, a comparator comprises a cell, tissue or subject which has not been contacted with an Activin-A and/or Activin-B antibody agent disclosed herein. In some embodiments, a level of Activin-A and/or Activin-B (e.g., free and/or active Activin-A and/or Activin-B) is reduced in blood, plasma, serum, tumor and/or urine.

In some embodiments, a level of Activin-A and/or Activin-B (e.g., free and/or active Activin-A and/or Activin-B) is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% or about 100%. In some embodiments, a level of Activin-A and/or Activin-B (e.g., free and/or active Activin-A and/or Activin-B) is reduced by about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 90% to about 100%, or about 95% to about 100%.

In some embodiments, a level of Activin-A and/or Activin-B (e.g., free and/or active Activin-A and/or Activin-B) is reduced by about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, or about 5% to about 10%.

In some embodiments, an Activin-A and/or Activin-B activity comprises an activity of an Activin receptor. In some embodiments, an Activin-A and/or Activin-B activity comprises an activity of one or more signaling pathways activated by an Activin receptor. In some embodiments, an Activin receptor mediated signaling pathway comprises one or more of: (i) a SMAD pathway, (ii) an ERK/MAPK pathway, (iii) a p38 MAPK pathway, (iv) NOX-2 dependent signaling; (v) STAT3 signaling, or (vi) Snail and/or Slug mediated pathways.

Disclosed herein are Activin A/B antibody agents that can modulate an activity of Activin-A and/or Activin-B In some embodiments, an Activin-A and/or Activin-B antibody agent specifically binds Activin-A and/or Activin-B and modulates one or more, or all, or any combination of detectable Activin-A and/or Activin-B activities such that the antibody agent: modulates an Activin receptor activity, modulates a SMAD pathway, modulates an ERK/MAPK pathway, modulates a p38 MAPK pathway, modulates NOX-2 dependent signaling, modulates Snail and/or Slug mediated pathways, modulates a STAT3 pathway, or a combination thereof.

In some embodiments, an Activin-A and/or Activin-B antibody agent specifically binds Activin-A and/or Activin-B such that the antibody agent inhibits Activin A and/or B signaling, e.g., SMAD. In some embodiments, SMAD comprises a SMAD1, SMAD3, SMAD5 receptor or combinations thereof.

In some embodiments, an Activin-A and/or Activin-B antibody agent specifically binds Activin-A and/or Activin-B such that the antibody agent inhibits signaling of Activin A and/or B via SMAD 2/3 signaling.

One with skill in the art will appreciate that, in some embodiments, an activity of Activin-A and/or Activin-B, e.g., as described herein can be assessed using one or more art recognized assays. For example, as disclosed in Example 2 herein, an Activin 2B Receptor/SMAD reporter assay can be used to evaluate Activin A activity.

Human Activin A protein sequence is highly conserved with primate, rodents, domestic mammals, birds, and other species such as panda, seal, sloth and whale. Table 15 provides a summary of the sequence homology between human Activin A and Activin A in various species. The list provided in Table 15 is an exemplary list of species having highly conserved Activin A as compared to human Activin A. Many other animals and birds, in addition to those disclosed in Table 15 have Activin A protein sequences that are highly homologous (e.g., >95%) to human Activin A. One with skill in the art will understand in reading this disclosure that known methods to determine sequence homology can be used to identify Activin A from other species which share a high sequence homology with human Activin A, e.g., as described herein.

Activin B protein sequence is highly conserved with primate, rodents, domestic mammals, birds, and other species such as panda, seal, sloth and whale. Table 16 provides a summary of the sequence homology between human Activin B and Activin B in various species. The list provided in Table 16 is an exemplary list of species having highly conserved Activin B as compared to human Activin B. Many other animals and birds, in addition to those disclosed in Table 16 have Activin B protein sequences that are highly homologous (e.g., >95%) to human Activin B. One with skill in the art will understand in reading this disclosure that known methods to determine sequence homology can be used to identify Activin B from other species which share a high sequence homology with human Activin B, e.g., as described herein.

TABLE 15
Activin A homology across various species

| | | Sequence | Homology | | |
|---|---|---|---|---|---|
| | Identifier | Length | to Human | Species | |
| | XP_016867664.1 | 116 | | Homo sapiens | Human |
| Primates | XP_519063.2 | 116 | 100% | Pan troglodytes | Chimpanzee |
| and | XP_003808743.1 | 116 | 100% | Pan paniscus | Chimpanzee, Pygmy |
| Monkeys | XP_028701686.1 | 116 | 100% | Macaca mulatta | Rhesus macaque |
| | XP_011760680.1 | 116 | 100% | Macaca nemestrina | Southern pig-tailed macaque |
| | XP_002751400.2 | 116 | 100% | Callithrix jacchus | Marmoset monkey |
| | XP_017369490.1 | 116 | 100% | Cebus imitator | Panamanian White-faced Capuchin |
| | XP_007979729.1 | 116 | 100% | Chlorocebus sabaeus | green monkey |
| Rodents | NP_058824.1 | 116 | 100% | Rattus norvegicus | Rat |
| | CAJ18587.1 | 116 | 100% | Mus musculus | Mouse |
| | XP_012970113.1 | 116 | 100% | Mesocricetus auratus | Golden hamster |
| | NP_001315998.1 | 116 | 100% | Oryctolagus cuniculus | Rabbit |
| | XP_013008060.1 | 116 | 100% | Cavia porcellus | Guinea pig |
| | XP_005397780.1 | 116 | 100% | Chinchilla lanigera | Chinchilla |
| Domestic | NP_001009856.1 | 116 | 100% | Felis catus | Cat |
| Mammals | XP_038279632.1 | 116 | 97% | Canis lupus familiaris | Dog |
| | XP_004739411.1 | 116 | 100% | Mustela putorius furo | Ferret |
| | NP_001075378.1 | 116 | 97% | Equus caballus | Horse |
| | AAA97415.1 | 116 | 100% | Bos taurus | Cow |
| | KAG5210458.1 | 116 | 100% | Ovis aries | Sheep |
| | NP_999193.1 | 116 | 100% | Sus scrofa | Pig |
| | XP_010945482.1 | 116 | 100% | Camelus bactrianus | Bactrian camel |
| | MXQ80611.1 | 116 | 100% | Bos mutus | Yak |
| Birds | XP_015136806.2 | 116 | 98% | Gallus gallus | Chicken |
| | NWZ62453.1 | 116 | 100% | Acrocephalus arundinaceus | Great Reed Warbler |
| | NXT88066.1 | 116 | 100% | Anhinga rufa | African darter (bird) |
| Other | XP_002914183.1 | 116 | 100% | Ailuropoda melanoleuca | Giant Panda |
| (from >100) | XP_025708917.1 | 116 | 100% | Callorhinus ursinus | Northern Fur Seal |
| | XP_037693065.1 | 116 | 100% | Choloepus didactylus | Two-toed Sloth |
| | MBV94806.1 | 116 | 100% | Eschrichtius robustus | California grey whale |

TABLE 16
Activin A homology across various species

| | | Sequence | Homology | | |
|---|---|---|---|---|---|
| | Identifier | Length | to Human | Species | |
| | | | | Homo sapiens | Human |
| Primates | XP_016805107.1 | 115 | 100% | Pan troglodytes | Chimpanzee |
| and | XP_003808743.1 | 115 | 100% | Pan paniscus | Chimpanzee, Pygmy |
| Monkeys | XP_001090729.4 | 115 | 99% | Macaca mulatta | Rhesus macaque |
| | XP_005573115.2 | 115 | 99% | Macaca fascicularis | Philippine long-tailed macaque |
| | XP_035160995.1 | 115 | 100% | Callithrix jacchus | Marmoset monkey |
| | XP_017355757.1 | 115 | 100% | Cebus imitator | Panamanian White-faced Capuchin |
| | XP_011920436.1 | 115 | 99% | Cercocebus atys | Sooty mangabey (monkey) |
| | XP_007962886.1 | 115 | 99% | Chlorocebus sabaeus | green monkey |
| Rodents | NP 542949.1 | 115 | 97% | Rattus norvegicus | Rat |
| | NP_032407.1 | 115 | 97% | Mus musculus | Mouse |
| | XP_005070349.1 | 115 | 97% | Mesocricetus auratus | Golden hamster |
| | XP_008256688.1 | 115 | 97% | Oryctolagus cuniculus | Rabbit |
| | XP_003478664.1 | 115 | 98% | Cavia porcellus | Guinea pig |
| | XP_013376547.1 | 115 | 98% | Chinchilla lanigera | Chinchilla |
| Dogs | XP_003990762.4 | 115 | 100% | Felis catus | Cat |
| and | XP_038281949.1 | 115 | 99% | Canis lupus familiaris | Dog |
| Cats | XP_025284868.1 | 115 | 100% | Canis lupus dingo | Dingo |
| | XP_039090823.1 | 115 | 100% | Hyaena hyaena | Hyaena |
| Domestic | XP_027820847.1 | 115 | 99% | Ovis aries | Sheep |
| Animals | XP_010952573.1 | 115 | 100% | Camelus bactrianus | Bactrian camel |
| | CAA27021.1 | 115 | 99% | Sus scrofa | Pig |
| | ELR51808.1 | 115 | 100% | Bos mutus | Yak |
| Birds | NP_990537.2 | 115 | 98% | Gallus gallus | Chicken |
| | NWZ61839.1 | 115 | 98% | Acrocephalus arundinaceus | Great Reed Warbler |
| | NXT89562.1 | 115 | 98% | Anhinga rufa | African darter (bird) |
| Other | EFB14628.1 | 115 | 100% | Ailuropoda melanoleuca | Giant Panda |
| (from >100) | XP_036714727.1 | 115 | 100% | Balaenoptera physalus | Fin Whale |
| | XP_025710928.1 | 115 | 100% | Callorhinus ursinus | Northern Fur Seal |
| | XP_037705870.1 | 115 | 97% | Choloepus didactylus | Two-toed Sloth |
| | XP_004276946.1 | 115 | 100% | Orcinus orca | Orca |

Based on the high homology between human Activin A and Activin A from other species as shown in Table 15, one with skill in the art would understand that an Activin A/B antibody agent disclosed herein would bind to Activin A from one or more species provided in Table 15.

Based on the high homology between human Activin B and Activin B from other species as shown in Table 16, one with skill in the art would understand that an Activin A/B antibody agent disclosed herein would bind to Activin B from one or more species provided in Table 16.

Based on the high homology between human Activin A and Activin A from other species as shown in Table 15, and the high homology between human Activin B and Activin B from other species as shown in Table 16, one with skill in the art would understand that an Activin A/B antibody agent disclosed herein would bind to Activin A from one or more species provided in Table 15 and to Activin B from the same species.

In some embodiments, an Activin-A and/or Activin-B antibody agent disclosed herein binds specifically to human Activin-A and/or Activin-B. In some embodiments, an Activin-A and/or Activin-B antibody agent disclosed herein binds specifically to primate Activin-A and/or Activin-B. In some embodiments, an Activin A/B antibody agent disclosed herein binds specifically to domestic mammals Activin A. In some embodiments, domestic mammals are chosen from: dog, cat, ferret, horse, cow, sheep, pig, Bactrian camel, and yak.

In some embodiments, a provided Activin A/B antibody agent may show preferential binding to Activin-A and/or Activin-B relative to one or more TGFbeta family members other than Activin-A and/or Activin-B. In some such embodiments, preferential binding may be assessed, for example, by simultaneously contacting an Activin A/B antibody agent with Activin-A and/or Activin-B and one or more other TGFbeta family members. Alternatively or additionally, in some embodiments, preferential binding may be assessed relative to an appropriate reference Activin A antibody agent (e.g., as described in WO2015017576) and, e.g., may reflect a higher level of binding to Activin A relative to the one or more other TGFbeta family member than is observed with the reference antibody.

In some embodiments, an Activin A/B antibody agent disclosed herein preferentially binds to Activin-A, Activin-B, and/or to one or more complexes that include one or both Activin-A and Activin-B. In some embodiments, an Activin A/B antibody agent disclosed herein does not bind to one or more members of the TGFbeta super family other than Activin-A and/or Activin-B. In some embodiments, an Activin A/B antibody agent disclosed herein does not bind to GDNF, GDF8, GDF10, GDF11, GDF15, BMP9, BMP 10 or a combination thereof.

In some embodiments, an Activin A/B antibody agent disclosed herein preferentially binds to Activin-A, Activin-B, and/or to one or more complexes that include one or both Activin-A and Activin-B. In some embodiments, an Activin A/B antibody agent disclosed herein binds to one or more members of the TGFbeta super family in addition to Activin-A and/or Activin-B. In some embodiments, an Activin A/B antibody agent disclosed herein binds to Activin-A, Activin-B, and/or to one or more complexes that include one or both Activin-A and Activin-B, and GDF10 or GDF11, or both. In some embodiments, an Activin A/B antibody agent disclosed herein binds to Activin-A, Activin-B, and/or to one or more complexes that include one or both Activin-A and Activin-B. In some embodiments, an Activin A/B antibody agent disclosed herein binds to Activin A, Activin B, GDF10 and GDF11.

In some embodiments, an Activin A/B binding agent which binds to Activin-A and/or Activin-B and GDF11 does not modulate an activity and/or level of GDF11, e.g., when characterized in an assay that evaluates a GDF11 activity and/or level.

One with skill in the art will appreciate that, in some embodiments, an activity of Activin A, Activin B, or GDF11, e.g., as described herein can be assessed using one or more art recognized assays. For example, as disclosed in Example 2 herein, an assay using cells expressing an Activin 2B Receptor/SMAD reporter can be used to evaluate Activin B, or GDF11 activity. Several assays can be used to measure activation of the Activin 2B Receptor and induction of SMAD signaling following stimulation with Activin B, or GDF11, e.g., a luciferase-based reporter system.

Without wishing to be bound by theory, even though provided Activin A/B antibody agents can bind to Activin-A and/or Activin-B and one or more members of the TGFbeta super family, a modulatory impact of provided Activin A/B antibody agents towards Activin-A and/or Activin-B may be independent of binding to one or more TGFbeta super family members.

Those skilled in the art, reading the present disclosure will appreciate that, in some embodiments, an antibody agent provided by the present disclosure comprises: (i) an intact IgA, IgG, IgD, IgE or IgM antibody; (ii) an antibody fragment (e.g., an antibody variable region, containing both heavy and light chain sequences, e.g., a Fab); (iii) a single domain antibody (e.g., a light chain antibody or a heavy chain antibody); (iv) a single chain antibody (e.g., a single chain Fv, a camelid antibody, etc); (v) an antibody-drug conjugate; (vi) a bi- or other multispecific antibody; (vii) a polypeptide comprising antigen binding specificity fused to an Fc region; etc.

Those skilled in the art, reading the present disclosure will further appreciate that provided contributions are not limited to intact antibodies or fragments thereof (e.g., including both heavy and light chain sequences).

For example, those skilled in the art will appreciate that individual light chains and/or individual heavy chains, or variable region sequences thereof, as described herein (e.g., as exemplified herein, for example as presented in Table 1) may be useful in combination with other light chains and/or heavy chains. In some embodiments, a single light chain described herein (or variable region sequences thereof) may be utilized together with two (or more) different heavy chains (e.g., which may be or comprise heavy chains exemplified herein), or variable region sequences thereof, in a “common light chain” bispecific format. In some embodiments, exemplified light and heavy chains (e.g., variable region sequences thereof) may be “mixed and matched” with one another in antibody agents provided by the present disclosure (e.g., antibody agents that specifically bind to Activin-A and/or Activin-B and/or have one or more other structural and/or functional properties as described herein.

Still further, those skilled in the art will appreciate that the present disclosure provides useful heavy and light chain antibody sequences, specifically including useful variable region sequences, including for example useful CDR and/or framework (FR) sequences.

In some embodiments, the present disclosure provides polypeptides (which may, for example, be, or be included in, an antibody agent that binds specifically to Activin-A and/or Activin-B) including one or more CDR and/or FR sequences as set forth in Table 1 or 2. In some embodiments, the present disclosure provides polypeptides including two or more CDR elements from Table 1 or 2, and in particular the present disclosure provides polypeptides including three or six CDR elements from Table 1 or 2.

In some embodiments, the present disclosure provides polypeptides (which may, for example, be, or be included in, an antibody agent that binds specifically to Activin-A and/or Activin-B) including one LC CDR1, one LC CDR2, and one LC CDR3 from Table 1; in some such embodiments, two or three of the CDRs are from the same LC in Table 1.

In some embodiments, the present disclosure provides polypeptides (which may, for example, be, or be included in, an antibody agent that binds specifically to Activin-A and/or Activin-B) including one HC CDR1, one HC CDR2, and one HC CDR3 from Table 2; in some such embodiments, two or three of the CDRs are from the same HC in Table 2.

In some embodiments, the present disclosure provides polypeptides (which may, for example, be, or be included in, an antibody agent that binds specifically to Activin-A and/or Activin-B) including one each of a LC CDR1, a LC CDR2, a LC CDR3, a HC CDR1, a HC CDR2, and a HC CDR3 from Table 1 or 2; in some such embodiments, two or more CDRs, and in some embodiments all LC CDRs, all HC CDRS, or both, are from the same antibody in Table 1 or 2.

Those skilled in the art will further appreciate that, in some embodiments, useful polypeptides as described herein that include one or more CDRs from Table 1 or 2 may include a heavy or light chain CDR set (i.e., each of a CDR1, a CDR2, and a CDR3) that includes one or two CDRs from a first antibody chain (i.e., LC or HC) in Table 1 or 2, and at least one from a second antibody chain (e.g., of the same type) in Table 1 or 2. Alternatively or additionally, in some embodiments, those skilled in the art will appreciate that useful polypeptides as described herein that include one or more CDRs from Table 1 or 2 may include a heavy or light chain CDR set (i.e., each of a CDR1, a CDR2, and a CDR3) that includes at least one CDR from a first antibody chain (i.e., LC or HC) in Table 1 or 2 and at least one other CDR that differs from its corresponding CDR in the relevant chain in Table 1 or 2. In some such embodiments, the differing CDR(s) will differ at not more than 3, not more than 2, or not more than 1 positions relative to the corresponding CDR(s); alternatively or additionally, in some such embodiments, the differing CDR(s) will differ only at terminal residue(s) relative to the corresponding CDR(s).

In some embodiments, an Activin A/B antibody agent disclosed herein which binds to Activin-A, Activin-B, and/or to one or more complexes that include one or both Activin-A and Activin-B comprises a LC CDR1, a LC CDR2 and a LC CDR3 provided in Table 1. In some embodiments, the presence of a LC CDR1, a LC CDR2 and a LC CDR3 is sufficient to confer binding and/or is otherwise useful in an antibody agent disclosed herein (i.e., that specifically Activin-A, Activin-B, and/or to one or more complexes that include one or both Activin-A and Activin-B). In some embodiments, an antibody agent comprising a LC CDR1, a LC CDR2 and a LC CDR3 can be in any format disclosed herein. For example, in some embodiments, an antibody agent comprising a LC CDR1, a LC CDR2 and a LC CDR3 can be a single chain antibody, and is capable of binding Activin-A and/or Activin-B.

In some embodiments, an Activin A/B antibody agent disclosed herein which binds to Activin-A, Activin-B, and/or to one or more complexes that include one or both Activin-A and Activin-B comprises a HC CDR1, a HC CDR2 and a HC CDR3 is sufficient to confer binding and/or is otherwise useful in an antibody agent disclosed herein to Activin-A and/or Activin-B. In some embodiments, an antibody agent comprising a HC CDR1, a HC CDR2 and a HC CDR3 can be in any format disclosed herein. For example, in some embodiments, an antibody agent comprising a HC CDR1, a HC CDR2 and a HC CDR3 can be a single chain antibody, and is capable of binding Activin-A and/or Activin-B.

In some embodiments, an Activin A/B antibody agent disclosed herein which binds to Activin-A, Activin-B, and/or to one or more complexes that include one or both Activin-A and Activin-B comprises a set of any three LC CDRs (e.g., LC CDR1, LC CDR2 and LC CDR3) provided in Table 1, and a set of any three HC CDRs (e.g., HC CDR1, HC CDR2 and HC CDR3) provided in Table 2. In some embodiments, the presence of a set of any three LC CDRs and a set of any three HC CDRs is sufficient to confer binding of any antibody agent disclosed herein to Activin-A and/or Activin-B. In some embodiments, such an Activin A/B antibody agent can be a fragment (e.g., an scFv, a Fab or other fragments), or an intact antibody, or a polypeptide comprising antigen binding specificity fused to an Fc.

In some embodiments, disclosed herein is an Activin A/B antibody agent that competes (e.g., when tested in a standard competition assay) for binding to human Activin-A and/or Activin-B with a different Activin A antibody agent, e.g., an Activin A antibody agent disclosed in WO2015017576. In some embodiments, an Activin A/B antibody agent disclosed herein competes for binding to human Activin-A and/or Activin-B with a different Activin antibody agent when assessed at more than one concentration (e.g., over a concentration range of at least 2-, 4-, 6-, 8-, 10-fold or more).

In some embodiments, disclosed herein is an Activin A/B antibody agent that does not compete (e.g., when tested in a standard competition assay) for binding to human Activin-A and/or Activin-B with a different Activin A antibody agent, e.g., an Activin A antibody agent disclosed in WO2015017576.

In some embodiments, disclosed herein is an Activin A/B antibody agent that binds to a sterically overlapping (e.g., partially or completely overlapping) epitope as an Activin A antibody agent disclosed in WO2015017576.

### Light Chain (e.g., Light Chain Variable Region) Polypeptides (LC Polypeptides)

The present disclosure provides polypeptides comprising light chain (LC) sequences (e.g., light chain variable region sequence(s)) that, for example, may be useful in antibody agents as described herein targeting Activin-A and/or Activin-B; in some such embodiments, such provided polypeptides are useful and/or included in such antibody agents as described herein. In some embodiments, a LC polypeptide comprises at least one LC CDR provided in Table 1 or a sequence with at least 85% identity thereto. In some embodiments, a LC polypeptide comprises one, two or three LC CDRs (e.g., a LC CDR1, a LC CDR2 and/or a LC CDR3). In some embodiments, a LC polypeptide comprises a LC CDR1. In some embodiments, a LC polypeptide comprises a LC CDR2. In some embodiments, a LC polypeptide comprises a LC CDR3. In some embodiments, a LC polypeptide comprises a LC CDR1, a LC CDR2 and a LC CDR3.

In some embodiments, a LC polypeptide having a LC CDR1, a LC CDR2 and a LC CDR3, e.g., in an Activin A/B antibody agent, is capable of binding (e.g., specifically binding) to Activin-A and/or Activin-B

In some embodiments, a LC polypeptide further comprises one or more framework regions, and/or a constant region.

In some embodiments, a LC polypeptide comprises a light chain constant region and/or a heavy chain constant region. In some embodiments, a LC polypeptide comprises a light chain constant region or a portion thereof, (e.g., a lambda light chain constant region or a variant or portion thereof; or a kappa light chain constant region or a variant or a portion thereof).

In some embodiments, a light chain kappa constant region comprises the sequence of SEQ ID NO: 19, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 19.

(SEQ ID NO: 19, light chain kappa)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

In some embodiments, a LC polypeptide comprises the sequence of SEQ ID NO: 19, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 19.

In some embodiments, a LC polypeptide disclosed herein further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, a LC polypeptide comprises: (i) an LC CDR1 sequence provided in Table 1, e.g., any one of SEQ ID NOs: 1 or 11; (ii) a sequence with at least 85%, 86%, 87%, 88%, 89%, 90%, 91%, 92%, 93%, 94%, 95%, 96%, 97%, 98%, 99% identity to an LC CDR1 sequence provided in Table 1, e.g., any one of SEQ ID NOs: 1 or 11; or (iii) a sequence having at least 5, 10, or 20 substitutions relative to an LC CDR1 sequence provided in Table 1, e.g., any one of SEQ ID NOs: 1 or 11.

In some embodiments, a LC polypeptide comprises: (i) an LC CDR2 sequence provided in Table 1, e.g., any one of SEQ ID NOs: 2 or 12; (ii) a sequence with at least 85%, 86%, 87%, 88%, 89%, 90%, 91%, 92%, 93%, 94%, 95%, 96%, 97%, 98%, 99% identity to an LC CDR2 sequence provided in Table 1 e.g., any one of SEQ ID NOs: 2 or 12; or (iii) a sequence having at least 5, 10, or 20 substitutions relative to an LC CDR2 sequence provided in Table 1, e.g., any one of SEQ ID NOs: 2 or 12.

In some embodiments, a LC polypeptide comprises: (i) an LC CDR3 sequence provided in Table 1, e.g., any one of SEQ ID NOs: 3 or 13; (ii) a sequence with at least 85%, 86%, 87%, 88%, 89%, 90%, 91%, 92%, 93%, 94%, 95%, 96%, 97%, 98%, 99% identity to an LC CDR3 sequence provided in Table 1, e.g., any one of SEQ ID NOs: 3 or 13; or (iii) a sequence having at least 5, 10, or 20 substitutions relative to an LC CDR3 sequence provided in Table 1, e.g., any one of SEQ ID NOs: 3 or 13.

In some embodiments, an antibody agent comprising a LC CDR1, LC CDR2 and/or LC CDR3 is capable of binding specifically to Activin-A and/or Activin-B.

In some embodiments, a LC polypeptide comprises (i) an LC CDR1, LC CDR2, and LC CDR3 sequence provided in Table 1; (ii) a sequence with at least 85%, 86%, 87%, 88%, 89%, 90%, 91%, 92%, 93%, 94%, 95%, 96%, 97%, 98%, 99% identity to an LC CDR1, LC CDR2, and LC CDR3 sequence provided in Table 1; or (iii) a sequence having at least 5, 10, or 20 substitutions relative to an LC CDR1, LC CDR2, and LC CDR3 sequence provided in Table 1.

In some embodiments, a LC polypeptide comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 1; (ii) an LC CDR2 of SEQ ID NO: 2, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 2; and/or (iii) an LC CDR3 of SEQ ID NO: 3, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 3.

In some embodiments, a LC polypeptide comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 11; (ii) an LC CDR2 of SEQ ID NO: 12, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 12; and/or (iii) an LC CDR3 of SEQ ID NO: 13, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 13.

In some embodiments, a LC polypeptide further comprises one or more framework regions (FR), e.g., as described herein. In some embodiments, a LC polypeptide comprises one, two, three or four FRs, e.g., as described herein. In some embodiments, a FR comprises a LC FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a LC FR sequence as described herein, or a sequence having at least 5, 10 or 20 alterations relative to a LC FR sequence as described herein.

In some embodiments, a LC polypeptide comprises: (i) a FR sequence provided in Table 1; (ii) a sequence with at least 92%, 93%, 94%, 95%, 96%, 97%, 98%, or 99% identity to a FR sequence provided in Table 1; or (iii) a sequence having at least 5, 10, or 20 alterations (e.g., substitutions, deletions or insertions (e.g., conservative substitutions)) compared to a FR sequence provided in Table 1.

In some embodiments, a LC polypeptide comprises a LC FR1 provided in Table 1, a sequence with at least 92%, 93%, 94%, 95%, 96%, 97%, 98%, or 99% identity to a LC FR1 sequence provided in Table 1, or a sequence having at least 5, 10, or 20 alterations (e.g., substitutions, deletions or insertions (e.g., conservative substitutions) compared to a LC FR1 sequence provided in Table 1.

In some embodiments, a LC polypeptide comprises a LC FR2 provided in Table 1, a sequence with at least 92%, 93%, 94%, 95%, 96%, 97%, 98%, or 99% identity to a LC FR2 sequence provided in Table 1, or a sequence having at least 5, 10, or 20 alterations (e.g., substitutions, deletions or insertions (e.g., conservative substitutions) compared to a LC FR2 sequence provided in Table 1.

In some embodiments a LC polypeptide comprises a LC FR3 provided in Table 1, a sequence with at least 92%, 93%, 94%, 95%, 96%, 97%, 98%, or 99% identity to a LC FR3 sequence provided in Table 1, or a sequence having at least 5, 10, or 20 alterations (e.g., substitutions, deletions or insertions (e.g., conservative substitutions) compared to a LC FR3 sequence provided in Table 1.

In some embodiments, a LC polypeptide comprises a LC FR4 provided in Table 1, a sequence with at least 92%, 93%, 94%, 95%, 96%, 97%, 98%, or 99% identity to a LC FR4 sequence provided in Table 1, or a sequence having at least 5, 10, or 20 alterations (e.g., substitutions, deletions or insertions (e.g., conservative substitutions) compared to a LC FR4 sequence provided in Table 1.

In some embodiments, a LC polypeptide comprises a LC CDR1, a LC CDR2 and LC CDR3 provided in Table 1 or a sequence with at least 85% identity thereto, and a LC FR1, LC FR2, LC FR3 and a LC FR4 of a provided in Table 1 or a sequence with at least 92% identity thereto.

In some embodiments, a LC polypeptide comprises the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8.

In some embodiments, a LC polypeptide comprises the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16.

In some embodiments, a LC polypeptide comprises an LC sequence provided in Table 1, e.g., any one of SEQ ID NOs: 9 or 17, or a sequence with at least 85%, 86%, 87%, 88%, 89%, 90%, 91%, 92%, 93%, 94%, 95%, 96%, 97%, 98%, 99% identity thereto; or a sequence having at least 5, 10, or 20 alterations (e.g., substitutions, deletions or insertions (e.g., conservative substitutions)) relative to any one of SEQ ID NOs: 9 or 17.

Exemplary useful LC polypeptides which may be included in Activin A/B antibody agents disclosed herein are disclosed in Table 1 below.

TABLE 1
Exemplary light chain sequences for Activin A/B antibody agents

| CLONE | SEQ ID NO | Feature | SEQUENCE |
|---|---|---|---|
| A | 1 | LC CDR1 | RASQSVGSNLA |
| | 2 | LC CDR2 | GASTRAT |
| | 3 | LC CDR3 | QQYNFYPPIT |
| | 4 | LC FR1 | EIVLTQSPATLSVSPGERATLSC |
| | 5 | LC FR2 | WYQQKPGQAPRLLIY |
| | 6 | LC FR3 | GIPARFSGSGSGTEFTLTISSLQSEDFAVYYC |
| | 7 | LC FR4 | FGGGTKVEIK |
| | 8 | VL amino | EIVLTQSPATLSVSPGERATLSCRASQSVGSNLAWYQQ |
| | | acid | KPGQAPRLLIYGASTRATGIPARFSGSGSGTEFTLTISSL |
| | | | QSEDFAVYYCQQYNFYPPITFGGGTKVEIK |
| | 9 | LC | EIVLTQSPATLSVSPGERATLSCRASQSVGSNLAWYQQ |
| | | | KPGQAPRLLIYGASTRATGIPARFSGSGSGTEFTLTISSL |
| | | | QSEDFAVYYCQQYNFYPPITFGGGTKVEIK |
| | | | RTVAAPSVFIFPPSDEQLKSGTASVVCLLNNFYPREAK |
| | | | VQWKVDNALQSGNSQESVTEQDSKDSTYSLSSTLTLS |
| | | | KADYEKHKVYACEVTHQGLSSPVTKSFNRGEC |
| | 10 | VL DNA | GAAATAGTGTTGACGCAGTCTCCAGCCACCCTGTCT |
| | | | GTGTCTCCAGGGGAAAGAGCCACCCTCTCCTGCAGG |
| | | | GCCAGTCAGAGTGTTGGCAGCAACTTAGCCTGGTAC |
| | | | CAGCAGAAACCTGGCCAGGCTCCCAGGCTCCTCATC |
| | | | TATGGTGCATCCACCAGGGCCACTGGTATCCCAGCC |
| | | | AGGTTCAGTGGCAGTGGGTCTGGGACAGAGTTCACT |
| | | | CTCACCATCAGCAGCCTGCAGTCTGAAGATTTTGCA |
| | | | GTTTATTACTGTCAGCAGTACAACTTCTACCCTCCTA |
| | | | TCACTTTTGGCGGAGGGACCAAGGTTGAGATCAAA |
| B | 1 | LC CDR1 | RASQSVGSNLA |
| | 2 | LC CDR2 | GASTRAT |
| | 3 | LC CDR3 | QQYNFYPPIT |
| | 4 | LC FR1 | EIVLTQSPATLSVSPGERATLSC |
| | 5 | LC FR2 | WYQQKPGQAPRLLIY |
| | 6 | LC FR3 | GIPARFSGSGSGTEFTLTISSLQSEDFAVYYC |
| | 7 | LC FR4 | FGGGTKVEIK |
| | 8 | VL amino | EIVLTQSPATLSVSPGERATLSCRASQSVGSNLAWYQQ |
| | | acid | KPGQAPRLLIYGASTRATGIPARFSGSGSGTEFTLTISSL |
| | | | QSEDFAVYYCQQYNFYPPITFGGGTKVEIK |
| | 9 | LC | EIVLTQSPATLSVSPGERATLSCRASQSVGSNLAWYQQ |
| | | | KPGQAPRLLIYGASTRATGIPARFSGSGSGTEFTLTISSL |
| | | | QSEDFAVYYCQQYNFYPPITFGGGTKVEIK |
| | | | RTVAAPSVFIFPPSDEQLKSGTASVVCLLNNFYPREAK |
| | | | VQWKVDNALQSGNSQESVTEQDSKDSTYSLSSTLTLS |
| | | | KADYEKHKVYACEVTHQGLSSPVTKSFNRGEC |
| | 10 | VL DNA | GAAATAGTGTTGACGCAGTCTCCAGCCACCCTGTCT |
| | | | GTGTCTCCAGGGGAAAGAGCCACCCTCTCCTGCAGG |
| | | | GCCAGTCAGAGTGTTGGCAGCAACTTAGCCTGGTAC |
| | | | CAGCAGAAACCTGGCCAGGCTCCCAGGCTCCTCATC |
| | | | TATGGTGCATCCACCAGGGCCACTGGTATCCCAGCC |
| | | | AGGTTCAGTGGCAGTGGGTCTGGGACAGAGTTCACT |
| | | | CTCACCATCAGCAGCCTGCAGTCTGAAGATTTTGCA |
| | | | GTTTATTACTGTCAGCAGTACAACTTCTACCCTCCTA |
| | | | TCACTTTTGGCGGAGGGACCAAGGTTGAGATCAAA |
| C | 11 | LC CDR1 | RASQSVSSSFLA |
| | 12 | LC CDR2 | GASSRAT |
| | 13 | LC CDR3 | QQYHSSPFT |
| | 14 | LC FR1 | EIVLTQSPGTLSLSPGERATLSC |
| | 5 | LC FR2 | WYQQKPGQAPRLLIY |
| | 15 | LC FR3 | GIPDRFSGSGSGTDFTLTISRLEPEDFAVYYC |
| | 7 | LC FR4 | FGGGTKVEIK |
| | 16 | VL amino | EIVLTQSPGTLSLSPGERATLSCRASQSVSSSFLAWYQQ |
| | | acid | KPGQAPRLLIYGASSRATGIPDRFSGSGSGTDFTLTISRL |
| | | | EPEDFAVYYCQQYHSSPFTFGGGTKVEIK |
| | 17 | LC | EIVLTQSPGTLSLSPGERATLSCRASQSVSSSFLAWYQQ |
| | | | KPGQAPRLLIYGASSRATGIPDRFSGSGSGTDFTLTISRL |
| | | | EPEDFAVYYCQQYHSSPFTFGGGTKVEIK |
| | | | RTVAAPSVFIFPPSDEQLKSGTASVVCLLNNFYPREAK |
| | | | VQWKVDNALQSGNSQESVTEQDSKDSTYSLSSTLTLS |
| | | | KADYEKHKVYACEVTHQGLSSPVTKSFNRGEC |
| | 18 | VL DNA | GAAATTGTGTTGACGCAGTCTCCAGGCACCCTGTCT |
| | | | TTGTCTCCAGGGGAAAGAGCCACCCTCTCCTGCAGG |
| | | | GCCAGTCAGAGTGTTAGCAGCAGCTTCTTAGCCTGG |
| | | | TACCAGCAGAAACCTGGCCAGGCTCCCAGGCTCCTC |
| | | | ATCTATGGTGCATCCAGCAGGGCCACTGGCATCCCA |
| | | | GACAGGTTCAGTGGCAGTGGGTCTGGGACAGACTTC |
| | | | ACTCTCACCATCAGCAGACTGGAGCCTGAAGATTTT |
| | | | GCAGTGTATTACTGTCAGCAGTACCACAGCTCCCCT |
| | | | TTCACTTTTGGCGGAGGGACCAAGGTTGAGATCAAA |
| CK | 19 | | RTVAAPSVFIFPPSDEQLKSGTASVVCLLNNFYPREAK |
| | | | VQWKVDNALQSGNSQESVTEQDSKDSTYSLSSTLTLS |
| | | | KADYEKHKVYACEVTHQGLSSPVTKSFNRGEC |

### Heavy Chain (e.g., Heavy Chain Variable Region) Polypeptides (HC Polypeptides)

The present disclosure provides polypeptides comprising heavy chain (HC) sequences (e.g., heavy chain variable region sequence(s)) that, for example, may be useful in antibody agents as described herein targeting Activin-A and/or Activin-B; in some such embodiments, such provided polypeptides are useful and/or included in such antibody agents as described herein. In some embodiments, a HC polypeptide comprises at least one HC CDR of an Activin A/B antibody agent as provided in Table 2 or a sequence with at least 85% identity thereto. In some embodiments, a HC polypeptide comprises one, two or three HC CDRs (e.g., a HC CDR1, a HC CDR2 and/or a HC CDR3). In some embodiments, a HC polypeptide comprises a HC CDR1. In some embodiments, a HC polypeptide comprises a HC CDR2. In some embodiments, a HC polypeptide comprises a HC CDR3. In some embodiments, a HC polypeptide comprises a HC CDR1, a HC CDR2 and a HC CDR3.

In some embodiments, a HC polypeptide comprising a HC CDR1, a HC CDR2 and a HC CDR3 is capable of binding (e.g., specifically binding) to Activin-A and/or Activin-B.

In some embodiments, a HC polypeptide further comprises one or more framework regions, and/or a heavy chain constant region, or a portion or a variant thereof (e.g., a CH1 domain, a CH2 domain and/or a CH3 domain). In some embodiments, a HC polypeptide comprises a CH1 domain, a CH2 domain or a CH3 domain, or a combination thereof. In some embodiments, a HC polypeptide comprises a CH2 domain and a CH3 domain, e.g., an Fc domain.

In some embodiments, a HC polypeptide comprises a CH3 domain or a variant thereof. In some embodiments, a CH3 variant is characterized in that when introduced in a HC polypeptide, a half-life of the HC polypeptide is extended without reducing other desirable characteristics, such as neutralization potency, effector function, and/or developability. In some embodiments, a HC polypeptide having a CH3 variant has an extended half-life compared to an otherwise similar HC polypeptide without a CH3 variant.

In some embodiments, a CH3 variant has an addition, substitution, or deletion of at least one amino acid residue compared to a reference CH3 domain, e.g., a wild-type CH3 domain.

In some embodiments, a CH3 variant has an amino acid residue at position 428 which differs from a reference CH3 domain, e.g., a wild-type CH3 domain. In some embodiments, a CH3 variant has an amino acid residue at position 434 which differs from a reference CH3 domain, e.g., a wild-type CH3 domain. In some embodiments, a CH3 variant has amino acid residues at positions 428 and 434 which differ from a reference CH3 domain, e.g., a wild-type CH3 domain.

In some embodiments, a CH3 variant has a leucine at position 428.

In some embodiments, a CH3 variant has an alanine at position 434.

In some embodiments, a HC polypeptide comprising a CH3 variant is characterized in that when administered to a subject, increased antibody dependent cellular cytotoxicity (ADCC) and/or antibody-dependent cellular phagocytosis (ADCP) is observed as compared to a subject administered a HC polypeptide without a CH3 variant. In some embodiments, increased ADCC is characterized by one or more of: increased surface expression of CD107α on natural killer (NK) cells, increased interferon γ (IFNγ) production by NK cells or increased tumor necrosis factor α (TNFα) production by NK cells. In some embodiments, increased ADCP is characterized by one or more of: co-localization of target cells and macrophages utilizing microscopy or flow cytometry; and the inclusion of a pH-sensitive dye to differentiate between cell-associated and internalized target cells.

In some embodiments, a HC polypeptide comprising a CH3 variant has improved developability as compared to a HC polypeptide without a CH3 variant. In some embodiments, improving the developability of a HC polypeptide comprising a CH3 variant comprises increasing expression, increasing solubility, increasing covalent integrity, increasing conformational stability, increasing colloidal stability, decreasing poly-specificity, and/or decreasing immunogenicity of a HC polypeptide comprising a CH3 variant relative to a HC polypeptide without a CH3 variant.

According to IMGT, the CH3 domain is the amino acid positions (or simply referred to as “positions” herein) 341-446 (EU numbering). The term “CH3 domain” is used in a broad sense herein to refer to a heavy chain region comprising at least seven consecutive amino acid positions of the heavy chain positions 341-446 (EU numbering)). A CH3 domain reference sequence, corresponding to the amino acid positions 341-446 according to EU numbering, is provided herein as SEQ ID NO: 59, which is an exemplary amino acid sequence of a wild-type (WT) CH3 domain.

Exemplary CH3 domain reference sequence:

(SEQ ID NO: 59)
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPG.

In some embodiments, a Fc domain comprises a mammalian Fc domain. In some embodiments, a Fc domain comprises a dog, a cat, a mouse, a rat, a rabbit, a primate, a cow, a horse, a sheep, or a human Fc domain. In some embodiments, a Fc domain comprises a human Fc domain. In some embodiments, a Fc domain comprises a dog Fc domain. In some embodiments, a Fc domain comprises a cat Fc domain. In some embodiments, an Fc domain is chosen from an Fc domain of an immunoglobulin isotype. In some embodiments, an immunoglobulin isotype comprises IgA, IgD, IgG, IgM, or IgE. In some embodiments, an Fc domain comprises an Fc domain of an IgG, e.g., a human IgG. In some embodiments, an IgG is or comprises IgG1, IgG2, IgG3, or IgG4.

In some embodiments, an Fc region is a wildtype Fc region, e.g., a wildtype human Fc region. In some embodiments, an Fc region comprises a variant, e.g., an Fc region comprising an addition, substitution, or deletion of at least one amino acid residue in an Fc region which results in, e.g., reduced or ablated affinity for at least one Fc receptor.

The Fc region of an antibody interacts with a number of receptors or ligands including Fc Receptors (e.g., FcyRI, FcyRIIA, FcyRIIIA), the complement protein C1q, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, a HC polypeptide comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region. In some embodiments, an Activin A/B antibody agent comprising a variant Fc region has reduced affinity to a human Fc receptor, e.g., FcyR I, FcyR II and/or FcyR III. Exemplary Fc region variants are disclosed in Saunders K.O., (2019) Frontiers in Immunology; vol 10, Article 296, the entire contents of which is hereby incorporated by reference. For example, a Fc region variant is or comprises a modification provided in Table 3 of Saunders KO (2019). In some embodiments, a Fc region variant comprises Leu234Ala/Leu235Ala (LALA) mutation, a Leu235Glu (LE) mutation, a Ser228Pro/Leu235Glu (SPLE) mutation, Leu234Ala/Leu235Ala/Pro239Gly (LALA-PG) mutation, Pro 331 Ser/Leu234Glu/Leu235Phe™, Asp265Ala (DA) mutation, Leu235Ala/Gly237Ala (LAGA) mutation, or a combination thereof.

In some embodiments, a HC polypeptide disclosed herein comprises a Leu234Ala/Leu235Ala (LALA) mutation.

In some embodiments, a HC polypeptide disclosed herein comprises a Leu235Ala/Gly237Ala (LAGA) mutation.

In some embodiments, a Fc region variant comprises a mutation relative to a wildtype Fc region, e.g., a IgG1 FcR wildtype region. In some embodiments, the hinge and CH2 sequence of an IgG1 FcR wildtype region comprises the sequence of: CPPCPAPELLGGPSVFLFPPK (SEQ ID NO: 43).

In some embodiments, a Fc region variant comprises a LAGA mutation, e.g., as shown in bold in SEQ ID NO: 44: CPPCPAPELAGAPSVFLFPPK. In some embodiments, a HC polypeptide comprises an Fc region having a LAGA mutation, e.g., as provided in SEQ ID NO: 44.

In some embodiments, a Fc region variant comprises a FEGG mutation, e.g., as shown in bold in SEQ ID NO: 45: CPPCPAPEFEGGPSVFLFPPK. In some embodiments, a HC polypeptide comprises an Fc region having a FEGG mutation, e.g., as provided in SEQ ID NO: 45.

In some embodiments, a Fc region variant comprises a AAGG mutation, e.g., as shown in bold in SEQ ID NO: 46: CPPCPAPEAAGGPSVFLFPPK. In some embodiments, a HC polypeptide comprises an Fc region having a AAGG mutation, e.g., as provided in SEQ ID NO: 46.

In some embodiments, a Fc region variant comprises a AAGA mutation, e.g., as shown in bold in SEQ ID NO: 47: CPPCPAPEAAGAPSVFLFPPK. In some embodiments, an AAGA mutation is also referred to as Leu234Ala/Leu235Ala/Glu237Ala (LALAGA). In some embodiments, a HC polypeptide comprises an Fc region having an AAGA mutation, e.g., as provided in SEQ ID NO: 47.

In some embodiments, an Fc region variant comprising an Fc mutation (e.g., as described herein) has reduced binding (e.g., no binding) to a neonatal Fc receptor (FcRn), e.g., when compared to an otherwise similar Fc region without the relevant Fc mutation. In some embodiments, an Activin A/B antibody agent comprising an Fc region having an Fc mutation has reduced binding (e.g., no binding) to FcRn and reduced placental transfer, compared to an otherwise similar Activin A/B antibody agent with an Fc region without the relevant Fc mutation (e.g., as described herein).

In some embodiments, an Fc region variant comprising a LAGA mutation, a FEGG mutation, an AAGG mutation, an AAGA mutation, a LALA mutation or a combination thereof, has reduced binding (e.g., no binding) to a neonatal Fc receptor (FcRn), e.g., when compared to an otherwise similar Fc region without the relevant mutation (e.g., LAGA mutation, FEGG mutation, AAGG mutation, AAGA mutation, LALA mutation or combination thereof). In some embodiments, a GDF15 antibody agent comprising an Fc region having a LAGA mutation, a FEGG mutation, an AAGG mutation, an AAGA mutation, a LALA mutation or a combination thereof has reduced binding (e.g., no binding) to FcRn and reduced placental transfer, compared to an otherwise similar GDF15 antibody agent with an Fc region without the relevant mutation (e.g., LAGA mutation, FEGG mutation, AAGG mutation, AAGA mutation, LALA mutation or combination thereof). In some embodiments, an Fc region variant comprising a I253A mutation, a H310A mutation, a H435R mutation, a H435A mutation or a combination thereof, has reduced binding (e.g., no binding) to a neonatal Fc receptor (FcRn), e.g., when compared to an otherwise similar Fc region without the relevant mutation (e.g., the relevant I253A mutation, H310A mutation, H435R mutation, H435A mutation or combination thereof). In some embodiments, a GDF15 antibody agent comprising an Fc region having a I253A mutation, a H310A mutation, a H435R mutation, a H435A mutation, or a combination thereof has reduced binding (e.g., no binding) to FcRn and reduced placental transfer, compared to an otherwise similar GDF15 antibody agent with an Fc region without the relevant mutation (e.g., the relevant I253A mutation, H310A mutation, H435R mutation, H435A mutation or combination thereof.

In some embodiments, a HC polypeptide disclosed herein further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, a HC polypeptide comprises: (i) a HC CDR 1 sequence provided in Table 2, e.g., any one of SEQ ID NOs: 20, 30, or 35; (ii) a sequence with at least 85%, 86%, 87%, 88%, 89%, 90%, 91%, 92%, 93%, 94%, 95%, 96%, 97%, 98%, 99% identity to a HC CDR1 sequence provided in Table 2, e.g., any one of SEQ ID NOs: 20, 30, or 35; or (iii) a sequence having at least 5, 10, or 20 substitutions compared to a HC CDR1 sequence provided in Table 2, e.g., any one of SEQ ID NOs: 20, 30, or 35.

In some embodiments, a HC polypeptide comprises: (i) a HC CDR2 sequence provided in Table 2, e.g., any one of SEQ ID NOs: 21, 31, or 36; (ii) a sequence with at least 85%, 86%, 87%, 88%, 89%, 90%, 91%, 92%, 93%, 94%, 95%, 96%, 97%, 98%, 99% identity to a HC CDR2 sequence provided in Table 2, e.g., any one of SEQ ID NOs: 21, 31, or 36; or (iii) a sequence having at least 5, 10, or 20 substitutions compared to a HC CDR2 sequence provided in Table 2, e.g., any one of SEQ ID NOs: 21, 31, or 36.

In some embodiments, a HC polypeptide comprises: (i) a HC CDR3 sequence provided in Table 2, e.g., any one of SEQ ID NOs: 22 or 37; (ii) a sequence with at least 85%, 86%, 87%, 88%, 89%, 90%, 91%, 92%, 93%, 94%, 95%, 96%, 97%, 98%, 99% identity to a HC CDR3 sequence provided in Table 2, e.g., any one of SEQ ID NOs: 22 or 37; or (iii) a sequence having at least 5, 10, or 20 substitutions compared to a HC CDR3 sequence provided in Table 2, e.g., any one of SEQ ID NOs: 22 or 37.

In some embodiments, a HC polypeptide comprising a HC CDR1, a HC CDR2 and/or a HC CDR3 is able to specifically bind to Activin-A and/or Activin-B.

In some embodiments, a HC polypeptide comprises: (i) an HC CDR1, HC CDR2, and HC CDR3 sequence provided in Table 2; (ii) a sequence with at least 85%, 86%, 87%, 88%, 89%, 90%, 91%, 92%, 93%, 94%, 95%, 96%, 97%, 98%, or 99% identity to an HC CDR1, HC CDR2, and HC CDR3 sequence provided in Table 2; (iii) a sequence having at least 5, 10, or 20 substitutions relative to an HC CDR1, HC CDR2, and HC CDR3 sequence provided in Table 2.

In some embodiments, a HC polypeptide comprises: (i) an HC CDR1 of SEQ ID NO: 20, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 20; (ii) an HC CDR2 of SEQ ID NO: 21, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 21; and/or (iii) an HC CDR3 of SEQ ID NO: 22, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 22.

In some embodiments, a HC polypeptide comprises: (i) an HC CDR1 of SEQ ID NO: 30, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 30; (ii) an HC CDR2 of SEQ ID NO: 31, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 31; and/or (iii) an HC CDR3 of SEQ ID NO: 22, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 22.

In some embodiments, a HC polypeptide comprises: (i) an HC CDR1 of SEQ ID NO: 35, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 35; (ii) an HC CDR2 of SEQ ID NO: 36, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 36; and/or (iii) an HC CDR3 of SEQ ID NO: 37, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 37.

In some embodiments, a HC polypeptide further comprises one or more framework regions (FR), e.g., as described herein. In some embodiments, such a HC polypeptide comprises one, two, three or four FRs, e.g., as described herein. In some embodiments, a FR comprises a HC FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a HC FR sequence as described herein, or a sequence having at least 5, 10 or 20 alterations relative to a HC FR sequence as described herein.

In some embodiments, a HC polypeptide comprises: (i) a FR sequence provided in Table 2; (ii) a sequence with at least 92%, 93%, 94%, 95%, 96%, 97%, 98%, or 99% identity to a FR sequence provided in Table 2; or (iii) a sequence having at least 5, 10, or 20 alterations (e.g., substitutions, deletions or insertions (e.g., conservative substitutions)) compared to a FR sequence provided in Table 2.

In some embodiments, a HC polypeptide comprises a HC FR1 provided in Table 2, a sequence with at least 92%, 93%, 94%, 95%, 96%, 97%, 98%, or 99% identity to a HC FR1 sequence provided in Table 2, or a sequence having at least 5, 10, or 20 alterations (e.g., substitutions, deletions or insertions (e.g., conservative substitutions)) compared to a HC FR1 sequence provided in Table 2.

In some embodiments, a HC polypeptide comprises a HC FR2 provided in Table 2, a sequence with at least 92%, 93%, 94%, 95%, 96%, 97%, 98%, or 99% identity to a HC FR2 sequence provided in Table 2, or a sequence having at least 5, 10, or 20 alterations (e.g., substitutions, deletions or insertions (e.g., conservative substitutions)) compared to a HC FR2 sequence provided in Table 2.

In some embodiments a HC polypeptide comprises a HC FR3 provided in Table 2, a sequence with at least 92%, 93%, 94%, 95%, 96%, 97%, 98%, or 99% identity to a HC FR3 sequence provided in Table 2, or a sequence having at least 5, 10, or 20 alterations (e.g., substitutions, deletions or insertions (e.g., conservative substitutions)) compared to a HC FR3 sequence provided in Table 2.

In some embodiments, a HC polypeptide comprises a HC FR4 provided in Table 2, a sequence with at least 92%, 93%, 94%, 95%, 96%, 97%, 98%, or 99% identity to a HC FR4 sequence provided in Table 2, or a sequence having at least 5, 10, or 20 alterations (e.g., substitutions, deletions or insertions (e.g., conservative substitutions)) compared to a HC FR4 sequence provided in Table 2.

In some embodiments, a HC polypeptide comprises a HC CDR1, a HC CDR2 and HC CDR3 provided in Table 2 or a sequence with at least 85% identity thereto, and a HC FR1, HC FR2, HCFR3 and a HC FR4 provided in Table 2 or a sequence with at least 92% identity thereto.

In some embodiments, a HC polypeptide comprises the sequence of SEQ ID NO: 27, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 27.

In some embodiments, a HC polypeptide comprises the sequence of SEQ ID NO: 32, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 32.

In some embodiments, a HC polypeptide comprises the sequence of SEQ ID NO: 39, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 39.

In some embodiments, a HC polypeptide comprises a HC amino acid sequence provided in Table 2, e.g., any one of SEQ ID NOs: 28, 33, or 40; or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to a HC amino acid sequence provided in Table 2, e.g., any one of SEQ ID NOs: 28, 33, or 40.

In some embodiments, a HC polypeptide disclosed herein comprises a terminal lysine, e.g., as provided in Table 2. In some embodiments, a HC polypeptide disclosed herein does not comprise a terminal lysine.

Exemplary useful HC polypeptides which may be included in an Activin-A and/or Activin-B antibody agents disclosed herein are disclosed in Table 2 below.

TABLE 2
Exemplary heavy chain sequences for Activin A/B antibody agents

| | SEQ ID | | |
|---|---|---|---|
| CLONE | NO | FEATURE | SEQUENCE |
| A | 20 | HC CDR1 | GSISSSSYYWG |
| | 21 | HC CDR2 | SISYSGSTYYNPSLKS |
| | 22 | HC CDR3 | AREGYDSYSYDV |
| | 23 | HC FR1 | QLQLQESGPGLVKPSETLSLTCTVSG |
| | 24 | HC FR2 | WIRQPPGKGLEWIG |
| | 25 | HC FR3 | RVTISVDTSKNQFSLKLSSVTAADTAVYYC |
| | 26 | HC FR4 | WGQGTMVTVSS |
| | 27 | VH amino | QLQLQESGPGLVKPSETLSLTCTVSGGSISSSS |
| | | acid | YYWGWIRQPPGKGLEWIGSISYSGSTYYNPSL |
| | | | KSRVTISVDTSKNQFSLKLSSVTAADTAVYYC |
| | | | AREGYDSYSYDVWGQGTMVTVSS |
| | 28 | HC | QLQLQESGPGLVKPSETLSLTCTVSGGSISSSS |
| | | | YYWGWIRQPPGKGLEWIGSISYSGSTYYNPSL |
| | | | KSRVTISVDTSKNQFSLKLSSVTAADTAVYYC |
| | | | AREGYDSYSYDVWGQGTMVTVSS |
| | | | ASTKGPSVFPLAPSSKSTSGGTAALGCLVKDY |
| | | | FPEPVTVSWNSGALTSGVHTFPAVLQSSGLYS |
| | | | LSSVVTVPSSSLGTQTYICNVNHKPSNTKVDK |
| | | | KVEPKSCDKTHTCPPCPAPEAAGAPSVFLFPP |
| | | | KPKDTLMISRTPEVTCVVVDVSHEDPEVKFN |
| | | | WYVDGVEVHNAKTKPREEQYNSTYRVVSVL |
| | | | TVLHQDWLNGKEYKCKVSNKALPAPIEKTISK |
| | | | AKGQPREPQVYTLPPSREEMTKNQVSLTCLV |
| | | | KGFYPSDIAVEWESNGQPENNYKTTPPVLDSD |
| | | | GSFFLYSKLTVDKSRWQQGNVFSCSVMHEAL |
| | | | HNHYTQKSLSLSPGK |
| | 29 | VH DNA | CAGCTGCAGCTGCAGGAGTCGGGCCCAGGA |
| | | | CTGGTGAAGCCTTCGGAGACCCTGTCCCTCA |
| | | | CCTGCACTGTCTCTGGTGGCTCCATCAGCAG |
| | | | TAGTAGTTACTACTGGGGCTGGATCCGCCA |
| | | | GCCCCCAGGGAAGGGGCTGGAGTGGATTGG |
| | | | GAGTATCTCCTATAGTGGGAGCACCTACTAC |
| | | | AACCCGTCCCTCAAGAGTCGAGTCACCATA |
| | | | TCCGTAGACACGTCCAAGAACCAGTTCTCCC |
| | | | TGAAGCTGAGTTCTGTGACCGCCGCAGACA |
| | | | CGGCGGTGTACTACTGCGCCAGAGAGGGAT |
| | | | ACGACAGCTACAGCTATGACGTATGGGGTC |
| | | | AGGGTACAATGGTCACCGTCTCCTCA |
| B | 30 | HC CDR1 | GSISSQQYYWG |
| | 31 | HC CDR2 | SISYAASTYYNPSLKS |
| | 22 | HC CDR3 | AREGYDSYSYDV |
| | 23 | HC FR1 | QLQLQESGPGLVKPSETLSLTCTVSG |
| | 24 | HC FR2 | WIRQPPGKGLEWIG |
| | 25 | HC FR3 | RVTISVDTSKNQFSLKLSSVTAADTAVYYC |
| | 26 | HC FR4 | WGQGTMVTVSS |
| | 32 | VH amino | QLQLQESGPGLVKPSETLSLTCTVSGGSISSQQ |
| | | acid | YYWGWIRQPPGKGLEWIGSISYAASTYYNPSL |
| | | | KSRVTISVDTSKNQFSLKLSSVTAADTAVYYC |
| | | | AREGYDSYSYDVWGQGTMVTVSS |
| | 33 | HC | QLQLQESGPGLVKPSETLSLTCTVSGGSISSQQ |
| | | | YYWGWIRQPPGKGLEWIGSISYAASTYYNPSL |
| | | | KSRVTISVDTSKNQFSLKLSSVTAADTAVYYC |
| | | | AREGYDSYSYDVWGQGTMVTVSS |
| | | | ASTKGPSVFPLAPSSKSTSGGTAALGCLVKDY |
| | | | FPEPVTVSWNSGALTSGVHTFPAVLQSSGLYS |
| | | | LSSVVTVPSSSLGTQTYICNVNHKPSNTKVDK |
| | | | KVEPKSCDKTHTCPPCPAPEAAGAPSVFLFPP |
| | | | KPKDTLMISRTPEVTCVVVDVSHEDPEVKFN |
| | | | WYVDGVEVHNAKTKPREEQYNSTYRVVSVL |
| | | | TVLHQDWLNGKEYKCKVSNKALPAPIEKTISK |
| | | | AKGQPREPQVYTLPPSREEMTKNQVSLTCLV |
| | | | KGFYPSDIAVEWESNGQPENNYKTTPPVLDSD |
| | | | GSFFLYSKLTVDKSRWQQGNVFSCSVMHEAL |
| | | | HNHYTQKSLSLSPGK |
| | 34 | VH DNA | CAGCTGCAGCTGCAGGAGTCGGGCCCAGGA |
| | | | CTGGTGAAGCCTTCGGAGACCCTGTCCCTCA |
| | | | CCTGCACTGTCTCTGGTGGCTCCATCAGCAG |
| | | | TCAACAATACTACTGGGGCTGGATCCGCCA |
| | | | GCCCCCAGGGAAGGGGCTGGAGTGGATTGG |
| | | | GAGTATCTCCTATGCCGCAAGCACCTACTAC |
| | | | AACCCGTCCCTCAAGAGTCGAGTCACCATA |
| | | | TCCGTAGACACGTCCAAGAACCAGTTCTCCC |
| | | | TGAAGCTGAGTTCTGTGACCGCCGCAGACA |
| | | | CGGCGGTGTACTACTGCGCCAGAGAGGGAT |
| | | | ACGACAGCTACAGCTATGACGTATGGGGTC |
| | | | AGGGTACAATGGTCACCGTCTCCTCA |
| C | 35 | HC CDR1 | GSISSYDPYWG |
| | 36 | HC CDR2 | SISYSGSTLYNPSLKS |
| | 37 | HC CDR3 | AREVSYLGSRRYGMDV |
| | 23 | HC FR1 | QLQLQESGPGLVKPSETLSLTCTVSG |
| | 24 | HC FR2 | WIRQPPGKGLEWIG |
| | 25 | HC FR3 | RVTISVDTSKNQFSLKLSSVTAADTAVYYC |
| | 38 | HC FR4 | WGQGTTVTVSS |
| | 39 | VH amino | QLQLQESGPGLVKPSETLSLTCTVSGGSISSYD |
| | | acid | PYWGWIRQPPGKGLEWIGSISYSGSTLYNPSL |
| | | | KSRVTISVDTSKNQFSLKLSSVTAADTAVYYC |
| | | | AREVSYLGSRRYGMDVWGQGTTVTVSS |
| | 40 | HC | QLQLQESGPGLVKPSETLSLTCTVSGGSISSYD |
| | | | PYWGWIRQPPGKGLEWIGSISYSGSTLYNPSL |
| | | | KSRVTISVDTSKNQFSLKLSSVTAADTAVYYC |
| | | | AREVSYLGSRRYGMDVWGQGTTVTVSS |
| | | | ASTKGPSVFPLAPSSKSTSGGTAALGCLVKDY |
| | | | FPEPVTVSWNSGALTSGVHTFPAVLQSSGLYS |
| | | | LSSVVTVPSSSLGTQTYICNVNHKPSNTKVDK |
| | | | KVEPKSCDKTHTCPPCPAPEAAGAPSVFLFPP |
| | | | KPKDTLMISRTPEVTCVVVDVSHEDPEVKFN |
| | | | WYVDGVEVHNAKTKPREEQYNSTYRVVSVL |
| | | | TVLHQDWLNGKEYKCKVSNKALPAPIEKTISK |
| | | | AKGQPREPQVYTLPPSREEMTKNQVSLTCLV |
| | | | KGFYPSDIAVEWESNGQPENNYKTTPPVLDSD |
| | | | GSFFLYSKLTVDKSRWQQGNVFSCSVMHEAL |
| | | | HNHYTQKSLSLSPGK |
| | 41 | VH DNA | CAGCTGCAGCTGCAGGAGTCGGGCCCAGGA |
| | | | CTGGTGAAGCCTTCGGAGACCCTGTCCCTCA |
| | | | CCTGCACTGTCTCTGGTGGCTCCATCAGCAG |
| | | | TTACGACCCCTACTGGGGCTGGATCCGCCA |
| | | | GCCCCCAGGGAAGGGGCTGGAGTGGATTGG |
| | | | GAGTATCTCCTATAGTGGGAGCACCCTCTAC |
| | | | AACCCGTCCCTCAAGAGTCGAGTCACCATA |
| | | | TCCGTAGACACGTCCAAGAACCAGTTCTCCC |
| | | | TGAAGCTGAGTTCTGTGACCGCCGCAGACA |
| | | | CGGCGGTGTACTACTGCGCCAGAGAGGTGA |
| | | | GCTATCTAGGTTCACGTAGATACGGAATGG |
| | | | ACGTATGGGGCCAGGGAACAACTGTCACCG |
| | | | TCTCCTCA |
| IgG | 42 | | ASTKGPSVFPLAPSSKSTSGGTAALGCLVKDY |
| | | | FPEPVTVSWNSGALTSGVHTFPAVLQSSGLYS |
| | | | LSSVVTVPSSSLGTQTYICNVNHKPSNTKVDK |
| | | | KVEPKSCDKTHTCPPCPAPEAAGAPSVFLFPP |
| | | | KPKDTLMISRTPEVTCVVVDVSHEDPEVKFN |
| | | | WYVDGVEVHNAKTKPREEQYNSTYRVVSVL |
| | | | TVLHQDWLNGKEYKCKVSNKALPAPIEKTISK |
| | | | AKGQPREPQVYTLPPSREEMTKNQVSLTCLV |
| | | | KGFYPSDIAVEWESNGQPENNYKTTPPVLDSD |
| | | | GSFFLYSKLTVDKSRWQQGNVFSCSVMHEAL |
| | | | HNHYTQKSLSLSPGK |

In some embodiments, a HC polypeptide disclosed herein comprises an IgG sequence of SEQ ID NO: 42 or a sequence with at least 85% identity thereto.

### Activin A/B Antibody Agents Comprising a Light Chain Polypeptide and a Heavy Chain Polypeptide

In some embodiments, an Activin A/B antibody agent disclosed herein, e.g., a Activin A/B antibody agent polypeptide, comprises a light chain comprising a variable region comprising one, two or three LC CDRs and a heavy chain comprising a variable region comprising one, two or three HC CDRs. In some embodiments, an Activin A/B antibody agent comprises a light chain comprising a LC CDR1, a LC CDR2 and a LC CDR3, and a heavy chain comprising a HC CDR1, a HC CDR2 and HC CDR3.

In some embodiments, an Activin A/B antibody agent comprising a LC CDR1, a LC CDR2 and a LC CDR3, and a heavy chain comprising a HC CDR1, a HC CDR2 and HC CDR3 is able to specifically bind to Activin-A and/or Activin-B, e.g., human, primate, or a domestic mammal Activin-A and/or Activin-B (e.g., a dog, a cat, a horse, a sheep, a cow, a yak and/or a camel).

In some embodiments, an Activin A/B antibody agent comprises one, two, or three LC CDRs provided in Table 1 or a sequence with at least 85%, 86%, 87%, 88%, 89%, 90%, 91%, 92%, 93%, 94%, 95%, 96%, 97%, 98%, or 99% identity thereto; and one, two, or three HC CDRs provided in Table 2, or a sequence with at least 85%, 86%, 87%, 88%, 89%, 90%, 91%, 92%, 93%, 94%, 95%, 96%, 97%, 98%, or 99% identity thereto.

In some embodiments, an Activin A/B antibody agent comprises: (a) a light chain comprising: (i) an LC CDR1 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR1 provided in Table 1; (ii) an LC CDR2 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR2 provided in Table 1; and/or (iii) an LC CDR3 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR3 provided in Table 1; and (b) a heavy chain comprising: (i) a HC CDR1 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR1 provided in Table 2; (ii) a HC CDR2 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR2 provided in Table 2; and/or (iii) a HC CDR3 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR3 provided in Table 2.

In some embodiments, an Activin A/B antibody agent comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 1; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 2; and an LC CDR3 of SEQ ID NO: 3, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 3; and (ii) a HC CDR1 of SEQ ID NO: 20, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 20; an HC CDR2 of SEQ ID NO: 21, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 21; and an HC CDR3 of SEQ ID NO: 22, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 22.

In some embodiments, an Activin A/B antibody agent comprises: (i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 1; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 2; and an LC CDR3 of SEQ ID NO: 3, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 3; and (ii) a HC CDR1 of SEQ ID NO: 30, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 30; an HC CDR2 of SEQ ID NO: 31, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 31; and an HC CDR3 of SEQ ID NO: 22, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 22.

In some embodiments, an Activin A/B antibody agent comprises: (i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 11; an LC CDR2 of SEQ ID NO: 12, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 12; and an LC CDR3 of SEQ ID NO: 13, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 13; and (ii) a HC CDR1 of SEQ ID NO: 35, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 35; an HC CDR2 of SEQ ID NO: 36, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 36; and an HC CDR3 of SEQ ID NO: 37, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 37.

In some embodiments, an Activin A/B antibody agent comprises a light chain polypeptide (LC polypeptide) as described herein.

In some embodiments, an Activin A/B antibody agent comprises a heavy chain polypeptide (HC polypeptide) as described herein. In some embodiments, a HC polypeptide in an Activin A/B antibody agent does not include a terminal lysine.

In some embodiments, an Activin A/B antibody agent comprises a light chain polypeptide (LC polypeptide) as described herein and a heavy chain polypeptide (HC polypeptide) as described herein. In some embodiments, a HC polypeptide in an Activin A/B antibody agent does not include a terminal lysine.

In some embodiments, an Activin A/B antibody agent comprises a light chain comprising a variable region (VL) comprising three LC CDRs and one or more framework regions (e.g., as described herein); and a heavy chain comprising a variable region (VH) comprising three HC CDRs and one or more framework regions (e.g., as described herein).

In some embodiments, a VL and/or a VH of an Activin A/B antibody agent further comprises one or more framework regions (FR), e.g., as described herein. In some embodiments, a VL and/or a VH of an Activin A/B antibody agent comprises one, two, three or four FRs, e.g., as described herein. In some embodiments, a FR comprises a FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a LC FR sequence as described herein, or a sequence having at least 5, 10 or 20 alterations relative to a LC FR sequence as described herein.

In some embodiments, a VL and/or a VH of an Activin A/B antibody agent comprises: (i) a FR sequence provided in Table 1 or Table 2; (ii) a sequence with at least 92%, 93%, 94%, 95%, 96%, 97%, 98%, or 99% identity to a FR sequence provided in Table 1 or 2; or (iii) a sequence having at least 5, 10, or 20 alterations (e.g., substitutions, deletions or insertions (e.g., conservative substitutions)) compared to a FR sequence provided in Table 1 or 2.

In some embodiments, a VL and/or a VH of an Activin A/B antibody agent comprises a FR1 provided in Table 1 or 2, a sequence with at least 92%, 93%, 94%, 95%, 96%, 97%, 98%, or 99% identity to a FR1 sequence provided in Table 1 or 2, or a sequence having at least 5, 10, or 20 alterations (e.g., substitutions, deletions or insertions (e.g., conservative substitutions)) compared to a FR1 sequence provided in Table 1 or 2.

In some embodiments, a VL and/or a VH of an Activin A/B antibody agent comprises a FR2 provided in Table 1 or 2, a sequence with at least 92%, 93%, 94%, 95%, 96%, 97%, 98%, or 99% identity to a FR2 sequence provided in Table 1 or 2, or a sequence having at least 5, 10, or 20 alterations (e.g., substitutions, deletions or insertions (e.g., conservative substitutions)) compared to a FR2 sequence provided in Table 1 or 2.

In some embodiments a VL and/or a VH of an Activin A/B antibody agent comprises a FR3 provided in Table 1 or 2, a sequence with at least 92%, 93%, 94%, 95%, 96%, 97%, 98%, or 99% identity to a FR3 sequence provided in Table 1 or 2, or a sequence having at least 5, 10, or 20 alterations (e.g., substitutions, deletions or insertions (e.g., conservative substitutions)) compared to a FR3 sequence provided in Table 1 or 2.

In some embodiments, a VL and/or a VH of an Activin A/B antibody agent comprises a FR4 provided in Table 1 or 2, a sequence with at least 92%, 93%, 94%, 95%, 96%, 97%, 98%, or 99% identity to a FR4 sequence provided in Table 1 or 2, or a sequence having at least 5, 10, or 20 alterations (e.g., substitutions, deletions or insertions (e.g., conservative substitutions)) compared to a FR4 sequence provided in Table 1 or 2.

In some embodiments an Activin A/B antibody agent comprises a VL comprising 3 LC CDRs and a LC FR1, LC FR2, LC FR3 and a LC FR4 of an Activin A/B antibody agent provided in Table 1 or a sequence with at least 92% identity thereto; and/or a VH comprising 3 HC CDRs and a HC FR1, HC FR2, HC FR3 and a HC FR4 of an Activin A/B antibody agent provided in Table 2 or a sequence with at least 92% identity thereto.

In some embodiments, an Activin A/B antibody agent comprises a VL sequence provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a VL sequence provided in Table 1; and a VH sequence provided in Table 2 or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to a VH sequence provided in Table 2.

In some embodiments, an Activin A/B antibody agent comprises: the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; and the sequence of SEQ ID NO: 27, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 27.

In some embodiments, an Activin A/B antibody agent comprises: the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; and the sequence of SEQ ID NO: 32, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 32.

In some embodiments, an Activin A/B antibody agent comprises: the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; and the sequence of SEQ ID NO: 39, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 39.

In some embodiments, an Activin A/B antibody agent comprises: the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16; and the sequence of SEQ ID NO: 27, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 27.

In some embodiments, an Activin A/B antibody agent comprises: the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16; and the sequence of SEQ ID NO: 32, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 32.

In some embodiments, an Activin A/B antibody agent comprises: the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16; and the sequence of SEQ ID NO: 39, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 39.

In some embodiments, an Activin A/B antibody agent comprises: a light chain comprising three LC CDRs, one or more framework regions (e.g., as described herein) and a constant region; and a heavy chain comprising three HC CDRs, one or more framework regions (e.g., as described herein), and at least one constant region.

In some embodiments, a light chain constant region comprises a light chain kappa or a light chain lambda constant region.

In some embodiments, a light chain kappa constant region comprises the sequence of SEQ ID NO: 19, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 19.

In some embodiments, a heavy chain constant region comprises a CH1 domain, a CH2 domain and/or a CH3 domain.

In some embodiments, an Activin A/B antibody agent comprises a CH3 domain or a variant thereof. In some embodiments, a CH3 variant is characterized in that when introduced in an Activin A/B antibody agent, a half-life of an Activin A/B antibody agent is extended without reducing other desirable characteristics, such as neutralization potency, effector function, and/or developability. In some embodiments, an Activin A/B antibody agent having a CH3 variant has an extended half-life compared to an otherwise similar Activin A/B antibody agent without a CH3 variant

In some embodiments, a CH3 variant has an addition, substitution, or deletion of at least one amino acid residue compared to a reference CH3 domain, e.g., a wild-type CH3 domain.

In some embodiments, a CH3 variant has an amino acid residue at position 428 which differs from a reference CH3 domain, e.g., a wild-type CH3 domain. In some embodiments, a CH3 variant has an amino acid residue at position 434 which differs from a reference CH3 domain, e.g., a wild-type CH3 domain. In some embodiments, a CH3 variant has amino acid residues at positions 428 and 434 which differ from a reference CH3 domain, e.g., a wild-type CH3 domain.

In some embodiments, a CH3 variant has a leucine at position 428.

In some embodiments, a CH3 variant has an alanine at position 434.

In some embodiments, an Activin A/B antibody agent comprising a CH3 variant is characterized in that when administered to a subject, increased antibody dependent cellular cytotoxicity (ADCC) or antibody-dependent cellular phagocytosis (ADCP) is observed as compared to a subject administered an Activin A/B antibody agent without a CH3 variant. In some embodiments, increased ADCC is characterized by one or more of: increased surface expression of CD107a on natural killer (NK) cells, increased interferon γ (IFNγ) production by NK cells or increased tumor necrosis factor α (TNFα) production by NK cells. In some embodiments, increased ADCP is characterized by one or more of: co-localization of target cells and macrophages utilizing microscopy or flow cytometry; and the inclusion of a pH-sensitive dye to differentiate between cell-associated and internalized target cells.

In some embodiments, an Activin A/B antibody agent comprising a CH3 variant has improved developability as compared to an Activin A/B antibody agent without a CH3 variant. In some embodiments, improving the developability of an Activin A/B antibody agent comprising a CH3 variant comprises increasing expression, increasing solubility, increasing covalent integrity, increasing conformational stability, increasing colloidal stability, decreasing poly-specificity, and/or decreasing immunogenicity of an Activin A/B antibody agent comprising a CH3 variant relative to an Activin A/B antibody agent without a CH3 variant.

In some embodiments, at least one constant region comprises an Fc domain. In some embodiments, an Fc domain comprises a mammalian Fc domain. In some embodiments, an Fc domain comprises a dog, a cat, a mouse, a rat, a rabbit, a primate, a cow, a horse or a human Fc domain. In some embodiments, an Fc domain is chosen from an Fc domain of an immunoglobulin isotype. In some embodiments, an immunoglobulin isotype comprises IgA, IgD, IgG, IgM, or IgE. In some embodiments, an Fc domain comprises an Fc domain of an IgG, e.g., a human IgG. In some embodiments, an IgG is or comprises IgG1, lgG2, lgG3, or lgG4.

In some embodiments, an Activin A/B antibody agent disclosed herein comprises an Fc region, e.g., as described herein. In some embodiments, an Fc region is a wildtype Fc region, e.g., a wildtype human Fc region. In some embodiments, an Fc region comprises a variant, e.g., an Fc region comprising an addition, substitution, or deletion of at least one amino acid residue in an Fc region which results in, e.g., reduced or ablated affinity for at least one Fc receptor.

In some embodiments, an Activin A/B antibody agent comprises one or more mutations in a constant region of an antibody. In some embodiments, an Activin A/B antibody agent comprises one or more mutations in a constant region as disclosed in U.S. Pat. No. 5,624,821, the entire contents of which is hereby incorporated by reference.

In some embodiments, an Activin A/B antibody agent comprising a variant Fc region. In some embodiments, a Fc region variant comprises Leu234Ala/Leu235Ala (LALA) mutation, a Leu235Glu (LE) mutation, a Ser228Pro/Leu235Glu (SPLE) mutation, Leu234Ala/Leu235Ala/Pro239Gly (LALA-PG) mutation, Pro 331Ser/Leu234Glu/Leu235Phe™, Asp265Ala (DA) mutation, Leu235Ala/Gly237Ala (LAGA) mutation, or a combination thereof.

In some embodiments, an Activin A/B antibody agent disclosed herein comprises a Leu234Ala/Leu235Ala (LALA) mutation.

In some embodiments, an Activin A/B antibody agent disclosed herein comprises a Leu235Ala/Gly237Ala (LAGA) mutation.

In some embodiments, a Fc region variant comprises a mutation relative to a wildtype Fc region, e.g., a IgG1 FcR wildtype region. In some embodiments, the hinge and CH2 sequence of an IgG1 FcR wildtype region comprises the sequence of: CPPCPAPELLGGPSVFLFPPK (SEQ ID NO: 43).

In some embodiments, a Fc region variant comprises a LAGA mutation, e.g., as shown in bold in SEQ ID NO: 44: CPPCPAPELAGAPSVFLFPPK. In some embodiments, an Activin A/B antibody agent comprises an Fc region having a LAGA mutation, e.g., as provided in SEQ ID NO: 44.

In some embodiments, a Fc region variant comprises a FEGG mutation, e.g., as shown in bold in SEQ ID NO: 45: CPPCPAPEFEGGPSVFLFPPK. In some embodiments, an Activin A/B antibody agent comprises an Fc region having a FEGG mutation, e.g., as provided in SEQ ID NO: 45.

In some embodiments, a Fc region variant comprises a AAGG mutation, e.g., as shown in bold in SEQ ID NO: 46: CPPCPAPEAAGGPSVFLFPPK. In some embodiments, an Activin A/B antibody agent comprises an Fc region having a AAGG mutation, e.g., as provided in SEQ ID NO: 46.

In some embodiments, a Fc region variant comprises a AAGA mutation, e.g., as shown in bold in SEQ ID NO: 47: CPPCPAPEAAGAPSVFLFPPK. In some embodiments, an AAGA mutation is also referred to as Leu234Ala/Leu235Ala/Glu237Ala (LALAGA). In some embodiments, an Activin A/B antibody agent comprises an Fc region having an AAGA mutation, e.g., as provided in SEQ ID NO: 47.

In some embodiments, a Fc region variant comprising an Fc mutation has reduced binding (e.g., no binding) to a neonatal Fc receptor (FcRn), e.g., when compared to an otherwise similar Fc region without an Fc mutation. In some embodiments, an Activin A/B antibody agent comprising an Fc region having an Fc mutation has reduced binding (e.g., no binding) to FcRn and reduced placental transfer, compared to an otherwise similar Activin A/B antibody agent with an Fc region without an Fc mutation.

In some embodiments, an Activin A/B antibody agent comprising an Fc region having a LAGA mutation, a FEGG mutation, an AAGG mutation, an AAGA mutation, a LALA mutation or a combination thereof, has reduced binding (e.g., no binding) to FcRn and reduced placental transfer, compared to an otherwise similar Activin A/B antibody agent with an Fc region without a LAGA mutation, a FEGG mutation, an AAGG mutation, an AAGA mutation, a LALA mutation or a combination thereof.

In some embodiments, a Fc region variant comprising a I253A mutation, a H310A mutation, a H435R mutation, a H435A mutation or a combination thereof, has reduced binding (e.g., no binding) to a neonatal Fc receptor (FcRn), e.g., when compared to an otherwise similar Fc region without a I253A mutation, a H310A mutation, a H435R mutation, a H435A mutation or a combination thereof.

In some embodiments, an Activin A/B antibody agent comprising an Fc region having a I253A mutation, a H310A mutation, a H435R mutation, a H435A mutation or a combination thereof, has reduced binding (e.g., no binding) to FcRn and reduced placental transfer, compared to an otherwise similar Activin A/B antibody agent with an Fc region without a I253A mutation, a H310A mutation, a H435R mutation, a H435A mutation or a combination thereof.

In some embodiments, an Activin A/B antibody agent disclosed herein comprises an IgG sequence of SEQ ID NO: 42 or a sequence with at least 85% identity thereto.

In some embodiments, an Activin A/B antibody agent disclosed herein further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, an Activin A/B antibody agent comprises a heavy chain (HC) provided in Table 2 (or a sequence having at least 85% identity thereto) and a light chain (HC) provided in Table 1 (or a sequence having at least 85% identity thereto).

In some embodiments, an Activin A/B antibody agent comprises: (i) a HC polypeptide comprising a HC amino acid sequence provided in Table 2, e.g., any one of SEQ ID NOs: 28, 33, 40; or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to a HC amino acid sequence provided in Table 2, e.g., any one of SEQ ID NOs: 28, 33, 40; and (ii) a LC polypeptide comprising an LC sequence provided in Table 1, e.g., any one of SEQ ID NOs: 9 or 17, or a sequence with at least 85%, 86%, 87%, 88%, 89%, 90%, 91%, 92%, 93%, 94%, 95%, 96%, 97%, 98%, 99% identity thereto; or a sequence having at least 5, 10, or 20 alterations (e.g., substitutions, deletions or insertions (e.g., conservative substitutions)) relative to any one of SEQ ID NOs: 9 or 17.

### Bispecific or Multispecific Activin A/B Antibody Agents

Disclosed herein, among other things, are bispecific or multispecific Activin A/B antibody agents and compositions comprising the same. In some embodiments, a bispecific Activin A/B antibody agent antibody agent comprises a first binding specificity for Activin-A and/or Activin-B and a second binding specificity for a second antigen.

In some embodiments, a second antigen is other than Activin-A and/or Activin-B.

In some embodiments, a second antigen is a member of the TGFbeta superfamily.

In some embodiments, a second antigen is not a member of the TGFbeta superfamily.

In some embodiments, a second antigen is an immune-related antigen.

In some embodiments, a second antigen is an immune checkpoint inhibitor.

In some embodiments, a bispecific Activin A/B antibody agent antibody agent comprises a first binding specificity for Activin-A and/or Activin-B and a second binding specificity for an immune checkpoint inhibitor. In some embodiments, an immune checkpoint inhibitor comprises CTLA4, PD1, PDL1, TIM-3, LAG-3, BTLA, TIGIT, B7-H3 and/or VISTA.

In some embodiments, a bispecific Activin A/B antibody comprises an Activin A/B binding agent provided herein and a second binding specificity to Activin A.

In some embodiments, a bispecific Activin A/B antibody comprises an Activin A/B binding agent provided herein and a second binding specificity to Activin B.

In some embodiments of an Activin-A and/or Activin-B bispecific antibody agent, a single chain Activin A polypeptide (e.g., a Activin A light chain polypeptide or a Activin A heavy chain polypeptide) provides binding specificity to Activin A. In some embodiments of an Activin A/B bispecific antibody agent, a single chain Activin B polypeptide (e.g., an Activin B light chain polypeptide or a Activin B heavy chain polypeptide) provides binding specificity to Activin B.

In some embodiments, an Activin A/B bispecific antibody agent comprises a light chain (LC) polypeptide comprising a LC CDR1, a LC CDR2, and a LC CDR3, e.g., as provided in Table 1). In some embodiments, an Activin A/B bispecific antibody agent comprising a LC polypeptide with a LC CDR1, a LC CDR2, and a LC CDR3 binds to Activin-A, Activin-B, and/or to one or more complexes that include one or both Activin-A and Activin-B.

In some embodiments, an Activin A/B bispecific antibody agent comprises a heavy chain (HC) polypeptide comprising a HC CDR1, a HC CDR2, and a HC CDR3, e.g., as provided in Table 2). In some embodiments, an Activin A/B bispecific antibody agent comprising a HC polypeptide with a HC CDR1, a HC CDR2, and a HC CDR3 binds to Activin-A, Activin-B, and/or to one or more complexes that include one or both Activin-A and Activin-B.

In some embodiments, an Activin A/B bispecific antibody agent comprises an Activin A/B antibody agent comprising a heavy chain (HC) comprising a HC CDR1, a HC CDR2, and a HC CDR3, e.g., as provided in Table 2; and a light chain (LC) comprising a LC CDR1, a LC CDR2, and a LC CDR3, e.g., as provided in Table 1.

In some embodiments, an Activin A/B bispecific antibody agent is or comprises: a heterodimer, a Crossmab, a DVD-Ig, a 2 in 1 IgG, an IgG-sc-FV, an scFv-scFv, a BiTE, a DART, a diabody, a Fab-scFv fusion, a Fab-Fab fusion, a tandem antibody, or any other art recognized formats for an antibody having dual-specificity and/or multi-specificity.

### Characterization of Activin A/B Antibody Agents

Activin A/B antibody agents disclosed herein specifically bind to Activin-A and/or Activin-B and have one or more characteristics disclosed herein, e.g., high binding affinity, favorable binding kinetics, binding specificity, favorable pharmacokinetics, reduced self-aggregation, favorable expression profile (e.g., in mammalian cells), and/or stability.

In some embodiments, a polypeptide provided herein, e.g., a LC polypeptide and/or a HC polypeptide, is characterized by including in an Activin A/B antibody agent.

In some embodiments, an Activin A/B antibody agent described herein binds to human Activin-A, Activin-B, and/or to one or more complexes that include one or both Activin-A and Activin-B. In some embodiments, an Activin A/B antibody agent described herein binds to Activin-A from a species having high homology with human Activin-A, e.g., as provided in Table 15. In some embodiments, an Activin A/B antibody agent described herein binds to Activin-B from a species having high homology with human Activin-B, e.g., as provided in Table 16. In some embodiments, an Activin A/B antibody agent described herein binds to primate Activin-A and/or Activin-B. In some embodiments, an Activin A/B antibody agent described herein binds to a domestic animal Activin-A and/or Activin-B, e.g., a dog, a cat, a ferret, a horse, a cow, a pig, a sheep, a yak or a camel Activin-A and/or Activin-B.

In some embodiments, an Activin A/B antibody agent or an Activin-A and/or Activin-B polypeptide binds to human Activin-A and/or Activin-B with a binding affinity (K_{D}) of about 83 pM to about 1640 pM. In some embodiments, an Activin A antibody agent or an Activin A polypeptide binds to human Activin A with a binding affinity (K_{D}) of about 83 pM, about 84 pM, about 85 pM, about 86 pM, about 87 pM, about 89 pM, about 90 pM, about 100 pM, about 200 pM, about 300 pM, about 400 pM, about 500 pM, about 600 pM, about 650 pM, about 700 pM, about 750 pM, about 800 pM, about 850 pM, about 900 pM, about 950 pM, about 1000 pM, about 1050 pM, about 1100 pM, about 1200 pM, about 1250 pM, about 1300 pM, about 1350 pM, about 1400 pM, about 1450 pM, about 1500 pM, about 1550 pM, about 1600 pM, about 1650 pM, about 1700 pM.

In some embodiments, an Activin A/B antibody agent or an Activin-A and/or Activin-B polypeptide binds to human Activin-A, Activin-B, and/or to one or more complexes that include one or both Activin-A and Activin-B with a binding affinity (K_{D}) of about 83 pM to about 1700 pM, about 84 pM to about 1700 pM, about 85 pM to about 1000 pM, about 8 pM to about 1000 pM, about 9 pM to about 1000 pM, about 10 pM to about 1700 pM, about 90 pM to about 1700 pM, about 100 pM to about 1700 pM, about 200 pM to about 1700 pM, about 200 pM to about 1700 pM, about 300 pM to about 1700 pM, about 400 pM to about 1700 pM, about 500 pM to about 1700 pM, about 600 pM to about 1700 pM, about 700 pM to about 1700 pM, about 750 pM to about 1700 pM, about 800 pM to about 1700 pM, about 850 pM to about 1700 pM, about 900 pM to about 1700 pM, about 1000 pM to about 1700 pM, about 1050 pM to about 1700 pM, about 1100 pM to about 1700 pM, about 1150 pM to about 1700 pM, about 1200 pM to about 1700 pM, about 1250 pM to about 1700 pM, about 1300 pM to about 1700 pM, about 1350 pM to about 1700 pM, about 1400 pM to about 1700 pM, about 1450 pM to about 1700 pM, about 1500 pM to about 1700 pM, about 1550 pM to about 1700 pM, about 1600 pM to about 1700 pM, about 1650 pM to about 1700 pM.

In some embodiments, an Activin A/B antibody agent or an Activin-A and/or Activin-B polypeptide binds to human Activin-A, Activin-B, and/or to one or more complexes that include one or both Activin-A and Activin-B with a binding affinity (Kp) of about 83 pM to about 1700 pM, about 83 pM to about 1650 pM, about 83 pM to about 1600 pM, about 83 pM to about 1550 pM, about 83 pM to about 1500 pM, about 83 pM to about 1450 pM, about 83 pM to about 1400 pM, about 83 pM to about 1350 pM, about 83 pM to about 1300 pM, about 83 pM to about 1250 pM, about 83 pM to about 1200 pM, about 83 pM to about 1150 pM, about 83 pM to about 1100 pM, about 83 pM to about 1050 pM, about 83 pM to about 1000 pM, about 83 pM to about 950 pM, about 83 pM to about 900 pM, about 83 pM to about 850 pM, about 83 pM to about 800 pM, about 83 pM to about 750 pM, about 83 pM to about 700 pM, about 83 pM to about 650 pM, about 83 pM to about 600 pM, about 83 pM to about 550 pM, about 83 pM to about 500 pM, about 83 pM to about 450 pM, about 83 pM to about 400 pM, about 83 pM to about 350 pM, about 300 pM to about 250 pM, about 83 pM to about 200 pM, about 83 pM to about 150 pM, about 83 pM to about 100 pM, about 83 pM to about 90 pM, about 83 pM to about 89 pM, about 83 pM to about 88 pM, about 83 pM to about 87 pM, about 83 pM to about 86 pM, about 83 pM to about 85 pM, about 83 pM to about 84 pM.

In some embodiments, binding of an Activin-A and/or Activin-B polypeptide agent or an Activin-A and/or Activin-B polypeptide to Activin-A and/or Activin-B is measured using a Surface Plasmon Resonance assay (e.g., Biacore assay) or an Octet assay as described herein. In some embodiments, binding is measured in a Fab format. In some embodiments, binding is measured in an IgG format.

In some embodiments, a binding affinity is determined with a binding affinity determining assay such as an Octet assay or a comparable assay

In some embodiments, an Activin A/B antibody agent does not bind to or has minimal binding affinity for one or more TGFbeta super family members other than Activin-A and/or Activin-B. In some embodiments, an Activin A/B antibody agent does not bind to or has minimal binding affinity for any one or all or a combination of GDNF, GDF8, GDF10, GDF11, BMP9, BMP10, GDF15 and/or Follistatin.

In some embodiments, an Activin A/B antibody agent disclosed herein binds to one or more members of the TGFbeta super family in addition to Activin-A and/or Activin-B. In some embodiments, an Activin A/B antibody agent disclosed herein binds to Activin-A and Activin-B also binds to GDF10 and/or GDF11. In some embodiments, an Activin-A and/or Activin-B binding agent which binds to Activin-A and/or Activin-B and GDF11 does not modulate an activity and/or level of GDF11, e.g., when characterized in an assay that evaluates an GDF11 activity and/or level.

In some embodiments, an Activin A/B antibody agent has high specificity for Activin-A and/or Activin-B and low polyreactivity, e,g., as measured with a poly-specificity reagent (PSR) or a comparable reagent that measures antibody binding specificity. In some embodiments an Activin A/B antibody agent has a clean PSR score of less than 0.1. In some embodiments, an Activin A/B antibody agent has a low PSR score of between 0.1 to 0.33. In some embodiments, an Activin A/B antibody agent has a low PSR score of about 0.1. In some embodiments, an Activin A/B antibody agent has a low PSR score of about 0.11. In some embodiments, an Activin A/B antibody agent has a low PSR score of about 0.12. In some embodiments, an Activin A/B antibody agent has a low PSR score of about 0.13. In some embodiments, an Activin A/B antibody agent has a low PSR score of about 0.14. In some embodiments, an Activin A/B antibody agent has a low PSR score of about 0.15. In some embodiments, an Activin A/B antibody agent has a low PSR score of about 0.16. In some embodiments, an Activin A/B antibody agent has a low PSR score of about 0.17. In some embodiments, an Activin A/B antibody agent has a low PSR score of about 0.18. In some embodiments, an Activin A/B antibody agent has a low PSR score of about 0.19. In some embodiments, an Activin A/B antibody agent has a low PSR score of about 0.2. In some embodiments, an Activin A/B antibody agent has a low PSR score of about 0.21. In some embodiments, an Activin A/B antibody agent has a low PSR score of about 0.22. In some embodiments, an Activin A/B antibody agent has a low PSR score of about 0.23. In some embodiments, an Activin A/B antibody agent has a low PSR score of about 0.24. In some embodiments, an Activin A/B antibody agent has a low PSR score of about 0.25. In some embodiments, an Activin A/B antibody agent has a low PSR score of about 0.26. In some embodiments, an Activin A/B antibody agent has a low PSR score of about 0.27. In some embodiments, an Activin A/B antibody agent has a low PSR score of about 0.28. In some embodiments, an Activin A/B antibody agent has a low PSR score of about 0.29. In some embodiments, an Activin A/B antibody agent has a low PSR score of about 0.3. In some embodiments, an Activin A/B antibody agent has a low PSR score of about 0.31. In some embodiments, an Activin A/B antibody agent has a low PSR score of about 0.32. In some embodiments, an Activin A/B antibody agent has a low PSR score of about 0.33.

In some embodiments, an Activin A/B antibody agent has low hydrophobicity as measured in a HIC assay or a comparable assay that measures hydrophobicity. In some embodiments, an Activin A/B antibody agent has a HIC retention time of less than 10.5 minutes indicating a clean to low HIC. In some embodiments, an Activin A/B antibody agent has a retention time of less than 10.5 minutes, less than 10 minutes, or less than 9.5 minutes. In some embodiments, an Activin A/B antibody agent has a retention time of about 9.2 minutes, about 9.4 minutes, about 9.5 minutes, about 9.6 minutes, about 9.7 minutes, about 9.8 minutes, about 9.9 minutes, about 10 minutes, about 10.1 minutes, about 10.2 minutes, about 10.3 minutes, about 10.4 minutes or about 10.5 minutes.

In some embodiments, an Activin A/B antibody agent has a retention time of between 9.2 to 10.5 minutes indicating a clean to low HIC. In some embodiments, an Activin A/B antibody agent has a retention time of about 9.2 minutes. In some embodiments, an Activin A/B antibody agent has a retention time of about 9.4 minutes. In some embodiments, an Activin A/B antibody agent has a retention time of about 9.6 minutes. In some embodiments, an Activin A/B antibody agent has a retention time of about 9.8 minutes. In some embodiments, an Activin A/B antibody agent has a retention time of about 10.0 minutes. In some embodiments, an Activin A/B antibody agent has a retention time of about 10.1 minutes. In some embodiments, an Activin A/B antibody agent has a retention time of about 10.2 minutes. In some embodiments, an Activin A/B antibody agent has a retention time of about 10.3 minutes. In some embodiments, an Activin A/B antibody agent has a retention time of about 10.4 minutes. In some embodiments, an Activin A/B antibody agent has a retention time of about 10.5 minutes.

In some embodiments, an Activin A/B antibody agent is produced in a bacterial cell, e.g., E. coli.

In some embodiments, an Activin A/B antibody agent is produced in a yeast cell, e.g., S. cerevisiae or S. pombe.

In some embodiments, an Activin A/B antibody agent is produced in an insect cell, e.g., Sf9.

In some embodiments, an Activin A/B antibody agent is produced in a mammalian cell. In some embodiments, a mammalian cell is chosen from a CHO cell, a COS cell, a HEK-293 cell, an NSO cell, a PER. C6 cell, or an Sp2.0 cell.

In some embodiments, an Activin A/B antibody agent can be produced at a concentration of about 10 mg/L to about 20,000 mg/L. In some embodiments, an Activin A/B antibody agent can be produced at a concentration of about 10 mg/L, about 20 mg/L, about 30 mg/L, about 40 mg/L, about 50 mg/L, about 60 mg/L, about 70 mg/L, about 80 mg/L, about 90 mg/L, about 100 mg/L, about 150 mg/L, about 200 mg/L, about 250 mg/L, about 300 mg/L, about 350 mg/L, about 400 mg/L, about 450 mg/L, about 500 mg/L, about 550 mg/L, about 600 mg/L, about 650 mg/L, about 700 mg/L, about 750 mg/L, about 800 mg/L, about 850 mg/L, about 900 mg/L, about 950 mg/L, about 1000 mg/L, about 2000 mg/L, about 2000 mg/L, about 3000 mg/L, about 4000 mg/L, about 5000 mg/L, about 6000 mg/L, about 7000 mg/L, about 8000 mg/L, about 9000 mg/L, about 10,000 mg/L, about 15,000 mg/L or about 20,000 mg/L. In some embodiments, an Activin A/B antibody agent can be produced at a concentration of more than 100 mg/L, more than 200 mg/L, more than 500 mg/L, more than 1000 mg/L, more than 2000 mg/L, more than 3000 mg/L, more than 4000 mg/L, more than 5000 mg/L, more than 6000 mg/L, more than 7000 mg/L, more than 8000 mg/L, more than 9000 mg/L, more than 10,000 mg/L. In some embodiments, an Activin A/B antibody agent can be produced at a concentration of about 1000 to 20,000 mg/L, about 2000 to 20,000 mg/L, about 5000 to 20,000 mg/L, about 6000 to 20,000 mg/L, about 7000 to 20,000 mg/L, about 8000 to 20,000 mg/L, about 9000 to 20,000 mg/L, 10,000 to 20,000 mg/L or about 15,000 to 20,000 mg/L.

In some embodiments, an Activin A/B antibody agent has a melting temperature (Tm) of about 65° C. to about 85° C., about 65° C. to about 80° C., about 65° C. to about 75° C., about 65° C. to about 70° C., about 70° C. to about 85° C., about 75° C. to about 80° C. In some embodiments, an Activin A/B antibody agent has a melting temperature (Tm) of about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., or about 83° C., or about 84° C., or about 85° C.

In some embodiments, an Activin A/B antibody agent has low self-association as measured with an AC-SINS assay or a comparable assay that measures self-association. In some embodiments, an Activin A/B antibody agent has an AC-SINS score less than 5, or between 5 and 20 indicating low self-association. In some embodiments, an Activin A/B antibody agent has an AC-SINS score of about 0.5. In some embodiments, an Activin A/B antibody agent has an AC-SINS score of about 0.6. In some embodiments, an Activin A/B antibody agent has an AC-SINS score of about 0.7. In some embodiments, an Activin A/B antibody agent has an AC-SINS score of about 0.8. In some embodiments, an Activin A/B antibody agent has an AC-SINS score of about 0.9. In some embodiments, an Activin A/B antibody agent has an AC-SINS score of about 1. In some embodiments, an Activin A/B antibody agent has an AC-SINS score of about 2. In some embodiments, an Activin A/B antibody agent has an AC-SINS score of about 3. In some embodiments, an Activin A/B antibody agent has an AC-SINS score of about 4. In some embodiments, an Activin A/B antibody agent has an AC-SINS score of about 5. In some embodiments, an Activin A/B antibody agent has an AC-SINS score of about 6. In some embodiments, an Activin A/B antibody agent has an AC-SINS score of about 7. In some embodiments, an Activin A/B antibody agent has an AC-SINS score of about 8. In some embodiments, an Activin A/B antibody agent has an AC-SINS score of about 9. In some embodiments, an Activin A/B antibody agent has an AC-SINS score of about 10. In some embodiments, an Activin A/B antibody agent has an AC-SINS score of about 11. In some embodiments, an Activin A/B antibody agent has an AC-SINS score of about 12. In some embodiments, an Activin A/B antibody agent has an AC-SINS score of about 13. In some embodiments, an Activin A/B antibody agent has an AC-SINS score of about 14. In some embodiments, an Activin A/B antibody agent has an AC-SINS score of about 15. In some embodiments, an Activin A/B antibody agent has an AC-SINS score of about 16. In some embodiments, an Activin A/B antibody agent has an AC-SINS score of about 17. In some embodiments, an Activin A/B antibody agent has an AC-SINS score of about 18. In some embodiments, an Activin A/B antibody agent has an AC-SINS score of about 19. In some embodiments, an Activin A/B antibody agent has an AC-SINS score of about 20.

In some embodiments, an Activin A/B antibody agent has an AC-SINS score of more than 20. In some embodiments, an Activin A/B antibody agent has an AC-SINS score of about 21, about 22, about 23, about 24, about 25, about 26, about 27, about 28, about 29 or about 30.

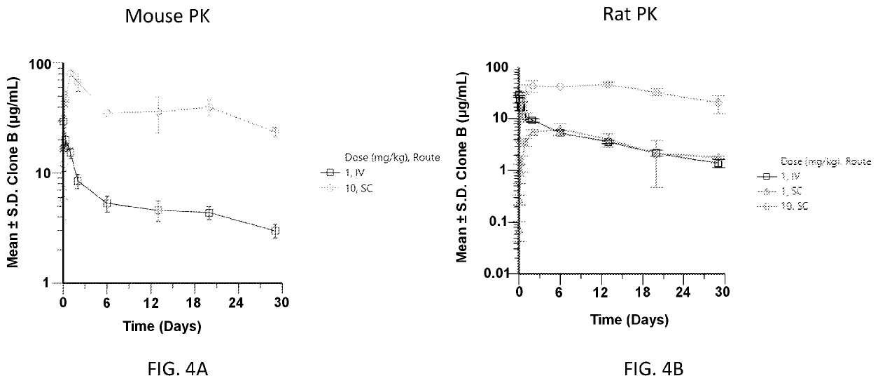

Target mediated drug disposition (TMDD) is a mechanism by which antibodies can be eliminated from the body. TMDD refers to receptor-mediated endocytosis of antibodies in which the binding event is facilitated by interaction of antigen recognition domains of the antibody fragment to its target antigen. Antibodies which are cleared by TMDD usually have dose-dependent non-linear elimination, e.g., as described in Ryman J T and Melbohm B (2017) CPT Pharmacometrics Syst. Pharmacol. 6, 576-588 the entire contents of which are hereby incorporated by reference. In some embodiments, an Activin A/B antibody agent has a pharmacokinetic profile with linear elimination, e.g., as shown in FIGS. 4A-4B. In some embodiments, an Activin A/B antibody agent has minimal or no target mediated-drug disposition (TMDD), e.g., an Activin A/B antibody agent is not eliminated by endocytosis. In some embodiments, an Activin A/B antibody agent without TMDD has increased bioavailability compared to an otherwise similar antibody agent with TMDD.

In some embodiments, an Activin-A and/or Activin-B antibody agent disclosed herein is characterized in that when administered to a subject it prevents, reduces and/or reverses weight loss (e.g., cachexia) induced by Activin A and/or Activin B, e.g., as compared to before administration of an Activin-A and/or Activin-B antibody agent.

In some embodiments, an Activin-A and/or Activin-B antibody agent disclosed herein is characterized in that when administered to a subject it prevents, reduces and/or reverses organ damage induced by Activin A and/or Activin B, e.g., as compared to before administration of an Activin-A and/or Activin-B antibody agent. In some embodiments, organ damage comprises: a change in function of an organ, a reduction in size of an organ (e.g., atrophy), a change in cellularity of an organ (e.g., increased infiltration of immune cells), a reduction in weight of an organ, or any combination thereof. In some embodiments, an organ that can be damaged by expression, e.g., overexpression, of Activin A and/or Activin B comprises: liver, heart, pancreas, kidney, or a combination thereof.

In some embodiments, an Activin-A and/or Activin-B antibody agent disclosed herein is characterized in that when administered to a subject it prevents, reduces and/or reverses liver damage induced by Activin A and/or Activin B, e.g., as compared to before administration of an Activin-A and/or Activin-B antibody agent. In some embodiments, administration of an Activin A/B antibody agent promotes liver regeneration. In some embodiments, liver damage comprises a change in function of a liver, a reduction in size of a liver (e.g., atrophy), a change in cellularity of a liver (e.g., increased infiltration of immune cells), or a reduction in weight of a liver, or any combination thereof. In some embodiments, liver damage comprises fibrosis. In some embodiments, liver damage comprises hepatitis.

In some embodiments, an Activin-A and/or Activin-B antibody agent disclosed herein is characterized in that when administered to a subject it prevents, reduces and/or reverses damage to a heart induced by Activin A and/or Activin B, e.g., as compared to before administration of an Activin-A and/or Activin-B antibody agent. In some embodiments, heart damage comprises a change in function of a heart, a reduction in size of a heart (e.g., atrophy), a change in cellularity of a heart (e.g., increased infiltration of immune cells), or a reduction in weight of a heart, or any combination thereof. In some embodiments, heart damage comprises fibrosis.

In some embodiments, an Activin-A and/or Activin-B antibody agent disclosed herein is characterized in that when administered to a subject it prevents, reduces and/or reverses kidney damage induced by Activin A and/or Activin B, e.g., as compared to before administration of an Activin-A and/or Activin-B antibody agent. In some embodiments, kidney damage comprises a change in function of a kidney, a reduction in size of a kidney (e.g., atrophy), a change in cellularity of a kidney (e.g., increased infiltration of immune cells), or a reduction in weight of a kidney, or any combination thereof. In some embodiments, kidney damage comprises fibrosis. In some embodiments, kidney damage comprises tubular degeneration.

In some embodiments, an Activin-A and/or Activin-B antibody agent disclosed herein is characterized in that when administered to a subject it prevents, reduces and/or reverses pancreatic damage induced by Activin A and/or Activin B, e.g., as compared to before administration of an Activin-A and/or Activin-B antibody agent. In some embodiments, pancreatic damage comprises a change in function of a pancreas, a reduction in size of a pancreas (e.g., atrophy), a change in cellularity of a pancreas (e.g., increased infiltration of immune cells), or a reduction in weight of a pancreas, or any combination thereof. In some embodiments, pancreatic damage comprises pancreatitis.

Nucleic Acids Encoding Activin A/B Antibody Agents and/or Provided Polypeptides

The present disclosure, among other things, provides nucleic acids encoding Activin A/B antibody agents described herein, or polypeptides provided herein (e.g., LC polypeptides and/or HC polypeptides). The present disclosure includes nucleic acids encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein. Such nucleic acids may be present in a vector. Such nucleic acids may be present in the genome of a cell, e.g., a cell of a subject in need of treatment or a cell for production of an antibody, e.g. a mammalian cell for production of an Activin A/B antibody agent described herein, or polypeptides provided herein (e.g., LC polypeptides and/or HC polypeptides).

Nucleic acids encoding an Activin A/B antibody agent, or polypeptides provided herein (e.g., LC polypeptides and/or HC polypeptides) may be modified to include codons that are optimized for expression in a particular cell type or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acids encoding an Activin A/B antibody agent described herein, or polypeptides provided herein (e.g., LC polypeptides and/or HC polypeptides), in whole or in part, may include an altered sequence to optimize codon usage for a particular cell type (e.g., a eukaryotic or prokaryotic cell). For example, a coding sequence for a humanized heavy (or light) chain variable region as described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host. Selection of vectors and methods to construct them are commonly known to persons of ordinary skill in the art (see, e.g., “Recombinant DNA Part D,” Methods in Enzymology, Vol. 153, Wu and Grossman, eds., Academic Press (1987)).

Conventionally used techniques including, for example, electrophoresis, calcium phosphate precipitation, DEAE-dextran transfection, or lipofection, may be used to introduce a foreign nucleic acid (e.g., DNA or RNA) into a prokaryotic or eukaryotic host cell. Desirably, a vector may include regulatory sequences, such as transcription and/or translation initiation and/or termination codons, which are specific to the type of host (e.g., bacterium, fungus, plant, or animal) into which a vector is to be introduced, as appropriate and taking into consideration whether a vector is DNA or RNA. In some embodiments, a vector comprises regulatory sequences that are specific to a genus of a host cell. In some embodiments, a vector comprises regulatory sequences that are specific to a species of a host.

In addition to a replication system and an inserted nucleic acid, a nucleic acid construct can include one or more marker genes, which allow for selection of transformed or transfected hosts. Exemplary marker genes include, e.g., biocide resistance (e.g., resistance to antibiotics or heavy metals) or complementation in an auxotrophic host to provide prototrophy.

An expression vector can comprise a native or nonnative promoter operably linked to an isolated or purified nucleic acid as described above. Selection of promoters, e.g., strong, weak, inducible, tissue-specific, and/or developmental-specific, is within the skill of one in the art. Similarly, combining a nucleic acid as described above with a promoter is also within the skill of one in the art.

Suitable vectors include those designed for propagation and expansion and/or for expression. For example, a cloning vector may be selected from the pUC series, the pBluescript series (Stratagene, LaJolla, Calif.), the pET series (Novagen, Madison, Wis.), the pGEX series (Pharmacia Biotech, Uppsala, Sweden), the pcDNA3 series (Invitrogen) or the pEX series (Clontech, Palo Alto, Calif.). Bacteriophage vectors, such as λGT10, λGT11, λZapII (Stratagene), λEMBL4, and λNM1149, may be used. Examples of plant expression vectors that can be used include pBI110, pBI101.2, pBI101.3, pBI121, or pBIN19 (Clontech). Examples of animal expression vectors that can be used include pEUK-C1, pMAM, or pMAMneo (Clontech). The TOPO cloning system (Invitrogen, Carlsbad, Calif.) also can be used in accordance with the manufacturer's recommendations.

Additional sequences can be added to such cloning and/or expression sequences to optimize their function in cloning and/or expression, to aid in isolation of a nucleic acid encoding an Activin A/B antibody agent described herein, or to improve introduction of a nucleic acid into a cell. Use of cloning vectors, expression vectors, adapters, and linkers is well known in the art (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding an Activin A/B antibody agent described herein, or polypeptides provided herein (e.g., LC polypeptides and/or HC polypeptides), under control of transcriptional and/or translational control signals. These methods may include in vitro recombinant DNA and synthetic techniques and in vivo recombination (see, e.g., Ausubel, supra; or Sambrook, supra).

In some embodiments, a nucleic acid encoding an Activin A/B antibody agent described herein or polypeptides provided herein (e.g., LC polypeptides and/or HC polypeptides), is or comprises DNA.

In some embodiments, a nucleic acid encoding an Activin A/B antibody agent described herein or polypeptides provided herein (e.g., LC polypeptides and/or HC polypeptides), is or comprises RNA, e.g., messenger RNA.

### Pharmaceutical Compositions

The present disclosure, among other things, provides pharmaceutical compositions that comprise or otherwise deliver an Activin A/B antibody agent; typically, such pharmaceutical compositions comprise an active agent (e.g., an antibody agent or portion thereof, or a nucleic acid that encodes such antibody agent or portion thereof, etc.) one or more pharmaceutically or physiologically acceptable carriers, diluents, or excipients.

When “a therapeutically effective amount, “an immunologically effective amount,” “an anti-immune response effective amount,” or “an immune response-inhibiting effective amount” is indicated, a precise amount of a pharmaceutical composition that comprises or delivers an Activin A/B antibody agent described herein can be determined by a physician with consideration, for example, of individual differences in age, weight, immune response, and condition of the patient (subject).

In some embodiments, pharmaceutical compositions described herein may comprise buffers including neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of a contaminant (e.g., an endotoxin).

In some embodiments, pharmaceutical compositions described herein may be administered in a manner appropriate to the disease, disorder, or condition to be treated or prevented. In some embodiments, quantity and/or frequency of administration may be determined by such factors as condition of a patient, and/or type and/or severity of a patient's disease, disorder, or condition, although appropriate dosages may be determined by clinical trials.

In some embodiments, a pharmaceutical composition provided by the present disclosure may be in a form such as, for example, liquid, semi-solid and solid dosage forms, such as liquid solutions (e.g., injectable and infusible solutions), dispersions or suspensions, liposomes, and suppositories. Typically, pharmaceutical compositions that comprise or deliver antibody agents are injectable or infusible solutions; in some such embodiments, such compositions can be formulated for administration intravenously, subcutaneously, intradermally, intratumorally, intranodally, intramedullary, intramuscularly, transarterially, sublingually, intranasally, topically or intraperitoneally. In some embodiments, provided pharmaceutical compositions are formulated for intravenous administration. In some embodiments, provided pharmaceutical compositions are formulated for subcutaneous administration.

Pharmaceutical compositions described herein can be formulated for administration by using infusion techniques that are commonly known in the field (See, e.g., Rosenberg et al., New Eng. J. of Med. 319:1676, 1988, which is hereby incorporated by reference in its entirety).

In some embodiments, pharmaceutical compositions described herein are administered in combination with (e.g., before, simultaneously, or following) an additional therapy for a symptom, disease or disorder, e.g., a SOC therapy for a symptom, disease or disorder. In some embodiments, pharmaceutical compositions described herein may be administered before or following surgery.

In some embodiments, a dosage of any aforementioned therapy to be administered to a subject will vary with a disease, disorder, or condition being treated and based on a specific subject. Scaling of dosages for human administration can be performed according to art-accepted practices.

Exemplary Identification, Characterization and/or Production of Activin A/B Antibody Agents or Components (e.g., Polypeptide Elements or Portions) Thereof

The present disclosure, among other things, provides production, identification, and/or characterization of an Activin A/B antibody agent described herein, or polypeptides provided herein (e.g., LC polypeptides and/or HC polypeptides). In some embodiments, an Activin A/B antibody agent described herein is identified, characterized, and/or produced using a display technology, such as yeast display, phage display, or ribosome display. In some embodiments, an Activin A/B antibody agent described herein is identified, characterized and/or producing using hybridoma technology. In some embodiments, identification and/or characterization of a provided antibody agent utilizes library screening (e.g., of a hybridoma library, a phage library, a ribosome library, a yeast library, etc.

Phage library based methods for identifying, characterizing, and/or producing antibodies are known in the art (as described in, e.g., Ladner et al. U.S. Pat. No. 5,223,409; Kang et al. International Publication No. WO 92/18619; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; each of which his hereby incorporated by reference in its entirety).

Yeast library based methods for identifying, characterizing, and/or producing antibodies are known in the art, e.g., as described in U.S. Pat. No. 8,691,730 and Chao G. et al (2006) Nature Protocols 1 (2): 755-68, each of which his hereby incorporated by reference in its entirety.

In some embodiments, an Activin A/B antibody agent described herein may be derived from another species (e.g., a species other than human). A humanized antibody is an antibody (typically produced by recombinant DNA technology), in which some or all amino acids of a human immunoglobulin light chain or heavy chain that are not required for antigen binding (e.g., constant regions and/or framework regions of variable domains) are used to substitute for the corresponding amino acids from light chain or heavy chain of the cognate, nonhuman antibody. By way of example, a humanized version of a murine antibody to a given antigen has on both heavy and light chains: (1) constant regions of a human antibody; (2) FRs from the variable domains of a human antibody; and (3) CDRs from the murine antibody. Human FRs may be selected based on their highest sequence homology to mouse FR sequence. When necessary or desirable, one or more residues in human FRs can be changed to residues at corresponding positions in a murine antibody so as to preserve binding affinity of the humanized antibody to a target. Such a change is sometimes called “back mutation.” Similarly, forward mutations may be made to revert back to murine sequence for a desired reason, e.g. stability or affinity to a target. Those skilled in the art are aware that humanized antibodies generally are less likely to elicit an immune response in humans as compared to chimeric human antibodies because the former contain considerably fewer non-human components.

Technologies for humanizing non-human antibodies are well known in the art. Suitable methods for making humanized antibodies in accordance with the present disclosure are described in, e.g., Winter EP 0 239 400; Jones et al., Nature 321:522-525 (1986); Riechmann et al., Nature 332:323-327 (1988); Verhoeyen et al., Science 239:1534-1536 (1988); Queen et al., Proc. Nat. Acad. ScL USA 86:10029 (1989); U.S. Pat. No. 6,180,370; and Orlandi et al., Proc. Natl. Acad. Sd. USA 86:3833 (1989); the disclosures of each of which are incorporated herein by reference in their entireties. Generally, transplantation of non-human (e.g., murine) CDRs onto a human antibody is achieved as follows. cDNAs encoding VH and VL are isolated from a hybridoma, and nucleic acid sequences encoding VH and VL including CDRs are determined by sequencing. Nucleic acid sequences encoding CDRs are inserted into corresponding regions of a human antibody VH or VL coding sequences and attached to human constant region gene segments of a desired isotype (e.g., γl for CH and κ for CL). Humanized heavy and light chain genes are co-expressed in mammalian host cells (e.g., CHO or NSO cells) to produce soluble humanized antibody. To facilitate large-scale production of antibodies, it is often desirable to select for a high expressor using, for example, a DHFR gene or GS gene in the producer line.

In some embodiments, an Activin A/B antibody agent described herein comprises or is a human antibody. Completely human antibodies may be particularly desirable for therapeutic treatment of human subjects. Human antibodies can be made by a variety of methods known in the art including phage display methods described above using antibody libraries derived from human immunoglobulin sequences (see, e.g., U.S. Pat. Nos. 4,444,887 and 4,716,111; and PCT publications WO 98/46645, WO 98/60433, WO 98/24893, WO 98/16664, WO 96/34096, WO 96/33735, and WO 91/10741; each of which is incorporated herein by reference in its entirety). Techniques are also available for the preparation of human monoclonal antibodies in, e.g., Cole et al., Monoclonal Antibodies and Cancer Therapy, Alan R. Riss, (1985); and Boerner et al., J. Immunol., 147 (1): 86-95, (1991), each of which is incorporated herein by reference in its entirety.

Also disclosed herein is a method of making an Activin A/B antibody agent, comprising culturing a host cell comprising a heterologous nucleic acid encoding an Activin A/B antibody polypeptide or combination thereof, under a condition wherein an Activin A/B antibody polypeptide or combination thereof (e.g., an Activin-A and/or Activin-B polypeptide agent) is expressed by said host cell. In some such embodiments, the heterologous nucleic acid is or comprises a vector comprising an Activin A/B antibody agent nucleic acid sequence.

In some embodiments, a host cell is a yeast cell, a bacterial cell, a mammalian cell or an insect cell.

In some embodiments, a host cell is a mammalian cell. In some embodiments, a mammalian cell is chosen from a CHO cell, a COS cell, a HEK-293 cell, an NSO cell, a PER.C6 cell, or an Sp2.0 cell.

### Exemplary Uses of Activin A/B Antibody Agents or Components (e.g., Polypeptide Elements or Portions) Thereof

Polypeptides disclosed herein, e.g., a LC polypeptide and/or a HC polypeptide, can be included in an Activin A/B antibody agent.

Activin A/B antibody agents are useful in a variety of contexts, including in research, diagnosis, and therapy. In some embodiments, an Activin A/B antibody agent disclosed herein can be used as a reference agent and/or a reagent in research, e.g., to understand Activin-A and/or Activin-B biology and/or biological processes directly or indirectly related to Activin-A and/or Activin-B. In some embodiments, an Activin A/B antibody agent disclosed herein can be used as a reference agent and/or a reagent in diagnosis and/or treatment (e.g., patient selection).

This disclosure provides methods of using an Activin A/B antibody agent for, e.g., inhibiting Activin-A and/or Activin-B (e.g., reducing an activity and/or level of Activin-A and/or Activin-B) in a cell, tissue or subject (e.g., in a subject or in a sample from a subject). In some embodiments, Activin-A and/or Activin-B comprises free and/or active Activin-A and/or Activin-B. In some embodiments, Activin-A and/or Activin-B is or comprises free Activin-A and/or Activin-B. In some embodiments, Activin-A and/or Activin-B is or comprises active Activin-A and/or Activin-B. In some embodiments, Activin-A and/or Activin-B is or comprises free and active Activin-A and/or Activin-B.

In some embodiments, an increased level of Activin-A and/or Activin-B is about 500 pg/ml or more. In some embodiments, a level and/or activity of Activin-A and/or Activin-B is evaluated in a subject, e.g., via imaging, or in a sample from a subject, e.g., a tissue sample (e.g., a biopsy such as a tumor biopsy), or a bodily fluid sample (e.g., a blood, plasma, serum, urine, CSF, saliva or other bodily fluid).

In some embodiments, an Activin-A and/or Activin-B antibody agent disclosed herein is characterized in that when administered to a subject it reduces a level and/or activity of Activin-A and/or Activin-B, e.g., as compared to before administration of an Activin-A and/or Activin-B antibody agent. In some embodiments, reduced Activin-A and/or Activin-B level and/or activity is assessed in a subject, e.g., via imaging, or in a sample from a subject, e.g., a tissue sample (e.g., a biopsy such as a tumor biopsy), or a bodily fluid sample (e.g., a blood, plasma, serum, urine, CSF, saliva or other bodily fluid).

In some embodiments, an Activin-A and/or Activin-B antibody agent reduces an Activin-A and/or Activin-B level to less than 500 pg/ml. In some embodiments, an Activin-A and/or Activin-B antibody agent reduces an Activin-A and/or Activin-B level to at least 1%-90% less than before administration of an Activin-A and/or Activin-B antibody agent.

Also disclosed herein are uses of an Activin-A and/or Activin-B antibody agent (or a composition comprising the same) for ameliorating (e.g., reducing) one or more symptoms associated with a disease or disorder, or one or more symptoms associated with (e.g., induced by) a therapy for a disease or disorder. In some embodiments, a symptom associated with a disease or disorder, or a symptom associated with (e.g., induced by) a therapy for a disease or disorder is weight loss, loss of appetite, fatigue, muscle mass loss, fat mass loss, organ mass loss, lean mass loss, lean mass atrophy, bone loss, anemia, or fibrosis or combinations thereof.

### Indications

Among other things, the present disclosure provides insights regarding usefulness of provided Activin A/B antibody agents in various contexts and/or indications, including in certain contexts and/or indications where therapeutic targeting of Activin-A and/or Activin-B has not previously been proposed.

In some embodiments, an Activin A/B antibody agent as described herein (e.g., an anti-Activin A/B antibody) may be useful in treating and/or preventing a disease or disorder associated with increased levels of Activin-A and/or Activin-B. In some embodiments, an increased level of Activin-A and/or Activin-B is a level of about 500 pg/ml or more, e.g., as evaluated in a sample from a subject, e.g., a blood, plasma, serum or urine sample. In some embodiments, Activin-A and/or Activin-B comprises free and/or active Activin-A and/or Activin-B. In some embodiments, Activin-A and/or Activin-B comprises free Activin-A and/or Activin-B. In some embodiments, Activin-A and/or Activin-B comprises active Activin-A and/or Activin-B. In some embodiments, Activin-A and/or Activin-B comprises free and active Activin-A and/or Activin-B.

In some embodiments, an Activin A/B antibody agent disclosed herein can be used to prevent and/or treat a condition or disease associated with increased Activin-A and/or Activin-B, e.g., any one or all or a combination of: (i) disorders of the hypothalamic pituitary gonadal axis (e.g., with increased FSH levels); (ii) fibrodysplasia ossificans progressive (FOP); (iii) pulmonary arterial hypertension (PAH) and/or hypertension (e.g., preeclampsia); (iv) Anorexia-Cachexia Disorders associated with chronic diseases such as cancer, chronic kidney disease, chronic heart failure, COPD, elderly and muscle immobility, HIV patients, ICU patients (e.g., loss of body weight, loss of food intake, fat mass loss, organ mass loss, muscle wasting (e.g., muscle atrophy), loss of functional muscle mass and strength, bone loss, anemia, fatigue, and/or organ damage); (v) anemia; (vi) metabolic disorders (e.g., obesity, type 2 diabetes, metabolic syndrome and/or type 1 diabetes, lipodystrophy); (vii) inflammatory disorders (e.g. inflammatory bowel disease, pancreatitis, allergic asthma, COVID, acute respiratory distress syndrome, atopic dermatitis, preeclampsia, acute liver injury, acute kidney injury, Tubulointerstitial nephritis, osteoarthritis and inflammatory-induced anemia); (viii) auto-immune disorders (e.g., SLE or rheumatoid arthritis); (ix) fibrotic diseases (e.g. idiopathic pulmonary fibrosis (IPF), NASH, liver cirrhosis, chronic kidney disease, viral hepatitis, biliary disorders, Alport Syndrome, Uterine Fibroids, Endometriosis); (x) cancer; (xi) cancer-treatment related toxicities (e.g., chemotherapy-induced nephrotoxicity, hepatotoxicity); (xii) cancer related metastases (e.g., changes in cell adhesion, migration and/or invasion); (xiii) aging (e.g., senescence); (xiv) Myalgic Encephalomyelitis or Chronic Fatigue Syndrome; (xv) reproductive disorders (e.g., with elevated FSH, hypogonadotropic hypothalamic amenorrhea, polycystic ovary syndrome (PCOS), menopause, ovarian aging and/or ovarian cancers); (xvi) chemoresistance; or (xvii) heart failure.

In some embodiments, an Activin-A and/or Activin-B antibody agent as described herein (e.g., an anti-Activin A/B antibody) may be useful in preventing and/or reversing mass loss in a subject. In some embodiments, mass comprises fat mass, lean mass, muscle mass, bone mass, or a combination thereof.

In some embodiments, an Activin A/B antibody agent as described herein (e.g., an anti-Activin A/B antibody) may be useful in preventing and/or reversing loss of functional muscle mass and/or loss of muscle strength in a subject.

In some embodiments, an Activin A/B antibody agent as described herein (e.g., an anti-Activin A/B antibody) prevents and/or reverses mass loss by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, mass loss is or comprises fat mass loss.

In some embodiments, mass loss is or comprises lean mass loss.

In some embodiments, mass loss is or comprises bone mass loss.

In some embodiments, mass loss is or comprises organ mass loss. In some embodiments, organ mass loss comprises mass loss in one or more organs. In some embodiments, one or more organs with mass loss comprise liver, heart, kidney, pancreas or combinations thereof.

In some embodiments, mass loss is or comprises loss of functional muscle mass.

In some embodiments, mass loss is or comprises loss of muscle strength.

In some embodiments, mass loss is or comprises muscle mass loss. In some embodiments, muscle is chosen from: cardiac muscle, smooth muscle, skeletal muscle, or a combination thereof. In some embodiments, a subject having muscle mass loss has a disease or disorder characterized by decreased muscle mass and/or strength. In some embodiments, a disease or disorder characterized by decreased muscle mass and/or strength is chosen from: sarcopenia, cachexia, muscle injury, muscle wasting/atrophy, cancer, obesity, diabetes, arthritis, multiple sclerosis, muscular dystrophy, immobility, HIV patients, ICU patients, elderly, amyotrophic lateral sclerosis, Parkinson's disease, osteoporosis, osteoarthritis, osteopenia, a metabolic syndrome, chronic renal failure, renal fibrosis, or chronic obstructive pulmonary disease. In some embodiments, a metabolic syndrome comprises a disease or disorder chosen from: diabetes, obesity, nutritional disorders, organ atrophy, chronic obstructive pulmonary disease, or anorexia.

In some embodiments, an Activin A/B antibody agent as described herein (e.g., an anti-Activin A/B antibody) may be useful in preventing and/or reversing weight loss (e.g., cachexia) in a subject.

In some embodiments, weight loss is involuntary weight loss.

In some embodiments, an Activin A/B antibody agent prevents and/or reverses weight loss by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A/B antibody agent as described herein (e.g., an anti-Activin A/B antibody) may be useful in preventing and/or reversing muscle atrophy in a subject.

In some embodiments, a muscle is chosen from: cardiac muscle, smooth muscle, skeletal muscle, or a combination thereof.

In some embodiments, an Activin A/B antibody agent prevents and/or reverses muscle atrophy by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A/B antibody agent as described herein (e.g., an anti-Activin A/B antibody) may be useful in preventing and/or reversing senescence in a subject.

In some embodiments, an Activin A/B antibody agent prevents and/or reverses senescence by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments of any of the methods disclosed herein, mass loss, weight loss, muscle atrophy, and/or senescence is a symptom of a disease or disorder.

In some embodiments of any of the methods disclosed herein, mass loss, weight loss, muscle atrophy, and/or senescence is not a symptom of a disease or disorder.

In some embodiments, an Activin A/B antibody agent as described herein (e.g., an anti-Activin A/B antibody) may be useful in preventing and/or reversing organ damage in a subject (e.g., damage to one or more organs). In some embodiments, organ damage comprises: a change in function of an organ, a reduction in size of an organ (e.g., atrophy), a change in cellularity of an organ (e.g., increased infiltration of immune cells), a reduction in weight of an organ, or any combination thereof. In some embodiments, organ damage comprises damage to one or more, or all of: a liver, kidney, heart, or pancreas.

In some embodiments, an Activin A/B antibody agent as described herein (e.g., an anti-Activin A/B antibody) may be useful in preventing and/or reversing liver damage in a subject. In some embodiments, liver damage is induced by Activin A and/or Activin B. In some embodiments, administration of an Activin A/B antibody agent promotes liver regeneration.

In some embodiments, liver damage comprises: liver necrosis, liver fibrosis, liver inflammation, alteration in liver function, elevation one or more liver enzymes, changes in one or more biomarkers of liver function, or a combination thereof.

In some embodiments, one or more liver enzymes comprise ALT, AST or both.

In some embodiments, the one or more biomarkers of liver function comprise albumin, CRP and/or bilirubin.

In some embodiments, liver damage is a symptom of liver disease.

In some embodiments, liver disease is or comprises cirrhosis.

In some embodiments, an Activin A/B antibody agent prevents and/or reverses liver damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A/B antibody agent as described herein (e.g., an anti-Activin A/B antibody) may be useful in preventing and/or reversing kidney damage in a subject.

In some embodiments, kidney damage comprises: kidney fibrosis, kidney inflammation, alteration in kidney function, or a combination thereof.

In some embodiments, alteration in kidney function is or comprises proteinuria and/or low glomerular filtration rate (GFR).

In some embodiments, kidney damage is or comprises tubular degeneration.

In some embodiments, kidney damage is a symptom of kidney disease.

In some embodiments, kidney disease is or comprises acute kidney disease or chronic kidney disease.

In some embodiments, an Activin A/B antibody agent prevents and/or reverses kidney damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A/B antibody agent as described herein (e.g., an anti-Activin A/B antibody) may be useful in preventing and/or reversing heart damage in a subject.

In some embodiments, heart damage comprises: heart fibrosis, heart inflammation, alteration in heart function, or a combination thereof.

In some embodiments, heart damage is a symptom of heart disease.

In some embodiments, heart disease is or comprises heart kidney disease or heart kidney disease.

In some embodiments, an Activin A/B antibody agent prevents and/or reverses heart damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments, an Activin A/B antibody agent as described herein (e.g., an anti-Activin A/B antibody) may be useful in preventing and/or reversing pancreatic damage in a subject.

In some embodiments, pancreatic damage comprises: pancreatic fibrosis, pancreatic inflammation, alteration in pancreatic function, or a combination thereof.

In some embodiments, pancreatic damage is a symptom of pancreatic disease.

In some embodiments, pancreatic disease is or comprises acute pancreatic disease or chronic pancreatic disease.

In some embodiments, an Activin A/B antibody agent prevents and/or reverses pancreatic damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

In some embodiments of any of the methods disclosed herein, mass loss, weight loss, muscle atrophy, senescence, liver damage, and/or kidney damage is induced by a therapy for a disease or disorder. In some embodiments, a therapy for a disease or disorder comprises a standard of care, e.g., as described herein.

In some embodiments, an Activin A/B antibody agent as described herein (e.g., an anti-Activin A/B antibody) may be useful in preventing and/or treating a SARS-COV-2 infection. In some embodiments, a SARS-COV-2 infection is or comprises a COVID-19 disease. In some embodiments, a SARS-COV-2 infection comprises acute respiratory distress syndrome (ARDS).

In some embodiments of any of the methods disclosed herein, mass loss, weight loss, muscle atrophy, senescence, liver damage, heart damage, pancreatic damage, and/or kidney damage is reduced relative to a comparator.

In some embodiments, a comparator comprises an otherwise similar cell, tissue or subject not administered an Activin-A and/or Activin-B pharmaceutical composition or administered an Activin-A and/or Activin-B inhibitor a different Activin A/B antibody agent.

In some embodiments, an Activin A/B antibody agent as described herein (e.g., an anti-Activin A/B antibody) may be useful in preventing and/or treating cancer in a subject.

In some embodiments of any of the methods disclosed herein, a subject has an increased level and/or activity of Activin-A and/or Activin-B.

In some embodiments of any of the methods disclosed herein, a method further comprises determining a level and/or activity of Activin-A and/or Activin-B in a sample from the subject.

In some embodiments, an increased level and/or activity of Activin-A and/or Activin-B is determined relative to a comparator. In some embodiments, a comparator comprises a predetermined reference sample such as a sample obtained from an otherwise similar subject who does not have a disease or disorder, or a symptom of a disease or disorder.

Several reports have noted a role for Activin A and/or Activin B in cancer metastases. For example, Mancinelli G et al., reported reduction in tumor metastases in a pancreatic ductal adenocarcinoma (PDAC) mouse model with administration of an Activin-A neutralizing antibody (See Mancinelli G et al., Sci Rep. 2021 Apr. 12; 11(1):7986). For Activin B, Xiong S. et al., noted that Activin B induces cancer cell adhesion, migration and invasion by upregulating intergrin beta 3 via SMAD 2/3-SMAD4 signaling (See Xiong S. et al., Oncotarget. 2015 Oct. 13; 6(31):31659-31673), and Kita A. et al., noted that knockdown of Activin B suppressed cellular invasiveness and migration in oral cancer (See Kita A. et al., J Cancer. 2017; 8(11):2033-2041). Additional reports have noted roles for Activin B in VHL/HIF-induced cellular transformation (see Wacker I. et al., (2009) “Key Role for Activin B in Cellular Transformation after Loss of the von Hippel-Lindau Tumor Suppressor.” Molecular and Cellular Biology, vol 29 (7)) and roles for Activin B in cell migration via a SMAD-independent pathway (See Xiong S et al., Oncotarget 2016 Jun. 28; 7 (26):40060-4007).

The present disclosure teaches that provided Activin A/B antibody agents may be useful to prevent and/or reduce cancer metastases. Those skilled in the art, reading the present disclosure in light of the art, will appreciate such use as described herein.

In some embodiments, an Activin A/B antibody agent as described herein (e.g., an anti-Activin A/B antibody) may be useful in preventing and/or reducing adhesion of cells, migration, and/or invasion of cells in vivo (e.g., preventing and/or reducing cancer metastases).

In some embodiments of any of the methods disclosed herein, a subject has a condition or disorder associated with increased Activin-A and/or Activin-B. In some embodiments of any of the methods disclosed herein, a condition or disorder is a hyperproliferative disorder, e.g., a cancer.

In some embodiments, a method prevents and/or reduces cell adhesion, migration and/or invasion of cells from a primary cancer to one or more secondary sites (e.g., prevents and/or reduced metastases). In some embodiments, changes in cell adhesion, migration of cells to one or more secondary sites, invasion of cells (e.g., intravasation into blood and/or extravasation into a different organ or tissue) is or comprises metastasis.

In some embodiments, a method prevents and/or reduces cell adhesion, migration, and/or invasion of cells by at least 5%, 10%, 20%, 30%, 40%, 50%, 60%, 70%, 80%, 90%, 95% or 99%.

In some embodiments, prevention and/or reduction cell adhesion, migration and/or invasion is assessed relative to a comparator. In some embodiments, a comparator comprises an otherwise similar subject not administered an Activin-A and/or Activin-B pharmaceutical composition or administered an Activin-A and/or Activin-B inhibitor or administered a different Activin A/B antibody agent.

In some embodiments, an Activin A/B antibody agent as described herein (e.g., an anti-Activin A/B antibody) may be useful in reducing metastasis, comprising administering to a subject an Activin A/B antibody agent. In some embodiments, administration of an Activin A/B antibody agent reduces and/or prevent liver metastasis.

In some embodiments, a cancer is chosen from: colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer.

In some embodiments, a cancer is colorectal cancer.

In some embodiments, a cancer has a TP53 mutation.

In some embodiments, a cancer has a SMAD4 mutation.

In some embodiments, a cancer has a TP53 mutation and a SMAD4 mutation.

In some embodiments, method reduces metastasis by at least 5%, 10%, 20%, 30%, 40%, 50%, 60%, 70%, 80%, 90%, 95% or 99%. In some embodiments, reduction of metastasis is assessed relative to a comparator. In some embodiments, a comparator comprises an otherwise similar subject not administered an Activin-A and/or Activin-B pharmaceutical composition or administered an Activin-A and/or Activin-B inhibitor or administered a different Activin A/B antibody agent.

In some embodiments, an Activin A/B antibody agent as described herein (e.g., an anti-Activin A/B antibody) may be useful in treating and/or preventing disorders of the hypothalamic pituitary gonadal axis (e.g., associated with increased FSH levels). In some embodiments, a subject having a disorder of the hypothalamic pituitary gonadal axis has an increased level of Activin-A and/or Activin-B, e.g., as compared to a subject who does not have a disorder of the hypothalamic pituitary gonadal axis In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

At least one report has noted a role for Activin-A in disorders of the hypothalamic pituitary gonadal axis (Namwanje and Brown (2016), Cold Spring Harbor Perspectives in Biology 8 (7): a021881). In the hypothalamic-pituitary-gonadal (HPG) axis, Activins, including Activin-A is an activator of pituitary FSH production and release (Namwanje and Brown 2016).

The present disclosure teaches that provided Activin A/B antibody agents may be useful to treat disorders of the hypothalamic pituitary gonadal axis. Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing fibrodysplasia ossificans progressive (FOP). In some embodiments, a subject having fibrodysplasia ossificans progressive has an increased level of Activin-A and/or Activin-B, e.g., as compared to a subject who does not have fibrodysplasia ossificans progressive. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin A/B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin-A in fibrodysplasia ossificans progressive (FOP). For example, Lin H. et al. noted that Activin-A has a prominent role in FOP and taught that “Activin A induces heterotopic bone formation via mutant ALK2, while inhibition of Activin A blocks spontaneous and trauma-induced HO,” whereby HO refers to heterotropic ossification (see Lin H. et al., (2019) Biosci Rep 39(8):BSR20190377). FOP is known to cause progressive HO. Another report noted a role for Activin-A in enhancing mTOR signaling in the pathogenesis of FOP (see Hino K et al. (2017) Journal of Clinical Investigation 127(9); 3339-3352). A further report teaches “the role of Activin A and inflammation in HO” (see Alessi Wolken D M et al., (2017) Bone vol. 109, pp. 210-217).

The present disclosure teaches that provided Activin A/B antibody agents may be useful to treat and/or prevent fibrodysplasia ossificans progressive (FOP). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing Pulmonary Arterial Hypertension (PAH) or hypertension. In some embodiments, a subject having Pulmonary Arterial Hypertension (PAH) or hypertension has an increased level of Activin-A and/or Activin-B, e.g., as compared to a subject who does not have Pulmonary Arterial Hypertension (PAH) or hypertension. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin-A in Pulmonary Arterial Hypertension (PAH) or hypertension. For example, one report notes that “anti-Activin A antibody reduced PAH PAVSMC growth without affecting canonical (Smads) or non-canonical (Akt, ERK1/2, p38 MAPK) effectors” (see Kurdyashova T V et al., (2018) Int J Mol Sci. 19 (10): 2957). Another report teaches a role for Activin-A in pulmonary vascular disease by using ACTRIIA-Fc, an Activin ligand trap (See Yung L M et al., (2020) Sci Transl. Med 12 (543) eaaz5660). Additional reports noting a role for Activin-A in Pulmonary Arterial Hypertension (PAH) or hypertension include Ryanto G. R. T. et al., (2021) Nature Communications 12:1720; and Tsai Y. et al., Am J Hypertens. 2018 Feb. 9; 31 (3): 369-374).

The present disclosure teaches that provided Activin A/B antibody agents may be useful to treat Pulmonary Arterial Hypertension (PAH) or hyprtension. Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing Anorexia-Cachexia Disorders associated with chronic diseases. In some embodiments, chronic diseases include COPD, chronic kidney disease, chronic heart failure, congestive heart failure, cancer, sarcopenia, muscular dystrophy (e.g., Duchenne's muscular dystrophy) elderly and muscle immobility, ICU patients (e.g., loss of body weight, loss of food intake, fat mass loss, muscle wasting, loss of functional muscle mass, loss of muscle strength, bone loss, anemia and/or fatigue). In some embodiments, Anorexia-Cachexia Disorders associated with chronic diseases include diseases with loss of food intake, fat mass loss, muscle wasting, loss functional muscle, loss of muscle strength, bone loss, anemia, fatigue, or a combination thereof.

In some embodiments, a subject having Anorexia-Cachexia Disorders associated with chronic diseases. has an increased level of Activin-A and/or Activin-B, e.g., as compared to a subject who does not have Anorexia-Cachexia Disorders associated with chronic diseases. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin-A in Anorexia-Cachexia Disorders associated with chronic diseases. For example, Chen et al., notes that “modified activin prodomains promote significant increases in muscle mass” (see Chen J L et al. (2015) Mol Ther. 23(3): 434-444). Another report teaches that “activin A reduces muscle mass and function by stimulating the ActRIIB pathway, leading to deleterious consequences, including increased transcription of atrophy-related ubiquitin ligases, decreased Akt/mTOR-mediated protein synthesis, and a profibrotic response” and that “the muscle wasting and fibrosis that ensues in response to excessive activin levels is fully reversible” thus suggesting the therapeutic potential of targeting activins in cachexia (see Chen J L et al., FASEB J. 2014 April; 28 (4): 1711-23).

In the cancer setting, one report teaches “high ActA level is an independent prognosis factor of survival in cancer patients” and that “ActA seems to influence survival by contributing to the development of cachexia and loss of skeletal muscle mass” (see Loumaye A et al., J Cachexia Sarcopenia Muscle. 2017 October; 8 (5): 768-777). Additional reports in the cancer setting describing the role of Activin-A in muscle wasting include the following: Walton K L et al., Endocrinology. 2019 Oct. 1; 160 (10): 2417-2426; Li Q et al., Mol Hum Reprod. 2007 September; 13 (9): 675-83; Zhou X et al., Cell. 2010 Aug. 20; 142 (4): 531-43; Huot J R et al. J Cachexia Sarcopenia Muscle. 2020 December; 11 (6): 1779-1798; and Zhong X et al. J Cachexia Sarcopenia Muscle. 2019 October; 10 (5): 1083-1101.

For muscle wasting or atrophy in chronic liver diseases, Kim J. Life (Basel) 2021 Mar. 17; 11 (3): 250, notes treatment options for sarcopenia in chronic liver disease. Addition reports noting a role for Activin-A in muscle wasting and/or loss of muscle mass include: Lee S J et al., Mol Endocrinol. 2010 October; 24 (10): 1998-2008; Coerver K A et al., Mol Endocrinol. 1996 May; 10 (5): 534-43; and Cipriano S C et al., Endocrinology, Volume 141, Issue 7, July 2000, Pages 2319-2327.

For a role of Activin-A in muscle wasting in COPD, a report teaches that “COPD patients had upregulated Activin A expression” and that Actigin A expression in COPD patients negatively correlated with fat-free mass index and body mass index (See Zhou G et al., Clinics (Sao Paulo). 2019; 74: e981.

In chronic kidney disease a report by Solgana F et al., J Clin Invest, 2021 Jun. 1; 131 (11): e135821, notes that “activin A is a key factor in CKD-induced cachexia.” An additional report teaches that systemic activation of Activin-A signaling causes chronic kidney disease-mineral bone disorder (CKD-MBD) (see, Sugatani T. Int J Mol Sci. 2018 September; 19(9): 2490). Several other reports report a role for Activin A in chronic kidney disease, see Cianciolo G et al., Nephrol Dial Transplant. 2021 May 27; 36 (6): 966-974, and Yonata A et al., Int J Nephrol. 2020 Nov. 9; 2020:8893653.

In Duchenne's muscular dystrophy, at least one report teaches that treating mice with AcRIIB-Fc results in increased bone mass in femurs (See Puolakkainen T. et al., BMC Musculoskelet Disord. 2017; 18:20).

The present disclosure teaches that provided Activin A/B antibody agents may be useful to treat and/or prevent Anorexia-Cachexia Disorders associated with chronic diseases, e.g., as described herein. Those skilled in the art, reading the present disclosure in light of the art, will appreciate such use as described herein.

Several reports have noted a role for Activin A in chronic kidney disease and associated cachexia, muscle wasting and/or anemia. For example, Solagna et al., teaches “increased production and elevated blood levels of soluble pro-cachectic factors, including activin A, directly linking experimental and human CKD to skeletal muscle wasting programs” (See Solagna F et al., J Clin Invest. 2021 Jun. 1; 131 (11): e135821. See also, Perens ET A et al., m J Kidney Dis. 2022 February; 79 (2): 302-304; Terker and Ikizler, Nephrology Digest Translational Science| vol: 101 (2), P211-213; Allison S. Nat Rev Nephrol. 2021 August; 17 (8): 511; Hruska K A et al., Bone. 2017 July; 100:80-86; doi: 10.1016. For a role of Activin in chronic kidney disease associated anemia, see Mehta and Krepinsky Current Opinion in Nephrology and Hypertension: January 2020—Volume 29—Issue 1—p 136-144.

The present disclosure teaches that provided Activin A/B antibody agents may be useful to treat and/or prevent chronic kidney disease and associated cachexia, muscle wasting, and/or anemia e.g., as described herein. Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing anemia. In some embodiments, a subject having anemia has an increased level of Activin-A and/or Activin-B, e.g., as compared to a subject who does not have anemia. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin-A in anemia. For example, one report notes that an activin receptor IIA ligand trap “may act to rescue growth differentiation factor 11/Activin A-induced inhibition of late-stage erythropoiesis” (See Carrancio S. et al. Br J Haematol. 2014 June; 165 (6): 870-882). An additional report notes the use of activin receptor type IIB fusion protein ligand trap to treat ineffective erythropoiesis (See Kubasch A S et al., Blood Adv. 2021 Mar. 9; 5 (5): 1565-1575). Yet another report teaches the “basis for biological activity of activin receptor ligand trap” in erythroid maturation and to alleviate anemia (Verma S. et al. J Clin Invest. 2020 Feb. 3; 130 (2): 582-589).

The present disclosure teaches that provided Activin A/B antibody agents may be useful to treat and/or prevent anemia. Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing metabolic disorders (e.g., obesity, type 2 diabetes, and/or metabolic syndrome). In some embodiments, a subject having a metabolic disorder has an increased level of Activin-A and/or Activin-B, e.g., as compared to a subject who does not have a metabolic disorder. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin-A in metabolic disorders. For example, one report notes “functional relationship between activin signaling and mitochondrial energy metabolism and further support the rationale to target this signaling pathway for the medical treatment of cachexia, obesity, and diabetes” (See Li L et al., Endocrinology. 2009 August; 150 (8): 3521-3529). A report by Peng L et al., teaches a “dose-dependent association between serum activin A levels, age, and metabolic syndrome, suggests activin A may be a biomarker of overall cardiometabolic risk” (Peng L. et al., Exp Gerontol. 2018 Oct. 1; 111:197-20). In type 2 diabetes and obesity an article by Heymsfield S B et al., JAMA Netw Open. 2021; 4 (1): e2033457, notes that “blockade of the activin receptor with bimagrumab [an ActRII blocker] could provide a novel pharmacologic approach for managing patients with type 2 diabetes with excess adiposity.”

The present disclosure teaches that provided Activin A/B antibody agents may be useful to treat and/or prevent metabolic disorders (e.g., obesity, type 2 diabetes, and/or metabolic syndrome). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing inflammatory disorders.

In some embodiments, inflammatory disorders include sepsis, endotoxemia, ARDS (Acute Respiratory Distress Syndrome)/cytokine Storm, COVID, allergic asthma, atopic dermatitis, acute and/or chronic pancreatitis, preeclampsia, inflammatory bowel diseases (e.g., Crohn's disease or ulcerative colitis), kidney disease (e.g., as described herein), or liver disease (e.g., as described herein).

In some embodiments, a subject having an inflammatory disorder has an increased level of Activin-A and/or Activin-B, e.g., as compared to a subject who does not have an inflammatory disorder. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin-A in inflammation and inflammatory disorders. For example, a report by Philips D J et al., Cytokine Growth Factor Rev. 2009 April; 20 (2): 153-64, is a review article teaching the role of Activin-A in inflammation and notes that “Activin modulates several aspects of the inflammatory response, including release of pro-inflammatory cytokines, nitric oxide production and immune cell activity.” For a report on the role of Activin-A in sepsis, see Lee J K et al., Respirology. 2016 July; 21 (5): 891-7.

For a report on the role of Activin-A in endotoxemia, see Jones K L et al., Proc Natl Acad Sci 2007 Oct. 9; 104 (41): 16239-44

For reports on the role of Activin-A in ARDS or cytokine storm see: Kim J et al BMC Pulm Med. 2019 Jun. 25; 19 (1): 115; Apostolou E et al., Am J Respir Crit Care Med. 2012 Feb. 15; 185 (4): 382-91; or Linko R et al., BMC Infect Dis. 2014 May 10; 14:253.

For a report on the role of Activin-A in COVID see McAleavy M et al., https://doi.org/10.1101/2021.02.04.429815, or Synolaki E et al. J Infect Dis. 2021 Feb. 24: jiab 108.

For a report on the role of Activin-A in allergic asthma see Hardy C L et al., Clin Exp Allergy. 2006 July; 36 (7): 941-50 or Hardy C L et al. Clin Exp Allergy. 2015 October; 45 (10): 1510-1522.

For a report on the role of Activin-A in atopic dermatitis Vittorakis S et al., Biomed Res Int. 2014; 2014:231036

For a report on the role of Activin-A in acute or chronic pancreatitis, see Thomas A L et al., Clin Transl Gastroenterol. 2020 May; 11 (5): e00152; or Staudacher J J et al., Sci Rep. 2017 Oct. 6; 7 (1): 12786.

For a report on the role of Activin-A in preeclampsia see Lim R et al., Am J Obstet Gynecol. 2015 January; 212 (1): 86.e1-12.

For a report on the role of Activin-A in inflammatory bowel diseases see, Hubner G et al., Lab Invest. 1997 October; 77 (4): 311-8; or Dohi T et al., Gastroenterology. 2005 February; 128 (2): 411-23

For a report on the role of Activin-A in acute kidney injury, see Takei Y et al., PLOS One. 2019; 14 (10): e0223703; Mehta and Krepinsky Current Opinion in Nephrology and Hypertension: January 2020-Volume 29-Issue 1-p 136-144; or Takahashi S et al., Sci Rep. 2018; 8:5176.

For a report on the role of Activin-A in acute liver injury, see Hughes and Evans Eur J Gastroenterol Hepatol. 2003 February; 15 (2): 127-31; or Wang D et al., World J Gastroenterol. 2013 Jun. 28; 19 (24): 3802-3809.

For a report on the role of Activin-A and/or Activin B in renal ischemic reperfusion injury see Fang DYP et al., Transplant Direct. 2016 Jun. 6; 2 (7): e87.

The present disclosure teaches that provided Activin A/B antibody agents may be useful to treat and/or prevent inflammatory disorders, e.g., sepsis, endotoxemia, ARDS (Acute Respiratory Distress Syndrome)/cytokine Storm, COVID, allergic asthma, atopic dermatitis, acute and/or chronic pancreatitis, preeclampsia, inflammatory bowel diseases (e.g., Crohn's disease or ulcerative colitis), kidney disease (e.g., acute kidney injury), or liver disease (e.g., acute liver injury). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing kidney disease, e.g., as described herein. In some embodiments, kidney disease comprises autosomal dominant polycystic kidney disease, Alports syndrome, kidney fibrosis, acute kidney injury, chronic kidney disease, or chronic kidney disease associated with cachexia, anemia, and/or muscle wasting.

In some embodiments, a subject having a kidney disease (e.g., as described herein) has an increased level of Activin-A and/or Activin-B, e.g., as compared to a subject who does not have a kidney disease. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin A and/or Activin B in kidney disease such as autosomal dominant polycystic kidney disease or Alports syndrome. For example, Williams M J et al., reports activation of activin receptor type IIA (ActRIIA) activation in chronic kidney disease (CKD) in a model of Alports syndrome (See Williams M J et al.: Kidney Int. 2018 January; 93 (1): 147-158. Another report teaches a role for activin signaling in polycystic kidney disease (See Leonhard W N et al., JASN December 2016, 27 (12) 3589-3599).

The present disclosure teaches that provided Activin A/B antibody agents may be useful to treat and/or prevent a kidney disease (e.g., autosomal dominant polycystic kidney disease, Alports syndrome, kidney fibrosis, acute kidney injury, chronic kidney disease, or chronic kidney disease associated with cachexia, anemia, and/or muscle wasting). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing heart disease, e.g., as described herein. In some embodiments, heart disease comprises cardiac aging, heart failure, or other known heart diseases.

In some embodiments, a subject having a heart disease (e.g., as described herein) has an increased level of Activin-A and/or Activin-B, e.g., as compared to a subject who does not have a heart disease. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

One report teaches a role for Activin A and/or Activin B in heart disease. See Roh J D et all., which teaches Sci Transl Med. 2019 Mar. 6; 11 (482): eaau8680, increased activin/ActRII signaling links aging and heart failure.

The present disclosure teaches that provided Activin A/B antibody agents may be useful to treat and/or prevent a heart disease (e.g., as described herein). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing lung disease, e.g., as described herein. In some embodiments, lung disease comprises lung fibrosis, lung inflammation, PAH, IPF, acute respiratory failure, or COVID-19 associated ARDS.

In some embodiments, a subject having a lung disease (e.g., as described herein) has an increased level of Activin-A and/or Activin-B, e.g., as compared to a subject who does not have a lung disease. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports teach the role of Activin A and/or B in lung disease. For example, for roles of Activin A and/or B in pulmonary hypertension, see: Yndstead A et al., J Appl Physiol (1985). 2009 April; 106 (4): 1356-64; Chu K Y et al., J Biol Chem. 2022 July; 298 (7): 102076; Yung L M et al., Sci Transl Med. 2020 May 13; 12 (543): eaaz5660.

For a role of Activin A and/or B in idiopathic pulmonary fibrosis (IPF), see Xie T et al., Lung. 2021 October; 199 (5): 467-473, or Myllarniemi M et al., BMC Pulm Med. 2014 Nov. 1; 14:170; or Maula L L et al., J Comp Pathol. 2015 February-Apr; 152 (2-3): 192-200.

For a role of Activin A and/or B in COVDI-19 with acute respiratory disease syndrome (ARDS), see McAleavy M et al., Mol Cell Biol. 2022 Jan. 20; 42 (1): e0046721.

For a role of Activin A and/or B in acute respiratory failure, see Synolaki E et a., J Infect Dis. 2021 May 20; 223 (9): 1544-1554, or see de Kretser D M et al., Crit Care. 2013 Oct. 31; 17 (5):R263. doi: 10.1186.

The present disclosure teaches that provided Activin A/B antibody agents may be useful to treat and/or prevent a lung disease (e.g., lung fibrosis, lung inflammation, PAH, IPF, acute respiratory failure, or COVID-19 associated ARDS). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing bone disorders, e.g., as described herein. In some embodiments, bone disorders comprise mineral bone disorders, osteoporosis, osteoarthritis, or FOP.

In some embodiments, a subject having a bone disease (e.g., as described herein) has an increased level of Activin-A and/or Activin-B, e.g., as compared to a subject who does not have a bone disease. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports teach the role of Activin A and/or B in bone disorders. For example, see Sugatani T Int J Mol Sci. 2018 Aug. 23; 19(9):2490, for a disclosure on Activin A in CKD-mineral bone disorder. For a report on the role of Activin A and/or B in osteoporosis see Lotinum S et al., Curr Mol Pharmacol. 2012 June; 5 (2): 195-204, or Zou L et al., J Musculoskelet Neuronal Interact. 2018 September; 18(3):320-322.

For a report on Activin A in Fibrodysplasia Ossificans Progressiva (FOP) see Pignolo R J et al., Front Endocrinol (Lausanne). 2020 Jan. 10; 10:908. doi: 10.3389.

The present disclosure teaches that provided Activin A/B antibody agents may be useful to treat and/or prevent a bone disease (e.g., mineral bone disorders, osteoporosis, osteoarthritis, or FOP). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing hypertension or related disorders, e.g., preeclampsia. In some embodiments, hypertension comprises preeclampsia.

In some embodiments, a subject having hypertension (e.g., as described herein) has an increased level of Activin-A and/or Activin-B, e.g., as compared to a subject who does not have hypertension. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports teach the role of Activin A in hypertension. For example, Tsai Y L et al., teaches that increased Activin A levels are associated with higher systolic blood pressure, diastolic blood pressure and pulse pressure (PP), see Tsai Y L et al., Am J Hypertens. 2018 Feb. 9; 31 (3): 369-374.

For reports on roles of Activin A in preeclampsia see Mylona I et al., Endocr Pathol. 2006 Spring; 17 (1): 19-33 which reports expression of Activin A in preeclampsia; and see Wong G P et al., Pregnancy Hypertens. 2022 March; 27:23-26. doi: 10.1016, which teaches increased levels of Activin A at 36 weeks of gestation in women who developed preeclampsia. Hobson S R et al., teach that Activin A contributes to the pathophysiology of preeclampsia and suggests targeting activin signaling in this disorder, see Pregnancy Hypertens. 2016 April; 6(2):130-3. doi: 10.1016.

The present disclosure teaches that provided Activin A/B antibody agents may be useful to treat and/or prevent hypertension or related disorders, e.g., preeclampsia. Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing pituitary gonadal disorders and/or reproductive disorders (e.g., as described herein).

In some embodiments, a subject having pituitary gonadal disorders and/or reproductive disorders has an increased level of Activin-A and/or Activin-B, e.g., as compared to a subject who does not have pituitary gonadal disorders and/or reproductive disorders. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports teach of Activin A and/or B in pituitary gonadal disorders and/or reproductive disorders. For example, see Hsun-Ming C. and Leung P. Journal of Bio-X Research: December 2018—Volume 1—Issue 3—p 111-119, which teaches that “[a]bnormal activin expression, an imbalanced activin/follistatin ratio, and the dysregulation of the activin signaling pathway have been observed in several ovarian pathologies, such as reproductive aging, polycystic ovary syndrome, and ovarian cancers.” See also Petragila F et al., which reports levels of Activin A in ovarian failure and hypogonadotropic hypothalamic amenorrhea (See Petragila F et al., Reproductive Biology, Vol: 70, ISSUE 5, P907-912, (1998)).

The present disclosure teaches that provided Activin A/B antibody agents may be useful to treat and/or prevent pituitary gonadal disorders and/or reproductive disorders (e.g., as described herein). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing Myalgic Encephalomyelitis/Chronic Fatigue Syndrome (ME/CFS).

In some embodiments, a subject having ME/CFS has an increased level of Activin-A and/or Activin-B, e.g., as compared to a subject who does not have ME/CFS. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

One report notes the role of Activin B as a biomarker for ME/CFS, see Lidbury B A et al., 2017 Mar. 16; 15 (1): 60. doi: 10.1186/s12967-017-1161-4. Lidbury et al teaches that patients with chronic fatigue syndrome/myalgic encephalomyelitis (CFS/ME) have elevated Activin B levels.

The present disclosure teaches that provided Activin A/B antibody agents may be useful to treat and/or prevent Myalgic Encephalomyelitis/Chronic Fatigue Syndrome (ME/CFS). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing aging or aging related disorders (e.g., aging in sarcopenia, hypertension and/or kidney disease in aging, heart failure in aging, pituitary goanadal axis disorders in aging, immunosenescence in aging, and/or ovarian disorders in aging).

In some embodiments, an aging subject has an increased level of Activin-A and/or Activin-B, e.g., as compared to a subject who is not aging. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports teach a role for Activin in aging and aging related disorders. See, e.g., Tsai Y L et al., Am J Hypertens. 2018 Feb. 9; 31(3):369-374; reporting a correlation between Activin A levels and higher systolic blood pressure (SBP), diastolic blood pressure and pulse pressure (PP). Roh J D et al., teaches a role for Activin type II receptor in heart failure and cardia aging (See Roh J D et al., Sci Transl Med, (2019) vol 11:482).

For a report on Activin A in aging of the pituitary gonadal axis, see Baccarelli A et al., Exp Gerontol. 2001 August; 36 (8): 1403-12.

For a report on Activin A in ovarian pathologies, such as reproductive aging, polycystic ovary syndrome, and ovarian cancers, see Hsun-Ming C. and Leung P. Journal of Bio-X Research: December 2018—Volume 1—Issue 3-p 111-119.

For a report on Activin A in senescence and aging, see Xu M et al., eLife. 2015; 4: e12997. Published online 2015 Dec. 19. doi: 10.7554/eLife.12997, which teaches that “primary human senescent fat progenitors secrete activin A and directly inhibit adipogenesis in non-senescent progenitors.”

The present disclosure teaches that provided Activin A/B antibody agents may be useful to treat and/or prevent aging or aging related disorders (e.g., aging in sarcopenia, hypertension and/or kidney disease in aging, heart failure in aging, pituitary goanadal axis disorders in aging, immunosenescence in aging, and/or ovarian disorders in aging). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing auto-immune disorders (e.g., SLE or rheumatoid arthritis). In some embodiments, a subject having an auto-immune disorder has an increased level of Activin-A and/or Activin-B, e.g., as compared to a subject who does not have an auto-immune disorder. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

At least one report has noted a role for Activin-A in auto-immune disorders. The report by El-Gendi et al., teaches that increased serum levels of Activin-A in patients with rheumatic diseases (RA, SLE and osteoarthritis) and that “serum levels correlated positively with disease activity parameters of R^{A} and SLE” (See El-Gendi S S et al., Int J Rheum Dis. 2010 August; 13(3):273-9).

The present disclosure teaches that provided Activin A/B antibody agents may be useful to treat and/or prevent auto-immune disorders (e.g., SLE or rheumatoid arthritis). Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing chronic diseases, e.g., associated with fibrosis. In some embodiments, chronic diseases associated with fibrosis include: lung fibrosis, liver fibrosis, kidney fibrosis, systemic sclerosis, uterine fibroids, endometriosis, and/or heart failure. In some embodiments, a subject having a chronic disease associated with fibrosis has an increased level of Activin-A and/or Activin-B, e.g., as compared to a subject who does not have a chronic disease associated with fibrosis. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin-A in chronic diseases associated with fibrosis. In lung fibrosis, the following reports noted a role for Activin-A in lung fibrosis: Matsuse T et al., Am J Pathol. 1996 March; 148 (3): 707-13; Aoki F et al., Am J Respir Crit Care Med. 2005 Sep. 15; 172 (6): 713-20; Ohga E et al., Biochem Biophys Res Commun. 1996 Nov. 12; 228 (2): 391-6; or Matsuse T et al., Am J Respir Cell Mol Biol. 1995 July; 13 (1): 17-24.

For a role of Activin-A in liver apoptosis, fibrosis and/or cirrhosis, see Patella S et al., Am J Physiol Gastrointest Liver Physiol. 2006 January; 290 (1): G137-44; De Bleser P J et al., Hepatology. 1997 October; 26 (4): 905-12; Kiagiadaki F et al., Biochimica et Biophysica Acta (BBA)-Molecular Basis of Disease; Volume 1864, Issue 3, March 2018, Pages 891-899; Wada W et al., Endocrinology. 2004 June; 145 (6): 2753-9; Ding Z et al., Int J Mol Sci. 2016 Mar. 22; 17 (3): 408; Zhnag H et al., Int J Mol Med. 2018 July; 42 (1): 279-289; Sugiyama M et al., Gastroenterology. 1998 March; 114 (3): 550-8; Huang X et al., World J Gastroenterol. 2001 February; 7(1):37-41; or Gold E J et al., Mol Cell Endocrinol. 2003 Mar. 28; 201 (1-2): 143-53.

For a role of Activin-A in Hepatitis C see Refaat B et al., Gastroenterology Research and Practice, vol. 2014, Article ID 628683.

For a role of Activin-A in alcoholic liver disease see Staudacher J J et al., Gastro Hep Adv. 2022; 1 (2): 147-149; and Voumvouraki A. et al., Eur J Clin Invest. 2012 August; 42 (8): 815-22. See also Voumvouraki A. et al., Eur J Clin Invest. 2012 August; 42 (8): 815-22 for a role of Activin A in liver cirrhosis.

For a role of Activin-A in kidney fibrosis (Alport, or PKD) see Ren X et al., Nephrology (Carlton). 2009 April; 14 (3): 311-20; Sugatani T., Int J Mol Sci. 2018 Aug. 23; 19 (9): 2490; Leonhard W N et al., J Am Soc Nephrol. 2016 December; 27 (12): 3589-3599.

For a role of Activin-A in systemic sclerosis see Takagi K et al., J Autoimmun. 2011 May; 36 (3-4): 181-8; or Ly T et al., Biomolecules. 2020 Apr. 14; 10 (4): 609.

For a role of Activin-A in uterine fibroids see Bao H et al., Biochem Biophys Res Commun. 2018 Oct. 2; 504 (2): 447-45; or Protic O et al., Semin Reprod Med. 2017 November; 35 (6): 499-509.

For a role of Activin-A in endometriosis see Kasai K et al., J Med

### Invest. 2019; 66 (1.2): 123-127; or Zhang Z et al., Cell Commun Signal. 2019 May 17; 17 (1): 45.

For a role of Activin-A in heart failure see Roh J D et al., Sci Transl Med. 2019 Mar. 6; 11 (482).

Several reports have also noted a role for Activin-B in fibrosis. For a role of Activin B in liver fibrosis and NASH see Wang Y et al., Hepatol Commun. 2022 Jul. 22. doi: 10.1002/hep4.2037. For a role of Activin B in kidney fibrosis see Krepinsky JC J Pathol. 2022 April; 256 (4): 363-365; or Sun Y et al., J Pathol. 2022 January; 256 (1): 25-37.

The present disclosure teaches that provided Activin A/B antibody agents may be useful to treat and/or prevent chronic diseases, e.g., associated with fibrosis. In some embodiments, chronic diseases associated with fibrosis include: lung fibrosis, liver fibrosis, kidney fibrosis, systemic sclerosis, uterine fibroids, Endometriosis, and/or heart failure. Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing cancer, e.g., as described herein. In some embodiments, administration of an Activin A/B antibody agent to a subject having a cancer can prevent and/or reverse any one, all or a combination of Activin-A and/or Activin-B induced characteristics: tumor cell growth, migration, invasion and metastasis, angiogenesis, stemness and drug resistance, or regulation of antitumor immunity. In some embodiments, a cancer is chosen from colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer.

In some embodiments, a subject having a cancer has an increased level of Activin-A and/or Activin-B, e.g., as compared to a subject who does not have a cancer. In some embodiments, an increased level of Activin-A and/or Activin-B is a level of about 500 pg/ml or more, e.g., as evaluated in a sample from a subject, e.g., a blood, plasma, serum, tumor or urine sample. In some embodiments, administration of an Activin-A and/or Activin-B antibody agent reduces a level of Activin-A and/or Activin-B to less than about 500 pg/ml. In some embodiments, administration of an Activin-A and/or Activin-B antibody agent prevents an increase of Activin-A and/or Activin-B level to more than about 500 pg/ml.

Several reports have noted a role for Activin-A in cancer and cancer treatment-related toxicities. For reports that teach a role for Activin-A in, e.g., inducing tumor cell growth, migration, angiogenesis, stemness and drug resistance, regulation of antitumor immunity, see: Deli A et al., World J Gastroenterol. 2008 Mar. 21; 14 (11): 1699-1709; Ries A et al., Expert Opin Ther Targets. 2020 October; 24 (10): 985-996; Guo and Liu, Journal of International Medical Research Volume 49 Issue 6; Hoda M A et al., Br J Cancer. 2012 Dec. 4; 107 (12): 1978-86; Seder C W et al., J Thorac Oncol. 2009 April; 4 (4): 455-62; Dignass A U et al., Scand J Gastroenterol. 2002 August; 37 (8): 936-43; Jung B H et al., Gastroenterology. 2007 February; 132 (2): 633-44; Chang K et al., Ann Surg Oncol. 2010 July; 17 (7): 1945-56; Tamminen J A et al., Exp Cell Res. 2015 Mar. 1; 332 (1): 102-15; Dean M et al., Cancer Lett. 2017 Apr. 10; 391:114-124; Yi Y et al., Exp Cell Res 2019 Sep. 15; 382 (2): 111471; Portale F et al., Haematologica. 2019 Mar; 104 (3): 533-545; Bao H et al., Biochem Biophys Res Commun. 2020 Aug. 20; 529 (2): 379-385; Wagner K et al., Gastroenterology. 2004 June; 126 (7): 1828-43.

For a report teaching a role of Activin-A in chemotherapy-induced nephropathy, see Koken E et al., Bratisl Lek Listy. 2020; 121 (2): 143-150, which teaches that administration of Follistatin (an Activin blocker) “showed a considerable nephroprotective effect against cisplatin-induced nephrotoxicity by preventing renal functional and structural abnormalities, apoptosis and inflammation.”

For reports teaching a role of Activin-A in cancer metastasis, see Staudacher J J et al., Sci Rep. 2017; 7:5569; Leto G et al., Clin Exp Metastasis. 2006; 23 (2): 117-22; Nomura M et al., Biochem Biophys Res Commun. 2013 Jan. 4; 430 (1): 340-6; Kalli M et al., Front Oncol. 2019 Feb. 5; 9:32; or Hyuga S et al., Cancer Lett. 2000 May 29; 153 (1-2): 137-43.

For a report on Activin-A polymorphism and treatment with regrofenib, see Miyamoto Y et al., PLOS One. 2020 Sep. 24; 15 (9): e0239439.

For a report summarizing findings on “the multifaceted anti-cachectic effects of ACVR2 blockade in preclinical models of cancer, as well as in combination with anticancer treatments,” see Hulmi J J et al., Cells. 2021 Feb. 28; 10 (3): 516.

For a report on immune escape mechanisms driven by Activin-A including promotion of T reg cell mediated immunosuppression following radiation therapy, see De Martino M et al., Cancer Immunol Res. 2021 January; 9 (1): 89-102.

The present disclosure teaches that provided Activin A/B antibody agents may be useful to treat and/or prevent cancer, e.g., as described herein. One of skill in the art would also understand that an antibody agent disclosed herein can be used to prevent and/or reverse any one, all or a combination of Activin-A and/or Activin-B induced characteristics: tumor cell growth, migration, invasion and metastasis, angiogenesis, stemness and drug resistance, or regulation of antitumor immunity. In some embodiments, a cancer is chosen from: colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer. Those skilled in the art, reading the present disclosure in light of the art, will appreciate such use as described herein.

In some embodiments, an Activin A/B antibody agent as described herein (e.g., an anti-Activin A/B antibody) may be useful in treating and/or preventing aging and/or senescence. In some embodiments, an aging subject or a subject who has senescence has an increased level of Activin-A and/or Activin-B, e.g., as compared to a subject who is not aging or does not have senescence. In some embodiments, an increased level of Activin-A and/or Activin-B is a level of about 500 pg/ml or more, e.g., as evaluated in a sample from a subject, e.g., a blood, plasma, serum or urine sample. In some embodiments, administration of an anti-Activin-A and/or Activin-B antibody agent reduces a level of Activin-A and/or Activin-B to less than about 500 pg/ml. In some embodiments, administration of an Activin A/B antibody agent prevents an increase of Activin-A and/or Activin-B level to more than about 500 pg/ml.

Several reports have noted a role for Activin-A in aging and/or senescence. For example a report by Barrios-Silva L V et al., note a role for Activin-A in normal human aging (See Barrios-Silva L V et al., Physiol Rep. 2018 September; 6 (17): e13823). Another report teaches that Activin-A is a Senescence-Associated Secretory Phenotype (SASP) protein (See Schafer M J et al., JCI Insight. 2020 Jun. 18; 5 (12): e133668. Yet another report noted increased plasman Activin-A levels in diabetic patients with diabetic kidney disease and suggest a role for “Activin A as a diagnostic marker and therapeutic target in DKD” (See Bian X et al., BMJ Open Diabetes Res Care. 2019 Dec. 15; 7 (1): e000720. A report by Roh D J et al., teaches that Activin-A induces up-regulation of the proteasome pathway in mammalian cardiomyocytes and that “increased activin/ActRII signaling links aging and HF pathobiology” (See Roh D J et al., Sci Transl Med. 2019 Mar. 6; 11 (482)).

The present disclosure teaches that provided an Activin A/B antibody agent may be useful to treat and/or prevent aging and/or senescence. Those skilled in the art, reading the present disclosure in light of the art, will appreciate such use as described herein.

In some embodiments of any of the methods disclosed herein, an Activin A/B antibody agent is characterized in that when administered to a subject it reduces a level and/or activity of Activin-A and/or Activin-B relative to a comparator. In some embodiments, a level Activin-A and/or Activin-B is reduced in a blood, plasma, serum, tumor or urine sample. In some embodiments, an Activin-A and/or Activin-B antibody agent reduces a level of Activin-A and/or Activin-B to less than 500 pg/mL.

In some embodiments, Activin-A and/or Activin-B is or comprises free and/or active Activin-A and/or Activin-B. In some embodiments, Activin-A and/or Activin-B is or comprises free Activin-A and/or Activin-B. In some embodiments, Activin-A and/or Activin-B is or comprises active Activin-A and/or Activin-B. In some embodiments, Activin-A and/or Activin-B is or comprises free and active Activin-A and/or Activin-B.

At least one report has noted a role for Activin A in lipodystrophy (Ming Xu, et al., (2015) Targeting senescent cells enhances adipogenesis and metabolic function in old age. eLife 4: e12997). Xu et al., reported that human senescent fat progenitors secrete Activin A and that blocking Activin A partially restored lipid accumulation and expression of adipogenic markers. Levels of Activin A was also shown to be increased with aging. Reducing circulating Activin A with a JAK1/2 inhibitor preserved fat mass, reduced lipotoxicity and increased insulin sensitivity suggesting that targeting products of senescent cells can be useful in treating and/or preventing lipodystrophy.

The present disclosure teaches that provided an Activin A/B antibody agent may be useful to treat and/or prevent lipodystrophy. Those skilled in the art, reading the present disclosure in light of the art, will appreciate such use as described herein.

In some embodiments of any of the methods disclosed herein, an Activin A/B antibody agent is characterized in that when administered to a subject it reduces a level and/or activity of Activin-A and/or Activin-B relative to a comparator. In some embodiments, a level Activin-A and/or Activin-B is reduced in a blood, plasma, serum, tumor or urine sample. In some embodiments, an Activin-A and/or Activin-B antibody agent reduces a level of Activin-A and/or Activin-B to less than 500 pg/mL.

In some embodiments, Activin-A and/or Activin-B is or comprises free and/or active Activin-A and/or Activin-B. In some embodiments, Activin-A and/or Activin-B is or comprises free Activin-A and/or Activin-B. In some embodiments, Activin-A and/or Activin-B is or comprises active Activin-A and/or Activin-B. In some embodiments, Activin-A and/or Activin-B is or comprises free and active Activin-A and/or Activin-B.

One with skill in the art, reading the present specification, will appreciate that provided Activin A/B antibody agents which bind to both Activin A and Activin B can be useful in treating and/or preventing symptoms and/or disorders which can be treated by anti-Activin A antibodies disclosed in International Patent Application PCT/US2022/47001 filed on Oct. 18, 2022, the entire contents of which is hereby incorporated by reference. Among other things, PCT/US2022/47001 documents therapeutic and/or preventative effects of anti-Activin A antibodies in a variety of disease settings. The present disclosure teaches that provided Activin A/B antibody agents which bind to both Activin A and Activin B, can be useful in treating and/or preventing diseases and/or disorders that can be treated and/or prevented with the anti-Activin A antibodies as described in PCT/US2022/47001.

### Dosing Regimens

Those skilled in the art will be able to determine, according to known methods, the appropriate amount, dose or dosage of an Activin A/B antibody agent, to administer to a patient, taking into account factors such as age, weight, general health, the route of administration, the nature of the symptom, disease or disorder requiring treatment, and the presence of other medications. For example, various dosing regimens for antibodies are disclosed in Hendrikx J et al. (2017) Oncologist 22 (10): 1212-1221, NMID: 28754722, the entire contents of which is hereby incorporated by reference.

In some embodiments, an Activin A/B antibody agent is administered at a fixed dose, i.e. independent of body weight. In some embodiments, a fixed dose reduces interpatient variability, e.g., efficacy and/or PK/PD parameters.

In some embodiments, an Activin A/B antibody agent is administered at a fixed dose of about of 0.1 mg to about 2000 mg. In some embodiments, an Activin A/B antibody agent is administered at a fixed dose of about 0.1 mg, about 0.2 mg, about 0.25 mg, about 0.5 mg, about 1 mg, about 5 mg, about 10 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, or about 2000 mg. In some embodiments, an Activin A/B antibody agent is administered intravenously (IV) or subcutaneously (SC) at a fixed dose of about 0.25 mg to about 2000 mg.

In some embodiments, an Activin A/B antibody agent administered at a fixed dose is administered daily, weekly or monthly. In some embodiments, an Activin A/B antibody agent administered at a fixed dose is administered once a week, once every 2 weeks, once every 3 weeks or once every 4 weeks.

In some embodiments, an Activin A/B antibody agent is administered based on body weight, e.g., in a mg/kg dosing. In some embodiments, an Activin A/B antibody agent is administered at a dose of about 0.025 mg/kg to about 20 mg/kg. In some embodiments, an Activin A/B antibody agent is administered at a dose of about 0.025 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg. In some embodiments, an Activin A/B antibody agent is administered intravenously (IV) or subcutaneously (SC) at a dose of about 0.025 mg/kg to about 20 mg/kg.

In some embodiments, an Activin A/B antibody agent is administered at an initial dose. In some embodiments, an initial dose may be followed by one or more subsequent doses. In some embodiments, one or more subsequent dose may be administered daily, weekly, or monthly, or at other intervals in between. In some embodiments, a dosing regimen disclosed herein may be repeated for one or more times.

### Combination Therapies

In some embodiments, an Activin A/B antibody agent disclosed herein, or a composition comprising the same is administered in combination with an additional agent, e.g., additional therapy.

In some embodiments, an additional therapy comprises a therapy for a disease or disorder, e.g., a standard of care (SOC) therapy, for a symptom, disease or disorder.

In some embodiments, an additional therapy comprises a GLP-1 receptor modulator (e.g., an agonist or an antagonist), a GIP receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of PYY, an agent that increases a level and/or activity of GDF15, an agent that increases a level and/or activity of GFRAL, or any combination thereof.

In some embodiments, an additional therapy comprises a GLP-1 receptor modulator (e.g., an agonist or an antagonist). In some embodiments, an additional therapy comprises a GIP receptor modulator (e.g., an agonist or an antagonist). In some embodiments, an additional therapy comprises a glucagon receptor modulator (e.g., an agonist or an antagonist). In some embodiments, an additional therapy comprises a GLP-1 receptor modulator (e.g., an agonist or an antagonist) and a GIP receptor modulator (e.g., an agonist or an antagonist). In some embodiments, an additional therapy comprises a GLP-1 receptor modulator (e.g., an agonist or an antagonist) and a glucagon receptor modulator (e.g., an agonist or an antagonist). In some embodiments, an additional therapy comprises a GIP receptor modulator (e.g., an agonist or an antagonist) and a glucagon receptor modulator (e.g., an agonist or an antagonist). In some embodiments, an additional therapy comprises an agent that increases a level and/or activity of GIP. In some embodiments, an additional therapy comprises an agent that increases a level and/or activity of GLP-1. In some embodiments, an additional therapy comprises an agent that increases a level and/or activity of glucagon. In some embodiments, an additional therapy comprises an agent that increases a level and/or activity of PYY. In some embodiments, an additional therapy comprises an agent that increases a level and/or activity of GDF-15. In some embodiments, an additional therapy comprises an agent that increases a level and/or activity of GFRAL.

GIP receptor modulator (e.g., an agonist or an antagonist), GLP-1 receptor modulator (e.g., an agonist or an antagonist) and glucagon receptor modulator (e.g., an agonist or an antagonist) are known in the field and can be readily ascertained by one with skill in the art.

In some embodiments, a GLP-1 receptor modulator (e.g., an agonist or an antagonist) is or comprises a GLP-1 polypeptide, or a variant, or fragment or analog thereof. Exemplary GLP-1 receptor modulators (e.g., agonist or antagonist) include: dulaglutide, exenatide, liraglutide, lixisenatide, semaglutide, tirzepatide, danuglipron, orforglipron, ECC5004, or variants, or fragments, or dosage variants (e.g., extended release, oral formulations, IV formulations, etc.) of any of the foregoing.

In some embodiments, a GIP receptor modulator (e.g., an agonist or an antagonist) is or comprises a GIP polypeptide, or a variant, or fragment or analog thereof.

In some embodiments, a glucagon receptor modulator (e.g., an agonist or an antagonist) is or comprises a glucagon polypeptide, or a variant, or fragment or analog thereof.

In some embodiments, an additional agent is a GIP receptor modulator (e.g., an agonist or an antagonist) and a GLP-1 receptor modulator (e.g., an agonist or an antagonist).

In some embodiments, an additional agent is a GIP receptor modulator (e.g., an agonist or an antagonist) and a glucagon receptor modulator (e.g., an agonist or an antagonist).

In some embodiments, an additional agent is a GLP-1 receptor modulator (e.g., an agonist or an antagonist) and a glucagon receptor modulator (e.g., an agonist or an antagonist).

In some embodiments, an additional agent is a GLP-1 receptor modulator (e.g., an agonist or an antagonist), a glucagon receptor modulator (e.g., an agonist or an antagonist) and a GIP receptor modulator (e.g., an agonist or an antagonist).

In some embodiments, an agent that increases a level and/or activity of GLP-1 is a GLP-1 polypeptide or a variant, or fragment or analog thereof.

In some embodiments, an agent that increases a level and/or activity of glucagon is a glucagon polypeptide or a variant, or fragment or analog thereof.

In some embodiments, an agent that increases a level and/or activity of GDF-15 is a GDF-15 polypeptide or a variant, or fragment or analog thereof, or a polypeptide that activates a GDF-15 pathway.

In some embodiments, an agent that increases a level and/or activity of GFRAL is a GFRAL polypeptide or a variant, or fragment or analog thereof, or a polypeptide that activates a GFRAL pathway.

In some embodiments, an additional agent can be delivered orally.

In some embodiments, an additional agent can be delivered via an injection, e.g., an intravenous injection.

In some embodiments, an additional agent is formulated for oral delivery.

In some embodiments, an additional agent is formulated for delivery via an injection,

e.g., an intravenous injection.

In some embodiments, an additional agent is formulated for extended release.

In some embodiments, an additional agent is formulated to be long-acting.

Exemplary dual modulators (e.g., agonist or antagonist) of a GLP-1 receptor and a GIP receptor include tirzepatide, or variants, or fragments, or dosage variants (e.g., extended release, oral formulations, IV formulations, etc.) thereof.

Exemplary dual modulators (e.g., agonist or antagonist) of a GLP-1 receptor and a glucaogon receptor include Mazdutide, MK-1642 or variants, or fragments, or dosage variants (e.g., extended release, oral formulations, IV formulations, etc.) thereof.

In some embodiments, an Activin A/B antibody agent is administered before, concurrently with or after administration of an additional therapy, e.g., a SOC therapy. In some embodiments of concurrent administration of an Activin A/B antibody agent disclosed herein, or a composition comprising the same and an additional therapy, an Activin A/B antibody agent disclosed herein, or a composition comprising the same is administered first followed by an additional therapy. In some embodiments of concurrent administration of an Activin A/B antibody agent disclosed herein, or a composition comprising the same and an additional therapy, an additional therapy is administered first followed by an Activin A/B antibody agent disclosed herein, or a composition comprising the same.

In some embodiments, when a subject has a metabolic disorder (e.g., diabetes), an additional therapy comprises a GLP-1 receptor modulator (e.g., an agonist or an antagonist), a GIP receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of PYY, an agent that increases a level and/or activity of GDF15, an agent that increases a level and/or activity of GFRAL, or any combination thereof.

In some embodiments, when a subject has excess weight (e.g., as compared to the weight of an otherwise comparable subject), an additional therapy comprises a GLP-1 receptor modulator (e.g., an agonist or an antagonist), a GIP receptor modulator (e.g., an agonist or an antagonist), a glucagon receptor modulator (e.g., an agonist or an antagonist), an agent that increases a level and/or activity of GIP, an agent that increases a level and/or activity of GLP-1, an agent that increases a level and/or activity of Glucagon, an agent that increases a level and/or activity of PYY, an agent that increases a level and/or activity of GDF15, an agent that increases a level and/or activity of GFRAL, or any combination thereof.

In some embodiments, when a subject has cancer, an additional therapy, e.g., a SOC, comprises one or more of surgery, chemotherapy, radiation therapy, small molecule therapy, targeted therapy such as antibody therapy, immunotherapy, hormonal therapy, stem cell based therapy or other therapies, e.g., as are known in the field and appreciated by one with skill in the art. In some particular embodiments, an additional therapy may be or comprise checkpoint inhibitor therapy, angiogenesis inhibitor therapy, etc.

In some embodiments, when a subject has weight loss, an additional therapy, e.g., a SOC, comprises nutrition management, nutritional supplements, and/or an appetite stimulant.

In some embodiments, when a subject has loss of appetite, an additional therapy, e.g., a SOC comprises an appetite stimulant, e.g., a supplement such as Zinc, a cannabinoid, a synthetic progestin, a testosterone derivative, and/or a steroid.

In some embodiments, when a subject has chronic kidney disease, an additional therapy, e.g., a SOC comprises a treatment for one or more complications associated with chronic kidney disease. In some embodiments, a SOC for chronic kidney disease comprises one or more of: high blood pressure treatment; therapy to reduce cholesterol levels; therapy to treat anemia; therapy to strengthen bones; a low protein diet; dialysis; or kidney transplant.

In some embodiments, when a subject has chronic heart disease, an additional therapy, e.g., a SOC comprises an angiotensin-converting enzyme (ACE) inhibitor; an Angiotensin II receptor blocker; a beta blocker; a diuretic; an aldosterone antagonist; digoxin; an inotrope; a hydralazine and isosorbide dinitrate; or surgery.

In some embodiments, when a subject has COPD, an additional therapy, e.g., a SOC comprises a bronchodilator; an inhaled steroid; a combination of a bronchodilator and an inhaled steroid; an oral steroid; theophylline; antibiotics; oxygen therapy; ventilation therapy or surgery.

In some embodiments, when a subject has peripheral arterial hypertension, an additional therapy, e.g., a SOC comprises vasodilators, guanylate cyclase stimulators, endothelin receptor antagonists, PDE5 inhibitors, calcium channel blockers, warfarin, digoxin, diuretics, oxygen therapy, or a combination thereof.

In some embodiments, when a subject has acute kidney injury, an additional therapy, e.g., a SOC comprises one or more of: fluid resuscitation, avoidance of nephrotoxic medications and contrast media exposure, correction of electrolyte imbalances, or renal replacement therapy (e.g., dialysis).

In some embodiments, when a subject has a liver disease (e.g., as described herein), an additional therapy, e.g., a SOC comprises a treatment for one or more symptoms and/or complications associated with liver disease. In some embodiments, when a subject has cirrhosis, an additional therapy, e.g., a SOC comprises one or more of: weight management, abstinence from alcohol, medications to control hepatitis, medications to control symptoms of cirrhosis (e.g., primary biliary cirrhosis), or liver transplant. In some embodiments, when a subject has acute liver failure, an additional therapy, e.g., a SOC comprises one or more of: medications to reverse poisoning (e.g., acetylcysteine if injury is due to acetaminophen poisoning), medications to relieve cerebral edema, liver transplant, blood transfusions, medications to prevent severe bleeding, or nutritional support.

### Diagnostic Technologies

In some embodiments, an Activin A/B antibody agent, or a polypeptide disclosed herein (e.g., a LC polypeptide and/or a HC polypeptide), or a composition that comprises and/or delivers the same may be used to detect the presence of Activin-A and/or Activin-B, in vivo or in vitro. By correlating the presence or level of these proteins with a medical condition, one of skill in the art can diagnose the associated medical condition. The medical conditions that may be diagnosed by the presently disclosed Activin A/B antibody agents or LC and/or HC polypeptides are set forth above.

Such detection methods are well known in the art and include ELISA, radioimmunoassay, immunoblot, Western blot, immunofluorescence, immunoprecipitation, and other comparable techniques. An Activin A/B antibody agent or a polypeptide disclosed herein (e.g., a LC polypeptide and/or a HC polypeptide), may further be provided in a diagnostic kit that incorporates one or more of these techniques to detect a protein (e.g., Activin-A and/or Activin-B). Such a kit may contain other components, packaging, instructions, or other material to aid the detection of the protein and use of the kit.

Where the antibodies are intended for diagnostic purposes, it may be desirable to modify them, for example, with a ligand group (such as biotin) or a detectable marker group (such as a fluorescent group, a radioisotope or an enzyme). If desired, the antibodies (whether polyclonal or monoclonal) may be labeled using conventional techniques. Suitable labels include fluorophores, chromophores, radioactive atoms, electron-dense reagents, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. Other suitable labels may include biotin and avidin or streptavidin, IgG and protein A, and the numerous receptor-ligand couples known in the art. Other permutations and possibilities will be readily apparent to those of ordinary skill in the art and are considered as equivalents within the scope of the instant invention.

Accordingly, disclosed herein is a method comprising, assessing a level and/or activity of Activin-A and/or Activin-B in a sample from a subject, and administering an Activin-A and/or Activin-B pharmaceutical composition to the subject if the level of Activin-A and/or Activin-B is higher than a comparator.

In some embodiments, a level of Activin-A and/or Activin-B is evaluated with an Activin A/B antibody agent or a polypeptide disclosed herein (e.g., a LC polypeptide and/or a HC polypeptide). In some embodiments, an increased level and/or activity of Activin-A and/or Activin-B is determined relative to a comparator. In some embodiments, a comparator comprises a predetermined reference sample such as a sample obtained from an otherwise similar subject who does not have a condition, disease or disorder, or a symptom of a disease or disorder.

### Examples

The following examples are provided so as to further illustrate to the skilled artisan particular ways in which provided methods and/or compositions described herein may be made and/or used, and are not intended to limit the scope of the present disclosure.

### Example 1: Identification of Anti-Activin Antibodies

This Example demonstrates the identification of anti-Activin antibodies (e.g., Activin A/B antibody agents) from human synthetic yeast libraries.

### Materials and Methods

### Antigen Preparation

Antigens were biotinylated using the EZ-Link Sulfo-NHS-Biotinylation Kit (Thermo Scientific, Cat #21425). The antigens were concentrated to ~1 mg/mL and buffer exchanged into PBS before addition of 1:7.5 molar ratio biotinylation reagent. The mixture was held at 4° C. overnight prior to another buffer exchange to remove free biotin in the solution. Biotinylation was confirmed through streptavidin sensor binding of the labeled proteins on a ForteBio.

### Naïve Library Selections

Eight naïve human synthetic yeast libraries each of ~109 diversity were propagated as previously described (see, e.g., Y. Xu et al, PEDS 26 (10), 663-70 (2013); WO2009036379; WO2010105256; and WO2012009568.)

For the first two rounds of selection, a magnetic bead sorting technique utilizing the Miltenyi MACS system was performed, as previously described (see, e.g., Siegel et al, J Immunol Methods 286 (1-2), 141-153 (2004).) Briefly, yeast cells (~10¹⁰ cells/library) were incubated with 10 nM biotinylated human Activin A-Fc fusion for 30 min at 30° C. in wash buffer (phosphate-buffered saline (PBS)/0.1% bovine serum albumin (BSA)). After washing once with 40 mL ice-cold wash buffer, the cell pellet was resuspended in 20 mL wash buffer, and Streptavidin MicroBeads (500 μl) were added to the yeast and incubated for 15 min at 4° C. Next the yeast were pelleted, resuspended in 5 mL wash buffer, and loaded onto a Miltenyi LS column. After the 5 mL were loaded, the column was washed 3 times with 3 mL wash buffer. The column was then removed from the magnetic field, and the yeast were eluted with 5 mL of growth media and then grown overnight.

The following rounds of selection were performed using flow cytometry (FACS). Yeast were pelleted, washed three times with wash buffer, and incubated at 30° C. with either 10 nM biotinylated human Activin A-Fc fusion, or with a polyspecificity reagent (PSR) to remove non-specific antibodies from the selection. For the PSR depletion, the libraries were incubated with a 1:10 dilution of biotinylated PSR reagent as previously described (see, e.g., Y. Xu et al, PEDS 26 (10), 663-70 (2013)). Yeast were then washed twice with wash buffer and stained with goat F(ab′)2 anti-human kappa-FITC (LC-FITC) diluted 1:100 (Southern Biotech, Cat #2062-02) and either Streptavidin-AF633 (SA-633) diluted 1:500 (Life Technologies, Cat #S21375) or Extravidin-phycoerthyrin (EA-PE) diluted 1:50 (Sigma-Aldrich, Cat #E4011), secondary reagents for 15 min at 4° C. After washing twice with ice-cold wash buffer, the cell pellets were resuspended in 0.3 mL wash buffer and transferred to strainer-capped sort tubes. Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast were plated and individual colonies were picked for characterization.

### Antibody Optimization

Optimization of antibodies was performed via a light chain batch shuffle, and then by introducing diversities into the heavy chain variable region using approaches described below.

Light chain batch shuffle: Heavy chains from the naïve output were used to prepare light chain diversification libraries. Selections were performed on these libraries as described above, i.e., with one round of MACS and four rounds of FACS. In the different FACS selection rounds, the libraries were evaluated for, e.g., PSR binding, and affinity pressure by antigen titration. Sorting was performed in order to obtain a population with the desired characteristics. Individual colonies from each terminal FACS selection round were picked for sequencing and characterization.

CDRH1 and CDRH2 selection: The CDRH3 of a single antibody was recombined into a premade library with CDRH1 and CDRH2 variants of a diversity of ~10⁸ and selections were performed with one round of MACS and four rounds of FACS as described in the naïve discovery. For each FACS round the libraries were looked at for PSR binding and affinity pressure, and sorting was performed in order to obtain a population with the desired characteristics. For these selections, affinity pressures were applied by preincubating the biotinylated antigen with parental IgG or Fab for 30 minutes and then applying that precomplexed mixture to the yeast library for a length of time which would allow the selection to reach an equilibrium. The higher affinity antibodies were then able to be sorted.

CDRH3 selection: Oligos were ordered from IDT which comprised the CDRH3 as well as a flanking region on either side of the CDRH3. Each oligo variegated two amino acids in the CDRH3 via NNK diversity. The CDRH3 oligos were recombined with heavy chain FR1-FR3 variable regions containing selected variants from the CDRH1 and CDRH2 selections. Selections were performed similar to previous cycles using FACS sorting for four rounds. For each FACS round the libraries were looked at for PSR binding and affinity pressure, and sorting was performed in order to obtain a population with the desired characteristics. Affinity pressures for these selections were performed as described above in the CDRH1 and CDRH2 selection.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 3.5.

### Results

This process resulted in the identification of human anti-Activin A/B antibodies including clones A-C. The selected antibodies had a low PSR score as provided in Table 3. The PSR score was determined by normalizing values against a set of control IgGs whereby a clean PSR is a score of less than 0.1, a low PSR is a score between 0.1 and 0.33, a medium PSR is a score between 0.33 and 0.66 and a high PSR is a score between 0.66 and 1. The data provided in Table 3 shows that the tested anti-Activin antibodies had low PSR scores indicating high specificity to the antigen and minimal polyreactivity.

TABLE 3
PSR scores

| Clone | Poly-Specificity Reagent (PSR) Score (0-1) |
|---|---|
| A | 0.06 |
| B | 0.21 |
| C | 0.00 |

### Example 2: Characterization of Anti-Activin A/B Antibodies

This Example describes the evaluation of binding kinetics, binding specificity, stability and aggregation behavior for anti-Activin antibodies identified in Example 1.

### Materials and Methods

### ForteBio Octet K_{D} Measurements

ForteBio affinity measurements were performed on an Octet HTX generally as previously described (see, e.g., Estep et al, Mabs 5 (2), 270-278 (2013)). Briefly, ForteBio affinity measurements were performed by loading IgGs on-line onto AHC sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model.

### ForteBio Octet Epitope Binning

Epitope binning was performed using a standard sandwich format cross-blocking assay. Control anti-target IgG was loaded onto AHQ sensors and unoccupied Fc-binding sites on the sensor were blocked with an irrelevant human IgG1 antibody. The sensors were then exposed to 100 nM human Activin A-Fc antigen or human Activin B antigen followed by a second anti-Activin A/B antibody. Additional binding by the second antibody after antigen association indicates an unoccupied epitope (non-competitor), while no binding indicates epitope blocking (competitor). In studies assessing selectivity, human Activin A-Fc was replaced with BMP-9, BMP-10, GDF-15-Fc, GDNF, GDF-8, GDF-10, GDF-11 or Follistain 300.

### Size Exclusion Chromatography

A TSKgel SuperSW mAb HTP column (22855) was used for fast SEC analysis of yeast and mammalian produced mAbs at 0.4 mL/min with a cycle time of 6 min/run. 200 mM Sodium Phosphate and 250 mM Sodium Chloride was used as the mobile phase.

### Dynamic Scanning Fluorimetry (DSF)

10 μL of 20× Sypro Orange is added to 20 μL of 0.2-1 mg/mL mAb or Fab solution. A RT-PCR instrument (BioRad CFX96 RT PCR) is used to ramp the sample plate temperature from 40 to 95° C. at 0.5° C. increment, with 2 min equilibrate at each temperature. The negative of first derivative for the raw data is used to extract Tm.

### HIC (Hydrophobic Interaction Chromatography)

The methodology for this assay was described previously (see Estep P, et al. (2015) An alternative assay to hydrophobic interaction chromatography for high-throughput characterization of monoclonal antibodies. MAbs 7 (3): 553-561). In brief, 5 μg IgG samples (1 mg/mL) were spiked in with a mobile phase A solution (1.8 M ammonium sulfate and 0.1 M sodium phosphate at pH 6.5) to achieve a final ammonium sulfate concentration of about 1 M before analysis. A Sepax Proteomix HIC butyl-NP5 column was used with a linear gradient of mobile phase A and mobile phase B solution (0.1 M sodium phosphate, pH 6.5) over 20 min at a flow rate of 1 mL/min with UV absorbance monitoring at 280 nm.

### Surface Plasmon Resonance Kinetics Experiments

Kinetic analysis was conducted at 25° C. in HBS-EP+running buffer (10 mM HEPES pH 7.4, 150 mM NaCl, 3 mM EDTA, 0.05% Surfactant P20) using a Biacore 8K optical biosensor (Cytiva, Marlborough, MA). The sample compartment was maintained at 10° C. for the duration of the experiment. The Biacore 8K can analyze up to 8 separate interactions in a single cycle.

Each experiment cycle began with an injection (300 s at 2 μL/min) overflow cells 1 and 2 of a 1:100 solution of biotin CAPture reagent (Cytiva) in running buffer. This was followed by an injection (45 s at 10.0 μL/min) of biotinylated human Activin A (5 nM) overflow cell 2. Upon capture of Activin A to the sensor surface, a single concentration of Fab ranging from 0.111-27 nM was injected (300 s at 30 μL/min) overflow cells 1 and 2. The dissociation of the antibodies were monitored for 3420 s. Finally, an injection (120 s at 10 μL/min) of regeneration solution (6 M Guanidine-HCl in 0.25 M NaOH) overflow cells 1 and 2 prepared the sensor surface for another cycle. Several blank buffer samples were injected (300 s at 30 μL/min) overflow cells 1 and 2 throughout the duration of the experiment and used for reference surface subtraction—each were monitored for the same amount of dissociation time (3420 s) and regenerated under the same conditions as those for the Fab sample injections.

The resulting sensorgrams were cropped to include only the association and dissociation steps of the experiment. This cropped data was subsequently aligned, double reference subtracted, and then non-linear least squares fit to a 1:1 binding model using Biacore Insight Evaluation software version 3.0.11.15423. (Myszka DG. Improving biosensor analysis. J Mol Recognit. 1999; 12 (5): 279-84. pmid: 10556875).

### AC-SINS

The AC-SINS assay was performed as described previously (Sule et al, Biophysical Journal 101, 1749-1757 (2011), Liu et al, mAbs 6 (2), 483-492 (2014)). In short, gold nanoparticles (15705; Ted Pella Inc.) were coated with 80% capturing anti-human goat IgG Fc (109-005-098; Jackson ImmunoResearch) and 20% with polyclonal goat nonspecific antibody (005-000-003; Jackson ImmunoResearch). The antibodies of interest were then incubated with the particles for 2 h and the wavelength shift was measured using Molecular Devices SpectraMax M2 with SoftMax Pro6 software. The self-interacting clones show a higher wavelength shift away from the PBS sample.

### Transient CHO Expression

Transient CHO expression was performed using standard methods known in the art. In general, CHO-K1 cells grown to about 4×10*6 cells/mL were pelleted and resuspended in transfection medium. DNA plasmids (1.5 μg total DNA/mL) were incubated with PEIpro (1:2 final, PolyPlus, Cat #115-100) in transfection medium at room temperature before addition to the CHO-K1 cell suspension. Transfected cultures were fed and maintained at 32° C. or 37° C., shaking until supernatant was harvested (at day 9) for purification.

Functional Assay using the Activin 2B Receptor/SMAD reporter assay

Compound information: Antibodies provided in HEPES buffer (25 mM HEPES, 150 mM NaCl pH 7.3). It was assumed that the molecular mass of the compounds is 150,000.

Antibody treatment condition: 100 nM, 10 nM, 1 nM, 100 pM, 10 pM, 1 pM, 0.1 pM, 0.01 pM, vehicle control 1 pM single concentration.

Storage: Stocks are stored in −80° C. freezer.

Protocol: HEK-Activin 2B Receptor/SMAD reporter cell line clone 2 was plated in white 96 well plates at 10K cells per well. After overnight incubation the medium was removed and replaced with 90 μL DMEM containing 0.2% FBS, 2 mM L-glutamine and 50 mM HEPES pH7.4 and cells were incubated for 4 h. Activin A was diluted in medium at 10× final concentration. A final concentration of an EC₈₀ of Activin A was used to assess the inhibitory activity of mAbs. This medium was used to prepare dilutions of the mAb at 10× the final required concentration. Activin A/mAb mixtures were incubated for 15 minutes at room temperature before adding 10 μL to the cells. Cells were incubated at 37° C. for 16 hours. Luciferase activity was detected by adding Bright Glo reagent and reading in an En Vision plate reader.

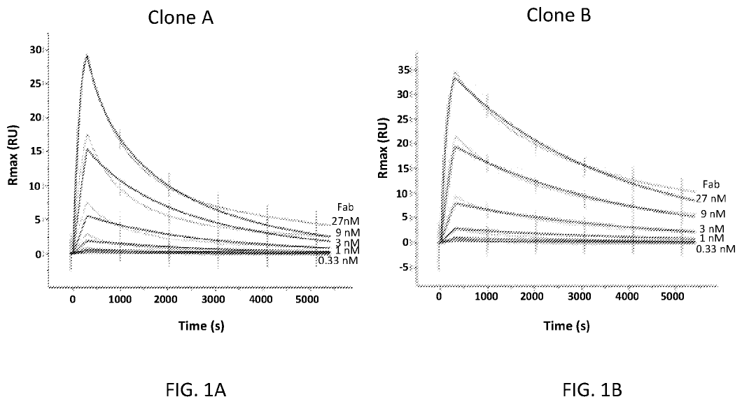

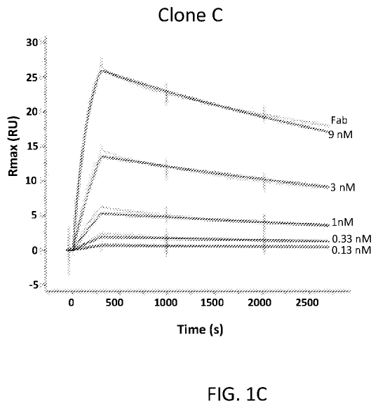

Results: The anti-Activin antibodies identified in Example 1 were characterized. First, binding kinetics of the anti-Activin A/B antibodies was assessed with a Surface Plasmon Resonance assay (Biacore). The binding kinetics of the anti-Activin A/B antibodies (Fab) are shown in Table 4 and representative graphs for Clones A-C are provided in FIGS. 1A-1C. The data shows that the anti-Activin A/B antibodies are potent and bind to Activin A with binding affinities within a range of about 83 pM to about 1600 pM as assessed by Biacore (e.g., Table 4).

TABLE 4
Binding kinetics of anti-Activin A/B antibodies

| | Biacore Fab K_{D} | | | |
|---|---|---|---|---|
| | biotinylated | | | |
| | Human Activin | | | |
| | A Fab K_{D} (M) | | | |
| Clone | Monovalent | kₒₙ (1/Ms) | k_{off} (1/s) | Rmax (RU)N |
| A | 1.64E−09 | 9.14E+05 | 1.49E−03 | 31.3 |
| B | 9.03E−10 | 3.30E+05 | 2.98E−04 | 37.5 |
| C | 8.32E−11 | 2.23E+06 | 1.85E−04 | 31.35 |

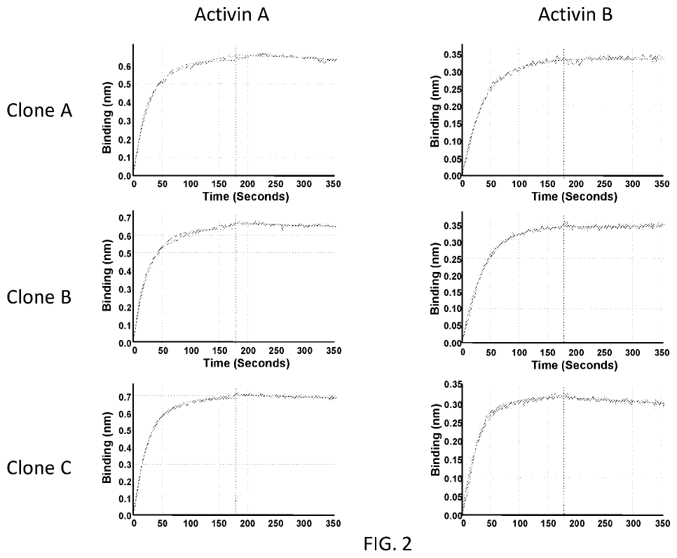

Binding affinity and epitope binning assays were performed with a ForteBio instrument as described herein. The results of the binding affinity (Octet) for all antibodies tested are provided in Table 5 and representative graphs for Clones A-C are provided in FIG. 2. Results of antibody selectivity are provided in Table 6.

TABLE 5
Binding affinity for anti-Activin A/B antibodies

| | Octet IgG K_{D} | Octet Fab K_{D} | |
|---|---|---|---|
| | Human | Human | Octet IgG K_{D} |
| | Activin A Fc | Activin A Fc | Activin B |
| Clone | (M) Avid | (M) Monovalent | (M) Avid |
| A | 6.61E−10 | 3.55E−09 | 7.60E−10 |
| B | 5.45E−10 | 2.10E−09 | 7.66E−10 |
| C | 5.52E−10 | 9.63E−10 | 8.61E−10 |

TABLE 6
Selectivity data for anti-Activin A/B antibodies

| | | | Octet IgG | Octet IgG | Octet IgG | Octet IgG | Octet IgG | Octet IgG |
|---|---|---|---|---|---|---|---|---|
| | Octet IgG | Octet IgG | K_{D} GDF15 | K_{D} Human | K_{D} Human | K_{D} Human | K_{D} Human | K_{D} Human |
| | K_{D} BMP9 | K_{D} BMP10 | Fc | GDNF | GDF8 | GDF10 | GDF11 | Follistatin 300 |
| Clone | (M) Avid | (M) Avid | (M) Avid | (M) Avid | (M) Avid | (M) Avid | (M) Avid | (M) Avid |
| A | N.B. | N.B. | N.B. | N.B. | N.B. | N.B. | N.B. | N.B. |
| B | N.B. | N.B. | N.B. | N.B. | N.B. | N.B. | N.B. | N.B. |
| C | N.B. | N.B. | N.B. | N.B. | N.B. | 2.58E−08 | 8.41E−09 | N.B. |

N.B. indicates a non-binder under the conditions tested.

As demonstrated in Tables 4 and 5, the anti-Activin A/B antibodies bound to human Activin A and/or Activin B with a high affinity. Clone C also had some detectable binding to one or both of GDF10 and GDF11 under the conditions tested, as shown in Table 6.

Further, the data in Table 6 shows that the anti-Activin A/B antibodies are highly specific to Activin A and/or Activin B. For example, the binding affinity of anti-Activin A/B antibodies in an IgG format to human Activin A was in the range of 5.52×10 (−10) M to 6.61×10(−10) M (see Table 5) and to human Activin B was in the range of 7.60×10(−10) M to 8.61×10(−10) M (see Table 5).

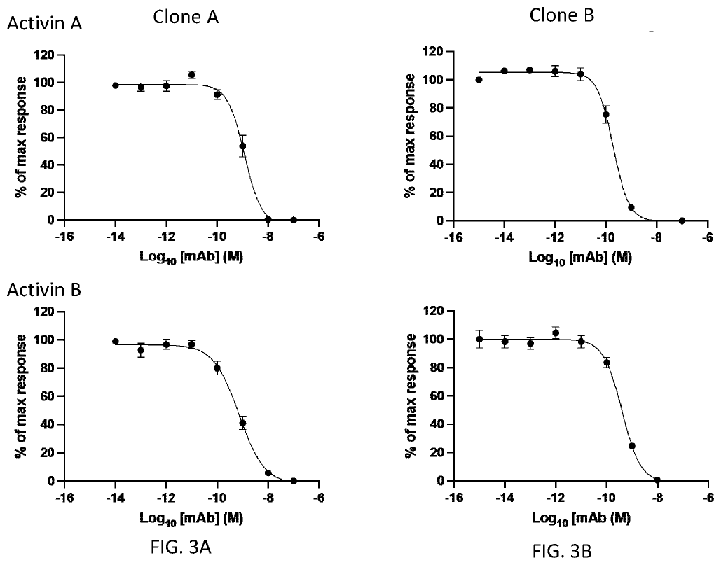

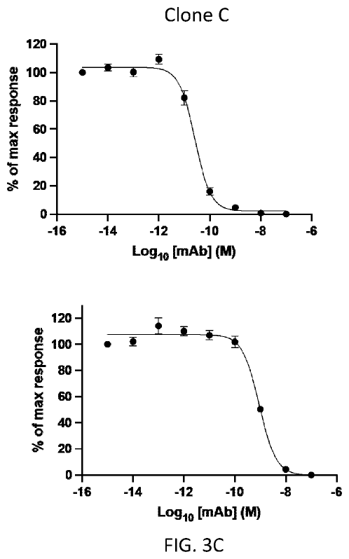

All of the Activin A antibodies had a strong inhibitory effect against Activin A and/or Activin B as measured in an Activin IIB Receptor/SMAD reporter assay (see Table 7 and FIGS. 3A-3C).

TABLE 7
Functional selectivity data for Activin A/B antibody agents

| Clone | Activin-A-ActRIIB SMAD IC₅₀ pM | | Activin-B-ActRIIB SMAD IC₅₀ pM | |
|---|---|---|---|---|
| A | 1176 pM | (C.I. 887-1671 pM, n = 4) | 719.1 pM | (C.I. 490-1082 pM, n = 6) |
| B | 194.2 pM | (C.I. 145.6-255 pM, n = 5) | 403.4 pM | (C.I. 299-537 pM, n = 7) |
| C | 18.1 pM | (C.I. 12.9-126.1 pM, n = 8) | 877 pM | (C.I. 651-1201 pM, n = 8) |

C.I.: Confidence Interval

None of the anti-Activin A/B antibodies functionally inhibited GDF11, BMP9 or BMP10 when assessed as described herein (e.g., as measured in an Activin IIB Receptor/SMAD reporter assay) (Table 8).

TABLE 8
Functional selectivity data for Activin A antibody agents

| | GDF-11- | BMP9 - | BMP10 - |
|---|---|---|---|
| | ActRIIB | ActRIIB | ActRIIB |
| | SMAD | SMAD; IgG | SMAD; IgG |
| Clone | IC₅₀ pM | IC₅₀ (pM) | IC₅₀ (pM) |
| A | | | |
| B | ND | ND | ND |
| C | ND | ND | ND |

ND: Not detected

Next, hydrophobicity of the anti-Activin A/B antibodies was tested using a HIC assay as described herein. Typically, hydrophobic antibodies show self-association and thus low hydrophobicity is preferred. The results of this assay for the anti-Activin A/B antibodies are shown in Table 9. Retention time of less than 10.5 minutes indicates a clean to low HIC, retention time of 10.5 to 11.5 indicates medium HIC and a retention time of more than 11.5 minutes indicates high HIC. The data in Table 9 demonstrates that all the anti-Activin A/B antibodies tested had low to medium hydrophobicity and are thus expected to have low self-association, with all of the clones showing retention times between 9 minutes to about 11 minutes.

TABLE 9
HIC Retention Time

| Clone | HIC Retention Time (min) |
|---|---|
| A | 9.2 |
| B | 9.6 |
| C | 10.5 |

Stability of the anti-Activin A/B antibodies was assessed with a DSF assay as described herein. The data shown in Table 10 shows that the melting temperature for the Fab fragments tested was between 81.0° C. to 84.5° C.

TABLE 10
Melting temperature for Fab fragments

| Clone | Fab Tₘ by DSF (° C.) |
|---|---|
| A | 82.0 |
| B | 84.5 |
| C | 81.0 |

As a further characterization, self-association of Clone A anti-Activin A/B antibodies was evaluated using the AC-SINS assay described herein using a CHO mammalian expression system. Transient CHO expression was first performed using standard methods, followed by purification of the resulting antibodies. The purified antibody was then subjected to an AC-SINS assay to evaluate self-interaction of the antibodies. The data from this experiment is provided in Table 11 and shows that the Clone A anti-Activin A/B antibody had a low AC-SINS value and thus has a low likelihood of self-associating.

TABLE 11
AC-SINS

| Clone | AC-SINS Δλmax (nM) |
|---|---|
| A | 3.89 |

Taken together, the data provided in this Example indicates that the human anti-Activin A/B antibodies identified in Example 1 have suitable “developability” characteristics and supports the development of these antibodies as therapeutics.

### Example 3: Expression of Activin A/B Antibody Agents in a Human Cell Line

This Example describes expression of anti-Activin antibody Clones A-C identified in Example 1 in a HEK293 expression system.

### Methods:

HEK293 cells were used to supply a transient expression of antibodies. Heavy and light chain of the antibody were individually cloned into an expression vector under CMV promotor. Equal amount of the heavy and light chain DNA constructs were mixed with polyethyleneimine (PEI). Transfection of HEK293 cells was performed by adding the PEI/DNA mixture to the suspension cells in exponential growth phase. The cells were then incubated at 37° C., 8% CO₂ and 120 rpm for up to 7 days. The culture was harvested by centrifuging the cell culture fluid and filtering the supernatant using a 0.2 μm depth filter.

SEC-HPLC was used to determine the molecular size variants in purified antibodies. Samples were diluted to 1-2 mg/mL with mobile phase and filtered through a 0.22 μm cellulose acetate membrane before analysis by YMC-Pack Diol column or equivalent. After peak integration, the main peak, aggregates (HMW), and fragments (LMW) were calculated based on the area normalization method.

### Results

As shown in Table 12, all antibodies tested were successfully expressed and purified in the HEK293 transient expression system. High expression of all clones was observed, with antibody concentrations in the range of about 100-200 mg/L.

TABLE 12
Antibody concentration and SEC peak

| | HEX-293 | HEK-293 produced |
|---|---|---|
| Clone | expression mg/L | SEC Main Peak (%) |
| A | 155.1 | 98.8 |
| B | 441 | 98.4 |
| C | 199.8 | 98.9 |

### Example 4: Pharmacokinetics of Anti-Activin A/B Antibodies

This Example describes pharmacokinetic analysis of exemplary anti-Activin A/B antibodies identified in Example 1, in animals (e.g., mice and rats)

### Methods

TABLE 13
IV Protocol in animals

| Category | Item | Description |
|---|---|---|
| Species | C57BL/6 Mice | Male |
| Formulation | Antibodies formulated in PBS | |
| In-life | mAb, IV, 1 mg/Kg | Dosing named Day 0 |
| | mAb, SC, 10 mg/Kg | PK sampling at 0, 1, 2, 6, 8, 24, 48, 144, |
| | | 312, 480, and 696 hours. ~0.2 mL (retro |
| | | orbital) or ~1 mL (cardiac puncture). |
| | | Clinical observations were conducted at |
| | | every time point. Blood samples were |
| | | collected from 3 mice at each specified |
| | | time point. |
| Bioanalytical | Method development | Bioanalytical method development using |
| | | Affinity-Capture Liquid Chromatography |
| | | Mass Spectrometry (LC/MS) |
| | Sample analysis | Plasma antibody concentrations were |
| | | measured using Affinity-Capture Liquid |
| | | Chromatography Mass Spectrometry |
| | | (LC/MS). |
| Species | Sprague Dawley Rats | Male |
| Formulation | Antibodies formulated in PBS | |
| In-life | mAb, IV, 10 mg/Kg | Dosing named Day 0 |
| | mAb, SC, 1 mg/Kg and 10 | PK sampling at 0, 1, 2, 6, 8, 24, 48, 144, |
| | mg/Kg | 312, 480, and 696 hours. ~0.3 mL (retro |
| | | orbital) or ~1 mL (cardiac puncture). |
| | | Clinical observations were conducted at |
| | | every time point. Blood samples were |
| | | collected from 3 mice at each specified |
| | | time point. |
| Bioanalytical | Method development | Bioanalytical method development using |
| | | Affinity-Capture Liquid Chromatography |
| | | Mass Spectrometry (LC/MS) |
| | Sample analysis | Plasma antibody concentrations were |
| | | measured using Affinity-Capture Liquid |
| | | Chromatography Mass Spectrometry |
| | | (LC/MS). |

Formulation: Anti-Activin A/B antibodies were diluted as appropriate to achieve the final concentrations. The doses were stored at −20° C.±5° C. upon arrival and following dose administration. The doses were brought to ambient temperature prior to dose administration.

Dosing: IV doses were delivered directly through the tail vein with an appropriately sized needle. SC doses were administered to the intrascapular region. Dose volumes were adjusted for the weight of the animal on the morning of dose administration.

Sample collection: Each mouse had at least 2 blood samples collected and in rat, blood was collected at all time points. Blood samples were collected from 3 animals at each specified time point with preference to the retro orbital sinus (first sample) or cardiac puncture (second or only sample) while anesthetized with inhaled Isoflurane. Blood samples were kept on ice until processing.

Sample Processing: All blood samples were centrifuged within 1 h of collection at settings of 3200 RPM, 10 min, and 5° C. Plasma samples were directly transferred into cluster tubes and stored at −20±5° C. until shipment.

Results: Among other things, in some embodiments, the present examples shows the mean±S.D serum concentration-time profile of anti-Activin A/B antibodies dosed intravenously in animals (e.g., mice (FIG. 4A. and/or rats (FIG. 4B.) In some embodiments, the present examples show the mean±S.D serum concentration-time profile of anti-Activin A/B antibodies dosed subcutaneously in animals (e.g., mice (FIG. 4A. and/or rats (FIG. 4B.). In some embodiments, the present example show the pharmacokinetics properties of exemplary anti-Activin A/B antibodies tested in vivo. The pharmacokinetic profiles of anti-Activin A/B antibodies dosed intravenously or subcutaneously show high concentrations of the antibodies in the serum up to 30 days after administration (e.g., demonstrating a linear elimination profile). This indicates that the tested anti-Activin A/B antibodies have minimal or no target mediated-drug disposition (TMDD) and are expected to have increased bioavailability.

### Example 5: Increased Activin B and Liver Enzymes in Mouse Model for Liver Fibrosis

This Example describes increased liver enzymes and Activin B in a mouse model of acute liver toxicity.

Methods: Male C57BL/6 mice (6 weeks) were obtained from the Jackson Laboratory and housed 4 animals per cage in a ventilated cage rack system. Animals received standard rodent chow and water ad libitum. After 6 days acclimation on a 12 hour light/dark cycle, all mice were randomized based on the bodyweights into groups of 6 mice. On Day 0, mice were intraperitoneally dosed with a single dose of CCl₄ (20% solution in corn oil) at 2.5 μL/g. Vehicle control mice received corn oil. Animal Health and mortality was recorded on a daily basis. On Day 3, all mice were sacrificed and collected for blood/serum. All serum samples were subjected to ALT and AST level analysis using colorimetric assay kits (Sigma, BioVision). Activin A and B levels were analyzed in serum samples using commercially available ELISA kits. For Activin A, the Mouse Activin A ELISA kit (R&D, cat #DACOOB) was used. For Activin B, the Mouse Activin B kit from Ansh Labs was used.

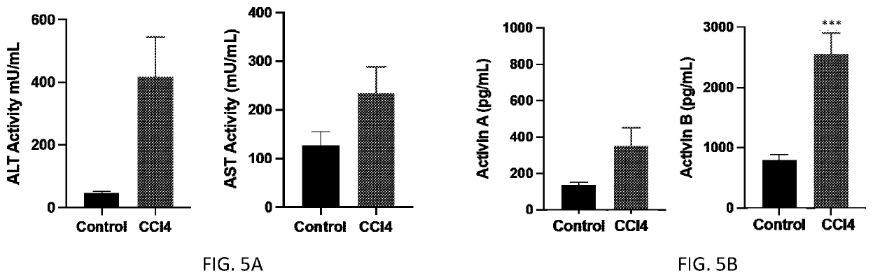

Results: As shown in FIG. 5A, mice treated with CCl₄ demonstrated increased ALT enzyme activity (left panel) and increased AST enzyme activity (right panel) compared to control animals. FIG. 5B shows that mice treated with CCl₄ had increased Activin B compared to control animals (right panel). A smaller increase in Activin A was observed (left panel).

### Example 6: Acute Overexpression of Activin-A and/or Activin-B May Cause Adverse Outcomes In-Vivo

Among other things, the present example demonstrates that acute AAV overexpression of Activin-A and/or Activin-B may cause rapid weight loss in a subject (e.g., mice).

Methods: Male C57BL/6J mice (>25 g upon arrival) were purchased from Jackson Laboratories. Mice were grouped (2-3 animals/cage) housed in clear polycarbonate cages with contact bedding meeting the requirements the Guide for the Care and Use of Laboratory Animals. Room in which the animals were kept was documented in the study records. No other species were housed in the same room. After randomization, 2-3 mice were housed together in one cage to prevent animal fighting. If signs of fighting are observed, single housing was provided. Animals were housed in a conventional housing room. Environmental controls will be set to maintain temperatures of 20° C. to 26° C. (68° F. to 79° F.) with a relative humidity of 30% to 70%. A 12-hour light/12-hour dark cycle will be maintained, except when interrupted to accommodate study procedures. Ten or greater air changes per hour were maintained in the animal room. All animals received environmental enrichment including manipulable devices, foraging opportunities, food items, structural and environmental enhancements, and positive human interaction

The week after arrival, all animals began a 3-day daily handling protocol where each animal was picked up but not dosed or weighed. On Day 0 animals were allocated to dosing groups based on body weight, body composition and daily food and water intake.

AAV dosing was undertaken on Day 0. Dosing was not time critical. Mice were dosed by the intravenous route and received 0.1 mL vector dose of either control vector AAV-Cag-h-Null (2.92×10¹³ GC/mL, Vector Biolabs), AAV8-Cag-h-INHBA (9.6×10¹¹ GC/mL, Vector Biolabs) or AAV8-Cag-h-INHBB (2.4×10¹² GC/mL, Vector Biolabs). Subsequent to dosing, mice were observed and weighed daily. Subchronic over-expression was up to 6 days and chronic overexpression was 4-6 weeks.

Once animals achieved 10-20% weight loss, the antibody effects are assessed. Mice either receive vehicle (IgG1 isotype control, PBS subcutaneously, n=5) or antibody (PBS, subcutaneously, n=5/dose). Mice are re-dosed with antibody once body weight plateaued with the previous dose (~5-6 days). During the vehicle or antibody dosing phases, mice are weighed daily.

All animals are euthanized by CO₂ asphyxiation followed by thoracotomy and exsanguination when body weight reversal was plateaued with repeat dosing. Maximal volume of blood are collected via cardiac puncture. The blood sample are collected into prechilled K₂-EDTA coated tubes. Blood was sent to IDEXX for clinical blood analysis. Remaining blood was gently inverted several times and processed to plasma. Plasma samples are then aliquoted and transferred to 1.5 mL centrifuge tubes (50 μL/tube). Plasma aliquots are stored at −80° C. Body organs are removed and weighted. Means are adjusted for differences between treatment groups in terminal body weight. For organ weights, SEMs are derived from the residuals of the statistical model. Results are analyzed by ANCOVA with terminal body weight as a covariate, followed by the multiple t test. Significant differences vs. AAV-Null: ***p<0.001. Significant differences vs IgG1 Isotype Control (same day): †††p<0.001

Plasma AST was measured using the Cobas C111 automated Chemistry analyzer and according to the manufacturer's instructions for estimation of Alkaline phosphatase.

Plasma ALT was measured using the Cobas C111 automated Chemistry analyzer and according to the manufacturer's instructions for Alanine transaminase (ALT) estimation.

For histology, liver and kidneys were collected at termination and placed into 10% NBF. All tissues were fixed for approximately 3 days prior to processing to wax block (FFPE). The tissues samples were sectioned at 5 μm thick and the slides stained using a method for Sirius Red, Haematoxylin and Eosin (H&E) or Masson's Trichrome. Examination by light microscopy was performed blinded and independently scored for fibrosis, necrosis and inflammatory cell infiltration by a Board-Certified histopathologist.

Body weight, food and water intake data were analyzed by mixed linear model with the group*day interaction as a fixed factor, and animal as the random subject using an AR (1)+RE covariance structure. Body weight gains and average food and water intake (weekly and for other relevant periods of the study) were derived from contrasts from the mixed models.

### Results:

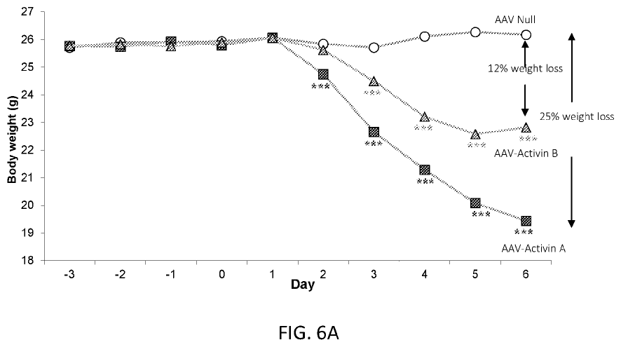

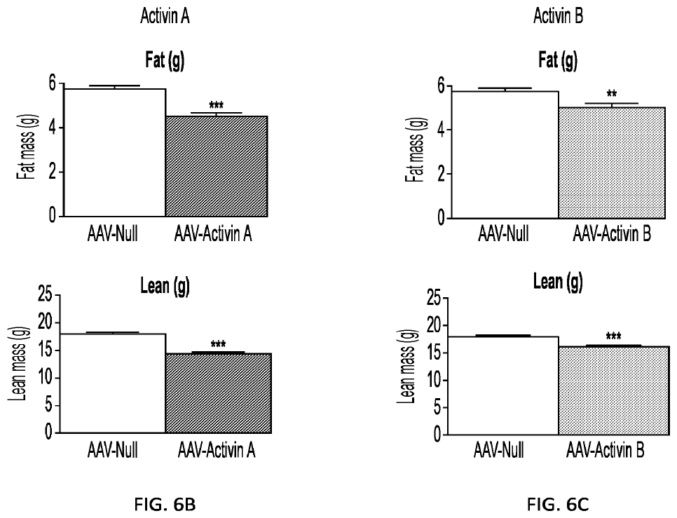

Among other things, the present example demonstrates that acute AAV-overexpression of Activin-A and/or Activin-B may cause rapid weight loss, fat and/or muscle loss. As demonstrated in FIG. 6A, mice administered viral vectors comprising Activin-A and/or Activin-B exhibited weight loss. Activin A exhibited 25% weight loss and Activin B exhibited by at least 12% weight loss, as compared to mice administered a null viral vector. Mice administered viral vectors comprising Activin-A and/or Activin-B exhibited weight loss by day 2 post administration, as compared to mice adminstered a null viral vector. As demonstrated in FIG. 6B and FIG. 6C, mice administered viral vectors comprising Activin-A and/or Activin-B exhibited weight loss associated with at least a 15% reduction in fat mass (measured in grams) and/or 10% reduction in lean muscle mass (measured in grams), as compared to mice administered a null viral vector.

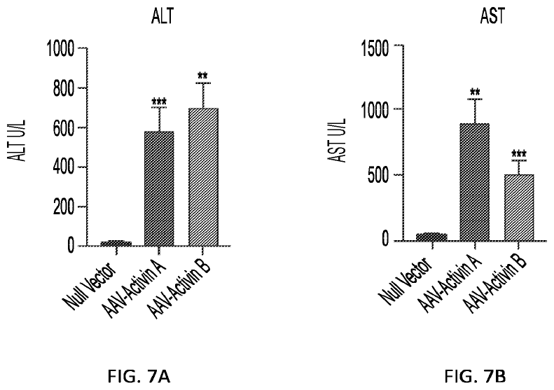

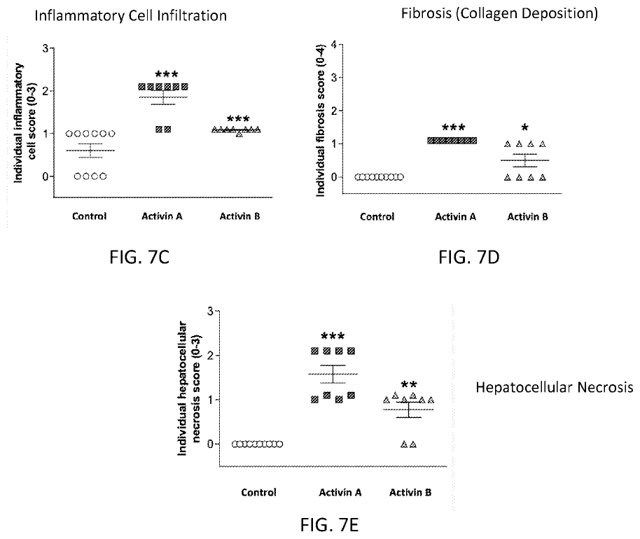

Among other things, the present example demonstrates that a 6 day acute AAV-overexpression of Activin-A and/or Activin-B may cause tissue damage (e.g., liver). As demonstrated in FIGS. 7A and 7B, mice administered viral vectors comprising Activin-A and/or Activin-B had increased levels of biomarkers associated with reduced liver function (e.g., ALT, AST), as compared to mice administered a null viral vector. As demonstrated in FIGS. 7C-7E, mice administered viral vectors comprising Activin-A and/or Activin-B exhibited worse tissue outcomes (e.g., as measured by inflammatory cells, fibrosis, and/or hepatocellular necrosis), as compared to mice administered a null viral vector.

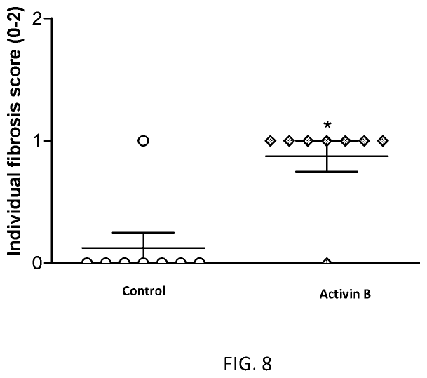

Among other things, the present example demonstrates that chronic overexpression of Activin B for 32 days causes liver fibrosis in mice. As shown in FIG. 8, mice administered a viral vector expressing Activin B had increased fibrosis compared to mice administered a control vector.

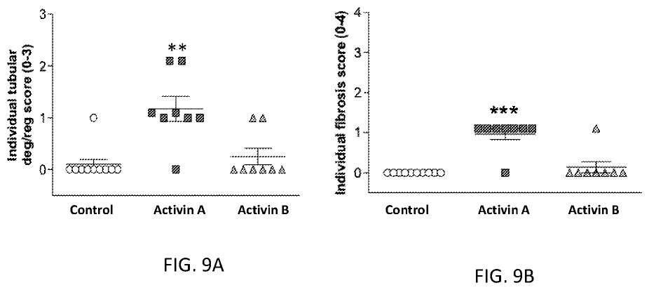

Among other things, the present example demonstrates that acute overexpression of Activin A for 6 days causes kidney damage in mice. Mice administered a viral vector expressing Activin A had increased tubular degeneration (FIG. 9A) and increase kidney fibrosis (FIG. 9B) as compared to mice administered a viral vector expressing Activin B or a control vector.

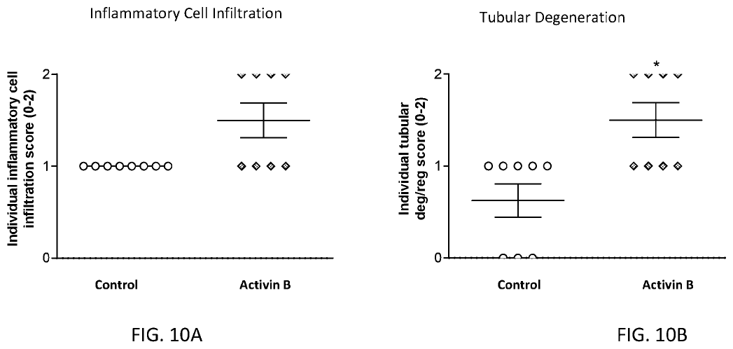

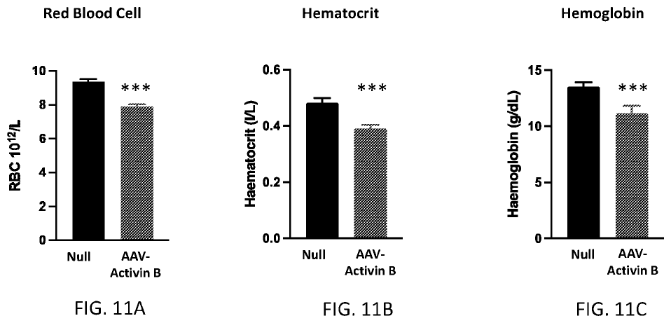

Among other things, the present example demonstrates that chronic overexpression of Activin B for 32 days causes kidney damage in mice. As demonstrated in FIGS. 10A and 10B, mice administered a viral vector expressing Activin B had increased infiltration of inflammatory cells and increased tubular degeneration as compared to mice administered a control vector. Among other things, the present example demonstrates that chronic overexpression of Activin B causes anemia. As demonstrated in FIGS. 11A-1C, mice administered a viral vector expressing Activin B had decreased red blood cell counts, decreased haematocrit, and decreased haemoglobin levels compared to mice administered a control vector.

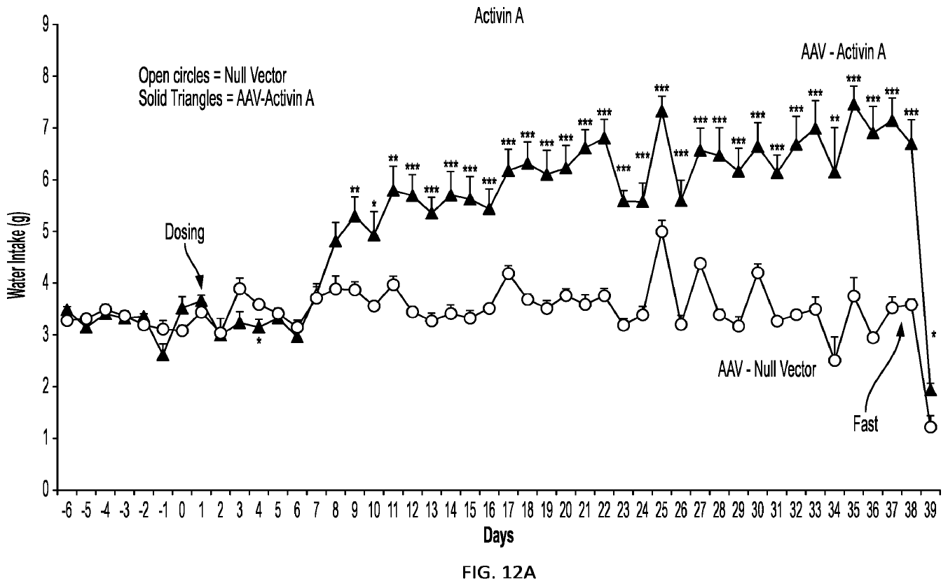

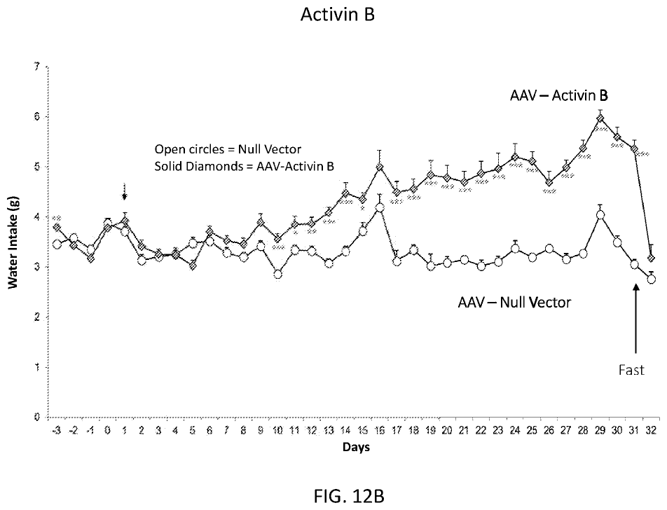

Among other things, the present example demonstrates that AAV-overexpression of Activin-A and/or Activin-B increases water intake, e.g., due to increased thirst and/or hypertension which was sustained). As demonstrated in FIG. 12A, mice administered viral vectors comprising Activin-A showed increased water intake by at least 20% by Day 9 post-administration, as compared to mice administered a null viral vector. As demonstrated in FIG. 12B, mice administered viral vectors comprising Activin-B showed increased water intake by at least 20% by Day 10 post-administration, as compared to mice administered a null viral vector.

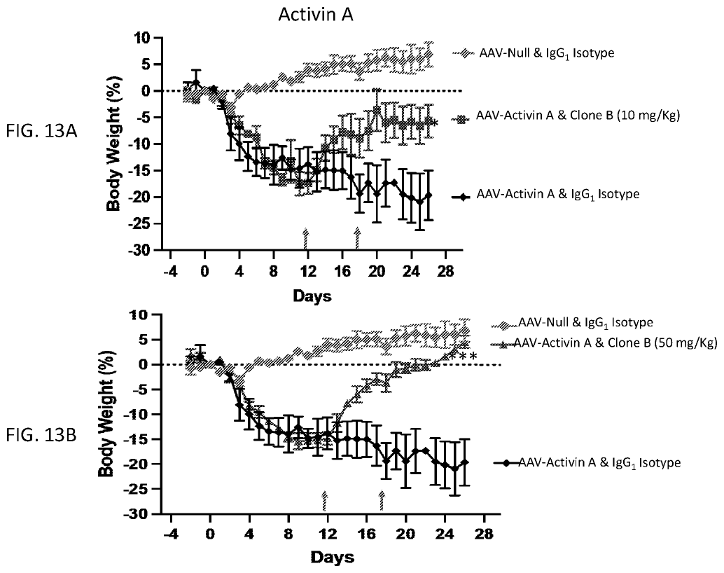

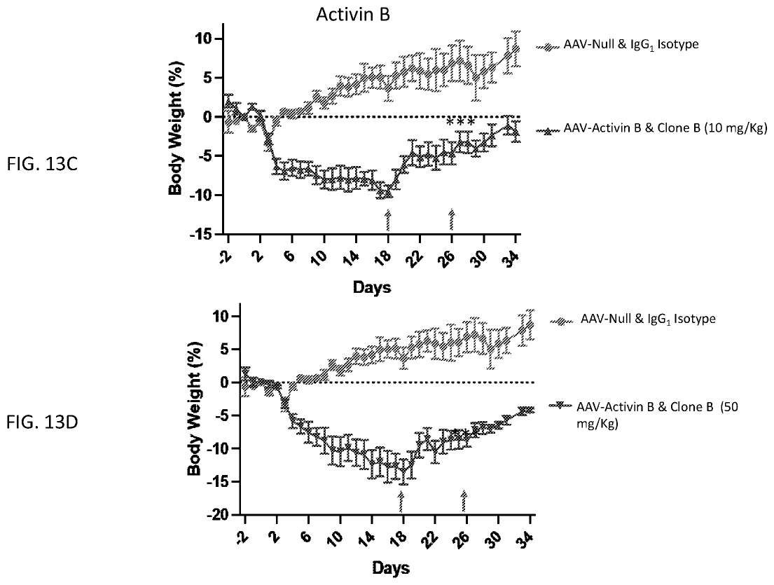

Among other things, the present example demonstrates that overexpression of Activin A or Activin B induces weight loss and cachexia which can be reversed with administration of an exemplary Activin A/B antibody agent. As demonstrated in FIGS. 13A-13B, mice administered control AAV vectors and treated with isotype control antibodies continue to gain weight throughout the course of the experiment. Mice administered viral vectors expressing Activin A showed weight loss induced by overexpression of Activin A. Subsequent administration of isotype control antibody to Activin A overexpressing animals at days 12 and 18 did not stop the weight loss and the animals continued to lose weight. In contrast, in Activin A overexpressing mice administration of an exemplary Activin A/B antibody agent on day 12 stopped the weight loss and induced weight gain. Administration of a second dose of the Activin A/B antibody agent on day 18 reversed the weight loss. This effect was observed with Activin A/B antibody agent administration at 10 mg/kg (FIG. 13A) and 50 mg/kg (FIG. 13B). A similar reversal of Activin B overexpression-induced weight loss and cachexia was observed was observed when administered at days 18 and 26 (FIGS. 13C-13D).

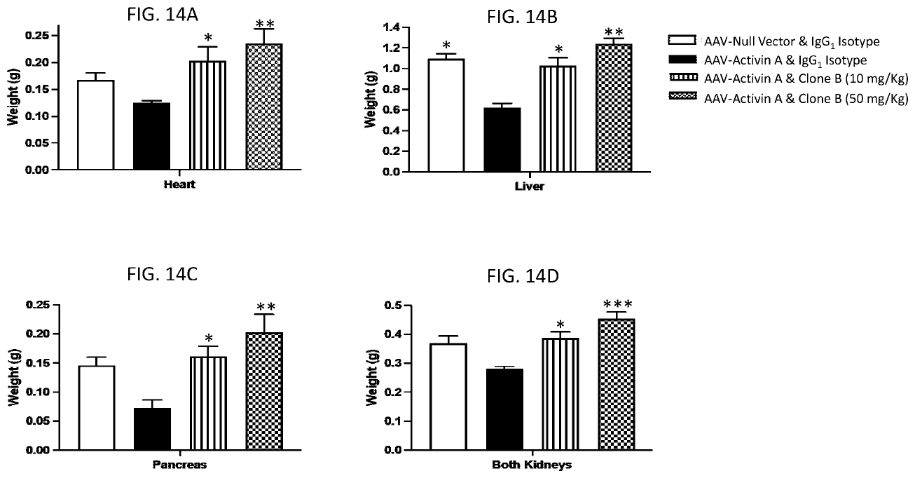

Among other things, the present example demonstrates that overexpression of Activin A causes multi-organ damage and decreased organ weights which can be reversed by administration of an exemplary Activin A/B antibody agent. As demonstrated in FIG. 14A, animals administered with an AAV vector expressing Activin A for 26 days and a control antibody (IgG1 isotype) administered at days 12 and day 18 showed a reduction in the weight of the heart as compared to animals administered with a control AAV vector at day 26. In comparison, administration of an AAV vector expressing Activin A and an exemplary Activin A/B antibody agent at 10 mg/kg or 50 mg/kg reversed the reduction in organ weight (heart). A similar effect was seen for other organs: liver (FIG. 14B), pancreas (FIG. 14C), and both kidneys (FIG. 14D).

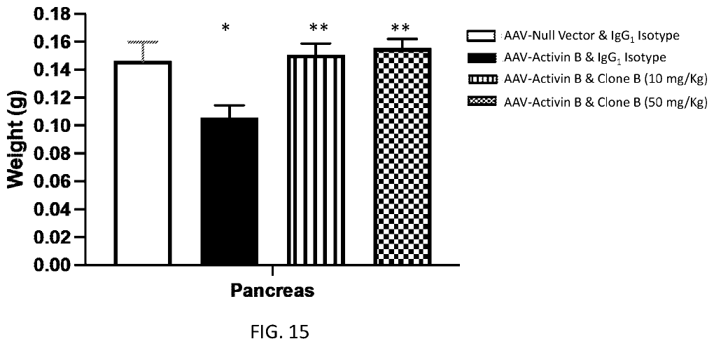

Among other things, the present example demonstrates that overexpression of Activin B causes decreased organ weights which can be reversed by administration of an exemplary Activin A/B antibody agent. As demonstrated in FIG. 15, animals administered with an AAV vector expressing Activin B for 33 days and a control antibody (IgG1 isotype) administered at days 18 and 26 showed a reduction in the weight of the pancreas as compared to animals administered with a control AAV vector at day 33. In comparison, administration of an AAV vector expressing Activin B and an exemplary Activin A/B antibody agent at 10 mg/kg or 50 mg/kg reversed the reduction in organ weight (pancreas).

Among other things, the present example demonstrates that chronic overexpression of Activin A causes increase liver damage which can be reversed by administration of an exemplary

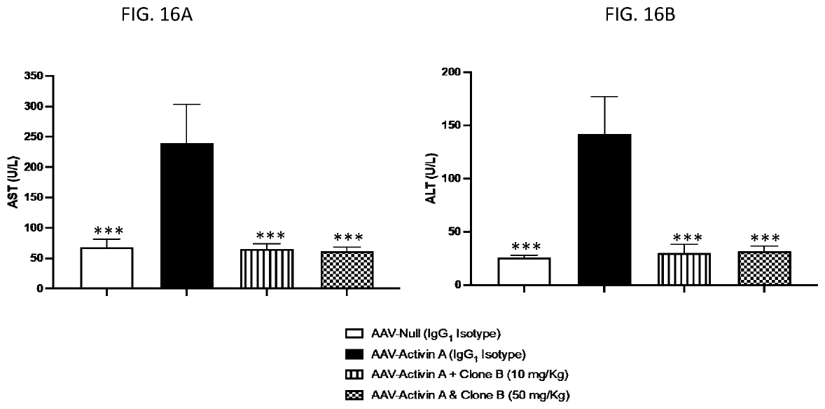

Activin A/B antibody agent. As demonstrated in FIG. 16A, animals administered with an AAV vector expressing Activin A and a control antibody (IgG1 isotype) showed an increase in AST, as compared to animals administered with a control AAV vector. In comparison, administration of an AAV vector expressing Activin A and an exemplary Activin A/B antibody agent at 10 mg/kg or 50 mg/kg reversed the increase in AST. As demonstrated in FIG. 16B, animals administered with an AAV vector expressing Activin A and a control antibody (IgG1 isotype) showed an increase in ALT, as compared to animals administered with a control AAV vector. In comparison, administration of an AAV vector expressing Activin A and an exemplary Activin A/B antibody agent at 10 mg/kg or 50 mg/kg reversed the increase in ALT.

### Example 7: In Vivo Anti-Cancer Activity of Anti-Activin a Antibody

This Example describes in vivo activity of an exemplary anti-Activin A antibody in an intrasplenically implanted MC38 model of colorectal cancer. Among other things, this Example shows inhibition of metastasis with an anti-Activin A antibody. The provided anti-Activin A-B antibodies of the present disclosure which bind both Activin A and Activin B are taught by the present disclosure to also be useful in preventing metastasis.

Methods: Metastatic MC38 Mouse In Vivo Model. 16-week-old male C57BL/6 mice (The Jackson Laboratory, Bar Harbor, ME, USA) were group-housed (4 per cage). Animals were injected with intrasplenic injection of 1.25×10⁵ MC38 tumor cells in sterile saline (MC38, n =16) or an isovolumetric intrasplenic injection of vehicle (Sham, n=8) as per Huot et al., (2021) Int. J. Mol. Sci. 22 (3): 1486. Sham and one MC38 group (n=8) received a subcutaneous injection of IgG1 isotype control (20 mg/Kg in PBS) on Day −2 prior to implantation and dosed every 4 days. Another MC38 group was dosed with Clone C (20 mg/Kg in PBS) on Day −2 prior to implantations, and dosed every 4 days. Food intake and body weight were measured daily and on day 18 post MC38 cell implantation, animals were euthanized under light isoflurane anesthesia. Following euthanasia tissues and tumors were harvested and weighed. Mice carcasses were weighed.

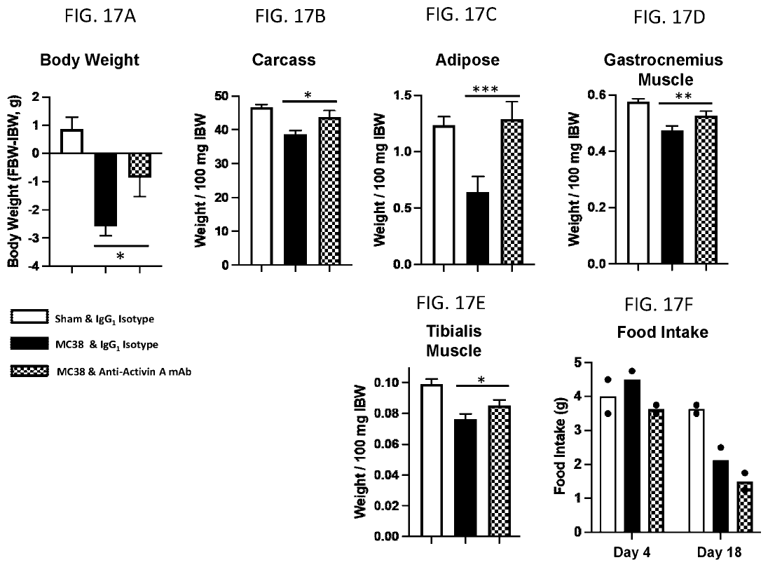

Results: In this MC38 model of colorectal cancer, the exemplary anti-Activin A antibody prevented cancer-induced weight loss, adipose loss and muscle loss in mice as compared to sham treated mice or isotype control treated mice (FIGS. 17A-17E). Notably, there was no effect on food intake with or without treatment with the exemplary anti-Activin A antibody (FIG. 17F). This data demonstrates that anti-Activin A antibodies can be used to prevent cancer-induced weight loss, adipose loss, and/or muscle loss in cancer patients.

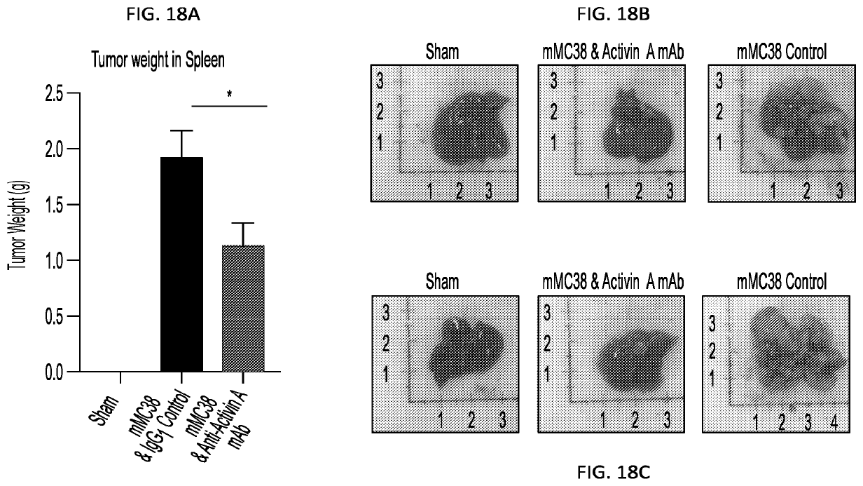

Further as shown in FIG. 18A, the exemplary anti-Activin A antibody prevented growth of the primary colorectal tumor in the spleen in mice implanted intrasplenically with MC38 colorectal cancer cells. In addition, the exemplary anti-Activin A antibody also prevented liver metastases in mice implanted with intrasplenic MC38 colorectal cancer cells (FIGS. 18B-18C). This data demonstrates that the anti-Activin A antibody can prevent: (1) growth of cancer cells; and/or (2) migration, invasion and/or colonization of cancer cells, e.g., to the liver thus reducing metastases in the liver. This data also suggests a therapeutic efficacy for anti-Activin A antibodies in reducing and/or preventing cancer growth and/or cancer metastases.

Without wishing to be bound by any particular theory, it is proposed that the observed inhibition of tumor growth arises from the activity of Anti-Activin A antibodies in preventing tumor growth. The present disclosure therefore demonstrates inhibition of growth of colorectal cancer tumor with an anti-Activin A antibody and teaches that the provided anti-Activin A-B antibodies which bind to both Activin A and Activin B can be used to treat and/or eliminate cancer.

Without wishing to be bound by any particular theory, it is proposed that the observed metastasis inhibition arises from the activity of Anti-Activin A antibodies in preventing cell migration, e.g., extravasation out of a tissue or organ into the bloodstream or lymphatic system, and/or intravasation into a secondary tissue or organ to establish a metastasis. The present disclosure therefore demonstrates inhibition of metastasis from a colorectal cancer tumor with an anti-Activin A antibody and teaches the provided anti-Activin A-B antibodies which bind to both Activin A and Activin B can be used to inhibit and/or reduce metastasis in other cancers.

### Example 8: Anti-Activin a Antibody Prevents Activin A-Induced Liver Damage In Vivo

This Example describes a prevention and reversal of liver damage with an exemplary anti-Activin A antibody in a mouse model of liver damage induced by Activin A overexpression.

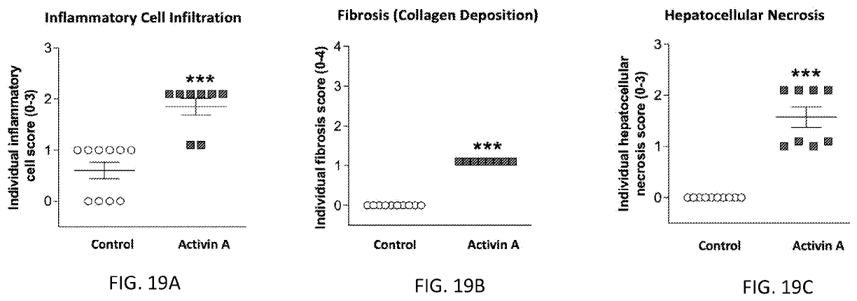

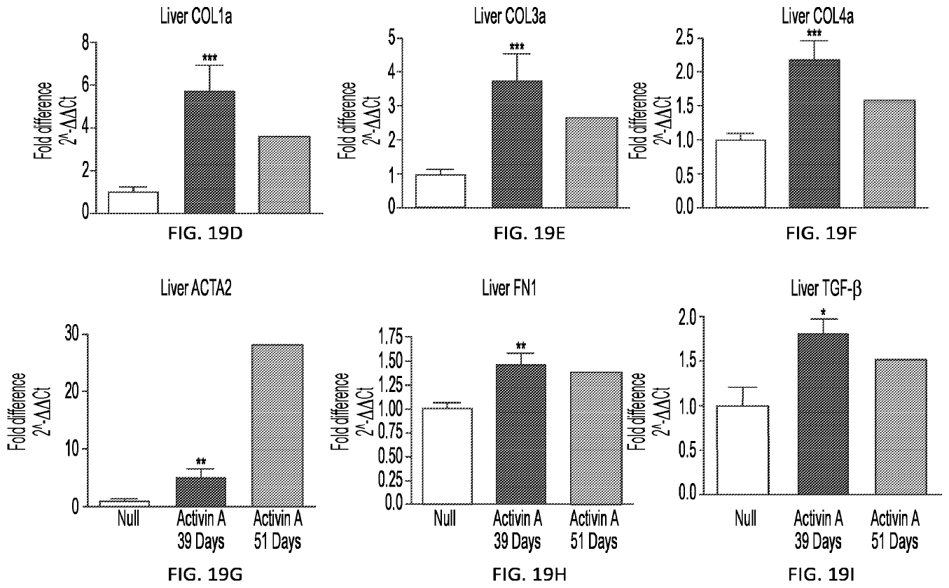

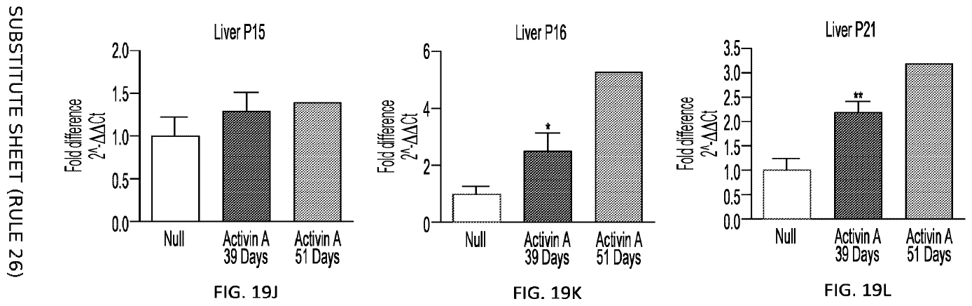

FIGS. 19A-19C show that acute Activin A overexpression induced liver weight loss attributed to acute liver injury. FIG. 19A shows increased inflammatory cell inflammation, FIG. 19B shows fibrosis, and FIG. 19C shows hepatocellular necrosis in mice overexpressing Activin A.

When Activin A was expressed chronically, an increase in fibrotic genes was observed. FIGS. 19D-19I show increased expression of Collagen (FIGS. 19D-19F), smooth muscle actin (FIG. 19G), fibronectin (FIG. 19H) and TGF-β (FIG. 19I) at 39 days and 51 days post-Activin A overexpression. Additionally, increased expression of p16 (FIG. 19K) and p21 (FIG. 19L) was also observed in liver cells after chronic overexpression of Activin A, indicating Activin-A induced senescence in the liver.

### Example 9. Bone loss associated with Activin A

This Example Describes Bone Loss Induced by Chronic Activin a Expression.

Methods: Male C57BL/6 mice (28 g at delivery) were dosed by the intravenous route and received either control null vector (9.6×10¹¹ GC/mL (0.1 mL), Vector Biolabs) or AAV8-Cag-h-INHBA (9.6×10¹¹ GC/mL (0.1 mL), Vector Biolabs). Mice were weighed daily and od libitum water intake was recorded 6 days pre-AAV administration and 39-51 days post-AAV administration. Body composition including bone density was measured at baseline and post AAV-administration using Dual-nergy X-ray absorptiometry (DEXA). Mice achieved 20% weight loss that was maintained throughout the duration of the study. Upon termination liver and femurs were harvested. Livers were snap frozen for gene expression studies. Femurs were fixed for 3 days in 10% neutral buffered formalin and then transferred to PBS containing 0.1% sodium azide and stored at 4° C. X-ray Computed Tomography was used to assess the microstructure of mouse femurs. The scans were conducted at 12 microns resolution. Bones were scanned using an X-ray energy of 75 kV & 110 μA current using a GE v|tome|x M 240 kV CT system. Data was reconstructed then processed using VGStudioMAX (Volume Graphics GmbH) software. Measurements from the trabecular bone (on a 0.7×0.7×3 mm volume at distal end of femur) included trabecular bone volume, trabecular bone volume/total volume, trabecular number, trabecular thickness & trabecular spacing.

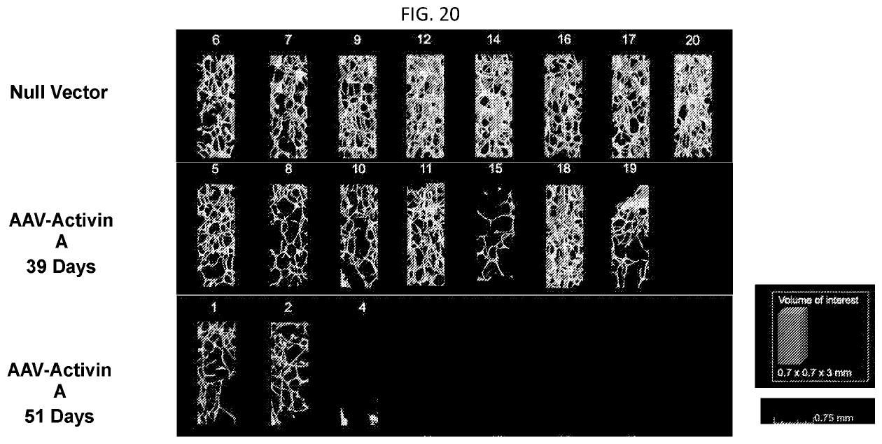

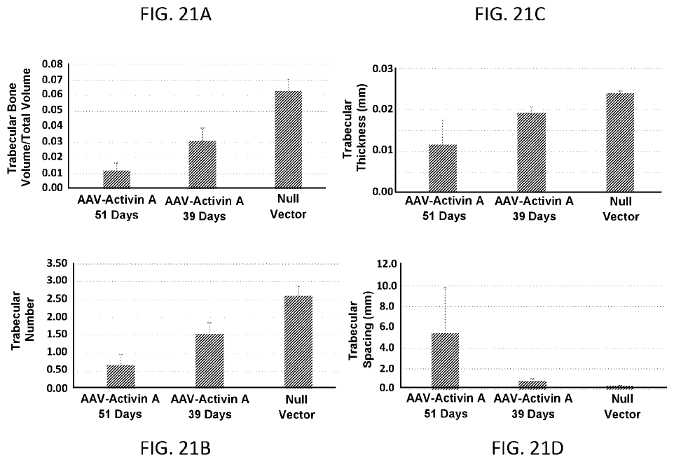

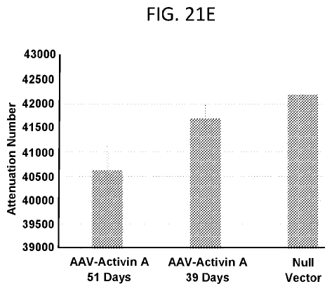

Results: Chronic Activin A expression also resulted in bone loss in mice. As shown in FIG. 20, femurs from mice overexpressing Activin A had reduced bone density as compared to control animals. The amount of bone loss observed was higher at 51 days compared to 39 days of chronic Activin A expression. FIGS. 21A-21E provide a quantification of the bone loss shown in FIG. 20.

This data demonstrates that anti-Activin A antibodies can be used to treat and/or prevent bone loss, which can be associated with elevated levels of Activin A.

### NUMBERED EMBODIMENTS

Embodiment 1. An antibody agent comprising a polypeptide that binds to Activin-A, Activin-B, and/or to one or more complexes that include one or both Activin-A and Activin-B comprising at least one light chain complementarity determining region (LC CDR) and/or at least one heavy chain complementary determining region (HC CDR).

Embodiment 2. The antibody agent of embodiment 1, wherein the antibody agent is or comprises: (i) an intact IgA, IgG, IgD, IgE or IgM antibody; (ii) an antibody fragment; (iii) a single domain antibody; (iv) a single chain Fv; or (v) a polypeptide comprising an antigen binding domain fused to a Fc domain.

Embodiment 3. The antibody agent of embodiment 1 or 2, comprising: (i) one, two, or three LC CDRs; or (ii) one, two or three HC CDRs.

Embodiment 4. The antibody agent of any one of embodiments 1-3, comprising one, two, or three LC CDRs and one, two, or three HC CDRs.

Embodiment 5. The antibody agent of any one of the preceding embodiments, comprising:
(i) an LC CDR1 sequence provided in Table 1, e.g., any one of SEQ ID NOs: 1 or 11;
(ii) a sequence with at least 85%, 86%, 87%, 88%, 89%, 90%, 91%, 92%, 93%, 94%, 95%, 96%, 97%, 98%, 99% identity to an LC CDR1 sequence provided in Table 1, e.g., any one of SEQ ID NOs: 1 or 11; or
(iii) a sequence having at least 5, 10, or 20 substitutions relative to an LC CDR1 sequence provided in Table 1, e.g., any one of SEQ ID NOs: 1 or 11.

Embodiment 6. The antibody agent of any one of the preceding embodiments comprising:
(i) an LC CDR2 sequence provided in Table 1, e.g., any one of SEQ ID NOs: 2 or 12;
(ii) a sequence with at least 85%, 86%, 87%, 88%, 89%, 90%, 91%, 92%, 93%, 94%, 95%, 96%, 97%, 98%, 99% identity to an LC CDR2 sequence provided in Table 1 e.g., any one of SEQ ID NOs: 2 or12; or
(iii) a sequence having at least 5, 10, or 20 substitutions relative to an LC CDR2 sequence provided in Table 1, e.g., any one of SEQ ID NOs: 2 or12.

Embodiment 7. The antibody agent of any one of the preceding embodiments, comprising:
(i) an LC CDR3 sequence provided in Table 1, e.g., any one of SEQ ID NOs: 3 or 13;
(ii) a sequence with at least 85%, 86%, 87%, 88%, 89%, 90%, 91%, 92%, 93%, 94%, 95%, 96%, 97%, 98%, 99% identity to an LC CDR3 sequence provided in Table 1, e.g., any one of SEQ ID NOs: 3 or 13; or
(iii) a sequence having at least 5, 10, or 20 substitutions relative to an LC CDR3 sequence provided in Table 1, e.g., any one of SEQ ID NOs: 3 or 13.

Embodiment 8. The antibody agent of any one of the preceding embodiments, wherein the antibody agent comprising a LC CDR1, LC CDR2 and/or LC CDR3 is capable of binding specifically to Activin A.

Embodiment 9. The antibody agent of any one of the preceding embodiments, wherein the antibody agent comprising a LC CDR1, LC CDR2 and/or LC CDR3 is capable of binding specifically to Activin B.

Embodiment 10. The antibody agent of any one of the preceding embodiments, comprising:
(i) an LC CDR1, LC CDR2, and LC CDR3 sequence provided in Table 1;
(ii) a sequence with at least 85%, 86%, 87%, 88%, 89%, 90%, 91%, 92%, 93%, 94%, 95%, 96%, 97%, 98%, 99% identity to an LC CDR1, LC CDR2, and LC CDR3 sequence provided in Table 1; or
(iii) a sequence having at least 5, 10, or 20 substitutions relative to an LC CDR1, LC CDR2, and LC CDR3 sequence provided in Table 1.

Embodiment 11. The antibody agent of any one of embodiments 1-10, comprising:
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 1;
(ii) an LC CDR2 of SEQ ID NO: 2, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 2; and/or
(iii) an LC CDR3 of SEQ ID NO: 3, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 3.

Embodiment 12. The antibody agent of any one of embodiments 1-10, comprising:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 11;
(ii) an LC CDR2 of SEQ ID NO: 12, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 12; and/or
(iii) an LC CDR3 of SEQ ID NO: 13, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 13.

Embodiment 13. The antibody agent of any one of the preceding embodiments, comprising:
(i) a HC CDR1 sequence provided in Table 2, e.g., any one of SEQ ID NOs: 20, 30, 35;
(ii) a sequence with at least 85%, 86%, 87%, 88%, 89%, 90%, 91%, 92%, 93%, 94%, 95%, 96%, 97%, 98%, 99% identity to a HC CDR1 sequence provided in Table 2, e.g., any one of SEQ ID NOs: 20, 30, 35; or
(iii) a sequence having at least 5, 10, or 20 substitutions compared to a HC CDR1 sequence provided in Table 2, e.g., any one of SEQ ID NOs: 20, 30, 35.

Embodiment 14. The antibody agent of any one of the preceding embodiments, comprising:
(i) a HC CDR2 sequence provided in Table 2, e.g., any one of SEQ ID NOs: 21, 31, or 36;
(ii) a sequence with at least 85%, 86%, 87%, 88%, 89%, 90%, 91%, 92%, 93%, 94%, 95%, 96%, 97%, 98%, 99% identity to a HC CDR2 sequence provided in Table 2, e.g., any one of SEQ ID NOs: 21, 31, or 36; or
(iii) a sequence having at least 5, 10, or 20 substitutions compared to a HC CDR2 sequence provided in Table 2, e.g., any one of SEQ ID NOs: 21, 31, or 36.

Embodiment 15. The antibody agent of any one of the preceding embodiments comprising:
(i) a HC CDR3 sequence provided in Table 2, e.g., any one of SEQ ID NOs: 22 or 37;
(ii) a sequence with at least 85%, 86%, 87%, 88%, 89%, 90%, 91%, 92%, 93%, 94%, 95%, 96%, 97%, 98%, 99% identity to a HC CDR3 sequence provided in Table 2, e.g., any one of SEQ ID NOs: 22 or 37; or
(iii) a sequence having at least 5, 10, or 20 substitutions compared to a HC CDR3 sequence provided in Table 2, e.g., any one of SEQ ID NOs: 22 or 37.

Embodiment 16. The antibody agent of any one of the preceding embodiments, wherein the antibody agent comprising a HC CDR1, a HC CDR2 and/or a HC CDR3 is able to specifically bind to Activin-A and/or Activin-B.

Embodiment 17. The antibody agent of any one of embodiments 1-16, comprising:
(i) an HC CDR1, HC CDR2, and HC CDR3 sequence provided in Table 2;
(ii) a sequence with at least 85%, 86%, 87%, 88%, 89%, 90%, 91%, 92%, 93%, 94%, 95%, 96%, 97%, 98%, or 99% identity to an HC CDR1, HC CDR2, and HC CDR3 sequence provided in Table 2;
(iii) a sequence having at least 5, 10, or 20 substitutions relative to an HC CDR1, HC CDR2, and HC CDR3 sequence provided in Table 2.

Embodiment 18. The antibody agent of any one of embodiments 1-17, comprising:
(i) an HC CDR1 of SEQ ID NO: 20, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 20;
(ii) an HC CDR2 of SEQ ID NO: 21, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 21; and/or
(iii) an HC CDR3 of SEQ ID NO: 22, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 22.

Embodiment 19. The antibody agent of any one of embodiments 1-17, comprising:
(i) an HC CDR1 of SEQ ID NO: 30, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 30;
(ii) an HC CDR2 of SEQ ID NO: 31, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 31; and/or
(iii) an HC CDR3 of SEQ ID NO: 22, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 22.

Embodiment 20. The antibody agent of any one of embodiments 1-17, comprising:
(i) an HC CDR1 of SEQ ID NO: 35, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 35;
(ii) an HC CDR2 of SEQ ID NO: 36, or a sequence with at least 85%, 86%, 87%,
88%, 89%, 90%, 91%, 92%, 93%, 94%, 95%, 96%, 97%, 98%, or 99% identity thereto or a sequence having at least 5, 10, or 20 substitutions relative to SEQ ID NO: 36; and/or
(iii) an HC CDR3 of SEQ ID NO: 37, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 37.

Embodiment 21. The antibody agent of any one of the preceding embodiments, comprising one, two, or three LC CDRs provided in Table 1 or a sequence with at least 85%, 86%, 87%, 88%, 89%, 90%, 91%, 92%, 93%, 94%, 95%, 96%, 97%, 98%, or 99% identity thereto; and one, two, or three HC CDRs provided in Table 2, or a sequence with at least 85%, 86%, 87%, 88%, 89%, 90%, 91%, 92%, 93%, 94%, 95%, 96%, 97%, 98%, or 99% identity thereto.

Embodiment 22. The antibody agent of embodiment 21, wherein the antibody agent comprises:
(a) a light chain comprising:
(i) an LC CDR1 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR1 provided in Table 1;
(ii) an LC CDR2 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR2 provided in Table 1; and/or
(iii) an LC CDR3 provided in Table 1 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to an LC CDR3 provided in Table 1; and
(b) a heavy chain comprising:
(i) a HC CDR1 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR1 provided in Table 2;
(ii) a HC CDR2 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR2 provided in Table 2; and/or
(iii) a HC CDR3 provided in Table 2 or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to a HC CDR3 provided in Table 2.

Embodiment 23. The antibody agent of embodiment 21 or 22, wherein the antibody agent comprises:
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 1; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 2; and an LC CDR3 of SEQ ID NO: 3, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 3; and
(ii) a HC CDR1 of SEQ ID NO: 20, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 20; an HC CDR2 of SEQ ID NO: 21, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 21; and an HC CDR3 of SEQ ID NO: 22, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 22.

Embodiment 24. The antibody agent of embodiment 21 or 22, wherein the antibody agent comprises:
(i) an LC CDR1 of SEQ ID NO: 1, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 1; an LC CDR2 of SEQ ID NO: 2, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 2; and an LC CDR3 of SEQ ID NO: 3, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 3; and
(ii) a HC CDR1 of SEQ ID NO: 30, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 30; an HC CDR2 of SEQ ID NO: 31, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 31; and an HC CDR3 of SEQ ID NO: 22, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 22.

Embodiment 25. The antibody agent of embodiment 21 or 22, wherein the antibody agent comprises:
(i) an LC CDR1 of SEQ ID NO: 11, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 11; an LC CDR2 of SEQ ID NO: 12, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 12; and an LC CDR3 of SEQ ID NO: 13, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 13; and
(ii) a HC CDR1 of SEQ ID NO: 35, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 35; an HC CDR2 of SEQ ID NO: 36, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 36; and an HC CDR3 of SEQ ID NO: 37, or a sequence with at least 85%, 86%, 87%, 88%, 89%, 90%, 91%, 92%, 93%, 94%, 95%, 96%, 97%, 98%, or 99% identity thereto or a sequence having at least 5, 10, or 20 substitutions relative to SEQ ID NO: 37.

Embodiment 26. The antibody agent of embodiment 67, wherein the VL region comprises one, two or three LC CDRs provided in Table 1 or a sequence with at least 85%, 86%, 87%, 88%, 89%, 90%, 91%, 92%, 93%, 94%, 95%, 96%, 97%, 98%, or 99% identity thereto, and one, two, three or four FR regions provided in Table 1 or a sequence with at least 92% identity thereto.

Embodiment 27. The antibody agent of embodiment 26, wherein the FR region comprises:
(i) a FR sequence provided in Table 1;
(ii) a sequence with at least 92%, 93%, 94%, 95%, 96%, 97%, 98%, or 99% identity to a FR sequence provided in Table 1; or
(iii) a sequence having at least 5, 10, or 20 substitutions compared to a FR sequence provided in Table 1.

Embodiment 28. The antibody agent of embodiment 27 or 28, comprising:
(i) the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; or
(ii) the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16.

Embodiment 29. The antibody agent of any one of embodiments 26-28, wherein the light chain further comprises a sequence for a constant region (CL).

Embodiment 30. The antibody agent of embodiment 29, wherein the light chain comprises a kappa CL or a lambda CL.

Embodiment 31. The antibody agent of any one of the preceding embodiments, wherein the antibody agent comprises a heavy chain comprising a variable region (VH) comprising at least one HC CDR provided in Table 2 or a sequence with at least 85%, 86%, 87%, 88%, 89%, 90%, 91%, 92%, 93%, 94%, 95%, 96%, 97%, 98%, or 99% identity thereto, and at least one framework region (FR) provided in Table 2 or a sequence with at least 92% identity thereto.

Embodiment 32. The antibody agent of embodiment 31, wherein the VH region one, two or three HC CDRs provided in Table 2 or a sequence with at least 85%, 86%, 87%, 88%, 89%, 90%, 91%, 92%, 93%, 94%, 95%, 96%, 97%, 98%, or 99% identity thereto, and one, two, three or four FR regions provided in Table 2 or a sequence with at least 92% identity thereto.

Embodiment 33. The antibody agent of embodiment 31 or 32, wherein the FR region comprises:
(i) a FR sequence provided in Table 2;
(ii) a sequence with at least 92%, 93%, 94%, 95%, 96%, 97%, 98%, or 99% identity to a FR sequence provided in Table 2; or
(iii) a sequence having at least 5, 10, or 20 substitutions compared to a FR sequence provided in Table 2.

Embodiment 34. The antibody agent of any one of embodiments 31-33, comprising:
(i) the sequence of SEQ ID NO: 27, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 27;
(ii) the sequence of SEQ ID NO: 32, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 32; or
(iii) the sequence of SEQ ID NO: 39, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 39.

Embodiment 35. The antibody agent of any one of embodiments 31-34, wherein the heavy chain region further comprises a sequence for at least one constant region (CH).

Embodiment 36. The antibody agent of embodiment 35, wherein the at least one constant region comprises an Fc domain.

Embodiment 37. The antibody agent of embodiment 36, wherein the Fc domain comprises a mammalian Fc domain, e.g., a mouse, a rat, a rabbit, a primate, a human, or a domestic animal Fc domain (e.g., a dog, a cat, a cow, or a horse Fc domain).

Embodiment 38. The antibody agent of embodiment 36 or 37, wherein the Fc domain is chosen from an Fc domain of an immunoglobulin isotype.

Embodiment 39. The antibody agent of embodiment 38, wherein the immunoglobulin isotype comprises IgA, IgG, IgM, or IgE.

Embodiment 40. The antibody agent of embodiment 38 or 39, wherein the Fc domain comprises an Fc domain of an IgG, e.g., a human IgG.

Embodiment 41. The antibody agent of embodiment 40, wherein the IgG is or comprises IgG1, 1gG2, lgG3, or lgG4.

Embodiment 42. The antibody agent of embodiment 41, wherein the IgG constant region comprises one or more modifications.

Embodiment 43. The antibody agent of embodiment 42, wherein the one or more modifications modulates one or more properties of the antibody agent.

Embodiment 44. The antibody agent of embodiment 42or 43, wherein the one or more modifications comprises:
(i) a LAGA mutation, a FEGG mutation, an AAGG mutation, an AAGA mutation, a LALA mutation or a combination thereof; and/or
(ii) a I253A mutation, a H310A mutation, a H435R mutation, a H435A mutation or a combination thereof.

Embodiment 45. The antibody agent of any the preceding embodiments, wherein the antibody agent comprises a VL comprising any one, two or three CDRs provided in Table 1 or a sequence with at least 85%, 86%, 87%, 88%, 89%, 90%, 91%, 92%, 93%, 94%, 95%, 96%, 97%, 98%, or 99% identity thereto and a VH comprising any one two or three CDRs provided in Table 2 or a sequence with at least 85%, 86%, 87%, 88%, 89%, 90%, 91%, 92%, 93%, 94%, 95%, 96%, 97%, 98%, or 99% identity thereto.

Embodiment 46. The antibody agent of any one of the preceding embodiments, wherein the antibody agent comprises a VL provided in Table 1 or a sequence with at least 85% identity thereto and a VH provided in Table 2 or a sequence with at least 85% identity thereto.

Embodiment 47. The antibody agent of embodiment 45 or 46, wherein the antibody agent comprises:
(i) the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; and the sequence of SEQ ID NO: 27, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 27;
(ii) the sequence of SEQ ID NO: 8, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 8; and the sequence of SEQ ID NO: 32, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 32; or
(iii) the sequence of SEQ ID NO: 16, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 16; and the sequence of SEQ ID NO: 39, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 39.

Embodiment 48. The antibody agent of any one of the preceding embodiments, wherein the antibody agent comprises:
(i) a light chain (LC) comprising: (a) one, two or three LC CDRs provided in Table 1 or a sequence with at least 85%, 86%, 87%, 88%, 89%, 90%, 91%, 92%, 93%, 94%, 95%, 96%, 97%, 98%, or 99% identity thereto; (b) at least one FR provided in Table 1 or sequence with at least 92% identity thereto; (c) a constant region (CL); and
(ii) a heavy chain (HC) comprising: (a) one, two or three HC CDRs provided in Table 2 or a sequence with at least 85%, 86%, 87%, 88%, 89%, 90%, 91%, 92%, 93%, 94%, 95%, 96%, 97%, 98%, or 99% thereof; (b) at least one FR provided in Table 1 or a sequence with at least 92% identity thereto; (c) at least one constant region.

Embodiment 49. The antibody agent of embodiment 48, wherein the antibody agent comprises:
(i) the sequence of SEQ ID NO: 9, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 9; and the sequence of SEQ ID NO: 28, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 28;
(ii) the sequence of SEQ ID NO: 9, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 9; and the sequence of SEQ ID NO: 33, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 33; or
(iii) the sequence of SEQ ID NO: 17, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 17; and the sequence of SEQ ID NO: 40, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 40.

Embodiment 50. The antibody agent of embodiment 48, wherein the antibody agent comprises:
(i) the sequence of SEQ ID NO: 9, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 9; and the sequence of SEQ ID NO: 28 without a terminal lysine, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 28 without a terminal lysine;
(ii) the sequence of SEQ ID NO: 9, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 9; and the sequence of SEQ ID NO: 33 without a terminal lysine, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 33 without a terminal lysine; or
(iii) the sequence of SEQ ID NO: 17, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 17; and the sequence of SEQ ID NO: 40 without a terminal lysine, or a sequence with at least 85%, 86%, 87%, 88%, 89%, 90%, 91%, 92%, 93%, 94%, 95%, 96%, 97%, 98%, or 99% identity thereto, or a sequence having at least 5, 10, or 20 substitutions relative to SEQ ID NO: 40 without a terminal lysine.

Embodiment 51. The antibody agent of any one of the preceding embodiments, wherein the antibody agent binds to human Activin-A:
(i) with a binding affinity (KD) of about 1.64 10 (−9) M to about 8.32 X 10 (−11) M. e.g., with a Fab format; and/or
(ii) with a binding affinity (KD) of about 5.45×10 (−10) M to about 6.61×10 (−10) M, e.g., with a IgG format.

Embodiment 52. The antibody agent of any one of the preceding embodiments, wherein the antibody agent binds to human Activin B with a binding affinity (KD) of about 7.6 X 10 (−10) M to about 8.61 X 10 (−10) M, e.g., with a IgG format.

Embodiment 53. The antibody agent of embodiment 51 or 52, wherein the binding affinity is determined with a binding affinity determining assay such as a surface plasmon resonance assay, an Octet assay or a comparable assay.

Embodiment 54. The antibody agent of any one of the preceding embodiments, characterized in that when tested in an assay that evaluates Activin-A and/or Activin-B activity and/or level, the antibody agent reduces Activin-A and/or Activin-B activity and/or level, or prevents an increase in Activin-A and/or Activin-B activity and/or level relative to a comparator.

Embodiment 55. The antibody agent of embodiment 54, wherein Activin-A and/or Activin-B is or comprises free and/or active Activin-A and/or Activin-B.

Embodiment 56. The antibody agent of embodiment 55, wherein Activin-A and/or Activin-B is or comprises free Activin-A and/or Activin-B.

Embodiment 57. The antibody agent of embodiment 55, wherein Activin-A and/or Activin-B is or comprises active Activin-A and/or Activin-B.

Embodiment 58. The antibody agent of embodiment 55, wherein Activin-A and/or Activin-B is or comprises free and active Activin-A and/or Activin-B.

Embodiment 59. The antibody agent of any one of embodiments 54-58, wherein the comparator is or comprises a sample that is not contacted with an Activin A/B antibody agent disclosed herein.

Embodiment 60. The antibody agent of any one of embodiments 54-59, wherein the antibody agent reduces a plasma, blood, serum, tumor and/or urine level of Activin-A and/or Activin-B.

Embodiment 61. The antibody agent of embodiment 60, wherein the antibody agent reduces the level of Activin-A and/or Activin-B to less than about 500 pg/mL.

Embodiment 62. The antibody agent of any one of embodiments 54-59, wherein the antibody agent prevents an increase in Activin-A and/or Activin-B levels in plasma, blood, serum, tumor, and/or urine.

Embodiment 63. The antibody agent of embodiment 58, wherein the antibody agent prevents an increase in Activin-A and/or Activin-B level above about 500 pg/mL.

Embodiment 64 The antibody agent of any one of embodiments 54-64, wherein the antibody agent reduces, e.g., inhibits, an Activin-A and/or Activin-B activity.

Embodiment 65. The antibody agent of any one of embodiments 54-64, wherein the antibody agent reduces, e.g., inhibits, the activity and/or level of Activin-A and/or Activin-B (e.g., plasma, blood, serum, tumor, and/or urine Activin-A and/or Activin-B, e.g., free and/or active Activin-A and/or Activin-B) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 66. The antibody agent of embodiment 65, wherein the antibody agent inhibits the activity and/or level of Activin-A and/or Activin-B (e.g., plasma, blood, serum, tumor and/or urine Activin-A and/or Activin-B, e.g., free and/or active Activin-A and/or Activin-B) by 100%.

Embodiment 67. The antibody agent of embodiment 65 or 66, wherein inhibition of Activin-A and/or Activin-B activity comprises inhibiting binding of Activin-A and/or Activin-B to:
(i) an Activin receptor (e.g., a Type II receptor, a Type I receptor or both);
(ii) a BMP receptor;
(iii) a TGF-beta receptor (e.g., a Type I receptor, a Type II receptor or both); or
(iv) any combination of (i)-(iii).

Embodiment 68. The antibody agent of embodiment 67, wherein inhibiting binding of Activin-A and/or Activin-B to an Activin receptor reduces, e.g., inhibits, an Activin receptor activity and/or an Activin receptor mediated signaling pathway.

Embodiment 69. The antibody agent of embodiment 67 or 68, wherein an Activin receptor is or comprises a Type II receptor.

Embodiment 70. The antibody agent of embodiment 69, wherein the Type II receptor is or comprises ACTRIIB or ACTRIIA, or both.

Embodiment 71. The antibody agent of embodiment 67 or 68, wherein an Activin receptor is or comprises a Type I receptor.

Embodiment 72. The antibody agent of embodiment 71, wherein the Type I receptor is or comprises Type I ACTRIA, ACTRIB, ACTR1C, or a combination thereof.

Embodiment 73. The antibody agent of any one of embodiments 71-72, wherein an Activin receptor mediated signaling pathway comprises one or more of: (i) a SMAD pathway, (ii) an ERK/MAPK pathway, (iii) a p38 MAPK pathway, (iv) NOX-2 dependent signaling, (v) STAT3 signaling, (vi) Snail and/or Slug mediated pathways.

Embodiment 74. The antibody agent of any one of the preceding embodiments, characterized in that when tested in an assay that evaluates activation of an Activin receptor, the antibody agent does not activate one or more signaling pathways activated by the Activin receptor.

Embodiment 75. The antibody agent of any one of the preceding embodiments, characterized in that when tested in an assay that evaluates activation of an Activin receptor, the antibody agent reduces, e.g., inhibits, Activin-A and/or Activin-B signaling via the Activin receptor, e.g., as assessed in an ActivinRIIB receptor/SMAD reporter assay in Example 2.

Embodiment 76. The antibody agent of embodiment 75, wherein the antibody agent reduces Activin-A and/or Activin-B signaling via the Activin receptor by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 77. The antibody agent of embodiment 75 or 76, the antibody agent inhibits Activin-A and/or Activin-B signaling via the Activin receptor by about 100%.

Embodiment 78. The antibody agent of any one of the preceding embodiments, wherein the antibody agent does not bind to or has minimal binding affinity for one or more TGFbeta super family members other than Activin-A and/or Activin-B.

Embodiment 79. The antibody agent of any one of the preceding embodiments, wherein the antibody agent does not bind to or has minimal binding affinity for any one, or all, or a combination of BMP-9, BMP-10, GDF-15, GDNF, GDF-8, GDF-10 or GDF-11.

Embodiment 80. The antibody agent of embodiment 78 or 79, which:

binds to GDF-11 or GDF-10 and is characterized in that when tested in an assay that evaluates GDF-11 or GDF-10 activity and/or level, the antibody agent does not modulate GDF-11 or GDF-10 activity and/or level.

Embodiment 81. The antibody agent of any one of the preceding embodiments, wherein the antibody agent has a KON, a KOFF or a KD provided in Table 4 as measured in a surface plasmon resonance assay.

Embodiment 82. The antibody agent of any one of the preceding embodiments, wherein the antibody agent competes for binding with a reference antibody agent that binds to Activin-A, Activin-B, and/or to one or more complexes that include one or both Activin-A and Activin-B.

Embodiment 83. The antibody agent of any one of the preceding embodiments, wherein the antibody agent has high specificity for Activin-A and/or Activin-B and low polyreactivity, as measured with a poly-specificity reagent (PSR) or a comparable reagent that measures antibody binding specificity.

Embodiment 84. The antibody agent of embodiment 83, wherein the antibody agent has a clean PSR score of less than 0.1, or has a low PSR score of between 0.1 to 0.22.

Embodiment 85. The antibody agent of any one of the preceding embodiments, wherein the antibody agent has low hydrophobicity as measured in a HIC assay or a comparable assay that measures hydrophobicity.

Embodiment 86. The antibody agent of embodiment 85, wherein antibody agent has a HIC retention time of less than 10.5 minutes indicating a clean to low HIC, or a retention time of between 9.2 to 10.5 indicating a medium HIC.

Embodiment 87. The antibody agent of any one of the preceding embodiments, wherein the antibody agent has low self-association as measured with an AC-SINS assay or a comparable assay that measures self-association.

Embodiment 88. The antibody agent of embodiment 87, wherein the antibody agent has an AC-SINS score 3.89 indicating no-low self-association.

Embodiment 89. The antibody agent of any one of the preceding embodiments, wherein the antibody agent has a melting temperature (Tm) of about 81.0° C. to about 84.5° C.

Embodiment 90. The antibody agent of any one of the preceding embodiments, wherein the antibody agent has a pharmacokinetic profile with linear elimination.

Embodiment 91. The antibody agent of embodiment 90, wherein the antibody agent has minimal or no target mediated-drug disposition (TMDD).

Embodiment 92. The antibody agent of embodiment 91, wherein the antibody agent has increased bioavailability compared to an antibody agent that has TMDD.

Embodiment 93. The antibody agent of any one of the preceding embodiments, wherein the antibody agent is produced in a bacterial cell, a yeast cell or a mammalian cell.

Embodiment 94. The antibody agent of any one of the preceding embodiments, wherein the antibody agent is produced in a mammalian cell.

Embodiment 95. The antibody agent of embodiment 94, wherein the mammalian cell is a CHO cell.

Embodiment 96. The antibody agent of any one of the preceding embodiments, wherein the antibody agent can be produced at a concentration of about 1000 to 20,000 mg/L, about 2000 to 20,000 mg/L, about 5000 to 20,000 mg/L, about 6000 to 20,000 mg/L, about 7000 to 20,000 mg/L, about 8000 to 20,000 mg/L, about 9000 to 20,000 mg/L, 10,000 to 20,000 mg/L or about 15,000 to 20,000 mg/L.

Embodiment 97. An isolated nucleic acid encoding an antibody agent of any one of embodiments 1-96.

Embodiment 98. An isolated nucleic acid comprising an Activin-A and/or Activin-B variable heavy (VH) sequence chosen from a VH DNA sequence provided in Table 2 or a sequence with at least 85%, 86%, 87%, 88%, 89%, 90%, 91%, 92%, 93%, 94%, 95%, 96%, 97%, 98%, or 99% identity thereto.

Embodiment 99. The isolated nucleic acid of embodiment 95, wherein the VH sequence comprises the nucleic acid sequence of SEQ ID NO: 29, SEQ ID NO: 34, SEQ ID NO: 41, or a nucleic acid sequence with at least 85%, 86%, 87%, 88%, 89%, 90%, 91%, 92%, 93%, 94%, 95%, 96%, 97%, 98%, or 99% identity thereto.

Embodiment 100. An isolated nucleic acid comprising an Activin-A and/or Activin-Bivariable light (VL) sequence chosen from a VL DNA sequence provided in Table 1, or a sequence with at least 85%, 86%, 87%, 88%, 89%, 90%, 91%, 92%, 93%, 94%, 95%, 96%, 97%, 98%, or 99% identity thereto.

Embodiment 101. The isolated nucleic acid of embodiment 100, wherein the VL sequence comprises the nucleic acid sequence of SEQ ID NO: 10, SEQ ID NO: 18, or a sequence with at least 85%, 86%, 87%, 88%, 89%, 90%, 91%, 92%, 93%, 94%, 95%, 96%, 97%, 98%, or 99% identity thereto.

Embodiment 102. An isolated nucleic acid comprising an Activin A/B antibody agent sequence, wherein the nucleic acid comprises:
(a) a variable heavy (VH) sequence chosen from: SEQ ID NO: 29, SEQ ID NO: 34, SEQ ID NO: 41, or a sequence with at least 85%, 86%, 87%, 88%, 89%, 90%, 91%, 92%, 93%, 94%, 95%, 96%, 97%, 98%, or 99% identity thereto; and
(b) a variable light (VL) sequence chosen from: SEQ ID NO: 10, SEQ ID NO: 18, or a sequence with at least 85%, 86%, 87%, 88%, 89%, 90%, 91%, 92%, 93%, 94%, 95%, 96%, 97%, 98%, or 99% identity thereto.

Embodiment 103. The isolated nucleic acid of embodiment 102, wherein the Activin A/B antibody agent further comprises a light chain constant region (CL) and/or at least one heavy chain constant region.

Embodiment 104. A vector comprising the nucleic acid of any one of embodiments 97-103.

Embodiment 105. A host cell comprising the vector of embodiment 104.

Embodiment 106. The host cell of embodiment 105, wherein said host cell is a yeast cell, a bacterial cell, a mammalian cell or an insect cell.

Embodiment 107. The host cell of embodiment 108, wherein the mammalian cell is chosen from a CHO cell, a COS cell, a HEK-293 cell, an NSO cell, a PER.C6 cell, or an Sp2.0 cell.

Embodiment 108. A method of making an antibody agent which binds to Activin-A, Activin-B, and/or to one or more complexes that include one or both Activin-A and Activin-B, comprising culturing the host cell of any one of embodiments 105-107 under a condition wherein the Activin A/B antibody agent is expressed by said host cell.

Embodiment 109. A composition comprising an Activin A/B antibody agent polypeptide of any one of embodiments 1-96.

Embodiment 110. A pharmaceutical composition comprising an Activin A/B antibody agent polypeptide of embodiment 109.

Embodiment 111. The pharmaceutical composition of embodiment 110, comprising an excipient and/or a pharmaceutically acceptable carrier.

Embodiment 112. The pharmaceutical composition of embodiment 110 or 111, formulated in one or more unit dosage forms.

Embodiment 113. A method comprising
contacting a pharmaceutical composition of any one of embodiments 110-112 to a cell, tissue or subject.

Embodiment 114. The method of embodiment 113, wherein contacting comprises administering the Activin-A and/or Activin-B pharmaceutical composition to the cell, tissue or subject.

Embodiment 115. The method of embodiment 113 or 114, wherein the method is a treatment method.

Embodiment 116. The method of any one of embodiments 113-115, wherein the subject has a condition or disorder associated with increased Activin-A and/or Activin-B.

Embodiment 117. The method of embodiment 116, wherein Activin-A and/or Activin-B comprises free and/or active Activin-A and/or Activin-B.

Embodiment 118. The method of embodiment 117, wherein Activin-A and/or Activin-B is or comprises free Activin-A and/or Activin-B.

Embodiment 119. The method of embodiment 117, wherein Activin-A and/or Activin-B is or comprises active Activin-A and/or Activin-B.

Embodiment 120. The method of embodiment 117, wherein Activin-A and/or Activin-B is or comprises free and active Activin-A and/or Activin-B.

Embodiment 121. The method of any one of embodiments 116-120, wherein increased Activin-A and/or Activin-B comprises a level of about 500 pg/ml or more, e.g., as evaluated in a sample from a subject.

Embodiment 122. The method of embodiment 121, wherein the sample is or comprises blood, plasma, serum, urine, or a combination thereof.

Embodiment 123. The method of any one of embodiments 116-122, wherein the condition or disorder is chosen from (i) disorders of the hypothalamic pituitary gonadal axis (e.g., with increased FSH levels); (ii) fibrodysplasia ossificans progressive (FOP); (iii) pulmonary arterial hypertension (PAH) and/or hypertension (e.g., preeclampsia); (iv) Anorexia-Cachexia Disorders associated with chronic diseases such as cancer, chronic kidney disease, chronic heart failure, COPD, elderly and muscle immobility, HIV patients, ICU patients (e.g., loss of body weight, loss of food intake, fat mass loss, organ mass loss, muscle wasting (e.g., muscle atrophy), loss of functional muscle mass and strength, bone loss, anemia, fatigue, and/or organ damage); (v) anemia; (vi) metabolic disorders (e.g., obesity, type 2 diabetes, metabolic syndrome and/or type 1 diabetes, lipodystrophy); (vii) inflammatory disorders (e.g. inflammatory bowel disease, pancreatitis, allergic asthma, COVID, acute respiratory distress syndrome, atopic dermatitis, preeclampsia, acute liver injury, acute kidney injury, Tubulointerstitial nephritis, osteoarthritis and inflammatory-induced anemia); (viii) auto-immune disorders (e.g., SLE or rheumatoid arthritis); (ix) fibrotic diseases (e.g. idiopathic pulmonary fibrosis (IPF), NASH, liver cirrhosis, chronic kidney disease, viral hepatitis, biliary disorders, Alport Syndrome, Uterine Fibroids, Endometriosis); (x) cancer; (xi) cancer-treatment related toxicities (e.g., chemotherapy-induced nephrotoxicity, hepatotoxicity); (xii) cancer related metastases (e.g., changes in cell adhesion, migration and/or invasion); (xiii) aging (e.g., senescence); (xiv) Myalgic Encephalomyelitis or Chronic Fatigue Syndrome; (xv) reproductive disorders (e.g., with elevated FSH, hypogonadotropic hypothalamic amenorrhea, polycystic ovary syndrome (PCOS), menopause, ovarian aging and/or ovarian cancers); (xvi) chemoresistance; or (xvii) heart failure.

Embodiment 124. The method of any one of embodiments 113-123, wherein the method ameliorates a symptom of a disorder in a subject.

Embodiment 125. The method of embodiment 124, wherein the symptom is weight loss, loss of appetite, fatigue, muscle mass loss, fat mass loss, lean mass loss, lean mass atrophy, bone loss, anemia, organ damage and/or fibrosis.

Embodiment 126. The method of embodiment 125, wherein the organ damage comprises: a change in function of an organ, a reduction in size of an organ (e.g., atrophy), a change in cellularity of an organ (e.g., increased infiltration of immune cells), a reduction in weight of an organ, or any combination thereof.

Embodiment 127. The method of embodiment 125 or 126, wherein the organ damage comprises damage to one or more organs.

Embodiment 128. The method of any one of embodiments 125-127, wherein the organ damage comprises damage to one or more of: liver, heart, kidney and pancreas.

Embodiment 129. A method of inhibiting Activin-A and/or Activin-B, comprising:
contacting a cell, tissue or subject with an Activin-A and/or Activin-B pharmaceutical composition of any one of embodiments 110-112 to a cell, tissue or subject, thereby inhibiting Activin-A and/or Activin-B in the cell, tissue or subject.

Embodiment 130. The method of embodiment 129, wherein inhibition of Activin-A and/or Activin-B comprises:
(a) a reduction in activity, level, and/or stability of Activin-A and/or Activin-B; and/or
(b) preventing an increase in level of Activin-A and/or Activin-B.

Embodiment 131. The method of embodiment 130, wherein Activin-A and/or Activin-B comprises free and/or active Activin-A and/or Activin-B.

Embodiment 132. The method of embodiment 131, wherein Activin-A and/or Activin-B is or comprises free Activin-A and/or Activin-B.

Embodiment 133. The method of embodiment 131, wherein Activin-A and/or Activin-B is or comprises active Activin-A and/or Activin-B.

Embodiment 134. The method of embodiment 131, wherein Activin-A and/or Activin-B is or comprises free and active Activin-A and/or Activin-B.

Embodiment 135. The method of any one of embodiments 129-134, wherein contacting comprises administering the Activin-A and/or Activin-B pharmaceutical composition to the cell, tissue or subject.

Embodiment 136. The method of any one of embodiments 129-135, wherein reducing the level of Activin-A and/or Activin-B comprises reducing it to less than 500 pg/mL.

Embodiment 137. The method of any one of embodiments 130-135, wherein the level of Activin-A and/or Activin-B is reduced in a blood, plasma, serum, tumor, and/or urine sample.

Embodiment 138. The method of embodiment 130 or 131, wherein preventing an increase in the level of Activin-A and/or Activin-B comprises preventing an increase in Activin-A and/or Activin-B level above about 500 pg/mL.

Embodiment 139. The method of any one of embodiments 130-138, wherein inhibition of Activin-A and/or Activin-B is assessed relative to a comparator.

Embodiment 140. The method of embodiment 139, wherein the comparator comprises an otherwise similar cell, tissue or subject not administered an Activin-A and/or Activin-B pharmaceutical composition or administered an Activin-A and/or Activin-B inhibitor or administered a different Activin A/B antibody agent.

Embodiment 141. The method of any one of embodiments 129-140, wherein Activin-A and/or Activin-B is inhibited, e.g., the level of Activin-A and/or Activin-B is reduced, by at least 5%, 10%, 20%, 30%, 40%, 50%, 60%, 70%, 80%, 90%, 95% or 99%.

Embodiment 142. The method of any one of embodiments 129-141, wherein the activity of Activin-A and/or Activin-B comprises one or more, or all, or any combination of the following: (a) monomerization (b) homodimerization; (c) heterodimerization (d) binding to an Activin receptor; (e) modulation of an Activin receptor mediated signaling pathway.

Embodiment 143. The method of embodiment 142, wherein the Activin receptor signaling pathway comprises one or more of: (i) a SMAD pathway, (ii) an ERK/MAPK pathway, (iii) a p38 MAPK pathway, (iv) NOX-2 dependent signaling; (v) STAT3 signaling or (vi) Snail and/or Slug mediated pathways.

Embodiment 144. The method of embodiment 142, wherein administration of an Activin A/B antibody agent inhibits one or more Activin-A and/or Activin-B activities provided in (a)-(c).

Embodiment 145. A method of preventing and/or reversing mass loss in a subject, comprising administering to the subject an Activin-A and/or Activin-B pharmaceutical composition of any one of embodiments 110-112.

Embodiment 146. The method of embodiment 145, wherein mass comprises fat mass, lean mass, muscle mass, bone mass, organ mass, or a combination thereof.

Embodiment 147. The method of embodiment 146, wherein the Activin-A and/or Activin-B pharmaceutical composition prevents and/or reverses mass loss by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 148. The method of any one of embodiments 145-147, wherein the mass loss is or comprises fat mass loss.

Embodiment 149. The method of any one of embodiments 145-147, wherein the mass loss is or comprises lean mass loss.

Embodiment 150. The method of any one of embodiments 145-147, wherein the mass loss is or comprises organ mass loss.

Embodiment 151. The method of embodiment 150, wherein organ mass loss is in one or more organs chosen from: liver, kidney, pancreas, heart or combinations thereof.

Embodiment 152. The method of any one of embodiments 145-147, wherein the mass loss is or comprises muscle mass loss.

Embodiment 153. The method of any one of embodiments 145-147, wherein the mass loss is or comprises loss of functional muscle mass.

Embodiment 154. The method of any one of embodiments 145-147, wherein the mass loss is or comprises loss of muscle strength.

Embodiment 155. The method of any one of embodiments 152-154, wherein the muscle is chosen from: cardiac muscle, smooth muscle, skeletal muscle, or a combination thereof.

Embodiment 156. The method of embodiment 155, wherein the subject having muscle mass loss has a disease or disorder characterized by decreased muscle mass and/or strength.

Embodiment 157. The method of embodiment 156, wherein the disease or disorder characterized by decreased muscle mass and/or strength is chosen from: sarcopenia, cachexia, muscle injury, muscle wasting/atrophy, muscle immobility, bed-ridden patients, HIV patients ICU patients, cancer, obesity, diabetes, arthritis, multiple sclerosis, muscular dystrophy, amyotrophic lateral sclerosis, Parkinson's disease, osteoporosis, osteoarthritis, osteopenia, a metabolic syndrome, chronic renal failure, renal fibrosis, chronic heart failure, or chronic obstructive pulmonary disease.

Embodiment 158. The method of embodiment 157, wherein the metabolic syndrome comprises a disease or disorder chosen from: diabetes, obesity, nutritional disorders, organ atrophy, chronic obstructive pulmonary disease, type 1 diabetes, type-2 diabetes, pancreatitis, or anorexia.

Embodiment 159. The method of embodiment 158, wherein the mass loss is or comprises bone mass loss.

Embodiment 160. A method of preventing and/or reversing weight loss in a subject, comprising administering to the subject an Activin-A and/or Activin-B pharmaceutical composition of any one of embodiments 110-112.

Embodiment 161. The method of embodiment 160, wherein the weight loss is involuntary.

Embodiment 162. The method of embodiment 160 or 161, wherein the Activin-A and/or Activin-B pharmaceutical composition prevents and/or reverses weight loss by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 163. A method of preventing and/or reversing muscle atrophy in a subject, comprising administering to the subject an Activin-A and/or Activin-B pharmaceutical composition of any one of embodiments 110-112.

Embodiment 164. The method of embodiment 163, wherein the muscle is chosen from: cardiac muscle, smooth muscle, skeletal muscle, or a combination thereof.

Embodiment 165. The method of embodiment 163 or 164, wherein the Activin-A and/or Activin-B pharmaceutical composition prevents and/or reverses muscle atrophy by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 166. A method of preventing and/or reversing organ damage comprising administering to the subject an Activin-A and/or Activin-B pharmaceutical composition of any one of embodiments 110-112.

Embodiment 167. The method of embodiment 166, wherein organ damage comprises: a change in function of an organ, a reduction in size of an organ (e.g., atrophy), a change in cellularity of an organ (e.g., increased infiltration of immune cells), a reduction in weight of an organ, or any combination thereof.

Embodiment 168. The method of embodiment 166 or 167, wherein administration of the Activin-A and/or Activin-B pharmaceutical composition to the subject promotes regeneration of an organ with organ damage.

Embodiment 169. The method of any one of embodiments 166-168, wherein organ damage comprises damage in one or more organs.

Embodiment 170. The method of embodiment 169, wherein the one or more organs comprise liver, heart, kidney, pancreas, or combinations thereof.

Embodiment 171. A method of preventing and/or reversing senescence in a subject, comprising administering to the subject an Activin-A and/or Activin-B pharmaceutical composition of any one of embodiments 110-112.

Embodiment 172. The method of embodiment 171, wherein the Activin-A and/or Activin-B pharmaceutical composition prevents and/or reverses senescence by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 173. The method of any one of embodiments 145-172, wherein the mass loss, weight loss, muscle atrophy, fibrosis, anemia and/or senescence is a symptom of a disease or disorder.

Embodiment 174. The method of embodiment 173, wherein mass loss comprises loss of functional muscle loss, and/or loss of muscle strength.

Embodiment 175. The method of any one of embodiments 145-174, wherein the mass loss, weight loss, muscle atrophy, fibrosis, anemia and/or senescence is not a symptom of a disease or disorder.

Embodiment 176. A method of preventing and/or reversing liver damage in a subject, comprising administering to the subject an Activin-A and/or Activin-B pharmaceutical composition of any one of embodiments 110-112.

Embodiment 177. The method of embodiment 176, wherein liver damage comprises: acute liver injury, liver necrosis, liver fibrosis, liver inflammation, alteration in liver function, elevation one or more liver enzymes, changes in one or more biomarkers of liver function, or a combination thereof.

Embodiment 178. The method of embodiment 176 or 177, wherein:
(i) the one or more liver enzymes comprise ALT, AST or both;
(ii) the one or more biomarkers of liver function comprise albumin, CRP and/or bilirubin.

Embodiment 179. The method of any one of embodiments 176-178, wherein liver damage is a symptom of liver disease.

Embodiment 180. The method of embodiment 179, wherein liver disease is or comprises NAFLD, NASH, cirrhosis, hepatitis, hepatotoxicity and cancer.

Embodiment 181. The method of any one of embodiments 176-180, wherein the Activin-A and/or Activin-B pharmaceutical composition prevents and/or reverses liver damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 182. A method of preventing and/or reversing kidney damage in a subject, comprising administering to the subject an Activin-A and/or Activin-B pharmaceutical composition of any one of embodiments 110-112.

Embodiment 183. The method of embodiment 182, wherein kidney damage comprises: acute kidney injury, kidney fibrosis, kidney inflammation, alteration in kidney function, or a combination thereof.

Embodiment 184. The method of embodiment 182 or 183, wherein alteration in kidney function is or comprises proteinuria and/or low glomerular filtration rate (GFR).

Embodiment 185. The method of embodiment 182 or 183, wherein the kidney damage is or comprises tubular degeneration.

Embodiment 186. The method of any one of embodiments 182-185, wherein the kidney damage is a symptom of kidney disease.

Embodiment 187. The method of any one of embodiments 182-186, wherein kidney disease is or comprises: acute kidney injury, chemotherapy-induced nephropathy, cancer-treatment-induced nephropathy, chronic kidney disease, diabetic nephropathy, Alport Syndrome, chronic kidney disease mineral bone disorder, chronic kidney disease anemia, chronic kidney disease protein energy wasting, tubulointerstitial nephritis, Focal Segmental Glomerulosclerosis, IgA nephropathy, Autosomal dominant polycystic kidney disease.

Embodiment 188. The method of any one of embodiments 182-187, wherein the Activin-A and/or Activin-B pharmaceutical composition prevents and/or reverses kidney damage by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 100%.

Embodiment 189. The method of any one of embodiments 146-188, wherein the mass loss, weight loss, muscle atrophy, fibrosis, senescence, liver damage, and/or kidney damage is induced by a therapy for a disease or disorder (e.g., a SOC for a disease or disorder).

Embodiment 190. The method of any one of embodiments 146-189, wherein mass loss, weight loss, muscle atrophy, fibrosis, senescence, liver damage, and/or kidney damage is reduced relative to a comparator.

Embodiment 191. The method of embodiment 190, wherein the comparator comprises an otherwise similar cell, tissue or subject not administered an Activin-A and/or Activin-B pharmaceutical composition or administered an Activin-A and/or Activin-B inhibitor a different Activin A/B antibody agent.

Embodiment 192. A method of preventing and/or treating a SARS-COV-2 infection comprising administering to the subject an Activin-A and/or Activin-B pharmaceutical composition of any one of embodiments 110-112.

Embodiment 193. The method of embodiment 192, wherein the SARS-COV-2 infection is or comprises a COVID-19 disease.

Embodiment 194. The method of embodiment 192 or 193, wherein the SARS-COV-2 infection comprises acute respiratory distress syndrome (ARDS).

Embodiment 195. A method of preventing and/or treating cancer in a subject, comprising administering to the subject an Activin-A and/or Activin-B pharmaceutical composition of any one of embodiments 110-112.

Embodiment 196. The method of any one of embodiments 113-195, wherein the subject has an increased level and/or activity of Activin-A and/or Activin-B.

Embodiment 197. The method of any one of embodiments 113-196, further comprising determining a level and/or activity of Activin-A and/or Activin-B in a sample from the subject.

Embodiment 198. The method of embodiment 197, wherein an increased level and/or activity of Activin-A and/or Activin-B is determined relative to a comparator.

Embodiment 199. The method of embodiment 198, wherein a comparator comprises a predetermined reference sample such as a sample obtained from an otherwise similar subject who does not have a disease or disorder, or a symptom of a disease or disorder.

Embodiment 200. The method of any one of embodiments 113-199, wherein the Activin-A and/or Activin-B pharmaceutical composition is characterized in that when administered to the subject it reduces the level and/or activity of Activin-A and/or Activin-B relative to a comparator.

Embodiment 201. The method of embodiment 200, wherein the level Activin-A and/or Activin-B is reduced in a blood, plasma, serum, tumor or urine sample.

Embodiment 202. The method of embodiment 201, wherein Activin-A and/or Activin-B is or comprises free and/or active Activin-A and/or Activin-B.

Embodiment 203. The method of embodiment 202, wherein Activin-A and/or Activin-B is or comprises free Activin-A and/or Activin-B.

Embodiment 204. The method of embodiment 202, wherein Activin-A and/or Activin-B is or comprises active Activin-A and/or Activin-B.

Embodiment 205. The method of embodiment 202, wherein Activin-A and/or Activin-B is or comprises free and active Activin-A and/or Activin-B.

Embodiment 206. The method of any one of embodiments 200-205, wherein the Activin-A and/or Activin-B pharmaceutical reduces the level of Activin-A and/or Activin-B to less than 500 pg/mL.

Embodiment 207. A method of preventing and/or reducing cell adhesion, migration and invasion of cells in vivo, comprising: administering a pharmaceutical composition of any one of embodiments 110-112 to a subject.

Embodiment 208. The method of embodiment 207, wherein the subject has a condition or disorder associated with increased Activin-A and/or Activin-B.

Embodiment 209. The method of embodiment 208, wherein the condition or disorder is a hyperproliferative disorder, e.g., a cancer.

Embodiment 210. The method of any one of embodiments 207-209, wherein the method prevents and/or reduces cell adhesion, migration and invasion of cells from a primary cancer to one or more secondary sites.

Embodiment 211. The method of embodiment 210, wherein migration of cells to one or more secondary sites is or comprises metastasis.

Embodiment 212. The method of any one of embodiments 207-211, wherein the method prevents and/or reduces migration of cells by at least 5%, 10%, 20%, 30%, 40%, 50%, 60%, 70%, 80%, 90%, 95% or 99%.

Embodiment 213. The method of any one of embodiments 207-212, wherein prevention and/or reduction cell migration is assessed relative to a comparator.

Embodiment 214. The method of embodiment 213, wherein the comparator comprises an otherwise similar subject not administered an Activin-A and/or Activin-B pharmaceutical composition or administered an Activin-A and/or Activin-B inhibitor or administered a different Activin A/B antibody agent.

Embodiment 215. The method of any one of embodiments 208-214, wherein increased Activin-A and/or Activin-B comprises a level of about 500 pg/ml or more, e.g., as evaluated in a sample from a subject.

Embodiment 216. The method of embodiment 215, wherein the sample is or comprises blood, plasma, serum, urine, tumor, or a combination thereof.

Embodiment 217. The method of any one of embodiments 208-216, wherein Activin-A and/or Activin-B is or comprises free and/or active Activin-A and/or Activin-B.

Embodiment 218. A method of preventing and reducing metastasis, the method comprising the step of administering to a subject with a tumor a pharmaceutical composition of any one of embodiments 110-112.

Embodiment 219. The method of any one of embodiments 209-218, wherein the cancer is chosen from: colorectal cancer, colon cancer, gastric cancer, sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, lymphoma, multiple myeloma, renal cell carcinoma, bladder cancer, cervical cancer, choriocarcinoma, oral cancer, skin cancer, melanoma, endometrial cancer, myleofibrosis, bone cancer or a brain cancer.

Embodiment 220. The method of embodiment 219, wherein the cancer is colorectal cancer, pancreatic cancer, melanoma/skin and/or breast cancer.

Embodiment 221. The method of embodiment 219 or 220, wherein the method reduces metastasis by at least 5%, 10%, 20%, 30%, 40%, 50%, 60%, 70%, 80%, 90%, 95% or 99%.

Embodiment 222. The method of any one of embodiments 218-221, wherein reduction of metastasis is assessed relative to a comparator.

Embodiment 223. The method of embodiment 222, wherein the comparator comprises an otherwise similar subject not administered an Activin-A and/or Activin-B pharmaceutical composition or administered an Activin-A and/or Activin-B inhibitor or administered a different Activin A/B antibody agent.

Embodiment 224. The method of any one of embodiments 218-223, wherein the subject having cancer has increased Activin-A and/or Activin-B levels.

Embodiment 225. The method of any one of embodiments 218-224, wherein increased Activin-A and/or Activin-B comprises a level of about 500 pg/ml or more, e.g., as evaluated in a sample from the subject.

Embodiment 226. The method of embodiment 225, wherein the sample is or comprises blood, plasma, serum, tumor, urine, or a combination thereof.

Embodiment 227. The method of any one of embodiments 207-226, wherein Activin-A and/or Activin-B is or comprises free and/or active Activin-A and/or Activin-B.

Embodiment 228. A method, comprising
assessing a level and/or activity of Activin-A and/or Activin-B in a sample from a subject,
administering an Activin-A and/or Activin-B pharmaceutical composition to the subject if the level of Activin-A and/or Activin-B is higher than a comparator.

Embodiment 229. The method of embodiment 228, wherein an increased level of Activin-A and/or Activin-B comprises at least 500 pg/mL or more as assessed in a sample from a subject.

Embodiment 230. The method of embodiment 229, wherein an increased level and/or activity of Activin-A and/or Activin-B is determined relative to a comparator.

Embodiment 231. The method of embodiment 229, wherein a comparator comprises a predetermined reference sample such as a sample obtained from an otherwise similar subject who does not have a disease or disorder, or a symptom of a disease or disorder.

Embodiment 232. The method of any one of embodiments 228-231, wherein the Activin-A and/or Activin-B pharmaceutical composition is characterized in that when administered to the subject it reduces the level and/or activity of Activin-A and/or Activin-B relative to a comparator.

Embodiment 233. The method of embodiment 232, wherein the comparator comprises an otherwise similar cell, tissue or subject not administered an Activin-A and/or Activin-B pharmaceutical composition or administered an Activin-A and/or Activin-B inhibitor or a different Activin A/B antibody agent.

Embodiment 234. The method of any one of embodiments 113-233, wherein the subject is a mammal.

Embodiment 235. The method of embodiment 234, wherein the mammal is a human.

Embodiment 236. The method of embodiment 235, wherein the human is an adult.

Embodiment 237. The method of embodiment 235, wherein the human is a child.

Embodiment 238. The method of embodiment 234, wherein the mammal is a domestic animal.

Embodiment 239. The method of embodiment 238, wherein the domestic animal is chosen from: a cat, a dog, a ferret, a horse, a cow, a sheep, a pig, a Bactrian camel, or a yak.

Embodiment 240. The method of any one of embodiments 209-239, wherein the subject has a cancer having a TP53 mutation, a SMAD4 mutation, or both.

### EQUIVALENTS

Those skilled in the art will recognize, or be able to ascertain using no more than routine experimentation, many equivalents to the embodiments described herein. It is to be understood that the technologies disclosed herein encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment can be explicitly excluded from the claims, regardless of whether the specific exclusion is recited in the specification. The scope of the present invention is not intended to be limited to the above Description, but rather is as set forth in the claims that follow.

1. An antibody agent comprising a polypeptide that binds to Activin-A and Activin-B, or to one or more complexes that include both Activin-A and Activin-B, wherein the antibody agent comprises a light chain comprising a light chain complementarity determining region 1 (LC CDR1), an LC CDR2 and an LC CDR3 provided in Table 1 and a heavy chain comprising a heavy chain complementarity determining region 1 (HC CDR1), an HC CDR2 and an HC CDR3 provided in Table.

2. The antibody agent of claim 1, wherein the antibody agent is or comprises:
(i) an intact IgA, IgG, IgD, IgE or IgM antibody;
(ii) an antibody fragment;
(iii) a single domain antibody;
(iv) a single chain Fv; or
(v) a polypeptide comprising an antigen binding domain fused to an Fc domain.

3-10. (canceled)

11. The antibody agent of claim 1, wherein the antibody agent comprises:
(i) an LC CDR1 of SEQ ID NO: 1, an LC CDR2 of SEQ ID NO: 2; and an LC CDR3 of SEQ ID NO: 3; and a HC CDR1 of SEQ ID NO: 20; an HC CDR2 of SEQ ID NO: 21; and an HC CDR3 of SEQ ID NO: 22;
(ii) an LC CDR1 of SEQ ID NO: 1; an LC CDR2 of SEQ ID NO: 2; and an LC CDR3 of SEQ ID NO: 3; and a HC CDR1 of SEQ ID NO: 30; an HC CDR2 of SEQ ID NO: 31; and an HC CDR3 of SEQ ID NO: 22; or
(iii) an LC CDR1 of SEQ ID NO: 11; an LC CDR2 of SEQ ID NO: 12; and an LC CDR3 of SEQ ID NO: 13; and a HC CDR1 of SEQ ID NO: 35; an HC CDR2 of SEQ ID NO: 36; and an HC CDR3 of SEQ ID NO: 37.

12-14. (canceled)

15. The antibody agent of claim 1, wherein the antibody agent comprises: (a) a light chain and the light chain further comprises a sequence for a constant region (CL), (b) a heavy chain and the heavy chain comprises a variable region comprising an HC CDR1, an HC CDR2 and an HC CDR3 provided in Table 2 and a sequence for at least one constant heavy chain region (CH), wherein the at least one CH comprises an Fc domain, or (c) both (a) and (b).

16-20. (canceled)

21. The antibody agent of claim 1, wherein the antibody agent comprises:
(i) the sequence of SEQ ID NO: 8, or a sequence with at least 85% identity thereto, or a sequence having at least 5substitutions relative to SEQ ID NO: 8; and the sequence of SEQ ID NO: 27, or a sequence with at least 85% identity thereto, or a sequence having at least 5substitutions relative to SEQ ID NO: 27;
(ii) the sequence of SEQ ID NO: 8, or a sequence with at least 85% identity thereto, or a sequence having at least 5 substitutions relative to SEQ ID NO: 8; and the sequence of SEQ ID NO: 32, or a sequence with at least 85% identity thereto, or a sequence having at least 5 substitutions relative to SEQ ID NO: 32; or
(iii) the sequence of SEQ ID NO: 16, or a sequence with at least 85% identity thereto, or a sequence having at least 5 substitutions relative to SEQ ID NO: 16; and the sequence of SEQ ID NO: 39, or a sequence with at least 85% identity thereto, or a sequence having at least 5 substitutions relative to SEQ ID NO: 39.

22. (canceled)

23. The antibody agent of claim 1, wherein the antibody agent comprises:
(i) the sequence of SEQ ID NO: 9, or a sequence with at least 85% identity thereto, or a sequence having at least 5 substitutions relative to SEQ ID NO: 9; and the sequence of SEQ ID NO: 28, or a sequence with at least 85% identity thereto, or a sequence having at least 5 substitutions relative to SEQ ID NO: 28;
(ii) the sequence of SEQ ID NO: 9, or a sequence with at least 85% identity thereto, or a sequence having at least 5 substitutions relative to SEQ ID NO: 9; and the sequence of SEQ ID NO: 33, or a sequence with at least 85% identity thereto, or a sequence having at least 5 substitutions relative to SEQ ID NO: 33; or
(iii) the sequence of SEQ ID NO: 17, or a sequence with at least 85% identity thereto, or a sequence having at least 5 substitutions relative to SEQ ID NO: 17; and the sequence of SEQ ID NO: 40, or a sequence with at least 85% identity thereto, or a sequence having at least 5 substitutions relative to SEQ ID NO: 40.

24. The antibody agent of claim 1, wherein the antibody agent binds to (a) human Activin-A:
(i) with a binding affinity (K_{D}) of about 1.64 10 (−9) M to about 8.32 X 10 (−11) M; or
(ii) with a binding affinity (K_{D}) of about 5.45×10 (−10) M to about 6.61×10 (−10) M, or
(b) human Activin B with a binding affinity (K_{D}) of about 7.6 X 10 (−10) M to about 8.61 X 10 (−10) M.

25. (canceled)

26. The antibody agent of claim 1, characterized in that when tested in an assay that evaluates Activin-A or Activin-B activity or level, the antibody agent
(i) reduces Activin-A or Activin-B activity or level, or both, or
(ii) prevents an increase in Activin-A or Activin-B activity or level,
relative to a comparator, wherein the comparator comprises a sample that is not contacted with the antibody agent.

27. (canceled)

28. The antibody agent of claim 1, wherein the antibody agent:
(i) reduces a plasma, blood, serum, tumor or urine level of Activin-A or Activin-B to less than about 500 pg/mL;
(ii) prevents an increase in Activin-A or Activin-B levels in plasma, blood, serum, tumor, or urine above about 500 pg/mL; and/or
(iii) the antibody agent reduces an Activin-A and/or Activin-B activity.

29. The antibody agent of claim 28, wherein inhibition of Activin-A or Activin-B activity comprises inhibiting binding of Activin-A and/or Activin-B to:
(i) an Activin receptor;
(ii) a BMP receptor;
(iii) a TGF-beta receptor; or
(iv) any combination of (i)-(iii).

30. The antibody agent of claim 1, characterized in that when tested in an assay that evaluates activation of an Activin receptor, the antibody agent:
(i) does not activate one or more signaling pathways activated by the Activin receptor; or
(ii) reduces, e.g., inhibits, Activin A signaling via the Activin receptor,
wherein the antibody agent does not bind to or has minimal binding affinity for one or more TGFbeta super family members other than Activin-A or Activin-B.

31. (canceled)

32. The antibody agent of claim 1, which:
binds to GDF-11 or GDF-10 and is characterized in that when tested in an assay that evaluates GDF-11 or GDF-10 activity or level, the antibody agent does not modulate GDF-11 or GDF-10 activity or level.

33. (canceled)

34. The antibody agent of claim 1, wherein the antibody agent is produced in a mammalian cell.

35. (canceled)

36. An isolated nucleic acid comprising an Activin A/B antibody agent sequence, wherein the nucleic acid comprises:
(a) a variable heavy (VH) sequence chosen from: SEQ ID NO: 29, SEQ ID NO: 34, SEQ ID NO: 41, or a sequence with at least 85% identity thereto; and
(b) a variable light (VL) sequence chosen from: SEQ ID NO: 10, SEQ ID NO: 18, or a sequence with at least 85% identity thereto.

37. A vector comprising the nucleic acid of claim 36.

38. A host cell comprising the vector of claim 37.

39. A method of making an antibody agent which binds to Activin-A, Activin-B, and/or to one or more complexes that include one or both Activin-A and Activin-B, comprising culturing the host cell of claim 38, under a condition wherein the Activin A/B antibody agent is expressed by said host cell.

40. A composition comprising the antibody agent of claim 1.

41. A pharmaceutical composition comprising the antibody agent of claim 1, and an excipient.

42. A method comprising
delivering the pharmaceutical composition of claim 41 to a cell, tissue or subject.

43. The method of claim 42, wherein contacting comprises administering the pharmaceutical composition to the cell, tissue or subject, wherein the method is a treatment method.

44-50. (canceled)

51. A method of preventing or reversing:
(a) mass loss in a subject;
(b) weight loss in a subject;
(c) muscle atrophy in a subject;
(d) organ damage in a subject;
(e) senescence in a subject;
(f) liver damage in a subject;
(g) kidney damage in a subject; or
(h) one or more side effects of a therapeutic agent chosen from a GLP-1 receptor modulator, a glucagon receptor modulator, an agent that increases a level or activity of GIP, an agent that increases a level or activity of GLP-1, an agent that increases a level or activity of Glucagon, an agent that increases a level or activity of PYY, an agent that increases a level or activity of GDF15, or an agent that increases a level or activity of GFRAL,
wherein the method comprises administering to the subject an Activin-A and/or Activin-B pharmaceutical composition of claim 41.

52-62. (canceled)

63. A method comprising: (i) preventing or treating cancer in a subject, or (ii) preventing and reducing metastasis, wherein the method comprises administering to the subject the pharmaceutical composition of claim 41.

64-73. (canceled)

74. The method of claim 43, further comprising determining a level or activity of Activin-A or Activin-B, or both, in a sample from the subject, wherein an increased level or activity of Activin-A or Activin-B, or both, is determined relative to a comparator,
wherein the comparator comprises a predetermined reference sample such as a sample obtained from an otherwise similar subject who does not have a disease or disorder, or a symptom of a disease or disorder.

75-78. (canceled)

79. A method, comprising
assessing a level or activity of Activin-A or Activin-B, or both, in a sample from a subject, and
administering an Activin-A or Activin-B, or an Activin-A and Activin B pharmaceutical composition to the subject if the level of Activin-A or Activin-B, or both, is higher than a comparator.

80-81. (canceled)
